SEQUENCE LISTING (162 sequences)

SEQ ID NO: 1 - protein, 19 aa, synthetic construct
  REGION 1..19: note = Description of sequence: Linker
GGGGSGGGGSGGGSGGGGS

SEQ ID NO: 2 - protein, 15 aa, synthetic construct
  REGION 1..15: note = Description of sequence: Linker
GGGGSGGGGSGGGGS

SEQ ID NO: 3 - protein, 24 aa, synthetic construct
  REGION 1..24: note = Description of sequence: Linker
GGGGSGGGGSGGGGSGGGSGGGGS

SEQ ID NO: 4 - protein, 29 aa, synthetic construct
  REGION 1..29: note = Description of sequence: Linker
GGGGSGGGGSGGGGSGGGGSGGGSGGGGS

SEQ ID NO: 5 - protein, 5 aa, synthetic construct
  REGION 1..5: note = Description of sequence: Linker
GGGGS

SEQ ID NO: 6 - protein, 10 aa, synthetic construct
  REGION 1..10: note = Description of sequence: Linker
GGGGSGGGGS

SEQ ID NO: 7 - protein, 20 aa, synthetic construct
  REGION 1..20: note = Description of sequence: Signal sequence
MYRMQLLSCIALSLALVTNS

SEQ ID NO: 8 - protein, 20 aa, synthetic construct
  REGION 1..20: note = Description of sequence: Signal sequence
MYSMQLASCVTLTLVLLVNS

SEQ ID NO: 9 - protein, 20 aa, synthetic construct
  REGION 1..20: note = Description of sequence: Signal sequence
METPAQLLFLLLLWLPDTTG

SEQ ID NO: 10 - protein, 20 aa, synthetic construct
  REGION 1..20: note = Description of sequence: Signal sequence
METDTLLLWVLLLWVPGSTG

SEQ ID NO: 11 - protein, 21 aa, synthetic construct
  REGION 1..21: note = Description of sequence: Signal sequence
MALPVTALLLPLALLLHAARP

SEQ ID NO: 12 - protein, 18 aa, synthetic construct
  REGION 1..18: note = Description of sequence: Signal sequence
MALPVTALLLPLALLLHA

SEQ ID NO: 13 - protein, 16 aa, synthetic construct
  REGION 1..16: note = Description of sequence: Signal sequence
MKWVTFISLLFSSAYS

SEQ ID NO: 14 - protein, 30 aa, synthetic construct
  REGION 1..30: note = Description of sequence: Signal sequence
MDSKGSSQKGSRLLLLLVVSNLLLCQGVVS

SEQ ID NO: 15 - protein, 290 aa, synthetic construct
  REGION 1..290: note = Description of sequence: human PD-L1 component
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 16 - protein, 5 aa, synthetic construct
  REGION 1..5: note = Description of sequence: Antibody component
DSWIH

SEQ ID NO: 17 - protein, 17 aa, synthetic construct
  REGION 1..17: note = Description of sequence: Antibody component
WISPYGGSTYYADSVKG

SEQ ID NO: 18 - protein, 9 aa, synthetic construct
  REGION 1..9: note = Description of sequence: Antibody component
RHWPGGFDY

SEQ ID NO: 19 - protein, 6 aa, synthetic construct
  REGION 1..6: note = Description of sequence: Antibody component
QDVSTA

SEQ ID NO: 20 - skipped

SEQ ID NO: 21 - protein, 9 aa, synthetic construct
  REGION 1..9: note = Description of sequence: Antibody component
QQYLYHPAT

SEQ ID NO: 22 - protein, 118 aa, synthetic construct [sequence omitted]

SEQ ID NO: 23 - protein, 110 aa, synthetic construct
  REGION 1..110: note = Description of sequence: Antibody component
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTV

SEQ ID NO: 24 - protein, 243 aa, synthetic construct
  REGION 1..243: note = Description of sequence: Antibody component
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTV

SEQ ID NO: 25 - DNA, 729 bp, synthetic construct
  misc_feature 1..729: note = Description of sequence: Antibody component coding sequence
  source 1..729: mol_type = other DNA
gaggtgcagctggtggagtctgggggtggcctggtgcagcctgggggctcactgaggctttcctgcgccgcttctggctttacattcagtgacagctggattcactgggtgaggcaggctcctggaaagggtcttgagtgggttgcatggatttctccttacggtggtagcacttactacgctgacagcgtgaagggtcgattcacaatctccgcagacacctccaagaacacagcctacctgcagatgaacagcctacgggccgaggacaccgcagtctattactgtgcaagaagacactggcctggtggatttgactactggggtcaagggaccctggtcaccgtctcctcaggtggaggtggatcaggtggaggtggatctggtggaggtggatctgacattcagatgacccagtctccatcatccctgtccgcatcagtaggagacagggtcaccatcacctgccgggccagtcaggatgtgtcaactgctgtagcctggtatcaacagaaaccaggaaaggcccctaaactgctgatttactcggccagctttctgtacagtggagtccctagtcgcttcagcggcagtggatctgggacagatttcactctcaccatctcttccctgcagcctgaggacttcgcaacctattactgtcaacaatatctgtaccacccggccacgttcggacaggggaccaaggtggagatcaaacggaccgtg

SEQ ID NO: 26 - protein, 20 aa, synthetic construct
  REGION 1..20: note = Description of sequence: Linker
GGGGSGGGGSGGGGSGGGGS

SEQ ID NO: 27 - protein, 8 aa, synthetic construct
  REGION 1..8: note = Description of sequence: Antibody component
GKMSSRRC

SEQ ID NO: 28 - protein, 8 aa, synthetic construct
  REGION 1..8: note = Description of sequence: Antibody component
LLTTSGST

SEQ ID NO: 29 - protein, 21 aa, synthetic construct
  REGION 1..21: note = Description of sequence: Antibody component
AADSFEDPTCTLVTSSGAFQY

SEQ ID NO: 30 - protein, 128 aa, synthetic construct
  REGION 1..128: note = Description of sequence: Antibody component
QVQLQESGGGLVQPGGSLRLSCAASGKMSSRRCMAWFRQAPGKERERVAKLLTTSGSTYLADSVKGRFTISQNNAKSTVYLQMNSLKPEDTAMYYCAADSFEDPTCTLVTSSGAFQYWGQGTQVTVSS

SEQ ID NO: 31 - DNA, 384 bp, synthetic construct
  misc_feature 1..384: note = Description of sequence: Antibody component coding sequence
  source 1..384: mol_type = other DNA
caggtccaactgcaagagtctggtgggggtcttgtccaacctgggggctccctgaggctctcatgtgcagctagtggcaaaatgagcagtaggaggtgcatggcctggttccggcaggcacccggtaaggaaagagaacgagtagctaaacttctgaccacttcaggctcaacctacctcgctgacagtgtaaaaggacgattcaccatatcacaaaataatgctaaatccactgtgtacctccaaatgaatagtctgaagcctgaggacaccgccatgtattactgtgccgcagactcatttgaagatcccacctgcacactcgtgaccagcagcggggccttccaatactggggtcaaggcacccaagtgacagtctccagt

SEQ ID NO: 32 - protein, 288 aa, synthetic construct
  REGION 1..288: note = Description of sequence: CD80 polypeptide
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 33 - protein, 208 aa, synthetic construct [sequence omitted]

SEQ ID NO: 34 - DNA, 624 bp, synthetic construct
  misc_feature 1..624: note = Description of sequence: CD80 polypeptide coding sequence
  source 1..624: mol_type = other DNA
gttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataac

SEQ ID NO: 35 - protein, 183 aa, synthetic construct
  REGION 1..183: note = Description of sequence: OX-40L polypeptide
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 36 - protein, 193 aa, synthetic construct
  REGION 1..193: note = Description of sequence: CD70 polypeptide
MPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 37 - protein, 329 aa, synthetic construct
  REGION 1..329: note = Description of sequence: CD86 polypeptide
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 38 - protein, 177 aa, synthetic construct
  REGION 1..177: note = Description of sequence: GITRL polypeptide
MCLSHLENMPLSHSRTQGAQRSSWKLWLFCSIVMLLFLCSFSWLIFIFLQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 39 - protein, 302 aa, synthetic construct
  REGION 1..302: note = Description of sequence: ICOSL polypeptide
MRLGSPGLLFLLFSSLRADTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATWSILAVLCLLVVVAVAIGWVCRDRCLQHSYAGAWAVSPETELTGHV

SEQ ID NO: 40 - protein, 261 aa, synthetic construct
  REGION 1..261: note = Description of sequence: CD40L polypeptide
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 41 - protein, 255 aa, synthetic construct
  REGION 1..255: note = Description of sequence: 4-1BB polypeptide
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 42 - protein, 42 aa, synthetic construct
  REGION 1..42: note = Description of sequence: 4-1BB polypeptide
KRGRKKLLYIFKQPFMRPVQTTQEEDGCSCRFPEEEEGGCEL

SEQ ID NO: 43 - protein, 220 aa, synthetic construct
  REGION 1..220: note = Description of sequence: CD28 polypeptide
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 44 - protein, 40 aa, synthetic construct
  REGION 1..40: note = Description of sequence: CD28 polypeptide
RSKRSRLLHSDYMNMTPRRPGPTRKHYQPYAPPRDFAAYR

SEQ ID NO: 45 - protein, 277 aa, synthetic construct
  REGION 1..277: note = Description of sequence: OX40 polypeptide
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 46 - protein, 199 aa, synthetic construct
  REGION 1..199: note = Description of sequence: ICOS polypeptide
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 47 - protein, 93 aa, synthetic construct
  REGION 1..93: note = Description of sequence: DAP-10 polypeptide
MIHLGHILFLLLLPVAAAQTTPGERSSLPAFYPGTSGSCSGCGSLSLPLLAGLVAADAVASLLIVGAVFLCARPRRSPAQEDGKVYINMPGRG

SEQ ID NO: 48 - protein, 260 aa, synthetic construct
  REGION 1..260: note = Description of sequence: CD27 polypeptide
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCHYSCPREEEGSTIPIQEDYRKPEPACSP

SEQ ID NO: 49 - protein, 277 aa, synthetic construct
  REGION 1..277: note = Description of sequence: CD40 polypeptide
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 50 - protein, 351 aa, synthetic construct
  REGION 1..351: note = Description of sequence: CD2 polypeptide
MSFPCKFVASFLLIFNVSSKGAVSKEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 51 - protein, 216 aa, synthetic construct
  REGION 1..216: note = Description of sequence: NKG2D polypeptide
MGWIRGRRSRHSWEMSEFHNYNLDLKKSDFSTRWQKQRCPVVKSKCRENASPFFFCCFIAVAMGIRFIIMVTIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 52 - protein, 21 aa, synthetic construct
  REGION 1..21: note = Description of sequence: CD 80 transmembrane domain
LLPSWAITLISVNGIFVICCL

SEQ ID NO: 53 - protein, 457 aa, synthetic construct
  REGION 1..457: note = Description of sequence: CD80 polypeptide
MGHTRRQGTSPSKCPYLNFFQLLVLAGLSHFCSGQVQLQESGGGLVQPGGSLRLSCAASGKMSSRRCMAWFRQAPGKERERVAKLLTTSGSTYLADSVKGRFTISQNNAKSTVYLQMNSLKPEDTAMYYCAADSFEDPTCTLVTSSGAFQYWGQGTQVTVSSGGGGSGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFKRGRKKLLYIFKQPFMRPVQTTQEEDGCSCRFPEEEEGGCEL

SEQ ID NO: 54 - DNA, 1371 bp, synthetic construct
  misc_feature 1..1371: note = Description of sequence: CD80 polypeptide coding sequence
  source 1..1371: mol_type = other DNA
atgggacacacacggaggcaaggaacctccccatccaaatgtccttacttgaattttttccaactgcttgtactcgcaggactcagccacttctgtagcggacaggtccaactgcaagagtctggtgggggtcttgtccaacctgggggctccctgaggctctcatgtgcagctagtggcaaaatgagcagtaggaggtgcatggcctggttccggcaggcacccggtaaggaaagagaacgagtagctaaacttctgaccacttcaggctcaacctacctcgctgacagtgtaaaaggacgattcaccatatcacaaaataatgctaaatccactgtgtacctccaaatgaatagtctgaagcctgaggacaccgccatgtattactgtgccgcagactcatttgaagatcccacctgcacactcgtgaccagcagcggggccttccaatactggggtcaaggcacccaagtgacagtctccagtggaggagggggatcaggaggcggtggatctggtgggggtggttcaggaggtggcggatctgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttaaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 55 - protein, 556 aa, synthetic construct
  REGION 1..556: note = Description of sequence: CD80 polypeptide
MALPVTALLLPLALLLHAEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVGGGGSGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFKRGRKKLLYIFKQPFMRPVQTTQEEDGCSCRFPEEEEGGCEL

SEQ ID NO: 56 - DNA, 1668 bp, synthetic construct
  misc_feature 1..1668: note = Description of sequence: CD80 polypeptide coding sequence
  source 1..1668: mol_type = other DNA
atggctctcccagtgactgccctactgcttcccctagcgcttctcctgcatgcagaggtgcagctggtggagtctgggggtggcctggtgcagcctgggggctcactgaggctttcctgcgccgcttctggctttacattcagtgacagctggattcactgggtgaggcaggctcctggaaagggtcttgagtgggttgcatggatttctccttacggtggtagcacttactacgctgacagcgtgaagggtcgattcacaatctccgcagacacctccaagaacacagcctacctgcagatgaacagcctacgggccgaggacaccgcagtctattactgtgcaagaagacactggcctggtggatttgactactggggtcaagggaccctggtcaccgtctcctcaggtggaggtggatcaggtggaggtggatctggtggaggtggatctgacattcagatgacccagtctccatcatccctgtccgcatcagtaggagacagggtcaccatcacctgccgggccagtcaggatgtgtcaactgctgtagcctggtatcaacagaaaccaggaaaggcccctaaactgctgatttactcggccagctttctgtacagtggagtccctagtcgcttcagcggcagtggatctgggacagatttcactctcaccatctcttccctgcagcctgaggacttcgcaacctattactgtcaacaatatctgtaccacccggccacgttcggacaggggaccaaggtggagatcaaacggaccgtgggaggcgggggtagcggaggcggcggatctggtgggggtggttcaggaggtggcggatctgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttaaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 57 - protein, 6 aa, synthetic construct
  REGION 1..6: note = Description of sequence: Antibody component
GYAFSS

SEQ ID NO: 58 - protein, 6 aa, synthetic construct
  REGION 1..6: note = Description of sequence: Antibody component
YPGDGD

SEQ ID NO: 59 - protein, 9 aa, synthetic construct
  REGION 1..9: note = Description of sequence: Antibody component
KTISSVVDF

SEQ ID NO: 60 - protein, 11 aa, synthetic construct
  REGION 1..11: note = Description of sequence: Antibody component
KASQNVGTNVA

SEQ ID NO: 61 - protein, 6 aa, synthetic construct
  REGION 1..6: note = Description of sequence: Antibody component
SATYRN

SEQ ID NO: 62 - protein, 9 aa, synthetic construct
  REGION 1..9: note = Description of sequence: Antibody component
QQYNRYPYT

SEQ ID NO: 63 - protein, 122 aa, synthetic construct
  REGION 1..122: note = Description of sequence: Antibody component
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSS

SEQ ID NO: 64 - protein, 120 aa, synthetic construct
  REGION 1..120: note = Description of sequence: Antibody component
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTV

SEQ ID NO: 65 - protein, 108 aa, synthetic construct
  REGION 1..108: note = Description of sequence: Antibody component
DIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 66 - protein, 106 aa, synthetic construct
  REGION 1..106: note = Description of sequence: Antibody component
DIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEI

SEQ ID NO: 67 - protein, 263 aa, synthetic construct
  REGION 1..263: note = Description of sequence: scFv
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 68 - protein, 164 aa, synthetic construct
  REGION 1..164: note = Description of sequence: CD3 Zeta
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 69 - protein, 112 aa, synthetic construct
  REGION 1..112: note = Description of sequence: CD3 Zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 70 - protein, 22 aa, synthetic construct
  REGION 1..22: note = Description of sequence: CD3 Zeta ITAM1
QNQLYNELNLGRREEYDVLDKR

SEQ ID NO: 71 - DNA, 66 bp, synthetic construct
  misc_feature 1..66: note = Description of sequence: CD3 Zeta ITAM1 coding sequence
  source 1..66: mol_type = other DNA
cagaaccagctctataacgagctcaatctaggacgaagagaggagtacgatgttttggacaagaga

SEQ ID NO: 72 - protein, 22 aa, synthetic construct
  REGION 1..22: note = Description of sequence: CD3 Zeta ITAM1
QNQLFNELNLGRREEFDVLDKR

SEQ ID NO: 73 - DNA, 66 bp, synthetic construct
  misc_feature 1..66: note = Description of sequence: CD3 Zeta ITAM1 coding sequence
  source 1..66: mol_type = other DNA
cagaaccagctctttaacgagctcaatctaggacgaagagaggagttcgatgttttggacaagaga

SEQ ID NO: 74 - protein, 23 aa, synthetic construct
  REGION 1..23: note = Description of sequence: CD3 Zeta ITAM2
QEGLYNELQKDKMAEAYSEIGMK

SEQ ID NO: 75 - DNA, 69 bp, synthetic construct
  misc_feature 1..69: note = Description of sequence: CD3 Zeta ITAM2 coding sequence
  source 1..69: mol_type = other DNA
caggaaggcctgtacaatgaactgcagaaagataagatggcggaggcctacagtgagattgggatgaaa

SEQ ID NO: 76 - protein, 23 aa, synthetic construct
  REGION 1..23: note = Description of sequence: CD3 Zeta ITAM2
QEGLFNELQKDKMAEAFSEIGMK

SEQ ID NO: 77 - DNA, 69 bp, synthetic construct
  misc_feature 1..69: note = Description of sequence: CD3 Zeta ITAM2 coding sequence
  source 1..69: mol_type = other DNA
caggaaggcctgttcaatgaactgcagaaagataagatggcggaggccttcagtgagattgggatgaaa

SEQ ID NO: 78 - protein, 22 aa, synthetic construct
  REGION 1..22: note = Description of sequence: CD3 Zeta ITAM3
HDGLYQGLSTATKDTYDALHMQ

SEQ ID NO: 79 - DNA, 66 bp, synthetic construct
  misc_feature 1..66: note = Description of sequence: CD3 Zeta ITAM3 coding sequence
  source 1..66: mol_type = other DNA
cacgatggcctttaccagggtctcagtacagccaccaaggacacctacgacgcccttcacatgcag

SEQ ID NO: 80 - protein, 22 aa, synthetic construct
  REGION 1..22: note = Description of sequence: CD3 Zeta ITAM3
HDGLFQGLSTATKDTFDALHMQ

SEQ ID NO: 81 - DNA, 66 bp, synthetic construct
  misc_feature 1..66: note = Description of sequence: CD3 Zeta ITAM3 coding sequence
  source 1..66: mol_type = other DNA
cacgatggccttttccaggggctcagtacagccaccaaggacaccttcgacgcccttcacatgcag

SEQ ID NO: 82 - protein, 112 aa, synthetic construct
  REGION 1..112: note = Description of sequence: modified CD3 Zeta polypeptide
RVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 83 - DNA, 336 bp, synthetic construct
  misc_feature 1..336: note = Description of sequence: modified CD3 Zeta polypeptide coding sequence
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccaggggctcagtacagccaccaaggacaccttcgacgcccttcacatgcaggccctgccccctcgc

SEQ ID NO: 84 - DNA, 123 bp, synthetic construct
  misc_feature 1..123: note = Description of sequence: CD28 polypeptide coding sequence
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 85 - protein, 218 aa, synthetic construct
  REGION 1..218: note = Description of sequence: CD28 polypeptide
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 86 - DNA, 657 bp, synthetic construct
  misc_feature 1..657: note = Description of sequence: CAR nucleotide sequence
  source 1..657: mol_type = other DNA
attgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 87 - DNA, 657 bp, synthetic construct
  misc_feature 1..657: note = Description of sequence: CAR nucleotide sequence
  source 1..657: mol_type = other DNA
attgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgc

SEQ ID NO: 88 - DNA, 657 bp, synthetic construct; identical to SEQ ID NO: 87
  misc_feature 1..657: note = Description of sequence: CAR nucleotide sequence
  source 1..657: mol_type = other DNA

SEQ ID NO: 89 - DNA, 675 bp, synthetic construct
  misc_feature 1..675: note = Description of sequence: CAR nucleotide sequence
  source 1..675: mol_type = other DNA
cccaccacgacgccagcgccgcgaccaccaaccccggcgcccacgatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaacaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 90 - DNA, 675 bp, synthetic construct
  misc_feature 1..675: note = Description of sequence: CAR nucleotide sequence
  source 1..675: mol_type = other DNA
cccaccacgacgccagcgccgcgaccaccaaccccggcgcccacgatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaacaaacggggcagaaagaagctcctgtatatattcaaacaaccatttatgagaccagtacaaactactcaagaggaagatggctgtagctgccgatttccagaagaagaagaaggaggatgtgaactgagagtgaagtttagccggagtgccgatgctccagcctatcagcaaggacaaaaccaactttacaacgaacttaatctcggtaggcgcgaggaatacgatgtgcttgataaacgccggggtcgcgatcccgaaatgggcgggaaacctcggcgcaagaatcctcaagaaggactctataatgagttgcagaaggacaagatggctgaggcatatagtgagatcggtatgaagggagaacggagaagggggaaagggcatgatggattgtaccaaggacttagcacagctactaaagatacatatgacgccctgcacatgcaagcattgcctccacgc

SEQ ID NO: 91 - protein, 140 aa, synthetic construct
  REGION 1..140: note = Description of sequence: TRAC polypeptide
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 92 - DNA, 420 bp, synthetic construct
  misc_feature 1..420: note = Description of sequence: TRAC polypeptide coding sequence
  source 1..420: mol_type = other DNA
atccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 93 - protein, 143 aa, synthetic construct
  REGION 1..143: note = Description of sequence: TRAC polypeptide
IPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 94 - DNA, 429 bp, synthetic construct
  misc_feature 1..429: note = Description of sequence: TRAC polypeptide coding sequence
  source 1..429: mol_type = other DNA
attcccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 95 - DNA, 4627 bp, synthetic construct
  misc_feature 1..4627: note = Description of sequence: TRAC polypeptide coding sequence
  source 1..4627: mol_type = other DNA
atatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacagtattctaaggacgccagaaagctgttgatcggcttcaagcaggggagggacacctaatttgcttttcttttttttttttttttttttttttttttttgagatggagttttgctcttgttgcccaggctggagtgcaatggtgcatcttggctcactgcaacctccgcctcccaggttcaagtgattctcctgcctcagcctcccgagtagctgagattacaggcacccgccaccatgcctggctaattttttgtatttttagtagagacagggtttcactatgttggccaggctggtctcgaactcctgacctcaggtgatccacccgcttcagcctcccaaagtgctgggattacaggcgtgagccaccacacccggcctgcttttcttaaagatcaatctgagtgctgtacggagagtgggttgtaagccaagagtagaagcagaaagggagcagttgcagcagagagatgatggaggcctgggcagggtggtggcagggaggtaaccaacaccattcaggtttcaaaggtagaaccatgcagggatgagaaagcaaagaggggatcaaggaaggcagctggattttggcctgagcagctgagtcaatgatagtgccgtttactaagaagaaaccaaggaaaaaatttggggtgcagggatcaaaactttttggaacatatgaaagtacgtgtttatactctttatggcccttgtcactatgtatgcctcgctgcctccattggactctagaatgaagccaggcaagagcagggtctatgtgtgatggcacatgtggccagggtcatgcaacatgtactttgtacaaacagtgtatattgagtaaatagaaatggtgtccaggagccgaggtatcggtcctgccagggccaggggctctccctagcaggtgctcatatgctgtaagttccctccagatctctccacaaggaggcatggaaaggctgtagttgttcacctgcccaagaactaggaggtctggggtgggagagtcagcctgctctggatgctgaaagaatgtctgtttttccttttagaaagttcctgtgatgtcaagctggtcgagaaaagctttgaaacaggtaagacaggggtctagcctgggtttgcacaggattgcggaagtgatgaacccgcaataaccctgcctggatgagggagtgggaagaaattagtagatgtgggaatgaatgatgaggaatggaaacagcggttcaagacctgcccagagctgggtggggtctctcctgaatccctctcaccatctctgactttccattctaagcactttgaggatgagtttctagcttcaatagaccaaggactctctcctaggcctctgtattcctttcaacagctccactgtcaagagagccagagagagcttctgggtggcccagctgtgaaatttctgagtcccttagggatagccctaaacgaaccagatcatcctgaggacagccaagaggttttgccttctttcaagacaagcaacagtactcacataggctgtgggcaatggtcctgtctctcaagaatcccctgccactcctcacacccaccctgggcccatattcatttccatttgagttgttcttattgagtcatccttcctgtggtagcggaactcactaaggggcccatctggacccgaggtattgtgatgataaattctgagcacctaccccatccccagaagggctcagaaataaaataagagccaagtctagtcggtgtttcctgtcttgaaacacaatactgttggccctggaagaatgcacagaatctgtttgtaaggggatatgcacagaagctgcaagggacaggaggtgcaggagctgcaggcctcccccacccagcctgctctgccttggggaaaaccgtgggtgtgtcctgcaggccatgcaggcctgggacatgcaagcccataaccgctgtggcctcttggttttacagatacgaacctaaactttcaaaacctgtcagtgattgggttccgaatcctcctcctgaaagtggccgggtttaatctgctcatgacgctgcggctgtggtccagctgaggtgaggggccttgaagctgggagtggggtttagggacgcgggtctctgggtgcatcctaagctctgagagcaaacctccctgcagggtcttgcttttaagtccaaagcctgagcccaccaaactctcctacttcttcctgttacaaattcctcttgtgcaataataatggcctgaaacgctgtaaaatatcctcatttcagccgcctcagttgcacttctcccctatgaggtaggaagaacagttgtttagaaacgaagaaactgaggccccacagctaatgagtggaggaagagagacacttgtgtacaccacatgccttgtgttgtacttctctcaccgtgtaacctcctcatgtcctctctccccagtacggctctcttagctcagtagaaagaagacattacactcatattacaccccaatcctggctagagtctccgcaccctcctcccccagggtccccagtcgtcttgctgacaactgcatcctgttccatcaccatcaaaaaaaaactccaggctgggtgcgggggctcacacctgtaatcccagcactttgggaggcagaggcaggaggagcacaggagctggagaccagcctgggcaacacagggagaccccgcctctacaaaaagtgaaaaaattaaccaggtgtggtgctgcacacctgtagtcccagctacttaagaggctgagatgggaggatcgcttgagccctggaatgttgaggctacaatgagctgtgattgcgtcactgcactccagcctggaagacaaagcaagatcctgtctcaaataataaaaaaaataagaactccagggtacatttgctcctagaactctaccacatagccccaaacagagccatcaccatcacatccctaacagtcctgggtcttcctcagtgtccagcctgacttctgttcttcctcattccagatctgcaagattgtaagacagcctgtgctccctcgctccttcctctgcattgcccctcttctccctctccaaacagagggaactctcctacccccaaggaggtgaaagctgctaccacctctgtgcccccccggcaatgccaccaactggatcctacccgaatttatgattaagattgctgaagagctgccaaacactgctgccaccccctctgttcccttattgctgcttgtcactgcctgacattcacggcagaggcaaggctgctgcagcctcccctggctgtgcacattccctcctgctccccagagactgcctccgccatcccacagatgatggatcttcagtgggttctcttgggctctaggtcctgcagaatgttgtgaggggtttatttttttttaatagtgttcataaagaaatacatagtattcttcttctcaagacgtggggggaaattatctcattatcgaggccctgctatgctgtgtatctgggcgtgttgtatgtcctgctgccgatgccttc

SEQ ID NO: 96 - protein, 178 aa, synthetic construct
  REGION 1..178: note = Description of sequence: TRBC2 polypeptide
DLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 97 - DNA, 534 bp, synthetic construct
  misc_feature 1..534: note = Description of sequence: TRBC2 polypeptide coding sequence
  source 1..534: mol_type = other DNA
gatctgaaaaacgtgttccctcctgaagtggctgtctttgaaccatccgaggccgagatttcccatacccagaaagcaactctggtctgtctggccactggattctaccccgatcacgtggaactgtcttggtgggtgaacggcaaggaagtccattccggagtctctaccgaccctcagcccctcaaggagcagcctgctctcaacgattctcggtactgcctgtcatctcgactgagagtgtctgccaccttctggcagaaccctagaaaccactttcggtgtcaggtccagttttacggcctgagcgagaacgatgagtggacacaggatagagccaaacctgtgacacagattgtgagcgccgaggcttggggacgagccgattgtggcttcacatccgagtcttaccagcagggagtgctgtctgctacaatcctctacgaaattctcctggggaaggccaccctgtacgctgtcctcgtgtctgctctggtgctcatggctatggtcaaacgaaaggactctagaggc

SEQ ID NO: 98 - protein, 180 aa, synthetic construct
  REGION 1..180: note = Description of sequence: TRBC2 polypeptide
LEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 99 - DNA, 540 bp, synthetic construct
  misc_feature 1..540: note = Description of sequence: TRBC2 polypeptide coding sequence
  source 1..540: mol_type = other DNA
ctggaggatctgaaaaacgtgttccctcctgaagtggctgtctttgaaccatccgaggccgagatttcccatacccagaaagcaactctggtctgtctggccactggattctaccccgatcacgtggaactgtcttggtgggtgaacggcaaggaagtccattccggagtctctaccgaccctcagcccctcaaggagcagcctgctctcaacgattctcggtactgcctgtcatctcgactgagagtgtctgccaccttctggcagaaccctagaaaccactttcggtgtcaggtccagttttacggcctgagcgagaacgatgagtggacacaggatagagccaaacctgtgacacagattgtgagcgccgaggcttggggacgagccgattgtggcttcacatccgagtcttaccagcagggagtgctgtctgctacaatcctctacgaaattctcctggggaaggccaccctgtacgctgtcctcgtgtctgctctggtgctcatggctatggtcaaacgaaaggactctagaggc

SEQ ID NO: 100 - protein, 175 aa, synthetic construct
  REGION 1..175: note = Description of sequence: TRBC1 polypeptide
LNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 101 - protein, 176 aa, synthetic construct
  REGION 1..176: note = Description of sequence: TRBC1 polypeptide
DLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 102 - DNA, 528 bp, synthetic construct
  misc_feature 1..528: note = Description of sequence: TRBC1 polypeptide coding sequence
  source 1..528: mol_type = other DNA
gacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 103 - DNA, 1448 bp, synthetic construct
  misc_feature 1..1448: note = Description of sequence: TRBC polypeptide coding sequence
  source 1..1448: mol_type = other DNA
aggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcaggtgagtggggcctggggagatgcctggaggagattaggtgagaccagctaccagggaaaatggaaagatccaggtagcagacaagactagatccaaaaagaaaggaaccagcgcacaccatgaaggagaattgggcacctgtggttcattcttctcccagattctcagcccaacagagccaagcagctgggtcccctttctatgtggcctgtgtaactctcatctgggtggtgccccccatccccctcagtgctgccacatgccatggattgcaaggacaatgtggctgacatctgcatggcagaagaaaggaggtgctgggctgtcagaggaagctggtctgggcctgggagtctgtgccaactgcaaatctgactttacttttaattgcctatgaaaataaggtctctcatttattttcctctccctgctttctttcagactgtggctttacctcgggtaagtaagcccttccttttcctctccctctctcatggttcttgacctagaaccaaggcatgaagaactcacagacactggagggtggagggtgggagagaccagagctacctgtgcacaggtacccacctgtccttcctccgtgccaacagtgtcctaccagcaaggggtcctgtctgccaccatcctctatgagatcctgctagggaaggccaccctgtatgctgtgctggtcagcgcccttgtgttgatggccatggtaagcaggagggcaggatggggccagcaggctggaggtgacacactgacaccaagcacccagaagtatagagtccctgccaggattggagctgggcagtagggagggaagagatttcattcaggtgcctcagaagataacttgcacctctgtaggatcacagtggaagggtcatgctgggaaggagaagctggagtcaccagaaaacccaatggatgttgtgatgagccttactatttgtgtggtcaatgggccctactactttctctcaatcctcacaactcctggctcttaataacccccaaaactttctcttctgcaggtcaagagaaaggatttctga

SEQ ID NO: 104 - DNA, 1489 bp, synthetic construct
  misc_feature 1..1489: note = Description of sequence: TRBC polypeptide coding sequence
  source 1..1489: mol_type = other DNA
aggacctgaaaaacgtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcaggtgagtggggcctggggagatgcctggaggagattaggtgagaccagctaccagggaaaatggaaagatccaggtagcggacaagactagatccagaagaaagccagagtggacaaggtgggatgatcaaggttcacagggtcagcaaagcacggtgtgcacttcccccaccaagaagcatagaggctgaatggagcacctcaagctcattcttccttcagatcctgacaccttagagctaagctttcaagtctccctgaggaccagccatacagctcagcatctgagtggtgtgcatcccattctcttctggggtcctggtttcctaagatcatagtgaccacttcgctggcactggagcagcatgagggagacagaaccagggctatcaaaggaggctgactttgtactatctgatatgcatgtgtttgtggcctgtgagtctgtgatgtaaggctcaatgtccttacaaagcagcattctctcatccatttttcttcccctgttttctttcagactgtggcttcacctccggtaagtgagtctctcctttttctctctatctttcgccgtctctgctctcgaaccagggcatggagaatccacggacacaggggcgtgagggaggccagagccacctgtgcacaggtgcctacatgctctgttcttgtcaacagagtcttaccagcaaggggtcctgtctgccaccatcctctatgagatcttgctagggaaggccaccttgtatgccgtgctggtcagtgccctcgtgctgatggccatggtaaggaggagggtgggatagggcagatgatgggggcaggggatggaacatcacacatgggcataaaggaatctcagagccagagcacagcctaatatatcctatcacctcaatgaaaccataatgaagccagactggggagaaaatgcagggaatatcacagaatgcatcatgggaggatggagacaaccagcgagccctactcaaattaggcctcagagcccgcctcccctgccctactcctgctgtgccatagcccctgaaaccctgaaaatgttctctcttccacaggtcaagagaaaggattccagaggctag

SEQ ID NO: 105 - protein, 173 aa, synthetic construct
  REGION 1..173: note = Description of sequence: TRGC1 polypeptide
DKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 106 - protein, 189 aa, synthetic construct
  REGION 1..189: note = Description of sequence: TRGC2 polypeptide
DKQLDADVSPKPTIFLPSIAETKLQKAGTYLCLLEKFFPDIIKIHWQEKKSNTILGSQEGNTMKTNDTYMKFSWLTVPEESLDKEHRCIVRHENNKNGIDQEIIFPPIKTDVTTVDPKYNYSKDANDVITMDPKDNWSKDANDTLLLQLTNTSAYYTYLLLLLKSVVYFAIITCCLLRRTAFCCNGEKS

SEQ ID NO: 107 - DNA, 5591 bp, synthetic construct
  misc_feature 1..5591: note = Description of sequence: TRGC polypeptide coding sequence
  source 1..5591: mol_type = other DNA
ataaacaacttgatgcagatgtttcccccaagcccactatttttcttccttcaattgctgaaacaaagctccagaaggctggaacatacctttgtcttcttgagaaatttttccctgatgttattaagatacattggcaagaaaagaagagcaacacgattctgggatcccaggaggggaacaccatgaagactaacgacacatacatgaaatttagctggttaacggtgccagaaaagtcactggacaaagaacacagatgtatcgtcagacatgagaataataaaaacggagttgatcaagaaattatctttcctccaataaagacaggtatgtgtttacgcatatcatctgtcagaacacttctttgaaagtgaatgctgcattttttcctttcagtattaatgaaaaacaaacataaatctttcttaaatattgttacatttaatggtagcataaatgccctgctacttttctatagaattaaaatggtataggttttggagaaaacaaaattgaaaaagttactgaaggtttgtcagcctcagctccattatccaaaataagaaagtcacgtgctggtttttagggttgttagatggattaaagaaacaacatacacagaagcatctagcaacgtgacacgtggtaaacgctcaaaaagtgttctcccttcttttgatgactttacttgatcaggaaataacatatatatgtctttcaggaatgttctgcccaagcaggagagtcactcacctcaatcttgctacccacaaagtttaacctaaaaacaacgggttcattgttgacaaaatgatgtttatctgttgttgacagaatgatgtttatctaaaaacagttccaattttctatttcctttgctgagacacaaaggggaggcaaatgtgcaaagcttgagggtagtcttaccactgtgcttaagtgttctgatttttctagtgatcagggcaaaataaaaagtatagtaagttccaaggcagtgaatattatacaggagagaagttacagttttataatgtgttttcctttacactaaattctaaaagtaaaaagtctttttttttttttgacagagtttcactcttgttgcccaagcaggtgtgctatggtatgatctcagctcactgcaacctccacctcccgggttcaagtgattctcttacttcagcctcccgacaggctgggattgcaggcgcctgccaccacacctggctaatttttgtgtttttagtagagatggggtttcaccatgttggccaggctggtctcaaattcctgacctcaagtgatccatccacctcggcctccaagtgctgggattatgggcgtcagccactgtgcccagcctaaaagtaaaatgtctttcatgagcttcccaaggcagctacgttaaggaggacacttctcttaatgtcattctacagtagatttctaatgctctttcttggaagtttgtttttctgagaaaagctaaaaatataacatggaagtgatcatattatataatcaatgaagtgcttttcaaggagataaaactaatctggtccacacttgcaaccaaccttgattgagagagagagagaactcaggatacacttgaagattttattatggggaacagttactttattctttttacctcaatcaatgcatggaaataagtgatagtcattttcatttatcttttaataaatgaagtcaccatgaggaaaataaaaagacattgaaaacccattaaagtcagcccttaaagatatttggacatgcagacttgataactaacgtttgcattcttgagacttacccaaaacccatacctcaagtccaagtttttagaattcatgaaataaagatctcagtgagtgcataaaattgcgcaccagaatcatatccgtatagacaagaacacatctactagaaaaataataaaccaacacaccaatgcaactgtgttttcttctgttttaaagtatgttgtctttgtatgcatgtttgcttcttcctttttttttttaacatcacagataaattcaactctcacctcaggttttattgagagaactgtcaatgtgacttggcctctgtctttctagtcccagaaagaattgcactgaaatctgagctcctgtaataaaaacaaccatttgctgagagtaattaacatactgaaagagattttcttagagtacacaatggtgacattatattgcctctttataaataactttctatctatttctgtggattattcctacaaagtacttttcatatgtccaatttcttttcttcccctacaactactgtctgaatactggctctgctatttgctgatatgattctcggcaagttgcctgcactttttaaactttatttcctcattcagaacatggggccatacataatacaactcacttcagtgttattggggaattaaacaaaaaatgcatgggaagcatttaacatagtgcctgacacaataatgagtactcagtagatgttagcttttattaatattgttgttgttatgtccagaaacactatacctccagaaaatcatgggtacttgctggggacattggggatatgcatgatttggaaaagaatgactgctttttttgcttagatgagaaatttttctaagccagactccttcaaatatgtaagattctgttgtggattcaaggactgaaagaattcttggccgagtgtggtggcttatccctgtaatcccagcattttgtgaggacaaggcaggaagattgcttgagtccaggagtttgaaaccagcctgcgcaacatggcgaaaccctgtctctacaaaaaatacaaacattagctcggagtgagtgctgacatgtgcctgtactcccagctactcagaaggctgagatgggaggatctcatgagcctggggagtttgaggcttcagtgagccgtgatgacaccgtactatactccactccagcctgggtgacagtgagaccctgcctcaaaaaacaaacaaacaaacaaacaaaacaaaattaatctttttgctgatgtcatgtcagcagtgtgtgttgaaggctgtaaagcagccatttgttcagtttatttttccattgaacaagtatttatcaaaaacatactttgtggcagtcactatgctaggagctatgaatacagaaggaaaagtaaatgctcttggatactacactccagttgtgataaaaaagaaaaaatgtattcttcaccaacttcaacatcttgatgtgcaaaaacataatacatgaattagatctacctaattacacagaattagaccaattgtttctggaattgtgggctcatatttttaataactgtcctcctgcctctctgtcgacaggttttataaatattcatttaattacacacacacacacgaacaattgactagtacttgctctcattcttctagatgtcatcacaatggatcccaaagacaattgttcaaaagatgcaaatggtaagcttttgtgtttttcccttcctcctgatcattttgttttgaacttctctggcttgaaaaatcagggaatggattttgctaggttggatgctgcagaatggacctagtgatattttaaattagtccctcattttctaggagttgtattaacaaacctaactactgctttggggtatgagatgactgtaaattagagagggtacagtggtatagtgatatgcttttaattatttcaaaaaaaagattttattcattcatgtgtcttttttctttttcttttcttttttttttttttttggacagagtcttgctctgtcacccaggctggagtgcggtggcagtatctcagctcaccacaacctccgcctcccggcttcaagtgattctcctgcctcagcttctcgagtagctgggactacaggcgcgtgccaccatgcccggctaatttttgtatttttagtagagttggggtttcaccatgttggccaggatggcctcgaatttgtgacctcgtgatctgccccctcgccctcccgaactgttgggattacaggcgtgagtcactgtgcccggcctcctgtcctgtcttttgtttaatgactgggaaaaacatgataccatgttgcttctcgagttgttttgttttagtctttggtctttgctagtagctaataacacgaactagtgtttatcaagtgctttttacacagaagggcttgggctgtgttctgcattttcttgtttaaccctcttaaaactcctataaaatggtacatatttttctcccaatttacagtccctttaaagcaaataattataaaaatccctatacatgtcacacagctagatctgggatttcaaatcaggccatcaaacaaagagtttatgtacttagtaagttttctgttctttttctacaatagagtcagatagcaagaaattaccaagccaggaacctgaaacaaaacggacatcatgtggggctgggtgggtgcatgggctttgcagactggactttcactccagctcttttaatgattaggtgtaagtgacctacattttgtgagcaacagttttctcatcagccaacaaagaataattacaccagattcacagttattgaagagataaaggcatgaatgtgagatgtctggcatagggcatctcatttagcagacacagaatgagtacttgtttctggctttttctctctacatatgcacaaagaatgcgactagaagcatgggctctagccctgctcaactttcctctatttccaataccaaggggctctgacttaggctgccacaccaggcaaggagggcagtaccacctcacttgaccaagggcagggagtcacggacacatcacttcttgagatccttttccacaccaaggactgatgtttctggaattctcactttatgaagacaaaacatataaatggaaattttctcaggtagagactcactcttgtagctcattgagtaggcactagtggtccacccccactgtctttacttattccttgacatcacatatctcttgcaaaacctcaaataatattaaatgcaatcacccaataatagcatagccataattagaggcatttaggaaagacaggtgagtgtgccacaactacctaacacatcagcaaatctggattaaccactttctttgattttccacaatgcaaccttactttttaatagttgggaatgttctaagtgaatttagcagaggttgttaatcaacttgaaagctgaattctgacttgtctgactcttggtggtgctggtagcagtagatgtttacttttaggttttggtggtggtggaatatcacttcaacgtaaatcatcagaaataagtatttgtgaacccctctcgcattaatgtatcttattctgtaaaaagaacatgtgcaatttctcttagatacactactgctgcagctcacaaacacctctgcatattacatgtacctcctcctgctcctcaagagtgtggtctattttgccatcatcacctgctgtctgcttagaagaacggctttctgctgcaatggagagaaatcataa

SEQ ID NO: 108 - DNA, 9549 bp, synthetic construct
  misc_feature 1..9549: note = Description of sequence: TRGC polypeptide coding sequence
  source 1..9549: mol_type = other DNA
ataaacaacttgatgcagatgtttcccccaagcccactatttttcttccttcgattgctgaaacaaaactccagaaggctggaacatacctttgtcttcttgagaaatttttcccagatattattaagatacattggcaagaaaagaagagcaacacgattctgggatcccaggaggggaacaccatgaagactaacgacacatacatgaaatttagctggttaacggtgccagaagagtcactggacaaagaacacagatgtatcgtcagacatgagaataataaaaacggaattgatcaagaaattatctttcctccaataaagacaggtatgtgtttacacatatcatctgtcagaacacttctttgaaagtgaatgctgcattttttcctttcagtattaatgaaaaacataaatctttcttaaaaattgttacatttaatggtagcgtaaatgccctgctacttttctatagaattaaaatggtataggttttggagaaaacaaaattgaaaaagttgctgaaggtttgtcagcctcagctccattatccaaaataagaaagtcacgtgctggtttttagggttgttagatggattaaagaaacaacatacacagaagcatctagcaacgtgacacgtggtaaacgctcaaaaagtgttctcccttcttttgatgactttacttgatcaggaaataacatatatatgtctttcaggaatgttctgcccaagcaggagagtcactcacctcaatcttgctacccacaaagtttaacctaaaaacaacgggttcattgttgacaaaataatgtttatctgaagataactgtagatcatatttatctgtagataatgtttatctgtggagtgtggctctacaaaacatagaatagtcttggtcactgcagttttatagaggccttgggtttttcagagtttcattttatatatcaccataaagtaacatttcataattacaggttggtaaggcttacatgtacaaacattcttccattttccataataaatgcatttcctgccattggtgaatgcagctcaataaacatttattgtacaattatgacacgccaggcttagtggaaatgtggatgaacagacaaggatgagttactgtcctaaggatgatgcatgacagtgcagagaatatactctcttcctgatcactcagggtcactcatgattcatgcgcgaggtcccaaaacagtgcctttgatgcagattctgtacatctctagacgattggtccaagggctgaatgtgctctggcccagtggtccagtctgtcactatatgtcaacatcctgaatatgaacataacagtccaacatctcaagagtgggcatgaaaaggactcattttgtgctttttcctgtggttaacaagtcctttttagcctgggggaacaagcattaacaaaatgtttgaagatctttgccacgtaccattccaaatttctagggtaagtctttagcttttcagatcctgagtttctgcaatgatcaaatgtgatttggacagttgcgttgactttctcctggggctataatggagtgcaaaggaaacaatggcagggaaaatgcttgctttcaaaatggtagcatggatgtgttcattcgtgtagttactgtattaggtatagcctttcctgaaactaactgaagtggggttataaaaacagtcccaattttctatttcctttgctgagacacaaagaggagacaaaagagcaaagcttgagggtagttttaccactgtgcttaagtgttctgatttttccagtgatcagggtgaaataaaaagcatagtaagttccagggcagtgaataccatacaggagacaagttacagttttataatgtgttttactttacactaaattctaaaagtaaaatgtctttttttttttccgagacagagtttcactcttgtagcccaggcaggagtgctatggtgtgatctcggctcacagcaacctccacctcccagtttcaagcgattcttctgcctcagcctcccgagaagttgaaattacaggtgcctggcaccatatctcgctaattattctatttttagtagagatcgggttttaccatgttggccaggctggtctcgaactcctgacttcaagtgatccacccgcctcagcctcccaaagtgctgggattacaggtgtgagtcactgtgccggacctaacagtaaaatgtctttcatgtgcttctcaaggcaactacattaaggaggacacatctcttaatgtcattctacagtagatttctaatgctctttcttggaagtttgtttttctgagaagagctaaaaatataataacatggaagtgatcatattatataatcaatgaagtgctttcaaaggagataaaactaacctggtctgcatttgcaaccagccttgattgagagagagagaactcaggatacacttagagattttattatggggaatagttactttattcattttacctcaatcaatgcatggaaataagtgacagtcattttcatttatcttttaataaataaagtcaccatgaggaaaatgaaaacccattaaagtcagtccttaaagatatttggacatgcagacatgataactaacatttccattcgtgagacttacccaaaacctatacctcaagtccatttcttagaatacatgaaataaagatctcagtgagtgtataaaactgcacaccagaatcatatccgtatagacaagaatacatctactagaaaaatataaaccaaaacaccaaggtgactctgtttttttctgttttaaaatatgttgtctttgtatgcatgtttgcttcttcctttttttttttaaacatcgcagataaattcaactctcacctcagttgagagagaactgtcaatgtgacttggcctctctctttctagtcccagaaagaattgcactgaaatgctgagctcctgtaataaaaatgaccatttgctgagagtaattaacatactgaaagagattttcttagaatagtgcacaatggcccaatggtgacattatattgtctctttataaattattttctatctatttctgtggattatttctacaaagcacttttcatatgtccaattccttttattcccctacaagtactgactgactactggctctgctgttcactgatatgactttcggcaagttgcctgcactttttaaacgttatttcctcattcagaacatggggccatacaaaatacaactcacttcagtgttattggggaattaaacaaataaatgcatgggaagcatttaacatagtgcctgacacaataatgagcactcagtagatgttagcttttattaatattgttgttgctatgtccagaaacactatacctccagaaaatcatgggtacttgctggggacgttggggatatgcatgattttgaaaggagtgactgctctttactgctcagatgagaaatttttctaagccagactccttcaaacatgtaagattctgttgtggattctaggactgaaagaattcttggccgagtgtggtggcttatcctggtaatctcatcatttgggaggacaaggcaggaagattgcttgagcccaggagttggaaacaagcctggacaacatggcgaaaccctgtctctacaaaaaatacaaacattagctggtcatgggagtgagtgcctgtactcccagctactcaggaggctaagataggaggatcacctgagcctgggcagtttgaggtttcagtgagccgtgatgacaccatactatactccactccagcctgggtgacagtgacatcctgcctcaaaaaaacccccaaaattattctttttgctgatttcatgtcagcagtgtgtgctgaaggctgtaaagtagccacttgttctgtttatttttccattgaacaagtatttatcaaaaacgtactttgtggaaggcactgtgctaggaactatgcatacagaaggaaaaccaaatgttcttggatactacactccagttgtgataaaaaagaaaaaagtattcttcacaaacttcaacattttgatgtgcaaaaacataatatatgaattagatctacctaactacacagaattagaccaattatttctgggattatgggctcatatttttaataactgtcctcctacctctctgttgacaggttttataaatattcatttaattacacacagtcacagacacactcagacacacacacatacacacacacacacaccttgacaaataatgggcatgaacaattgactggtacttgctctcattcttctagatgtcaccacagtggatcccaaatacaattattcaaaggatgcaaatggtaagtttttgtgttttttatttcctcctgatcattttaagttttgaacttctctggcttgaaaaatcagggaatggattttgctaggttggatgctgcagaatggacctaatcatattttaaattagtccctctttttctaggagttgtattaacaaacctaactactgcttcatgtaagagatgactgtaaattgaagggtacagtgatatgctttcagttatttcaaaaaacagactttactcatccatgtgtcttttttcttttcttttttttcttttttgagacggagtctcgctctgttgaacaggctggattgcagtgacgcgatctcacctcactacaacctccgcctctggagttcaagcgattctccagcctcagcttctcaagtagctgggactacaggcacatgccaccatgtccgggtcatctttgtatttttagcagagaccgggtttcactatgttggccaggctggtctagaattcctgacttcgtgatctgccccctcagccctccgaagtgctgggattacagacgtgagtcactgtgcccggcctaacagtaaaatgtctttcatgcgcttctcaaggcaactacgttaaggaggacacttctcttaatgtcattctacagtagatttctaatgctctttcttggaagtttgtttttctgagaaaagctaaaaatataacatggaagtgatcatattgtataatcaatgaagtgcttttcaaggagataaaactaatctggtccacgtttgcaaccaaccttgattgagagagagagagaactcaggatacacttggagattttattatggggaatagttactttattcttttttcctcaatcaattcatggaaataagtgatagtcatattcatttatcttttaataaatgaagtcaccatgaggaaaataaaaagacattgaaaacccattaaagttagcccttaaagatatttggacatgcagacttgataactaacgtttgcattcttgagacttacccaaaacccatacctcaagtccatgtttttagaattcatgaaataaagatctcagtgagtgcataaaattgcgcaccagaatcatatccgtatagacaagaacacatctactagaaaaataataaaccaacacaccaatgcaactgtgttttcttctgttttaaaatatgttgtctttgtatgcatgtttgcttcttccttttttttttttaacatcacagataaattcaactctcacctcaggttttattgagagaactgtcaatgtgacttggcctctgtctttctagtcccagaaagaatcgcactgaaatgctgagctcctgtaataaaaatgaccatttgctgagagtaattaacatactgaaagagattttcttagagtacacaatggtgacattatattgtctctttataaataactttctatctatttctgtggattattcctacaaagtacttttcatatgtccagtttcttttcttcccctacaactaccgtctgaatactggctctgctatttgctgatatgattctcggcaagttgcctgcactttttaaactttatttcctcattcagaacatggggccatgtaatactcatgtacgtgagtattacgtaataatgctcacttaagtgttactggggaattaaacaaaaaaatgcatggcaagcatttaacatagtgcctgacacaataatgagcactcagtagatgttagattttattaatattgttgttgttatgtccggaaacactatacctccagaaaatcatgggtacttgcttgggatgttggggatatgcatgatttggaaaggtatgactgcttttttctgcttagatgagaaatttttctaagccagactccttcaaatatgtaagattctgttgtggattctaggacggaaagaattcttggtcaggtgtggtttcttatccctgtaatcccagaattttgggaggacaaggcaggaagattgcttgagcccaggagtttgaaaccagcctgggcaacaagacgaaaccctgtctctacaaaagtacataaattagcttggcttggtggtgtgtgcctgtattaccagctattcgggagactgagatgggaggatctcctgaacctgtgaagtttgaggcttcagtgagccgtgatgacaccatactatactcgactccagcctgtgcgacagtgagactctgcgtcaaaaaaaaaaccccaaaattattgtttttgctgatttcaggtcagcagtgtgtgctgaagggtgtaaagtagccacttgatcagtttatttttccactgaacaagtatttatcaaaaacatactttgtggtctgtttttgataaataaaaaggcactgtgctaggagccatgaatacagaaggaaaaccaaatgttcttggatactacactccagttgtgataaaaaagaaaaatgtattcttcacgaacttcaacattttgatatgcaaaaacatagtatataaattagatctacctgattacgtagaatcagaccaattatttctggaattgagggctcatatttttaataactgtcctcctgcctctctgttgacaggttttataaatattcatttaattacacacacacacacacacaccttgacaaataatggacatgaacaattgactagtacttgctctcattcttctagatgtcatcacaatggatcccaaagacaattggtcaaaagatgcaaatggtaagcttttgtgtttttcctttcctcctgatcattttaagttttgaacttctctggcttgaaaaatcagggaatgggccgggtgcggtggctcacgcctgtaatcccagcactttgggaggccgaggcgggcggatcacgaggtcaggagatcgagaccatcccggctaaaacggtgaaaccccgtctctactaaaaatacaaaaaattagccgggcttagtggcgggcgcctgtagtcccagctacttgggaggctgaggcaggagaatggcgtgaacccgggaggcggagcttgcagtgagccgagattgcgccactgcactccactccagcctgggcgacagagcgagactccgtctcaaaaaaaaaaaaaaaaaaaaaaaaagaaaaatcagggaatggattttgctaggttggatgctgcagaatggacctagtgatattttaaattagtccctctttttctaggagttgtattaacaaacctaactactgcttcgggtatgagatgactgtaaattagagggtacagtgatatgctttcagttatttcaaaaaacagactttattcatccgtctgtcttttttttttttttttttttttttttttgagacggaggagtctcactctatcacccaggctggagtgcagtggcgcgatctcggctcaccataacctccgccttactggttcaagcgattctccagcctcagcttctcaagtagctgggactacaggtgcacaccaccatacctggctaatttttgtatttttaatagagatggggtttcaccacgctggccaggatggtcttgaattcttgacctcgtgatctgccccctcgggctcccaaacttctgggattataggcgtgagccactgtgcccggccttctgtcttttgttataatgactggggaaaacatgataccatgttgcttcttgagttgttttgttttagtctttggtctttgctagtagctaataacacgaactagtgtttatcaagtgctttttacacagaagggcttgttctgcattttctagtttaatcatcttaatactcctataaagtagtacaatatattttctcccattttacagtccctttaaagtaaataactataaaaatcccttatacatgtcacacagctaggtctggcatttcaaatcaggacatcaaacaaagaattcgtgcagttactaagtcctctattttttctacaatagaaaaaatagcaagaattacagatagcaagacattacaaggcaggaatctgaaacgaaagggacataatgtggggctgggtgggtgcatgagctttgcagactagactttcattccagctcttttaatgattaggtgtaagtgacctacattttgtgagtaacagttttctcatcagccaactaagaataattacaccagattcacagttattgaagagataagggcatgaatgtgagatgtctggcgtagggtatctcatttagcagacacagaatgaatacttgtttctggctttttctctctacatatgcacaaagaatgtgactagaagcattggctctagccctgctcaactttcctctatttccaataccaaggggctctgacttaggctgccacaccaggcaaggaggggcagtaccacctcacttgaccaagggcagggagtcacggacacatcacttcctgagatccttttccacaccaaggactgatgtttctggaattctcactttatgaagacaaaacatataaatggaaatttctgcaggaagagactcactcttgtagctcattgagtaggcactagtggtccacccccactgtctttacttattccttgacatcacatatctcttgtaaaacctcaaataatgttaaatgcaatcacccaataatagcatagccataattagaggcatttaggaaagacaggtgagtgtgccacaactacctaacacatcagcaaatctggattaaccactttctttgattttccacaatgcaaccttactttttaatagttgggaatgttctaagtgaatttagcagaggttgttaatcaacttgaaagctgaattctgacttgtctgactcttggtggtgctggtagcagtagatgtttacttttaggttttggtggtggtggaatatcacttcaacgtaaatcatcagaaataagtatttgtgaacccctctcgcattaatatatcttattctgtaaaaagaacatgtgcaatttctcttagatacactactgctgcagctcacaaacacctctgcatattacacgtacctcctcctgctcctcaagagtgtggtctattttgccatcatcacctgctgtctgcttagaagaacggctttctgctgcaatggagagaaatcataa

SEQ ID NO: 109 - protein, 153 aa, synthetic construct
  REGION 1..153: note = Description of sequence: TRDC polypeptide
SQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 110 - protein, 10 aa, synthetic construct
  REGION 1..10: note = Description of sequence: TRAJ region
IPNIQNPDPA

SEQ ID NO: 111 - DNA, 30 bp, synthetic construct
  misc_feature 1..30: note = Description of sequence: TRAJ region coding sequence
  source 1..30: mol_type = other DNA
attcccaatatccagaaccctgaccctgcc

SEQ ID NO: 112 - protein, 11 aa, synthetic construct
  REGION 1..11: note = Description of sequence: TRAJ region
LEDLKNVFPPE

SEQ ID NO: 113 - DNA, 33 bp, synthetic construct
  misc_feature 1..33: note = Description of sequence: TRAJ region coding sequence
  source 1..33: mol_type = other DNA
ctggaggatctgaaaaacgtgttccctcctgaa

SEQ ID NO: 114 - protein, 182 aa, synthetic construct
  REGION 1..182: note = Description of sequence: CD3 gamma chain
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 115 - protein, 171 aa, synthetic construct
  REGION 1..171: note = Description of sequence: CD3 delta chain
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 116 - protein, 127 aa, synthetic construct
  REGION 1..127: note = Description of sequence: CD3 delta chain
MEHSTFLSGLVLATLLSQVSPFKIPIEELEDRVFVNCNTSITWVEGTVGTLLSDITRLDLGKRILDPRGIYRCNGTDIYKDKESTVQVHYRTADTQALLRNDQVYQPLRDRDDAQYSHLGGNWARNK

SEQ ID NO: 117 - protein, 207 aa, synthetic construct
  REGION 1..207: note = Description of sequence: CD3 epsilon chain
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 118 - protein, 5 aa, synthetic construct
  REGION 1..5: note = Description of sequence: Antibody component
SYWMN

SEQ ID NO: 119 - protein, 17 aa, synthetic construct
  REGION 1..17: note = Description of sequence: Antibody component
QIYPGDGDTNYNGKFKG

SEQ ID NO: 120 - protein, 13 aa, synthetic construct
  REGION 1..13: note = Description of sequence: Antibody component
KTISSVVDFYFDY

SEQ ID NO: 121 - protein, 11 aa, synthetic construct
  REGION 1..11: note = Description of sequence: Antibody component
KASQNVGTNVA

SEQ ID NO: 122 - protein, 7 aa, synthetic construct
  REGION 1..7: note = Description of sequence: Antibody component
SATYRNS

SEQ ID NO: 123 - protein, 9 aa, synthetic construct
  REGION 1..9: note = Description of sequence: Antibody component
QQYNRYPYT

SEQ ID NO: 124 - protein, 138 aa, synthetic construct
  REGION 1..138: note = Description of sequence: Antibody component
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTV

SEQ ID NO: 125 - DNA, 414 bp, synthetic construct
  misc_feature 1..414: note = Description of sequence: Antibody component coding sequence
  source 1..414: mol_type = other DNA
atggctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtc

SEQ ID NO: 126 - protein, 124 aa, synthetic construct
  REGION 1..124: note = Description of sequence: Antibody component
MALPVTALLLPLALLLHADIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEI

SEQ ID NO: 127 - DNA, 372 bp, synthetic construct
  misc_feature 1..372: note = Description of sequence: Antibody component coding sequence
  source 1..372: mol_type = other DNA
atggcactgcccgtgaccgctctgcttctcccactagctctgcttctccacgcagacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatc

SEQ ID NO: 128 - DNA, 55 bp, synthetic construct
  misc_feature 1..55: note = Description of sequence: Signal sequence coding sequence
  source 1..55: mol_type = other DNA
atggctctcccagtgactgccctactgcttcccctagcgcttctcctgcatgcag

SEQ ID NO: 129 - skipped

SEQ ID NO: 130 - DNA, 63 bp, synthetic construct
  misc_feature 1..63: note = Description of sequence: CD 80 transmembrane domain coding sequence
  source 1..63: mol_type = other DNA
ctgctcccatcctgggccattaccttaatctcagtaaatggaatttttgtgatatgctgcctg

SEQ ID NO: 131 - DNA, 12 bp, synthetic construct
  misc_feature 1..12: note = Description of sequence: CD 80 fragment coding sequence
  source 1..12: mol_type = other DNA
acctactgcttt

SEQ ID NO: 132 - DNA, 129 bp, synthetic construct
  misc_feature 1..129: note = Description of sequence: 4-1BB polypeptide coding sequence
  source 1..129: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactgtga

SEQ ID NO: 133 - protein, 13 aa, synthetic construct
  REGION 1..13: note = Description of sequence: Antibody component
AASGFTFSSYIMH

SEQ ID NO: 134 - protein, 10 aa, synthetic construct
  REGION 1..10: note = Description of sequence: Antibody component
VISYDGRNKY

SEQ ID NO: 135 - protein, 11 aa, synthetic construct
  REGION 1..11: note = Description of sequence: Antibody component
ARDTDGYDFDY

SEQ ID NO: 136 - protein, 11 aa, synthetic construct
  REGION 1..11: note = Description of sequence: Antibody component
RASQSVSSYLA

SEQ ID NO: 137 - protein, 8 aa, synthetic construct
  REGION 1..8: note = Description of sequence: Antibody component
YDASNRAT

SEQ ID NO: 138 - protein, 9 aa, synthetic construct
  REGION 1..9: note = Description of sequence: Antibody component
QQRTNWPLT

SEQ ID NO: 139 - protein, 118 aa, synthetic construct
  REGION 1..118: note = Description of sequence: Antibody component
QVQLVESGGGVVQPGRSLRLSCAASGFTFSSYIMHWVRQAPGKGLEWVAVISYDGRNKYYADSVKGRFTISRDNSKNTLYLQMNSLRAEDTAVYYCARDTDGYDFDYWGQGTLVTVSS

SEQ ID NO: 140 - protein, 107 aa, synthetic construct
  REGION 1..107: note = Description of sequence: Antibody component
EIVLTQSPATLSLSPGERATLSCRASQSVSSYLAWYQQKPGQAPRLLIYDASNRATGIPARFSGSGSGTDFTLTISSLEPEDFAVYYCQQRTNWPLTFGGGTKVEIK

SEQ ID NO: 141 - protein, 240 aa, synthetic construct
  REGION 1..240: note = Description of sequence: Antibody component
QVQLVESGGGVVQPGRSLRLSCAASGFTFSSYIMHWVRQAPGKGLEWVAVISYDGRNKYYADSVKGRFTISRDNSKNTLYLQMNSLRAEDTAVYYCARDTDGYDFDYWGQGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRTNWPLTFGGGTKVEIK

SEQ ID NO: 142 - skipped

SEQ ID NO: 143 - protein, 4 aa, synthetic construct
  REGION 1..4: note = Description of sequence: STAT3 signaling domain
YRHQ

SEQ ID NO: 144 - protein, 4 aa, synthetic construct
  REGION 1..4: note = Description of sequence: STAT3 signaling domain
YLRQ

SEQ ID NO: 145 - protein, 4 aa, synthetic construct
  REGION 1..4: note = Description of sequence: STAT3 signaling domain
YHNQ

SEQ ID NO: 146 - protein, 4 aa, synthetic construct
  REGION 1..4: note = Description of sequence: STAT3 signaling domain
YFKQ

SEQ ID NO: 147 - protein, 4 aa, synthetic construct
  REGION 1..4: note = Description of sequence: STAT3 signaling domain
YLPQ

SEQ ID NO: 148 - protein, 4 aa, synthetic construct
  REGION 1..4: note = Description of sequence: STAT3 signaling domain
YMPQ

SEQ ID NO: 149 - protein, 4 aa, synthetic construct
  REGION 1..4: note = Description of sequence: STAT3 signaling domain
YVLQ

SEQ ID NO: 150 - protein, 4 aa, synthetic construct
  REGION 1..4: note = Description of sequence: STAT3 signaling domain
YQPQ

SEQ ID NO: 151 - protein, 4 aa, synthetic construct
  REGION 1..4: note = Description of sequence: STAT3 signaling domain
YKPQ

SEQ ID NO: 152 - protein, 4 aa, synthetic construct
  REGION 1..4: note = Description of sequence: STAT3 signaling domain
YRPQ

SEQ ID NO: 153 - protein, 4 aa, synthetic construct
  REGION 1..4: note = Description of sequence: STAT3 signaling domain
YTHQ

SEQ ID NO: 154 - protein, 4 aa, synthetic construct
  REGION 1..4: note = Description of sequence: STAT3 signaling domain
YLKQ

SEQ ID NO: 155 - skipped

SEQ ID NO: 156 - protein, 4 aa, synthetic construct
  REGION 1..4: note = Description of sequence: STAT5 signaling domain
YFFF

SEQ ID NO: 157 - protein, 4 aa, synthetic construct
  REGION 1..4: note = Description of sequence: STAT5 signaling domain
YCTF

SEQ ID NO: 158 - skipped

SEQ ID NO: 159 - protein, 262 aa, synthetic construct
  REGION 1..262: note = Description of sequence: LAT polypeptide
MEEAILVPCVLGLLLLPILAMLMALCVHCHRLPGSYDSTSSDSLYPRGIQFKRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELN

SEQ ID NO: 160 - protein, 509 aa, synthetic construct
  REGION 1..509: note = Description of sequence: LCK polypeptide
MGCGCSSHPEDDWMENIDVCENCHYPIVPLDGKGTLLIRNGSEVRDPLVTYEGSNPPASPLQDNLVIALHSYEPSHDGDLGFEKGEQLRILEQSGEWWKAQSLTTGQEGFIPFNFVAKANSLEPEPWFFKNLSRKDAERQLLAPGNTHGSFLIRESESTAGSFSLSVRDFDQNQGEVVKHYKIRNLDNGGFYISPRITFPGLHELVRHYTNASDGLCTRLSRPCQTQKPQKPWWEDEWEVPRETLKLVERLGAGQFGEVWMGYYNGHTKVAVKSLKQGSMSPDAFLAEANLMKQLQHQRLVRLYAVVTQEPIYIITEYMENGSLVDFLKTPSGIKLTINKLLDMAAQIAEGMAFIEERNYIHRDLRAANILVSDTLSCKIADFGLARLIEDNEYTAREGAKFPIKWTAPEAINYGTFTIKSDVWSFGILLTEIVTHGRIPYPGMTNPEVIQNLERGYRMVRPDNCPEELYQLMRLCWKERPEDRPTFDYLRSVLEDFFTATEGQYQPQP

SEQ ID NO: 161 - protein, 480 aa, synthetic construct [sequence omitted]

SEQ ID NO: 162 - protein, 533 aa, synthetic construct
  REGION 1..533: note = Description of sequence: SLP-76 polypeptide
MALRNVPFRSEVLGWDPDSLADYFKKLNYKDCEKAVKKYHIDGARFLNLTENDIQKFPKLRVPILSKLSQEINKNEERRSIFTRKPQVPRFPEETESHEEDNGGWSSFEEDDYESPNDDQDGEDDGDYESPNEEEEAPVEDDADYEPPPSNDEEALQNSILPAKPFPNSNSMYIDRPPSGKTPQQPPVPPQRPMAALPPPPAGRNHSPLPPPQTNHEEPSRSRNHKTAKLPAPSIDRSTKPPLDRSLAPFDREPFTLGKKPPFSDKPSIPAGRSLGEHLPKIQKPPLPPTTERHERSSPLPGKKPPVPKHGWGPDRRENDEDDVHQRPLPQPALLPMSSNTFPSRSTKPSPMNPLPSSHMPGAFSESNSSFPQSASLPPYFSQGPSNRPPIRAEGRNFPLPLPNKPRPPSPAEEENSLNEEWYVSYITRPEAEAALRKINQDGTFLVRDSSKKTTTNPYVLMVLYKDKVYNIQIRYQKESQVYLLGTGLRGKEDFLSVSDIIDYFRKMPLLLIDGKNRGSRYQCTLTHAAGYP

(19) United States
(12) Patent Application Publication
(10) Pub. No.: US 2026/0041715 A1
(43) Pub. Date: Feb. 12, 2026

(54) ENHANCED FUSION POLYPEPTIDE FOR IMMUNOTHERAPY

(71) Applicants: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(72) Inventors: Michel SADELAIN, New York, NY (US); Mohamad HAMIEH, New York, NY (US)

(73) Assignees: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(21) Appl. No.: 19/369,538

(22) Filed: Oct. 27, 2025

Related U.S. Application Data
(63) Continuation of application No. PCT/US24/26787, filed on Apr. 29, 2024.
(60) Provisional application No. 63/498,647, filed on Apr. 27, 2023.

(51) Int. Cl. A61K 35/17; A61K 40/11; A61K 40/31; A61K 40/42; A61P 35/00; C07K 14/705; C07K 16/28; C12N 5/0783; C12N 15/85

(52) U.S. Cl. CPC A61K 35/17; A61K 40/11; A61K 40/31; A61K 40/4211; A61P 35/00; C07K 14/70521; C07K 14/70532; C07K 14/70578; C07K 16/2827; C12N 5/0636; C12N 15/85; A61K 2239/13; A61K 2239/21; A61K 2239/29; C07K 2317/31; C07K 2317/565; C07K 2317/569; C07K 2317/622; C07K 2319/02

(57) ABSTRACT

The presently disclosed subject matter provides methods and compositions for enhancing immune responses toward tumor and pathogen antigens. It relates to fusion polypeptide that can be expressed in cells (e.g., immunoresponsive cells comprising an antigen-recognizing receptor) to improve the activity and/or efficiency of the cells. In certain embodiments, the fusion polypeptide comprises an extracellular domain comprising an antigen-binding fragment and a co-stimulatory ligand polypeptide, and an intracellular domain comprising a first co-stimulatory molecule polypeptide.

### CROSS-REFERENCE TO RELATED APPLICATIONS

This application is a continuation of International Patent Application No. PCT/US24/26787, filed Apr. 29, 2024, which claims priority to U.S. Provisional Application No. 63/498,647, filed Apr. 27, 2024, the contents of each of which are incorporated by reference in their entireties, and to each of which priority is claimed.

### SEQUENCE LISTING

A Sequence Listing conforming to the rules of WIPO Standard ST.26 is hereby incorporated by reference. Said Sequence Listing has been filed as an electronic document via Patent Center encoded as XML in UTF-8 text. The electronic document, created on Oct. 27, 2025, is entitled “0727341881SL.xml”, and is 249,005 in size.

### INTRODUCTION

The presently disclosed subject matter provides fusion polypeptides that can improve the activity and efficacy for immunotherapy.

### BACKGROUND OF THE INVENTION

Cell-based immunotherapy is a therapy with curative potential for the treatment of cancer. T cells and other immune cells may be modified to target tumor antigens through the introduction of genetic material coding for natural or modified T cell receptors (TCR) or synthetic receptors for antigen, termed Chimeric Antigen Receptors (CARs), specific to selected antigens. Patient-engineered CAR T cells have demonstrated remarkable efficacy against a range of liquid and solid malignancies. However, treatment failure and relapses occur in a large fraction of patients. Therefore, there remain needs of improved immunotherapy.

### SUMMARY OF THE INVENTION

The presently disclosed subject matter provides fusion polypeptides that can enhance the activity and efficacy of immunotherapy (e.g., T-cell immunotherapy). In certain embodiments, the fusion polypeptide comprises an extracellular domain comprising an antigen-binding fragment and a co-stimulatory ligand polypeptide, and an intracellular domain comprising a first co-stimulatory molecule polypeptide.

In certain embodiments, the antigen-binding fragment binds to an immune checkpoint molecule. In certain embodiments, the immune checkpoint molecule is selected from the group consisting of PD-L1, PD-L2, VISTA, B7-H3, B7-H4, B7-H7, herpesvirus entry mediator (HVEM), CD155, CD112, CD200, galectin 9 (GAL9), TIGIT, BTLA, LAG-3, TIM-3, and 2B4 (CD224).

In certain embodiments, the antigen-binding fragment binds to PD-L1. In certain embodiments, the antigen-binding fragment is a Fab, a Fab′, a F(ab′)2, a variable fragment (Fv), a single chain variable region (scFv), a nanobody, a microantibody, an affibody molecule, an affilin, an affimer, an affitin, an alphabody, an anticalin protein, an avimer, a DARPin (designed ankyrin repeat proteins), or an aptamer.

In certain embodiments, the antigen-binding fragment is a nanobody. In certain embodiments, the nanobody comprises a CDR1 comprising the amino acid sequence set forth in SEQ ID NO: 27 or a conservative modification thereof, a CDR2 comprising the amino acid sequence set forth in SEQ ID NO: 28 or a conservative modification thereof, and CDR3 comprising the amino acid sequence set forth in SEQ ID NO: 29 or a conservative modification thereof. In certain embodiments, the nanobody comprises a CDR1 comprising the amino acid sequence set forth in SEQ ID NO: 27, a CDR2 comprising the amino acid sequence set forth in SEQ ID NO: 28, and CDR3 comprising the amino acid sequence set forth in SEQ ID NO: 29. In certain embodiments, the nanobody comprises or consists of an amino acid sequence that is at least about 80% identical to the amino acid sequence set forth in SEQ ID NO: 30. In certain embodiments, the nanobody comprises the amino acid sequence set forth in SEQ ID NO: 30.

In certain embodiments, the antigen-binding fragment is an scFv. In certain embodiments, the scFv comprises a heavy chain variable region (VH) comprising a CDR1 comprising the amino acid sequence set forth in SEQ ID NO: 16 or a conservative modification thereof, a CDR2 comprising the amino acid sequence set forth in SEQ ID NO: 17 or a conservative modification thereof, and a CDR3 comprising the amino acid sequence set forth in SEQ ID NO: 18 or a conservative modification thereof, and a light chain variable region (VL) comprising a CDR1 comprising the amino acid sequence set forth in SEQ ID NO: 19 or a conservative modification thereof, a CDR2 comprising the amino acid sequence set forth in SEQ ID NO: 20 or a conservative modification thereof, and a CDR3 comprising the amino acid sequence set forth in SEQ ID NO: 21 or a conservative modification thereof. In certain embodiments, the VH comprises a CDR1 comprising the amino acid sequence set forth in SEQ ID NO: 16, a CDR2 comprising the amino acid sequence set forth in SEQ ID NO: 17, and a CDR3 comprising the amino acid sequence set forth in SEQ ID NO: 18; and the VL comprises a CDR1 comprising the amino acid sequence set forth in SEQ ID NO: 19, a CDR2 comprising the amino acid sequence set forth in SEQ ID NO: 20, and a CDR3 comprising the amino acid sequence set forth in SEQ ID NO: 21. In certain embodiments, the scFv comprises a heavy chain variable region (VH) that is at least about 80% identical to the amino acid sequence set forth in SEQ ID NO: 22, and a light chain variable region (VL) that is at least about 80% identical to the amino acid sequence set forth in SEQ ID NO: 23. In certain embodiments, the VH comprises the amino acid sequence set forth in SEQ ID NO: 22, and the VL comprises the amino acid sequence set forth in SEQ ID NO: 23.

In certain embodiments, the scFv comprises or consists of an amino acid sequence that is at least about 80% identical to the amino acid sequence set forth in SEQ ID NO: 24. In certain embodiments, the scFv comprises the amino acid sequence set forth in SEQ ID NO: 24. In certain embodiments, the co-stimulatory ligand polypeptide is selected from the group consisting of a tumor necrosis factor (TNF) family member, an immunoglobulin (Ig) superfamily member, and combinations thereof. In certain embodiments, the TNF family member is selected from the group consisting of 4-1BBL, OX40L, CD70, GITRL, CD40L, and combinations thereof. In certain embodiments, the Ig superfamily member is selected from the group consisting of CD80, CD86, ICOSLG, and combinations thereof.

In certain embodiments, the co-stimulatory ligand polypeptide is a CD80 polypeptide. In certain embodiments, the CD80 polypeptide comprises or consists of an amino acid sequence that is at least about 80% identical to the amino acid sequence set forth in SEQ ID NO: 32, or a functional fragment thereof. In certain embodiments, the CD80 polypeptide comprises or consists of an amino acid sequence that is at least about 80% identical to the amino acid sequence set forth in SEQ ID NO: 33. In certain embodiments, the extracellular domain of CD80 comprises the amino acid sequence set forth in SEQ ID NO: 33.

In certain embodiments, the first co-stimulatory molecule polypeptide is selected from the group consisting of CD28, 4-1BB, OX40, ICOS, DAP-10, CD27, CD40, NKG2D, CD2, and combinations thereof. In certain embodiments, the first co-stimulatory molecule polypeptide is a 4-1BB polypeptide. In certain embodiments, the 4-1BB polypeptide comprises or consists of an amino acid sequence that is at least about 80% identical to the amino acid sequence set forth in SEQ ID NO: 41, or a functional fragment thereof. In certain embodiments, the 4-1BB polypeptide comprises or consists of an amino acid sequence that is at least about 80% identical to the amino acid sequence set forth in SEQ ID NO: 42. In certain embodiments, the 4-1BB polypeptide comprises the amino acid sequence set forth in SEQ ID NO: 42.

In certain embodiments, the intracellular domain further comprises a second co-stimulatory molecule polypeptide. In certain embodiments, the second co-stimulatory molecule polypeptide is selected from the group consisting of CD28, 4-1BB, OX40, ICOS, DAP-10, CD27, CD40, NKG2D, CD2, and combinations thereof. In certain embodiments, the second co-stimulatory molecule polypeptide is a CD28 polypeptide. In certain embodiments, the CD28 polypeptide comprises or consists of an amino acid sequence that is at least about 80% identical to the amino acid sequence set forth in SEQ ID NO: 43, or a functional fragment thereof. In certain embodiments, the CD28 polypeptide comprises or consists of an amino acid sequence that is at least about 80% identical to the amino acid sequence set forth in SEQ ID NO: 44. In certain embodiments, the CD28 polypeptide comprises the amino acid sequence set forth in SEQ ID NO: 44.

In certain embodiments, the intracellular domain further comprises a spacer region. In certain embodiments, the spacer region comprises a CD80 polypeptide. In certain embodiments, the spacer region comprises a CD80 polypeptide comprising or consisting of amino acids 264 to 267 of SEQ ID NO: 32. In certain embodiments, the fusion polypeptide further comprises a transmembrane domain. In certain embodiments, the transmembrane domain comprises a CD80 polypeptide. In certain embodiments, the transmembrane domain comprises a CD80 polypeptide comprising or consisting of amino acids 243 to 263 of SEQ ID NO: 32. In certain embodiments, the transmembrane domain comprises a CD80 polypeptide comprising or consisting of SEQ ID NO: 52.

In certain embodiments, the antigen-binding fragment binds to PD-L1, the co-stimulatory ligand polypeptide is selected from the group consisting of 4-1BBL, OX40L, CD70, GITRL, CD40L, CD80, CD86, ICOSLG, and the first co-stimulatory molecule polypeptide is selected from the group consisting of CD28, 4-1BB, OX40, ICOS, DAP-10, CD27, CD40, NKG2D, and CD2. In certain embodiments, the antigen-binding fragment binds to PD-L1, the co-stimulatory ligand polypeptide is a CD80 polypeptide, and the first co-stimulatory molecule polypeptide is selected from the group consisting of CD28, 4-1BB, OX40, ICOS, DAP-10, CD27, CD40, NKG2D, and CD2.

In certain embodiments, the antigen-binding fragment binds to PD-L1, the co-stimulatory ligand polypeptide is a CD80 polypeptide, and the first co-stimulatory molecule polypeptide is a 4-1BB polypeptide. In certain embodiments, the fusion polypeptide comprises or consists of an amino acid sequence that is at least about 80% identical to the amino acid sequence set forth in SEQ ID NO: 53. In certain embodiments, the fusion polypeptide comprises the amino acid sequence set forth in SEQ ID NO: 53. In certain embodiments, the fusion polypeptide comprises or consists of an amino acid sequence that is at least about 80% identical to the amino acid sequence set forth in SEQ ID NO: 55. In certain embodiments, the fusion polypeptide comprises the amino acid sequence set forth in SEQ ID NO: 55.

In certain embodiments, the antigen-binding fragment binds to PD-L1, the co-stimulatory ligand is CD80, the first co-stimulatory molecule is 4-1BB, and the second co-stimulatory molecule is CD28.

In certain embodiments, the fusion polypeptide further comprises a signal peptide. In certain embodiments, the signal peptide is selected from the group consisting of a human IL-2 signal sequence, a mouse IL-2 signal sequence, a human kappa leader sequence, a mouse kappa leader sequence, a human CD8 leader sequence, a truncated human CD8 signal peptide, a human albumin signal sequence, and a human prolactin signal sequence. In certain embodiments, the fusion polypeptide further comprises a signaling domain of a cytokine receptor. In certain embodiments, the cytokine receptor is selected from the group consisting of CD121a, CDw121b, IL-18Ra, IL18Rb, CD122, CD25, CD132, CD124, CD213a13, CD127, IL-9R, IL15Ra, CDw125, CDw131, CD126, CD130, IL11Ra, Cd114, CD212, CD4, CDw217, CD118, and CDw119.

In certain embodiments, the fusion polypeptide further comprises a JAK-STAT signaling domain. In certain embodiments, the JAK-STAT signaling domain is a STAT3 signaling domain. In certain embodiments, the JAK-STAT signaling domain is a STAT5 signaling domain.

In certain embodiments, the fusion polypeptide further comprises a T cell signaling molecule. In certain embodiments, the fusion polypeptide is capable of stimulating a cell comprising an antigen-recognizing receptor. In certain embodiments, the fusion polypeptide is capable of enhancing the activity of an immunoresponsive cell comprising an antigen-recognizing receptor. In certain embodiments, the activity comprises cytotoxicity, cell proliferation, and cell persistence. In certain embodiments, the antigen-recognizing receptor is a chimeric antigen receptor (CAR), a T-Cell Receptor (TCR), or a TCR like fusion molecule.

The presently disclosed subject matter also provides nucleic acids encoding a fusion polypeptide disclosed herein. In certain embodiments, the nucleic acid comprises a first polynucleotide encoding a fusion polypeptide disclosed herein, and a second polynucleotide encoding an antigen-recognizing receptor that binds to an antigen.

In certain embodiments, the nucleic acid further comprises a first promoter that is operably linked to the fusion polypeptide. In certain embodiments, the nucleic acid further comprises a second promoter that is operably linked to the antigen-recognizing receptor. In certain embodiments, one or both of the first and second promoters are endogenous or exogenous. In certain embodiments, the exogenous promoter is selected from the group consisting of an elongation factor (EF)-1 promoter, a CMV promoter, a SV40 promoter, a PGK promoter, and a metallothionein promoter.

In certain embodiments, one or both of the first and second promoters are inducible promoters. In certain embodiments, the inducible promoter is selected from the group consisting of a NFAT transcriptional response element (TRE) promoter, a CD69 promoter, a CD25 promoter, and an IL-2 promoter.

Additionally, the presently disclosed subject matter provides vectors and lipid nanoparticles including the nucleic acids disclosed herein.

The presently disclosed subject matter further provides cells comprising the fusion polypeptides, the nucleic acids, the vectors, or the lipid nanoparticles disclosed herein.

In certain embodiments, the cell further comprises an antigen-recognizing receptor that binds to an antigen. In certain embodiments, the antigen-recognizing receptor is a chimeric antigen receptor (CAR), a T cell receptor (TCR), or a TCR like fusion molecule.

In certain embodiments, the antigen is a tumor antigen or a pathogen antigen. In certain embodiments, the antigen is a tumor antigen. In certain embodiments, the tumor antigen is selected from CD19, carbonic anhydrase IX (CAIX), carcinoembryonic antigen (CEA), CD8, CD7, CD10, CD20, CD22, CD30, CD33, CLL1, CD34, CD38, CD41, CD44, CD49f, CD56, CD74, CD133, CD138, CD123, CD44V6, an antigen of a cytomegalovirus (CMV) infected cell (e.g., a cell surface antigen), epithelial glycoprotein-2 (EGP-2), epithelial glycoprotein-40 (EGP-40), epithelial cell adhesion molecule (EpCAM), receptor tyrosine-protein kinases Erb-B2,3,4 (erb-B2,3,4), folate-binding protein (FBP), fetal acetylcholine receptor (AChR), folate receptor-α, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Ra2), κ-light chain, kinase insert domain receptor (KDR), Lewis Y (LeY), L1 cell adhesion molecule (LlCAM), melanoma antigen family A, 1 (MAGE-A1), Mucin 16 (MUC16), Mucin 1 (MUC1), Mesothelin (MSLN), ERBB2, MAGEA3, p53, MART1, GP100, Proteinase3 (PR1), Tyrosinase, Survivin, hTERT, EphA2, NKG2D ligands, cancer-testis antigen NY-ESO-1, oncofetal antigen (h5T4), prostate stem cell antigen (PSCA), prostate-specific membrane antigen (PSMA), ROR1, tumor-associated glycoprotein 72 (TAG-72), vascular endothelial growth factor R2 (VEGF-R2), Wilms tumor protein (WT-1), BCMA, NKCS1, EGF1R, EGFR-VIII, CD99, CD70, ADGRE2, CCR1, LILRB2, PRAME CCR4, CD5, CD3, TRBC1, TRBC2, TIM-3, Integrin B7, ICAM-1, CD70, Tim3, CLEC12A, and ERBB. In certain embodiments, the tumor antigen is CD19.

In certain embodiments, the antigen-recognizing receptor is exogenous or endogenous. In certain embodiments, the antigen-recognizing receptor is recombinantly expressed. In certain embodiments, the antigen-recognizing receptor is expressed from a vector.

In certain embodiments, the cell is a cell of the lymphoid lineage or a cell of the myeloid lineage. In certain embodiments, the cell of the lymphoid lineage is selected from T cells, B cells, Natural Killer (NK) cells, dendritic cells. In certain embodiments, the cell is a T cell. In certain embodiments, the T cell is derived from an induced pluripotent stem cell. In certain embodiments, the T cell is a CD8+ T cell. In certain embodiments, the CD8+ T cell is CD4 independent. In certain embodiments, the T cell is selected from the group consisting of a cytotoxic T lymphocyte (CTL), a γδ T cell, a tumor-infiltrating lymphocyte (TIL), a virus-specific T cell (VST), a regulatory T cell, and a Natural Killer T (NKT) cell. In certain embodiments, the T cell is a tumor-infiltrating lymphocyte (TIL). In certain embodiments, the T cell is a virus-specific T cell (VST).

In certain embodiments, the fusion polypeptide is integrated at a locus within the genome of the T cell. In certain embodiments, the fusion polypeptide and the antigen-recognizing receptor are integrated at a locus within the genome of the T cell. In certain embodiments, the locus is selected from the group consisting of a TRAC locus, a TRBC locus, a TRDC locus, and a TRGC locus. In certain embodiments, the locus is a TRAC locus or a TRBC locus. In certain embodiments, the locus is a TRAC locus.

In certain embodiments, the antigen-recognizing receptor is a chimeric antigen receptor (CAR). In certain embodiments, the antigen-recognizing receptor is a TCR like fusion protein.

In certain embodiments, the expressions of fusion polypeptide is under the control of an endogenous promoter. In certain embodiments, the expressions of the fusion polypeptide and antigen-recognizing receptor is under the control of an endogenous promoter. In certain embodiments, the endogenous promoter is selected from the group consisting of an endogenous TRAC promoter, an endogenous TRBC promoter, an endogenous TRDC promoter, an endogenous TRGC promoter, and a combination thereof. In certain embodiments, the endogenous promoter is a TRAC promoter.

In certain embodiments, the cell is autologous. In certain embodiments, the cell is allogeneic.

The presently disclosed subject matter further provides compositions comprising a nucleic acid, a vector, a lipid nanoparticle, or a cell disclosed herein. In certain embodiments, the composition is a pharmaceutical composition further comprising a pharmaceutically acceptable excipient. In certain embodiments, the composition is for treating and/or preventing a neoplasm, an autoimmune disease, and/or an infectious disease.

In certain embodiments, the composition further comprises a regulator that is capable of regulating or modulating expression and/or activity of the fusion polypeptide. In certain embodiments, the regulator is selected from the group consisting of promoters that are capable of controlling the expression of the fusion polypeptide, molecules that are capable of regulating or modulating expression and/or activity of the co-stimulatory ligand, molecules that are capable of regulating or modulating expression and/or activity of the co-stimulatory molecule. In certain embodiments, the molecules that are capable of regulating or modulating expression and/or activity of the co-stimulatory ligand are selected from the group consisting of antibodies that bind to the co-stimulatory ligand, and fusion proteins that bind to the co-stimulatory ligand and regulate or modulate the expression and/or activity of the co-stimulatory ligand.

In certain embodiments, the regulator is an anti-CD80 antibody and the fusion polypeptide comprises an extracellular domain comprising a CD80 polypeptide and a transmembrane domain comprising a CD80 polypeptide. In certain embodiments, the regulator is a fusion protein that binds to CD80 and modulates the activity of CD80 and the fusion polypeptide comprises an extracellular domain comprising a CD80 polypeptide and a transmembrane domain comprising a CD80 polypeptide. In certain embodiments, the fusion protein comprises an CTLA-4 fragment that binds to CD80. In certain embodiments, the CTLA-4 fragment that binds to CD80 is abatacept or belatacept.

In certain embodiments, the molecules that are capable of regulating or modulating expression and/or activity of the co-stimulatory molecule are selected from the group consisting of antibodies that bind to the co-stimulatory molecule, and fusion proteins that bind to the co-stimulatory molecule and regulate or modulate the expression and/or activity of the co-stimulatory molecule.

Moreover, the presently disclosed subject matter provides methods of reducing tumor burden in a subject, methods of treating a subject having a relapse of a neoplasm, and/or methods of treating and/or preventing a neoplasm in a subject. In certain embodiments, the methods comprise administering to the subject an effective amount of the cells, the compositions, the nucleic acids, the vectors, or the lipid nanoparticles disclosed herein.

In certain embodiments, the methods reduce the number of tumor cells, reduce tumor size, and/or eradicate the tumor in the subject. In certain embodiments, the subject received an immunotherapy prior to said administration of the cells or the composition.

In certain embodiments, the neoplasm or tumor is cancer. In certain embodiments, the neoplasm or tumor is selected from the group consisting of blood cancers and solid tumors.

In certain embodiments, the blood cancer is multiple myeloma, myeloid disorder, leukemia, or lymphoma. In certain embodiments, the leukemia is acute myeloid leukemia (AML), chronic myeloid leukemia (CML), acute lymphocytic leukemia (ALL), chronic lymphocytic leukemia (CLL), acute promyelocytic leukemia (APL), mixed-phenotype acute leukemia (MLL), hairy cell leukemia, or B cell prolymphocytic leukemia. In certain embodiments, the lymphoma is Hodgkin's lymphoma, non-Hodgkin's lymphoma, B-cell non-Hodgkin's lymphoma, or T-cell non-Hodgkin's lymphoma. In certain embodiments, the solid tumor is selected from the group consisting of renal cell carcinoma, non-small-cell lung cancer, lung adenocarcinoma, lung squamous cell carcinoma, lung neuroendocrine carcinoma, small-cell lung cancer, pancreatic cancer, breast cancer, astrocytoma, glioblastoma, laryngeal/pharyngeal carcinoma, EBV-associated nasopharyngeal carcinoma, and ovarian carcinoma.

In certain embodiments, the methods further comprise administering to the subject a regulator that is capable of regulating or modulating expression, activity of the fusion polypeptide. In certain embodiments, the regulator is selected from the group consisting of promoters that are capable of controlling the expression of the fusion polypeptide, molecules that are capable of regulating or modulating expression and/or activity of the co-stimulatory ligand, molecules that are capable of regulating or modulating expression and/or activity of the co-stimulatory molecule. In certain embodiments, the molecules that are capable of regulating or modulating expression and/or activity of the co-stimulatory ligand are selected from the group consisting of antibodies that bind to the co-stimulatory ligand, and fusion proteins that bind to the co-stimulatory ligand and regulate or modulate the expression and/or activity of the co-stimulatory ligand.

In certain embodiments, the regulator is an anti-CD80 antibody and the fusion polypeptide comprises an extracellular domain comprising a CD80 polypeptide and a transmembrane domain comprising a CD80 polypeptide. In certain embodiments, the regulator is a fusion protein that binds to CD80 and modulates the activity of CD80 and the fusion polypeptide comprises an extracellular domain comprising a CD80 polypeptide and a transmembrane domain comprising a CD80 polypeptide. In certain embodiments, the fusion protein is a CTLA-4 fragment that binds to CD80. In certain embodiments, the CTLA-4 fragment that binds to CD80 is abatacept or belatacept.

In certain embodiments, the molecules that are capable of regulating or modulating expression, activity of the co-stimulatory molecule are selected from the group consisting of antibodies that bind to the co-stimulatory molecule, fusion proteins that bind to the co-stimulatory molecule and regulate or modulate the expression, activity of the co-stimulatory molecule. In certain embodiments, the regulator is capable of depleting the cell.

Further, the presently disclosed subject matter provides methods for producing a cell. In certain embodiments, the methods comprise introducing into a cell the nucleic acids, the vectors, or the lipid nanoparticles disclosed herein.

Also provided herein are kits comprising the cells, the compositions, the nucleic acids, the vectors, or the lipid nanoparticles disclosed herein. In certain embodiments, the kit further comprises written instructions for treating and/or preventing a neoplasm, a pathogen infection, and/or an infectious disease.

### BRIEF DESCRIPTION OF THE FIGURES

The following Detailed Description, given by way of example, but not intended to limit the presently disclosed subject matter to specific embodiments described, may be understood in conjunction with the accompanying drawings.

FIG. 1 depicts the anatomy of a PRIME-costimulatory fusion polypeptide in accordance with certain embodiments of the presently disclosed subject matter.

FIG. 2 is a schematic depicting the trans and cis interaction networks of CD80/4-1BB fusion protein compared to the presently disclosed PRIME-costimulatory fusion polypeptide.

FIGS. 3A-C depict PRIME-costimulatory fusion polypeptide expression on the surface of T cells. FIG. 3A shows CD80 surface expression as assessed by flow cytometry staining of T cells expressing assorted constructs (Left) and HLA independent TCR (HIT) expression as assessed by flow cytometry staining of Goat Anti-Mouse Fab fragment (GAM) (Right). FIG. 3B shows the quantification of CD80 expression. FIG. 3C shows the quantification of HIT expression.

FIGS. 4A-C depict the proliferation of cells expressing PRIME-costimulatory fusion polypeptide or CD80/4-1BB fusion protein upon antigen stimulation. FIG. 4A shows the antigen stimulation protocol. FIG. 4B shows the fold expansion of HIT cells cultured with NALM6 tumor cells. FIG. 4C shows the fold expansion of HIT cells cultured with NALM6 tumor cells expressing PD-L1 (NALM6-PD-L1).

FIGS. 5A-C depict the effects of cells expressing PRIME-costimulatory fusion polypeptide fusion protein and CD80-4-1BB fusion protein on PD-L1 dependent tumor growth in vitro. FIG. 5A shows serial in vitro cytotoxicity assay with NALM6 cells. FIG. 5B shows serial in vitro cytotoxicity assay with NALM6-PD-L1 cells. FIG. 5C shows serial in vitro cytotoxicity assay with a 1:1 mix of NALM6:NALM6 PD-L1 cells.

FIGS. 6A-6D depict comparable effects of cells expressing PRIME-costimulatory fusion polypeptide and CD80/4-1BB fusion protein on PD-L1 negative tumor cells. Mice were injected with NALM6-GFP-FFLuc cells and treated with TRAC-HIT-CD80/4-1BB or TRAC-HIT-PRIME-costim cells. Mice were re-challenged with 4×10⁶ tumor cells 59 days post 1^{st} T cell infusion (n=4 mice for TRAC-HIT-CD80/4-1BB and 5 mice for TRAC-HIT-PRIME-costim group). FIG. 6A shows tumor burden quantification observed in presence of 10⁵ cells expressing TRAC-HIT-CD80/4-1BB or TRAC-HIT-PRIME-costim. FIG. 6B shows Kaplan-Meier survival analysis in presence of 10⁵ cells expressing TRAC-HIT-CD80/4-1BB or TRAC-HIT-PRIME-costim. FIG. 6C shows tumor burden quantification observed in presence of 0.25×10⁵ cells expressing TRAC-HIT-CD80/4-1BB or TRAC-HIT-PRIME-costim. FIG. 7B shows Kaplan-Meier survival analysis observed in presence of 0.25×10⁵ cells expressing TRAC-HIT-CD80/4-1BB or TRAC-HIT-PRIME-costim.

FIGS. 7A and 7B depicts the effects of cells expressing PRIME-costimulatory fusion polypeptide and CD80/4-1BB fusion protein on PD-L1 positive tumor cells. FIG. 7A shows tumor burden quantification and Kaplan-Meier survival analysis of mice injected with 0.5×10⁶ PD-L1 expressing NALM6-GFP-FFLuc cells followed by treatment with 10⁵ cells expressing TRAC-HIT, TRAC-HIT-CD80/4-1BB, TRAC-HIT-PRIME-costim, or TRAC-HIT-CD80-P2A-4-1BBL 4 days later (n=5 mice per group). FIG. 7B shows tumor burden quantification and Kaplan-Meier survival analysis of mice injected with 0.5×10⁶ PD-L1 expressing NALM6-GFP-FFLuc cells followed by treatment with 0.25×10⁵ TRAC-HIT, TRAC-HIT-CD80/4-1BB, TRAC-HIT-PRIME-costim, or TRAC-HIT-CD80-P2A-4-1BBL cells 4 days later (n=5 mice per group).

FIGS. 8A-8D depict the effects of cells expressing PRIME-costimulatory fusion polypeptide and CD80/4-1BB fusion protein on PD-L1 positive tumor cells. Mice were injected with NALM6-GFP-FFLuc cells and treated with TRAC-HIT-CD80/4-1BB or TRAC-HIT-PRIME-costim cells. Mice were re-challenged with 4×10⁶ tumor cells 59 days post 1^{st} T cell infusion (n=4 mice for TRAC-HIT-CD80/4-1BB and 5 mice for TRAC-HIT-PRIME-costim group). FIG. 8A shows tumor burden quantification observed in presence of 10⁵ cells expressing TRAC-HIT-CD80/4-1BB or TRAC-HIT-PRIME-costim. FIG. 8B shows Kaplan-Meier survival analysis in presence of 10⁵ cells expressing TRAC-HIT-CD80/4-1BB or TRAC-HIT-PRIME-costim. FIG. 8C shows tumor burden quantification observed in presence of 0.25×10⁵ cells expressing TRAC-HIT-CD80/4-1BB or TRAC-HIT-PRIME-costim. FIG. 8B shows Kaplan-Meier survival analysis observed in presence of 0.25×10⁵ cells expressing TRAC-HIT-CD80/4-1BB or TRAC-HIT-PRIME-costim.

FIGS. 9A-9E show that CTLA-4 binding to HIT-PRIME-costim is preserved and required for enhanced tumor control in vivo. FIG. 9A shows the titration of human CTLA-4-FC fusion protein binding to cells expressing CD80/4-1BB or PRIME-costimulatory fusion polypeptide T cells. FIG. 9B shows flow cytometry profiles of cells expressing CD80/4-1BB or PRIME-costimulatory fusion polypeptide followed by transduction with SFG-truncated-CTLA-4 to assess CTLA-4/CD80 binding. FIG. 9C shows histograms of cells presented in FIG. 11B. FIG. 9D shows tumor burden quantification of mice injected with 0.5×10⁶ NALM6-GFP-FFLuc cells followed by treatment with 10⁵ TRAC-HIT-PRIME-costim with TRBC-KO (control) or CTLA-4− KO T cells 4 days later (n=5 mice for CTLA-4-KO condition, n=4 mice for TRBC-KO condition). FIG. 9E shows Kaplan-Meier survival analysis.

FIGS. 10A-10E show that CD28 binding to HIT-PRIME-costim is preserved and required for enhanced tumor control in vivo. FIG. 10A shows flow cytometry profiles of cells expressing CD80/4-1BB or PRIME-costimulatory fusion polypeptide to assess CD28/CD80 binding. FIG. 10B shows histograms of cells presented in FIG. 10A. FIG. 10C shows tumor burden quantification of mice injected with 0.5×10⁶ NALM6-GFP-FFLuc cells followed by treatment with 10⁵ TRAC-HIT-PRIME-costim with TRBC (control) or CD28 CRISPR-cas9 KO T cells 4 days later. FIG. 10D shows tumor burden quantification of mice injected with 0.5×10⁶ PD-L1 expressing NALM6-GFP-FFLuc cells followed by treatment with 105 TRAC-HIT-PRIME-costim with TRBC (control) or CD28 CRISPR-cas9 KO T cells 4 days later. Mice were rechallenged with the same tumor cells at day 59 post T cell injection. FIG. 10E shows Kaplan-Meier survival analysis of mice presented in FIG. 10D.

FIGS. 11A-11C show that PD-L1 binds to PRIME-Costim. FIG. 11A shows the titration of human PD-L1-FC fusion protein binding to cells expressing CD80-4-1BB or PRIME-costimulatory fusion polypeptide. FIG. 11B shows flow cytometry profiles of cells expressing CD80/4-1BB or PRIME-costimulatory fusion polypeptide followed by transduction with SFG-PD-L1 to assess PD-L1/CD80 binding. FIG. 11C shows histograms of cells presented in FIG. 11B.

FIGS. 12A-12D demonstrate enhanced in vivo tumor control of T cells expressing a HIT receptor and the PRIME-costimulatory fusion polypeptide. FIG. 12A shows tumor burden quantification of mice injected with 0.5×10⁶ NALM6-PD-L1 cells followed by treatment with 105 TRAC-HIT-CD80/4-1BB in TRBC-KO T cells 4 days later. FIG. 12B shows tumor burden quantification of mice injected with 0.5×10⁶ NALM6-PD-L1 cells followed by treatment with 105 TRAC-HIT-CD80/4-1BB in PD1-KO T cells 4 days later. FIG. 12C shows tumor burden quantification of mice injected with 0.5×10⁶ NALM6-PD-L1 cells followed by treatment with 105 TRAC-HIT-PRIME-costim in TRBC-KO T cells 4 days later. FIG. 12C shows Kaplan-Meier survival analysis of mice presented in FIGS. 12A-12C.

### DETAILED DESCRIPTION OF THE INVENTION

The presently disclosed subject matter provides fusion polypeptides that are capable of enhancing the activity and/or efficacy of immunotherapy (e.g., T cell immunotherapy). The fusion polypeptide can enhance the activity and/or efficacy of cells (e.g., T cells or NK cells) comprising an antigen-recognizing receptor (e.g., a CAR, a TCR, or a TCR like fusion molecule). The presently disclosed subject matter also provides methods of using such fusion polypeptide for inducing and/or enhancing an immune response of a cell to a target antigen, and/or treating and/or preventing neoplasms or other diseases/disorders (e.g., autoimmune diseases and infectious diseases), e.g., where an increase in an antigen-specific immune response is desired. The presently disclosed subject matter is based, at least in part, on the discovery that a fusion polypeptide disclosed herein can enhance the activity (e.g., cytotoxicity) of a cell comprising an antigen-recognizing receptor (e.g., a CAR, a TCR, or a TCR like fusion molecule).

Non-limiting embodiments of the present disclosure are described by the present specification and Examples.

For purposes of clarity of disclosure and not by way of limitation, the detailed description is divided into the following subsections:
1. Definitions;
2. Fusion Polypeptides;
3. Cells;
4. Nucleic Acids and Compositions;
5. Formulations and Administration;
6. Methods of Treatment;
7. Kits; and
8. Exemplary Embodiments.

### 1. DEFINITIONS

Unless defined otherwise, all technical and scientific terms used herein have the meaning commonly understood by a person skilled in the art. The following references provide one of skill with a general definition of many of the terms used in the presently disclosed subject matter: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991).

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold or within 2-fold, of a value.

“Antibody” and “antibodies” as those terms are known in the art refer to antigen binding proteins of the immune system. The term “antibody” as referred to herein includes whole, full length antibodies having an antigen-binding region, and any fragment thereof in which the “antigen-binding fragment” or “antigen-binding region” is retained, or single chains, for example, single chain variable fragment (scFv), thereof. A naturally occurring “antibody” is a glycoprotein comprising at least two heavy (H) chains and two light (L) chains inter-connected by disulfide bonds. Each heavy chain is comprised of a heavy chain variable region (abbreviated herein as VH) and a heavy chain constant (CH) region. The heavy chain constant region is comprised of three domains, CH1, CH2 and CH3. Each light chain is comprised of a light chain variable region (abbreviated herein as VL) and a light chain constant CL region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antigen-binding fragment” or “antigen-binding region” of an antibody, as used herein, refers to that region or fragment of the antibody that binds to the antigen and which confers antigen specificity to the antibody; fragments of antigen-binding proteins, for example, antibodies include one or more fragments of an antibody that retain the ability to specifically bind to an antigen (e.g., a CD3 polypeptide). It has been shown that the antigen-binding function of an antibody can be performed by fragments of a full-length antibody. Examples of antigen-binding fragments encompassed within the term “antibody fragments” of an antibody include a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., Nature 1989; 341:544-546), which consists of a VH domain; and an isolated complementarity determining region (CDR).

Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules. These are known as single chain Fv (scFv); see e.g., Bird et al., Science (1988); 242:423-426; and Huston et al., Proc Natl Acad Sci (1998); 85:5879-5883. These antibody fragments are obtained using conventional techniques known to those of skill in the art, and the fragments are screened for utility in the same manner as are intact antibodies.

The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the presently disclosed subject matter may include amino acid residues not encoded by human germline immunoglobulin sequences (e.g., mutations introduced by random or site-specific mutagenesis in vitro or by somatic mutation in vivo).

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring the production of the antibody by any particular method. For example, the monoclonal antibodies to be used in accordance with the presently disclosed subject matter may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “recombinant human antibody”, as used herein, includes all human antibodies that are prepared, expressed, created, or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “humanized antibody” is intended to refer to antibodies in which CDR sequences derived from the germline of another mammalian species, such as a mouse, have been grafted onto human framework sequences. Additional framework region modifications may be made within the human framework sequences.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

As used herein, the term “specifically binds to,” when referring to an antibody or an antigen-binding fragment thereof, means that the antibody or antigen-binding fragment thereof binds to a desired target (e.g., human PD-L1) with a dissociation constant (KD) of about 1×10⁻⁸ M or less, about 5×10⁻⁹ M or less, about 1×10⁻⁹ M or less, about 5×10⁻¹⁰ M or less, about 1×10⁻¹⁰ M or less, about 5×10⁻¹¹ M or less, or about 1×10⁻¹¹ M or less. An “antibody that competes for binding” or “antibody that cross-competes for binding” with a reference antibody for binding to an antigen, e.g., PD-L1, refers to an antibody that blocks binding of the reference antibody to the antigen (e.g., PD-L1) in a competition assay by 50% or more, and conversely, the reference antibody blocks binding of the antibody to the antigen (e.g., PD-L1) in a competition assay by 50% or more. An exemplary competition assay is described in “Antibodies”, Harlow and Lane (Cold Spring Harbor Press, Cold Spring Harbor, NY).

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen (e.g., a PD-L1 polypeptide).”

As used herein, the term “single-chain variable fragment” or “scFv” is a fusion protein of the variable regions of the heavy (VH) and light chains (VL) of an immunoglobulin (e.g., mouse or human) covalently linked to form a VH::VL heterodimer. The heavy (VH) and light chains (VL) are either joined directly or joined by a peptide-encoding linker (e.g., 10, 15, 20, 25 amino acids), which connects the N-terminus of the VH with the C-terminus of the VL, or the C-terminus of the VH with the N-terminus of the VL. The linker is usually rich in glycine for flexibility, as well as serine or threonine for solubility. The linker can link the heavy chain variable region and the light chain variable region of the extracellular antigen-binding domain.

Non-limiting examples of linkers, e.g., for use in generating an scFv, are disclosed in Shen et al., Anal Chem (2008); 80(6):1910-1917 and WO 2014/087010, the contents of which are hereby incorporated by reference in their entireties. In certain embodiments, the linker is a G4S linker. In certain embodiments, the linker comprises or consists of the amino acid sequence set forth in SEQ ID NO: 1, which is provided below:

[SEQ ID NO: 1]
GGGGSGGGGSGGGSGGGGS

In certain embodiments, the linker comprises or consists of the amino acid sequence set forth in SEQ ID NO: 2, which is provided below:

[SEQ ID NO: 2]
GGGGSGGGGSGGGGS

In certain embodiments, the linker comprises or consists of the amino acid sequence set forth in SEQ ID NO: 3, which is provided below:

[SEQ ID NO: 3]
GGGGSGGGGSGGGGSGGGSGGGGS

In certain embodiments, the linker comprises or consists of the amino acid sequence set forth in SEQ ID NO: 4, which is provided below:

[SEQ ID NO: 4]
GGGGSGGGGSGGGGSGGGGSGGGSGGGGS

In certain embodiments, the linker comprises or consists of the amino acid sequence set forth in SEQ ID NO: 5, which is provided below:

[SEQ ID NO: 5]
GGGGS

In certain embodiments, the linker comprises or consists of the amino acid sequence set forth in SEQ ID NO: 6, which is provided below:

[SEQ ID NO: 6]
GGGGSGGGGS

In certain embodiments, the linker comprises or consists of the amino acid sequence set forth in SEQ ID NO: 26, which is provided below:

[SEQ ID NO: 26]
GGGGSGGGGSGGGGSGGGGS

Despite removal of the constant regions and the introduction of a linker, scFv proteins retain the specificity of the original immunoglobulin. Single chain Fv polypeptide antibodies can be expressed from a nucleic acid comprising VH- and VL-encoding sequences as described by Huston, et al. (Proc. Nat. Acad. Sci. USA, 1988; 85:5879-5883). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008; 27(6):455-51; Peter et al., J Cachexia Sarcopenia Muscle 2012 Aug. 12; Shieh et al., J Imunol 2009; 183(4):2277-85; Giomarelli et al., Thromb Haemost 2007; 97(6):955-63; Fife eta., J Clin Invst 2006; 116(8):2252-61; Brocks et al., Immunotechnology 1997; 3(3):173-84; Moosmayer et al., Ther Immunol 1995; 2(10:31-40). Agonistic scFvs having stimulatory activity have been described (see, e.g., Peter et al., J Bio. Chem 2003; 25278(38):36740-7; Xie et al., Nat Biotech 1997; 15(8):768-71; Ledbetter et al., Crit Rev Immunol 1997; 17(5-6):427-55; Ho et al., BioChim Biophys Acta 2003; 1638(3):257-66).

As used herein, “F(ab)” refers to a fragment of an antibody structure that binds to an antigen but is monovalent and does not have a Fc portion, for example, an antibody digested by the enzyme papain yields two F(ab) fragments and an Fc fragment (e.g., a heavy (H) chain constant region; Fc region that does not bind to an antigen).

As used herein, “F(ab′)₂” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)₂” fragment can be split into two individual Fab′ fragments.

As used herein, the term “nanobody” or “single-domain antibody” refers to small antigen-binding fragments that are derived from heavy chain only antibodies present in camelids (VHH, from camels and llamas), and cartilaginous fishes (VNAR, from sharks). Nanobodies are useful alternatives to conventional antibodies due to their small size, and high solubility and stability.

As used herein, the term “vector” refers to any genetic element, such as a plasmid, phage, transposon, cosmid, chromosome, virus, virion, etc., which is capable of replication when associated with the proper control elements and which can transfer gene sequences into cells. Thus, the term includes cloning and expression vehicles, as well as viral vectors and plasmid vectors.

“CDRs” are defined as the complementarity determining region amino acid sequences of an antibody which are the hypervariable regions of immunoglobulin heavy and light chains. See, e. g., Kabat et al., Sequences of Proteins of Immunological Interest, 4th U. S. Department of Health and Human Services, National Institutes of Health (1987), or IMGT numbering system (Lefranc, The Immunologist (1999); 7:132-136; Lefranc et al., Dev. Comp. Immunol. (2003); 27:55-77). The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise three heavy chain and three light chain CDRs or CDR regions in the variable region. CDRs provide the majority of contact residues for the binding of the antibody to the antigen or epitope region. In certain embodiments, the CDRs are identified according to the IMGT system. In certain embodiments, the CDRs are identified using the IMGT numbering system accessible at http://www.imgt.org/IMGT_vquest/input.

As used herein, a “co-stimulatory molecule polypeptide” refers to a polypeptide of a cell surface molecule other than an antigen receptor or its ligand that can provide an efficient response of lymphocytes to an antigen. In certain embodiments, a co-stimulatory molecule polypeptide can provide optimal lymphocyte activation.

As used herein, a “co-stimulatory ligand polypeptide” refers to a polypeptide of a molecule that upon binding to its receptor (e.g., a co-stimulatory molecule) produces a co-stimulatory response, e.g., an intracellular response that effects the stimulation provided when an antigen-recognizing receptor (e.g., a chimeric antigen receptor (CAR)) binds to its target antigen.

As used herein, “a functional fragment” of a molecule or polypeptide includes a fragment of the molecule or polypeptide that retains at least about 80%, at least about 85%, at least about 90%, at least about 95%, or at least about 100% of the primary function of the molecule or polypeptide.

The term “antigen-recognizing receptor” as used herein refers to a receptor that is capable of activating an immune or immunoresponsive cell (e.g., a T-cell) in response to its binding to an antigen.

The term “chimeric antigen receptor” or “CAR” as used herein refers to a molecule comprising an extracellular antigen-binding domain that is fused to an intracellular signaling domain that is capable of activating or stimulating an immune or immunoresponsive cell, and a transmembrane domain. In certain embodiments, the extracellular antigen-binding domain of a CAR comprises a scFv. The scFv can be derived from fusing the variable heavy and light regions of an antibody. Alternatively or additionally, the scFv may be derived from Fab's (instead of from an antibody, e.g., obtained from Fab libraries). In certain embodiments, the scFv is fused to the transmembrane domain and then to the intracellular signaling domain. In certain embodiments, the CAR is selected to have high binding affinity or avidity for the antigen.

The term “antigen-binding domain” as used herein refers to a domain capable of specifically binding a particular antigenic determinant or set of antigenic determinants present on a cell.

By “receptor” is meant a polypeptide, or portion thereof, present on a cell membrane that selectively binds one or more ligands.

By “recognize” is meant selectively binds to a target. A T cell that recognizes a tumor can express a receptor (e.g., a TCR or CAR) that binds to a tumor antigen.

By “immunoresponsive cell” is meant a cell that functions in an immune response or a progenitor, or progeny thereof. In certain embodiments, the immunoresponsive cell is a cell of lymphoid lineage. Non-limiting examples of cells of lymphoid lineage include T cells, Natural Killer (NK) cells, B cells, and stem cells from which lymphoid cells may be differentiated. In certain embodiments, the immunoresponsive cell is a cell of myeloid lineage.

By “isolated cell” is meant a cell that is separated from the molecular and/or cellular components that naturally accompany the cell.

By “activates an immunoresponsive cell” is meant induction of signal transduction or changes in protein expression in the cell resulting in the initiation of an immune response. For example, when CD3 Chains cluster in response to ligand binding and immunoreceptor tyrosine-based inhibition motifs (ITAMs) a signal transduction cascade is produced. In certain embodiments, when an endogenous TCR or an exogenous CAR binds to an antigen, a formation of an immunological synapse occurs that includes clustering of many molecules near the bound receptor (e.g. CD4 or CD8, CD3γ/δ/ε/ζ, etc.). This clustering of membrane-bound signaling molecules allows for ITAM motifs contained within the CD3 chains to become phosphorylated. This phosphorylation in turn initiates a T cell activation pathway ultimately activating transcription factors, such as NF-κB and AP-1. These transcription factors induce global gene expression of the T cell to increase IL-2 production for proliferation and expression of master regulator T cell proteins in order to initiate a T cell mediated immune response.

By “stimulates an immunoresponsive cell” is meant a signal that results in a robust and sustained immune response. In various embodiments, this occurs after immune cell (e.g., T-cell) activation or concomitantly mediated through receptors including, but not limited to, CD28, CD137 (4-1BB), OX40, CD40, and ICOS. Receiving multiple stimulatory signals can be important to mount a robust and long-term T cell mediated immune response. T cells can quickly become inhibited and unresponsive to antigen. While the effects of these co-stimulatory signals may vary, they generally result in increased gene expression in order to generate long lived, proliferative, and anti-apoptotic T cells that robustly respond to antigen for complete and sustained eradication.

As used herein, the term “affinity” is meant as a measure of binding strength. Affinity can depend on the closeness of stereochemical fit between antibody combining sites and antigen determinants, on the size of the area of contact between them, and/or on the distribution of charged and hydrophobic groups. As used herein, the term “affinity” also includes “avidity”, which refers to the strength of the antigen-antibody bond after the formation of reversible complexes. Methods for calculating the affinity of an antibody for an antigen are known in the art, including, but not limited to, various antigen-binding experiments, e.g., functional assays (e.g., flow cytometry assay).

As used herein, the term “antigen heterogeneity” refers to the differential expression of a number of antigens (e.g., tumor antigens, e.g., CD70, CD312) which results in variation in the tumor cell phenotype and distribution of tumor antigen-positive cells.

As used herein, the term “low antigen density” refers to a target molecule (e.g., an antigen) having a cell surface density of less than about 5,000 molecules per cell. In certain embodiments, the low antigen density is a cell surface density that is less than about 4,000 molecules per cell, less than about 3,000 molecules per cell, less than about 2,000 molecules per cell, less than about 1,500 molecules per cell, less than about 1,000 molecules per cell, less than about 500 molecules per cell, less than about 200 molecules per cell, or less than about 100 molecules per cell. In certain embodiments, the low antigen density is a cell surface density is less than about 2,000 molecules per cell. In certain embodiments, the low antigen density is a cell surface density is less than about 1,500 molecules per cell. In certain embodiments, the low antigen density is a cell surface density is less than about 1,000 molecules per cell. In certain embodiments, the low antigen density is a cell surface density is between about 4,000 molecules per cell and about 2,000 molecules per cell, between about 2,000 molecules per cell and about 1,000 molecules per cell, between about 1,500 molecules per cell and about 1,000 molecules per cell, between about 2,000 molecules per cell and about 500 molecules per cell, between about 1,000 molecules per cell and about 200 molecules per cell, or between about 1,000 molecules per cell and about 100 molecules per cell.

As used herein, the term “low tumor cell frequency” refers to a target cell having a target cell frequency of less than about 50% per tumor. In certain embodiments, the low tumor cell frequency is less than about 40% per tumor, less than about 30% per tumor, less than about 20% per tumor, less than about 15% per tumor, less than about 10% per tumor, less than about 5% per tumor, less than about 2% per tumor, or less than about 1% per tumor. In certain embodiments, the low tumor cell frequency is less than about 2% per tumor. In certain embodiments, the low tumor cell frequency is less than about 1.5% per tumor. In certain embodiments, the low tumor cell frequency is less than about 1% per tumor. In certain embodiments, the low tumor cell frequency is between about 40% per tumor and about 20% per tumor, between about 20% per tumor and about 10% per tumor, between about 15% per tumor and about 10% per tumor, between about 20% per tumor and about 5% per tumor, between about 10% per tumor and about 2% per tumor, or between about 10% per tumor and about 1% per tumor.

As used herein, the term “substantially identical” or “substantially homologous” refers to a polypeptide or a nucleic acid molecule exhibiting at least about 50% identical or homologous to a reference amino acid sequence (for example, any of the amino acid sequences described herein) or a reference nucleic acid sequence (for example, any of the nucleic acid sequences described herein). In certain embodiments, such a sequence is at least about 60%, at least about 65%, at least about 70%, at least about 75%, at least about 80%, at least about 85%, at least about 90%, at least about 95%, at least about 99%, or at least about 100% identical or homologous to the amino acid sequence or the nucleic acid sequence used for comparison.

Sequence identity can be measured by using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6. Additionally or alternatively, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences (e.g., heavy and light chain variable region sequences of scFv703) disclosed herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

As used herein, the term “a conservative sequence modification” refers to an amino acid modification that does not significantly affect or alter the binding characteristics of the presently disclosed polypeptides. Conservative modifications can include amino acid substitutions, additions and deletions. Modifications can be introduced into the presently disclosed polypeptides by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function (i.e., the functions set forth in (c) through (1) above) using the functional assays described herein. In certain embodiments, no more than one, no more than two, no more than three, no more than four, no more than five residues within a specified sequence or a CDR region are altered.

By “analog” is meant a structurally related polypeptide or nucleic acid molecule having the function of a reference polypeptide or nucleic acid molecule.

The term “ligand” as used herein refers to a molecule that binds to a receptor. In certain embodiments, the ligand binds to a receptor on another cell, allowing for cell-to-cell recognition and/or interaction.

The term “constitutive expression” or “constitutively expressed” as used herein refers to expression or expressed under all physiological conditions.

By “disease” is meant any condition, disease, or disorder that damages or interferes with the normal function of a cell, tissue, or organ, e.g., neoplasm, and pathogen infection of a cell.

By “effective amount” is meant an amount sufficient to have a therapeutic effect. In certain embodiments, an “effective amount” is an amount sufficient to arrest, ameliorate, or inhibit the continued proliferation, growth, or metastasis (e.g., invasion, or migration) of a neoplasm.

By “endogenous” is meant a nucleic acid molecule or polypeptide that is normally expressed in a cell or tissue.

By “exogenous” is meant a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example, an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

By “modulate” is meant positively or negatively alter. Exemplary modulations include a about 1%, about 2%, about 5%, about 10%, about 25%, about 50%, about 75%, or about 100% change.

By “increase” is meant to alter positively by at least about 5%. An alteration may be by about 5%, about 10%, about 25%, about 30%, about 50%, about 75%, about 100%, or more.

By “reduce” is meant to alter negatively by at least about 5%. An alteration may be by about 5%, about 10%, about 25%, about 30%, about 50%, about 75%, or even by about 100%.

The terms “isolated,” “purified,” or “biologically pure” refer to material that is free to varying degrees from components that normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “neoplasm” is meant a disease characterized by the pathological proliferation of a cell or tissue and its subsequent migration to or invasion of other tissues or organs. Neoplasm growth is typically uncontrolled and progressive, and occurs under conditions that would not elicit, or would cause cessation of, multiplication of normal cells. Neoplasm can affect a variety of cell types, tissues, or organs, including but not limited to an organ selected from bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasms include cancers, such as sarcomas, carcinomas, or plasmacytomas (malignant tumor of the plasma cells). In certain embodiments, the neoplasm is cancer.

By “reference” or “control” is meant a standard of comparison. For example, the level of scFv-antigen binding by a cell expressing a CAR and an scFv may be compared to the level of scFv-antigen binding in a corresponding cell expressing CAR alone.

By “secreted” is meant a polypeptide that is released from a cell via the secretory pathway through the endoplasmic reticulum, Golgi apparatus, and as a vesicle that transiently fuses at the cell plasma membrane, releasing the proteins outside of the cell.

By “signal sequence” or “leader sequence” is meant a peptide sequence (e.g., 5, 10, 15, 20, 25 or 30 amino acids) present at the N-terminus of newly synthesized proteins that directs their entry to the secretory pathway. Exemplary leader sequences include, but is not limited to, a human IL-2 signal sequence (e.g., a human IL-2 signal sequence comprising or consisting of the amino acid sequence set forth in SEQ ID NO: 7), a mouse IL-2 signal sequence (e.g., a mouse IL-2 signal sequence comprising or consisting of the amino acid sequence set forth in SEQ ID NO: 8); a human kappa leader sequence (e.g., a human kappa leader sequence comprising or consisting of the amino acid sequence set forth in SEQ ID NO: 9), a mouse kappa leader sequence (e.g., a mouse kappa leader sequence comprising or consisting of the amino acid sequence set forth in SEQ ID NO: 10); a human CD8 leader sequence (e.g., a human CD8 leader sequence comprising or consisting of the amino acid sequence set forth in SEQ ID NO: 11); a truncated human CD8 signal peptide (e.g., a truncated human CD8 signal peptide comprising or consisting of the amino acid sequence set forth in SEQ ID NO: 12); a human albumin signal sequence (e.g., a human albumin signal sequence comprising or consisting of the amino acid sequence set forth in SEQ ID NO: 13); and a human prolactin signal sequence (e.g., a human prolactin signal sequence comprising or consisting of the amino acid sequence set forth in SEQ ID NO: 14). SEQ ID Nos: 7-14 are provided below.

[SEQ ID NO: 7]
MYRMQLLSCIALSLALVINS
[SEQ ID NO: 8]
MYSMQLASCVTLTLVLLVNS
[SEQ ID NO: 9]
METPAQLLFLLLLWLPDTTG
[SEQ ID NO: 10]
METDTLLLWVLLLWVPGSTG
[SEQ ID NO: 11]
MALPVTALLLPLALLLHAARP
[SEQ ID NO: 12]
MALPVTALLLPLALLLHA
[SEQ ID NO: 13]
MKWVTFISLLESSAYS
[SEQ ID NO: 14]
MDSKGSSQKGSRLLLLLVVSNLLLCQGVVS

By “soluble” is meant a polypeptide that is freely diffusible in an aqueous environment (e.g., not membrane bound).

By “specifically binds” is meant a polypeptide or fragment thereof that recognizes and binds to a biological molecule of interest (e.g., a polypeptide), but which does not substantially recognize and bind other molecules in a sample, for example, a biological sample, which naturally includes a presently disclosed polypeptide.

The term “tumor antigen” as used herein refers to an antigen (e.g., a polypeptide) that is uniquely or differentially expressed on a tumor cell compared to a normal or non-neoplastic cell. In certain embodiments, a tumor antigen includes any polypeptide expressed by a tumor that is capable of activating or inducing an immune response via an antigen recognizing receptor or capable of suppressing an immune response via receptor-ligand binding.

As used herein, “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys. The term “immunocompromised” as used herein refers to a subject who has an immunodeficiency. The subject is very vulnerable to opportunistic infections, infections caused by organisms that usually do not cause disease in a person with a healthy immune system, but can affect people with a poorly functioning or suppressed immune system.

The terms “comprises”, “comprising”, and are intended to have the broad meaning ascribed to them in U.S. Patent Law and can mean “includes”, “including” and the like.

Other aspects of the presently disclosed subject matter are described in the following disclosure and are within the ambit of the presently disclosed subject matter.

### 2. FUSION POLYPEPTIDES

The presently disclosed subject matter provides fusion polypeptides that are capable of enhancing the activity and/or efficacy of a cell comprising an antigen-recognizing receptor (e.g., a CAR, a TCR, or a TCR like fusion molecule).

The inventors of the presently disclosed subject matter developed a novel Platform for Rewiring Immune Molecules and Enhance co-stimulation. The presently disclosed subject matter provides fusion polypeptides, also designated as “PRIME-costim” or “PRIME-costimulatory fusion polypeptide,” which repurpose the extracellular domain of a co-stimulatory ligand polypeptide (e.g., a CD80 polypeptide) to bind a tumor abundant immune checkpoint molecule (e.g., PD-L1). The presently disclosed fusion polypeptides can target immune checkpoint molecules without compromising the interactions between the co-stimulatory ligand polypeptide and its ligands (e.g., interactions of CD80 with CD28 or CTLA-4). Importantly, the presently disclosed fusion polypeptides are capable of delivering costimulatory signaling through its intracellular domain (e.g., including a co-stimulatory molecule polypeptide, e.g., a 4-1BB polypeptide). Without being bound by any theory, the inventors of the presently disclosed subject matter hypothesized that the activity in T cells of the presently disclosed fusion polypeptides would be greatly increased by combining the blockade of key immune checkpoint molecules (e.g., CTLA-4 or PD-L1) with the delivery of signaling upon receptors interactions. The multifaceted antigen-independent stimulatory features of the presently disclosed fusion polypeptide combined with OR-gate function (i.e., PRIME-costimulatory fusion polypeptide can function in the presence of any of its ligands) greatly benefit adoptive cell therapies in an antigen-receptor agnostic manner (i.e., CAR, TCR, or HIT).

### 2.1. Extracellular Domain of the Fusion Polypeptides

In certain embodiments, the fusion polypeptide comprises an extracellular domain and an intracellular domain. In certain embodiments, the extracellular domain comprises an antigen-binding fragment and a co-stimulatory ligand polypeptide.

### 2.1.1. Antigen-Binding Fragments

In certain embodiments, the extracellular domain of the presently disclosed fusion polypeptides include an antigen-binding fragment of an antibody. In certain embodiments, the antigen-binding fragment is a Fab, a Fab′, a F(ab′)2, a variable fragment (Fv), a single chain variable region (scFv), a nanobody, a microantibody, an affibody molecule, an affilin, an affimer, an affitin, an alphabody, an anticalin protein, an avimer, a DARPin (designed ankyrin repeat proteins), or an aptamer. In certain embodiments, the antigen-binding fragment is an scFv. In certain embodiments, the antigen-binding fragment is a nanobody.

In certain embodiments, the antigen-binding fragment binds to an immune checkpoint molecule. As used herein, the term “immune checkpoint molecules” refers to ligand-receptor pairs that exert inhibitory or stimulatory effects on immune responses. The immune checkpoint molecules can inhibitory and stimulatory immune checkpoint molecules and so far have been predominately described in the regulation of the adaptive immune system (e.g., T cell physiology). These molecules are important for the regulation of self-tolerance and immune responses. Further, it has been observed that the modulation of these molecule is associated with immune evasion of the tumors. In certain embodiments, the immune checkpoint molecules is selected from the group consisting of PD-L1, PD-L2, VISTA, B7-H3, B7-H4, B7-H7, herpesvirus entry mediator (HVEM), CD155, CD112, CD200, galectin 9 (GAL9), TIGT, BTLA, LAG-3, TIM-3, and 2B4 (CD224). In certain embodiments, the immune checkpoint molecule is PD-L1.

### 2.1.1.1. Anti-PD-L1 Antigen-binding Fragments

In certain embodiments, the antigen-binding fragment binds to Programmed death-ligand 1 (PD-L1). PD-L1 is an immune checkpoint inhibitor that binds to its receptor PD-1 expressed by immune cells (e.g., T cells). It has been observed that many tumors over-express PD-L1 and/or under-express PD-1. The binding of PD-L1 to PD-1 on immune cells can increase immune evasion and tumor progression by inhibiting cytotoxic T lymphocytes.

In certain embodiments, the presently disclosed anti-PD-L1 antigen-binding fragments bind to human PD-L1. In certain embodiments, the human PD-L1 comprises or consists of the amino acid sequence with a UniProt Reference No: Q9NZQ7 (SEQ ID NO: 15) or a fragment thereof. SEQ ID NO: 15 is provided below. In certain embodiments, the human PD-L1 comprises an extracellular domain, a transmembrane domain, and a cytoplasmic domain. In certain embodiments, the extracellular domain of the human PD-L1 comprises or consists of amino acids 19 to 238 of SEQ ID NO: 15. In certain embodiments, the transmembrane domain comprises or consists of amino acids 239 to 259 of SEQ ID NO: 15. In certain embodiments, the cytoplasmic domain comprises or consists of amino acids 260 to 290 of SEQ ID NO: 15.

[SEQ ID NO: 15]
MRIFAVFIFMTYWHLLNAFTVTVPKDLYVVEYGSNMTIECKFPVEKQLDLAALIVYWEMEDKNIIQFV
HGEEDLKVQHSSYRQRARLLKDQLSLGNAALQITDVKLQDAGVYRCMISYGGADYKRITVKVNAPYNK
INQRILVVDPVTSEHELTCQAEGYPKAEVIWTSSDHQVLSGKTTTTNSKREEKLFNVTSTLRINTTTN
EIFYCTFRRLDPEENHTAELVIPELPLAHPPNERTHLVILGAILLCLGVALTFIFRLRKGRMMDVKKC
GIQDTNSKKQSDTHLEET

In certain embodiments, the human PD-L1 comprises or consists of an amino acid sequence that is at least about 80%, at least about 85%, at least about 90%, at least about 95%, at least about 96%, at least about 97%, at least about 98%, or at least about 99%, at least about 100% identical to the amino acid sequence set forth in SEQ ID NO: 15 or a fragment thereof.

In certain embodiments, the presently disclosed anti-PD-L1 antigen-binding fragments bind to a portion of human PD-L1. In certain embodiments, the presently disclosed anti-PD-L1 antigen-binding fragments bind to the extracellular domain of PD-L1 (e.g., the extracellular domain of the human PD-L1). In certain embodiments, the presently disclosed anti-PD-L1 antigen-binding fragments bind to amino acids 19 to 238 of SEQ ID NO: 15.

In certain embodiments, the anti-PD-L1 antigen-binding fragment comprises antigen-binding fragment from atezolizumab, avelumab, or durvalumab. In certain embodiments, the anti-PD-L1 antigen-binding fragment is an scFv. In certain embodiments, the scFv is atezolizumab scFv. In certain embodiments, the anti-PD-L1 antigen-binding fragment comprises CDRs or VH and VL regions selected from Table 1. In certain embodiments, the anti-PD-L1 antigen-binding fragment comprises a VH comprising a CDR1 comprising the amino acid sequence set forth in SEQ ID NO: 16 or a conservative modification thereof, a CDR2 comprising the amino acid sequence set forth in SEQ ID NO: 17 or a conservative modification thereof, and a CDR3 comprising the amino acid sequence set forth in SEQ ID NO: 18 or a conservative modification thereof. SEQ ID NOs: 16-18 are provided in Table 1.

In certain embodiments, the anti-PD-L1 antigen-binding fragment comprises a VL comprising a CDR1 comprising the amino acid sequence set forth in SEQ ID NO: 19 or a conservative modification thereof, a CDR2 comprising the amino acid sequence set forth in SEQ ID NO: 20 or a conservative modification thereof, and a CDR3 comprising the amino acid sequence set forth in SEQ ID NO: 21 or a conservative modification thereof. SEQ ID NOs: 19-21 are provided in Table 1.

In certain embodiments, the anti-PD-L1 antigen-binding fragment comprises a VH comprising a CDR1 comprising the amino acid sequence set forth in SEQ ID NO: 16 or a conservative modification thereof, a CDR2 comprising the amino acid sequence set forth in SEQ ID NO: 17 or a conservative modification thereof, a CDR3 comprising the amino acid sequence set forth in SEQ ID NO: 18 or a conservative modification thereof, and a VL comprising a CDR1 comprising the amino acid sequence set forth in SEQ ID NO: 19 or a conservative modification thereof, a CDR2 comprising the amino acid sequence set forth in SEQ ID NO: 20 or a conservative modification, and a CDR3 comprising the amino acid sequence set forth in SEQ ID NO: 21 or a conservative modification thereof.

In certain embodiments, the anti-PD-L1 antigen-binding fragment comprises a VH comprising a CDR1 comprising the amino acid sequence set forth in SEQ ID NO: 16, a CDR2 comprising the amino acid sequence set forth in SEQ ID NO: 17, a CDR3 comprising the amino acid sequence set forth in SEQ ID NO: 18; and a VL comprising a CDR1 comprising the amino acid sequence set forth in SEQ ID NO: 19, a CDR2 comprising the amino acid sequence set forth in SEQ ID NO: 20, and a CDR3 comprising the amino acid sequence set forth in SEQ ID NO: 21.

In certain embodiments, the anti-PD-L1 antigen-binding fragment comprises a VH comprising the amino acid sequence set forth in SEQ ID NO: 22. In certain embodiments, the anti-PD-L1 antigen-binding fragment comprises a VL comprising the amino acid sequence set forth in SEQ ID NO: 23. SEQ ID NO: 22 and 23 are provided in Table 1.

In certain embodiments, the anti-PD-L1 antigen-binding fragment comprises a VH comprising the amino acid sequence set forth in SEQ ID NO: 22 and a VL comprising the amino acid sequence set forth in SEQ ID NO: 23.

In certain embodiments, the anti-PD-L1 antigen-binding fragment comprises a VH comprising the amino acid sequence set forth in SEQ ID NO: 22, and a VL comprising the amino acid sequence set forth in SEQ ID NO: 23. In certain embodiments, the VH and VL are linked via a linker. In certain embodiments, the linker comprises the amino acid sequence set forth in SEQ ID NO: 1, SEQ ID NO: 2, SEQ ID NO: 3, SEQ ID NO: 4, SEQ ID NO: 5, SEQ ID NO: 6, or SEQ ID NO: 26. In certain embodiments, the linker comprises the amino acid sequence set forth in SEQ ID NO: 26.

In certain embodiments, the variable regions are linked one after another such that a heavy chain variable region (VH) is positioned at the N-terminus. In certain embodiments, the variable regions are positioned from the N- to the C-terminus: VH-VL. In certain embodiments, a light chain variable region (VL) is positioned at the N-terminus. In certain embodiments, the variable regions are positioned from the N- to the C-terminus: VL-VH.

TABLE 1

| CDRS | 1 | 2 | 3 |
|---|---|---|---|
| V_{H} | DSWIH | WISPYGGSTYYADSVKG | RHWPGGFDY |
| | [SEQ ID NO: 16] | [SEQ ID NO: 17] | [SEQ ID NO: 18] |
| V_{L} | QDVSTA | SA | QQYLYHPAT |
| | [SEQ ID NO: 19] | [SEQ ID NO: 20] | [SEQ ID NO: 21] |

| | |
|---|---|
| FULL | EVQLVESGGGLVQPGGSLRLSCAASGFTFSDSWIHWVRQAPGKGLEWVAWISPYGGSTY |
| V_{H} | YADSVKGRFTISADTSKNTAYLQMNSLRAEDTAVYYCARRHWPGGFDYWGQGTLVTVSS |
| | [SEQ ID NO: 22] |
| FULL | DIQMTQSPSSLSASVGDRVTITCRASQDVSTAVAWYQQKPGKAPKLLIYSASFLYSGVP |
| V_{L} | SRFSGSGSGTDFTLTISSLQPEDFATYYCQQYLYHPATFGQGTKVEIKRTV [SEQ ID |
| | NO: 23] |
| SCFV | EVQLVESGGGLVQPGGSLRLSCAASGFTFSDSWIHWVRQAPGKGLEWVAWISPYGGSTY |
| | YADSVKGRFTISADTSKNTAYLQMNSLRAEDTAVYYCARRHWPGGFDYWGQGTLVTVSS |
| | GGGGSGGGGSGGGGSDIQMTQSPSSLSASVGDRVTITCRASQDVSTAVAWYQQKPGKAP |
| | KLLIYSASFLYSGVPSRFSGSGSGTDFTLTISSLQPEDFATYYCQQYLYHPATFGQGTK |
| | VEIKRTV [SEQ ID NO: 24] |
| DNA | GAGGTGCAGCTGGTGGAGTCTGGGGGTGGCCTGGTGCAGCCTGGGGGCTCACTGAGGCT |
| SCFV | TTCCTGCGCCGCTTCTGGCTTTACATTCAGTGACAGCTGGATTCACTGGGTGAGGCAGG |
| | CTCCTGGAAAGGGTCTTGAGTGGGTTGCATGGATTTCTCCTTACGGTGGTAGCACTTAC |
| | TACGCTGACAGCGTGAAGGGTCGATTCACAATCTCCGCAGACACCTCCAAGAACACAGC |
| | CTACCTGCAGATGAACAGCCTACGGGCCGAGGACACCGCAGTCTATTACTGTGCAAGAA |
| | GACACTGGCCTGGTGGATTTGACTACTGGGGTCAAGGGACCCTGGTCACCGTCTCCTCA |
| | GGTGGAGGTGGATCAGGTGGAGGTGGATCTGGTGGAGGTGGATCTGACATTCAGATGAC |
| | CCAGTCTCCATCATCCCTGTCCGCATCAGTAGGAGACAGGGTCACCATCACCTGCCGGG |
| | CCAGTCAGGATGTGTCAACTGCTGTAGCCTGGTATCAACAGAAACCAGGAAAGGCCCCT |
| | AAACTGCTGATTTACTCGGCCAGCTTTCTGTACAGTGGAGTCCCTAGTCGCTTCAGCGG |
| | CAGTGGATCTGGGACAGATTTCACTCTCACCATCTCTTCCCTGCAGCCTGAGGACTTCG |
| | CAACCTATTACTGTCAACAATATCTGTACCACCCGGCCACGTTCGGACAGGGGACCAAG |
| | GTGGAGATCAAACGGACCGTG [SEQ ID NO: 25] |

The VH and/or VL amino acid sequences having at least about 80%, at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94% about 95% about 96%, about 97% about 98%, or about 99%) homology or identity to a specific sequence (e.g., SEQ ID NOs: 22 and 23) may contain substitutions (e.g., conservative substitutions), insertions, or deletions relative to the specified sequence(s), but retain the ability to bind to a target antigen (e.g., PD-L1). In certain embodiments, a total of 1 to 10 amino acids are substituted, inserted and/or deleted in a specific sequence (e.g., SEQ ID NOs: 22 and 23). In certain embodiments, substitutions, insertions, or deletions occur in regions outside the CDRs (e.g., in the FRs) of the antigen-binding fragment. In certain embodiments, the antigen-binding fragment comprises V_{H} and/or V_{L} sequence selected from SEQ ID NOs: 22 and 23, including post-translational modifications of that sequence (SEQ ID NO: 22 and 23).

In certain embodiments, the anti-PD-L1 antigen-binding fragment is a nanobody. In certain embodiments, the anti-PD-L1 antigen-binding fragment comprises CDRs selected from Table 2. In certain embodiments, the anti-PD-L1 antigen-binding fragment comprises a CDR1 comprising the amino acid sequence set forth in SEQ ID NO: 27 or a conservative modification thereof, a CDR2 comprising the amino acid sequence set forth in SEQ ID NO: 28 or a conservative modification thereof, and a CDR3 comprising the amino acid sequence set forth in SEQ ID NO: 29 or a conservative modification thereof. SEQ ID NOs: 27-29 are provided in Table 2.

In certain embodiments, the anti-PD-L1 antigen-binding fragment comprises a CDR1 comprising the amino acid sequence set forth in SEQ ID NO: 27, a CDR2 comprising the amino acid sequence set forth in SEQ ID NO: 28, and a CDR3 comprising the amino acid sequence set forth in SEQ ID NO: 29.

In certain embodiments, the anti-PD-L1 antigen-binding fragment comprises the amino acid sequence set forth in SEQ ID NO: 30 or a conservative modification thereof. In certain embodiments, the anti-PD-L1 antigen-binding fragment comprises the amino acid sequence set forth in SEQ ID NO: 30. SEQ ID NO: 30 is provided in Table 2.

TABLE 2

| CDRS | 1 | 2 | 3 |
|---|---|---|---|
| | GKMSSRRC | LLTTSGST | AADSFEDPTCTLVT |
| | [SEQ ID NO: 27] | [SEQ ID NO: 28] | SSGAFQY [SEQ ID |
| | | | NO: 29] |

| | |
|---|---|
| FULL | QVQLQESGGGLVQPGGSLRLSCAASGKMSSRRCMAWFRQAPGKERERVAKLLTTSGST |
| LENGTH | YLADSVKGRFTISQNNAKSTVYLQMNSLKPEDTAMYYCAADSFEDPTCTLVTSSGAFQ |
| | YWGQGTQVTVSS [SEQ ID NO: 30] |
| DNA | CAGGTCCAACTGCAAGAGTCTGGTGGGGGTCTTGTCCAACCTGGGGGCTCCCTGAGGC |
| FULL | TCTCATGTGCAGCTAGTGGCAAAATGAGCAGTAGGAGGTGCATGGCCTGGTTCCGGCA |
| LENGTH | GGCACCCGGTAAGGAAAGAGAACGAGTAGCTAAACTTCTGACCACTTCAGGCTCAACC |
| | TACCTCGCTGACAGTGTAAAAGGACGATTCACCATATCACAAAATAATGCTAAATCCA |
| | CTGTGTACCTCCAAATGAATAGTCTGAAGCCTGAGGACACCGCCATGTATTACTGTGC |
| | CGCAGACTCATTTGAAGATCCCACCTGCACACTCGTGACCAGCAGCGGGGCCTTCCAA |
| | TACTGGGGTCAAGGCACCCAAGTGACAGTCTCCAGT [SEQ ID NO: 31] |

The amino acid sequences having at least about 80%, at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homology or identity to a specific sequence (e.g., SEQ ID NOs: 30 and 31) may contain substitutions (e.g., conservative substitutions), insertions, or deletions relative to the specified sequence(s), but retain the ability to bind to a target antigen (e.g., PD-L1). In certain embodiments, a total of 1 to 10 amino acids are substituted, inserted and/or deleted in a specific sequence (e.g., SEQ ID NOs: 30 and 31). In certain embodiments, substitutions, insertions, or deletions occur in regions outside the CDRs (e.g., in the FRs) of the antigen-binding fragment. In certain embodiments, the extracellular antigen-binding fragment comprises sequence selected from SEQ ID NOs: 30 and 31, including post-translational modifications of that sequence (SEQ ID NO: 30 and 31).

### 2.1.2. Co-Stimulatory Ligand Polypeptides

In certain embodiments, the extracellular domain of the presently disclosed fusion polypeptides includes a costimulatory ligand polypeptide. In certain embodiments, the co-stimulatory ligand polypeptide can be a polypeptide selected from the group consisting of tumor necrosis factor (TNF) family members, immunoglobulin (Ig) superfamily members, and combinations thereof. In certain embodiments, the TNF family member can be selected from the group consisting of a 4-1BBL polypeptide, an OX40L polypeptide, a CD70 polypeptide, a GITRL polypeptide, a CD40L polypeptide, and combinations thereof. In certain embodiments, the Ig superfamily member can be selected from the group consisting of a CD80 polypeptide, a CD86 polypeptide, an ICOS ligand (ICOSLG (also known as “CD275”) polypeptide, and combinations thereof.

In certain embodiments, the co-stimulatory ligand is a CD80 polypeptide. In certain embodiments, the CD80 polypeptide is a human CD80 polypeptide. In certain embodiments, the CD80 polypeptide comprises or consists of an amino acid sequence that is at least about 85%, about 90%, about 95%, about 96%, about 97%, about 98%, about 99%, or about 100% identical or homologous to the amino acid sequence having a UniProt Reference No: P33681 (SEQ ID NO: 32) or a fragment thereof, and/or may optionally comprise up to one or up to two or up to three conservative amino acid substitutions. In certain embodiments, the CD80 polypeptide comprises or consists of an amino acid sequence that is a consecutive portion of SEQ ID NO: 32, which is at least about 20, or at least about 30, or at least about 40, at least about 50, at least about 100, at least about 150, at least about 200, at least about 208, at least about 242, at least about 250, and up to about 288 amino acids in length. In certain embodiments, the CD80 polypeptide comprises or consists of the amino acid sequence of amino acids 1 to 288, 1 to 242, 1 to 263, 35 to 288, 35 to 263, or 35 to 242 of SEQ ID NO: 32. In certain embodiments, the CD80 polypeptide comprises or consists of the amino acid sequence of amino acids 35 to 242 of SEQ ID NO: 32. SEQ ID NO: 32 is provided below.

[SEQ ID NO: 32]
MGHTRRQGTSPSKCPYLNFFQLLVLAGLSHFCSGVIHVTKEVKEVATLSCGHNVSVEELAQTRIYWQK
EKKMVLTMMSGDMNIWPEYKNRTIFDITNNLSIVILALRPSDEGTYECVVLKYEKDAFKREHLAEVTL
SVKADFPTPSISDFEIPTSNIRRIICSTSGGFPEPHLSWLENGEELNAINTTVSQDPETELYAVSSKL
DFNMTTNHSFMCLIKYGHLRVNQTFNWNTTKQEHFPDNLLPSWAITLISVNGIFVICCLTYCFAPRCR
ERRRNERLRRESVRPV

In certain embodiments, the CD80 polypeptide comprises the amino acid sequence set forth in SEQ ID NO: 33. In certain embodiments, the CD80 polypeptide consists of the amino acid sequence set forth in SEQ ID NO: 33. SEQ ID NO: 33 is provided below.

[SEQ ID NO: 33]
VIHVTKEVKEVATLSCGHNVSVEELAQTRIYWQKEKKMVLTMMSGDMNIWPEYKNRTIFDITNNLSIV
ILALRPSDEGTYECVVLKYEKDAFKREHLAEVTLSVKADFPTPSISDFEIPTSNIRRIICSTSGGFPE
PHLSWLENGEELNAINTTVSQDPETELYAVSSKLDFNMTTNHSFMCLIKYGHLRVNQTFNWNTTKQEH
FPDN

An exemplary nucleotide sequence encoding the amino acid sequence of SEQ ID NO: 33 is set forth in SEQ ID NO: 129, which is provided below.

[SEQ ID NO: 129]
GTTATCCACGTGACCAAGGAAGTGAAAGAAGTGGCAACGCTGTCCTGTGGTCACAATGTTTCTGTTGA
AGAGCTGGCACAAACTCGCATCTACTGGCAAAAGGAGAAGAAAATGGTGCTGACTATGATGTCTGGGG
ACATGAATATATGGCCCGAGTACAAGAACCGGACCATCTTTGATATCACTAATAACCTCTCCATTGTG
ATCCTGGCTCTGCGCCCATCTGACGAGGGCACATACGAGTGTGTTGTTCTGAAGTATGAAAAAGACGC
TTTCAAGCGGGAACACCTGGCTGAAGTGACGTTATCAGTCAAAGCTGACTTCCCTACACCTAGTATAT
CTGACTTTGAAATTCCAACTTCTAATATTAGAAGGATAATTTGCTCAACCTCTGGAGGTTTTCCAGAG
CCTCACCTCTCCTGGTTGGAAAATGGAGAAGAATTAAATGCCATCAACACAACAGTTTCCCAAGATCC
TGAAACTGAGCTCTATGCTGTTAGCAGCAAACTGGATTTCAATATGACAACCAACCACAGCTTCATGT
GTCTCATCAAGTATGGACATTTAAGAGTGAATCAGACCTTCAACTGGAATACAACCAAGCAAGAGCAT
TTTCCTGATAAC

In certain embodiments, the CD80 polypeptide retains at least about 80%, at least about 85%, at least about 90%, at least about 95%, or at least about 100% of the primary function of a native CD80 polypeptide. Non-limiting examples of the primary functions of the native CD80 polypeptide include binding to/interacting with CD28, binding to/interacting with CTLA-4, binding to/interacting with PD-L1, and contributing to CD80 homodimerization.

In certain embodiments, the co-stimulatory ligand is a 4-1BBL polypeptide. In certain embodiments, the 4-1BBL polypeptide is a human 4-1BBL polypeptide. In certain embodiments, the 4-1BBL polypeptide comprises or consists of an amino acid sequence that is at least about 85%, about 90%, about 95%, about 96%, about 97%, about 98%, about 99%, or about 100% identical or homologous to the amino acid sequence having a UniProt Reference No: P41273 (SEQ ID NO: 34) or a fragment thereof, and/or may optionally comprise up to one or up to two or up to three conservative amino acid substitutions. In certain embodiments, the 4-1BBL polypeptide comprises or consists of an amino acid sequence that is a consecutive portion of SEQ ID NO: 34, which is at least about 20, or at least about 30, or at least about 40, at least about 50, at least about 100, at least about 150, at least about 200, at least about 250, and up to about 254 amino acids in length. In certain embodiments, the 4-1BBL polypeptide comprises or consists of the amino acid sequence of amino acids 1 to 254, 29 to 254, or 50 to 254 of SEQ ID NO: 34. In certain embodiments, the 4-1BBL polypeptide comprises or consists of the amino acid sequence of amino acids 50 to 254 of SEQ ID NO: 34. SEQ ID NO: 34 is provided below.

[SEQ ID NO: 34]
MEYASDASLDPEAPWPPAPRARACRVLPWALVAGLLLLLLLAAACAVFLACPWAVSGARASPGSAASP
RLREGPELSPDDPAGLLDLRQGMFAQLVAQNVLLIDGPLSWYSDPGLAGVSLTGGLSYKEDTKELVVA
KAGVYYVFFQLELRRVVAGEGSGSVSLALHLQPLRSAAGAAALALTVDLPPASSEARNSAFGFQGRLL
HLSAGQRLGVHLHTEARARHAWQLTQGATVLGLFRVTPEIPAGLPSPRSE

In certain embodiments, the co-stimulatory ligand is an OX-40L polypeptide. In certain embodiments, the OX-40L polypeptide is a human OX-40L polypeptide. In certain embodiments, the OX-40L polypeptide comprises or consists of an amino acid sequence that is at least about 85%, about 90%, about 95%, about 96%, about 97%, about 98%, about 99%, or about 100% identical or homologous to the amino acid sequence having a UniProt Reference No: P23510 (SEQ ID NO: 35) or a fragment thereof, and/or may optionally comprise up to one or up to two or up to three conservative amino acid substitutions. In certain embodiments, the OX-40L polypeptide comprises or consists of an amino acid sequence that is a consecutive portion of SEQ ID NO: 35, which is at least about 20, or at least about 30, or at least about 40, or at least about 50, and up to about 183 amino acids in length. In certain embodiments, the OX-40L polypeptide comprises or consists of the amino acid sequence of amino acids 1 to 183, 24 to 183, or 51 to 183 of SEQ ID NO: 35. In certain embodiments, the OX-40L polypeptide comprises or consists of the amino acid sequence of amino acids 51 to 183 of SEQ ID NO: 35. SEQ ID NO: 35 is provided below.

[SEQ ID NO: 35]
MERVQPLEENVGNAARPRFERNKLLLVASVIQGLGLLLCFTYICLHFSALQVSHRYPRIQSIKVQFTE
YKKEKGFILTSQKEDEIMKVQNNSVIINCDGFYLISLKGYFSQEVNISLHYQKDEEPLFQLKKVRSVN
SLMVASLTYKDKVYLNVTTDNTSLDDFHVNGGELILIHQNPGEFCVL

In certain embodiments, the co-stimulatory ligand is a CD70 polypeptide. In certain embodiments, the CD70 polypeptide is a human CD70 polypeptide. In certain embodiments, the CD70 polypeptide comprises or consists of an amino acid sequence that is at least about 85%, about 90%, about 95%, about 96%, about 97%, about 98%, about 99%, or about 100% identical or homologous to the amino acid sequence having a UniProt Reference No: P32970 (SEQ ID NO: 36) or a fragment thereof, and/or may optionally comprise up to one or up to two or up to three conservative amino acid substitutions. In certain embodiments, the CD70 polypeptide comprises or consists of an amino acid sequence that is a consecutive portion of SEQ ID NO: 36, which is at least about 20, or at least about 30, or at least about 40, or at least about 50, and up to about 193 amino acids in length. In certain embodiments, the CD70 polypeptide comprises or consists of the amino acid sequence of amino acids 1 to 193, 18 to 193, or 39 to 193 of SEQ ID NO: 36. In certain embodiments, the CD70 polypeptide comprises or consists of the amino acid sequence of amino acids 39 to 193 of SEQ ID NO: 36. SEQ ID NO: 36 is provided below.

[SEQ ID NO: 36]
MPEEGSGCSVRRRPYGCVLRAALVPLVAGLVICLVVCIQRFAQAQQQLPLESLGWDVAELQLNHTGPQ
QDPRLYWQGGPALGRSFLHGPELDKGQLRIHRDGIYMVHIQVTLAICSSTTASRHHPTTLAVGICSPA
SRSISLLRLSFHQGCTIASQRLTPLARGDTLCTNLTGTLLPSRNTDETFFGVQWVRP

In certain embodiments, the co-stimulatory ligand is a CD86 polypeptide. In certain embodiments, the CD86 polypeptide is a human CD86 polypeptide. In certain embodiments, the CD86 polypeptide comprises or consists of an amino acid sequence that is at least about 85%, about 90%, about 95%, about 96%, about 97%, about 98%, about 99%, or about 100% identical or homologous to the amino acid sequence having a UniProt Reference No: P42081 (SEQ ID NO: 37) or a fragment thereof, and/or may optionally comprise up to one or up to two or up to three conservative amino acid substitutions. In certain embodiments, the CD86 polypeptide comprises or consists of an amino acid sequence that is a consecutive portion of SEQ ID NO: 37, which is at least about 20, or at least about 30, or at least about 40, or at least about 50, and up to about 247 amino acids in length. In certain embodiments, the CD86 polypeptide comprises or consists of the amino acid sequence of amino acids 1 to 329, 24 to 329, 24 to 268, or 24 to 247 of SEQ ID NO: 37. In certain embodiments, the CD86 polypeptide comprises or consists of the amino acid sequence of amino acids 24 to 247 of SEQ ID NO: 37. SEQ ID NO: 37 is provided below.

[SEQ ID NO: 37]
MDPQCTMGLSNILFVMAFLLSGAAPLKIQAYFNETADLPCQFANSQNQSLSELVVFWQDQENLVLNEV
YLGKEKFDSVHSKYMGRTSFDSDSWTLRLHNLQIKDKGLYQCIIHHKKPTGMIRIHQMNSELSVLANF
SQPEIVPISNITENVYINLTCSSIHGYPEPKKMSVLLRTKNSTIEYDGVMQKSQDNVTELYDVSISLS
VSFPDVTSNMTIFCILETDKTRLLSSPFSIELEDPQPPPDHIPWITAVLPTVIICVMVFCLILWKWKK
KKRPRNSYKCGTNTMEREESEQTKKREKIHIPERSDEAQRVFKSSKTSSCDKSDTCF

In certain embodiments, the co-stimulatory ligand is a GITRL polypeptide. In certain embodiments, the GITRL polypeptide is a human GITRL polypeptide. In certain embodiments, the GITRL polypeptide comprises or consists of an amino acid sequence that is at least about 85%, about 90%, about 95%, about 96%, about 97%, about 98%, about 99% or about 100% identical or homologous to the amino acid sequence having a UniProt Reference No: Q9UNG2 (SEQ ID NO: 38) or a fragment thereof, and/or may optionally comprise up to one or up to two or up to three conservative amino acid substitutions. In certain embodiments, the GITRL polypeptide comprises or consists of an amino acid sequence that is a consecutive portion of SEQ ID NO: 38, which is at least about 20, or at least about 30, or at least about 40, or at least about 50, and up to about 177 amino acids in length. In certain embodiments, the GITRL polypeptide comprises or consists of the amino acid sequence of amino acids 1 to 177, 28 to 177, or 49 to 177 of SEQ ID NO: 38. In certain embodiments, the GITRL polypeptide comprises or consists of the amino acid sequence of amino acids 49 to 177 of SEQ ID NO: 38. SEQ ID NO: 38 is provided below.

[SEQ ID NO: 38]
MCLSHLENMPLSHSRTQGAQRSSWKLWLFCSIVMLLFLCSFSWLIFIFLQLETAKEPCMAKFGPLPSK
WQMASSEPPCVNKVSDWKLEILQNGLYLIYGQVAPNANYNDVAPFEVRLYKNKDMIQTLINKSKIQNV
GGTYELHVGDTIDLIFNSEHQVLKNNTYWGIILLANPQFIS

In certain embodiments, the co-stimulatory ligand is an ICOSL polypeptide. In certain embodiments, the ICOSL polypeptide is a human ICOSL polypeptide. In certain embodiments, the ICOSL polypeptide comprises or consists of an amino acid sequence that is at least about 85%, about 90%, about 95%, about 96%, about 97%, about 98%, about 99%, or about 100% identical or homologous to the amino acid sequence having a UniProt Reference No: 075144 (SEQ ID NO: 39) or a fragment thereof, and/or may optionally comprise up to one or up to two or up to three conservative amino acid substitutions. In certain embodiments, the ICOSL polypeptide comprises or consists of an amino acid sequence that is a consecutive portion of SEQ ID NO: 39, which is at least about 20, or at least about 30, or at least about 40, or at least about 50, and up to about 256 amino acids in length. In certain embodiments, the ICOSL polypeptide comprises or consists of the amino acid sequence of amino acids 1 to 302, 1 to 256, or 19 to 256 of SEQ ID NO: 39. In certain embodiments, the ICOSL polypeptide comprises or consists of the amino acid sequence of amino acids 19 to 256 of SEQ ID NO: 39. SEQ ID NO: 39 is provided below.

[SEQ ID NO: 39]
MRLGSPGLLFLLFSSLRADTQEKEVRAMVGSDVELSCACPEGSRFDLNDVYVYWQTSESKTVVTYHIP
QNSSLENVDSRYRNRALMSPAGMLRGDFSLRLFNVTPQDEQKFHCLVLSQSLGFQEVLSVEVTLHVAA
NFSVPVVSAPHSPSQDELTFTCTSINGYPRPNVYWINKTDNSLLDQALQNDTVFLNMRGLYDVVSVLR
IARTPSVNIGCCIENVLLQQNLTVGSQTGNDIGERDKITENPVSTGEKNAATWSILAVLCLLVVVAVA
IGWVCRDRCLQHSYAGAWAVSPETELTGHV

In certain embodiments, the co-stimulatory ligand is a CD40L polypeptide. In certain embodiments, the CD40L polypeptide is a human CD40L polypeptide. In certain embodiments, the CD40L polypeptide comprises or consists of an amino acid sequence that is at least about 85%, about 90%, about 95%, about 96%, about 97%, about 98%, about 99%, or about 100% identical or homologous to the amino acid sequence having a UniProt Reference No: P29965 (SEQ ID NO: 40) or a fragment thereof, and/or may optionally comprise up to one or up to two or up to three conservative amino acid substitutions. In certain embodiments, the CD40L polypeptide comprises or consists of an amino acid sequence that is a consecutive portion of SEQ ID NO: 40, which is at least about 20, or at least about 30, or at least about 40, or at least about 50, and up to about 261 amino acids in length. In certain embodiments, the CD40L polypeptide comprises or consists of the amino acid sequence of amino acids 1 to 261, 23 to 261, or 47 to 261 of SEQ ID NO: 40. In certain embodiments, the CD40L polypeptide comprises or consists of the amino acid sequence of amino acids 47 to 261 of SEQ ID NO: 40. SEQ ID NO: 40 is provided below.

[SEQ ID NO: 40]
MIETYNQTSPRSAATGLPISMKIFMYLLTVFLITQMIGSALFAVYLHRRLDKIEDERNLHEDFVFMKT
IQRCNTGERSLSLLNCEEIKSQFEGFVKDIMLNKEETKKENSFEMQKGDQNPQIAAHVISEASSKTTS
VLQWAEKGYYTMSNNLVTLENGKQLTVKRQGLYYIYAQVTFCSNREASSQAPFIASLCLKSPGRFERI
LLRAANTHSSAKPCGQQSIHLGGVFELQPGASVFVNVTDPSQVSHGTGFTSFGLLKL

In certain embodiments, the extracellular domain of the presently disclosed fusion polypeptides comprises a second co-stimulatory ligand polypeptide. In certain embodiments, the extracellular domain of the presently disclosed fusion polypeptides comprises a third co-stimulatory ligand polypeptide. In certain embodiments, the extracellular domain of the presently disclosed fusion polypeptides comprises a fourth co-stimulatory ligand polypeptide. In certain embodiments, the extracellular domain of the presently disclosed fusion polypeptides comprises a fifth co-stimulatory ligand polypeptide. In certain embodiments, the first, second, third, fourth, and fifth co-stimulatory ligand polypeptides can be the same or different from each other.

In certain embodiments, the extracellular domain of the presently disclosed fusion polypeptides comprises a CD80 polypeptide.

In certain embodiments, the antigen-binding fragment and the co-stimulatory ligand polypeptide are linked via a linker. In certain embodiments, the linker comprises the amino acid sequence set forth in SEQ ID NO: 1, SEQ ID NO: 2, SEQ ID NO: 3, SEQ ID NO: 4, SEQ ID NO: 5, SEQ ID NO: 6, or SEQ ID NO: 26. In certain embodiments, the linker comprises the amino acid sequence set forth in SEQ ID NO: 26. In certain embodiments, the antigen-binding fragment and the co-stimulatory ligand polypeptide are linked one after another such that the antigen-binding fragment is positioned at the N-terminus.

### 2.1.3. Signal Peptide

In addition, the extracellular domain of the presently disclosed fusion polypeptide can comprise a leader or a signal peptide that directs the nascent protein into the endoplasmic reticulum. A signal peptide or leader can be essential if the fusion polypeptide is to be glycosylated and anchored in the cell membrane. The signal sequence or leader can be a peptide sequence (about 5, about 10, about 15, about 20, about 25, or about 30 amino acids long) present at the N-terminus of newly synthesized proteins that directs their entry to the secretory pathway. In certain embodiments, the signal peptide is covalently joined to the 5′ terminus (N-terminus) of the extracellular domain of the presently disclosed fusion polypeptide. In certain embodiments, the signal peptide comprises a CD8 polypeptide, e.g., the fusion polypeptide comprises a truncated CD8 signal peptide. In certain embodiments, the signal peptide comprises or consists of the amino acid sequence set forth in SEQ ID NO: 12. An exemplary nucleotide sequence encoding the amino acid sequence of SEQ ID NO: 12 is set forth in SEQ ID NO: 128, which is provided below.

[SEQ ID NO:128]
ATGGCTCTCCCAGTGACTGCCCTACTGCTTCCCCTAGCGCTTCTCCTGC
ATGCAG

### 2.2. Intracellular Domain of the Fusion Polypeptides

In certain embodiments, the fusion polypeptide comprises an extracellular domain and an intracellular domain. In certain embodiments, the intracellular domain comprises a first co-stimulatory molecule polypeptide.

### 2.2.1. Co-stimulatory Molecule Polypeptides

In certain embodiments, the intracellular domain of the presently disclosed fusion polypeptides includes a costimulatory molecule polypeptide. Non-limiting examples of co-stimulatory molecule polypeptides include CD28, 4-1BB, OX40, ICOS, DAP-10, CD27, CD40, NKG2D, CD2, and combinations thereof.

In certain embodiments, the co-stimulatory molecule polypeptide is a 4-1BB polypeptide. In certain embodiments, the 4-1BB polypeptide is a human 4-1BB polypeptide. In certain embodiments, the 4-1BB polypeptide comprises or consists of an amino acid sequence that is at least about 85%, about 90%, about 95%, about 96%, about 97%, about 98%, about 99%, or about 100% identical or homologous to the amino acid sequence having a UniProt Reference No: Q07011 (SEQ ID NO: 41) or a fragment thereof, and/or may optionally comprise up to one or up to two or up to three conservative amino acid substitutions. In certain embodiments, the 4-1BB polypeptide comprises or consists of an amino acid sequence that is a consecutive portion of SEQ ID NO: 41, which is at least about 20, or at least about 30, at least about 40, at least about 42, at least about 50, at least about 100, at least about 150, at least about 200, at least about 214, at least about 242, at least about 250, and up to about 255 amino acids in length. In certain embodiments, the 4-1BB polypeptide comprises or consists of the amino acid sequence of amino acids 1 to 255, 24 to 255, 187 to 255, or 214 to 255 of SEQ ID NO: 41. In certain embodiments, the 4-1BB polypeptide comprises or consists of the amino acid sequence of amino acids 214 to 255 of SEQ ID NO: 41. SEQ ID NO: 41 is provided below.

[SEQ ID NO: 41]
MGNSCYNIVATLLLVLNFERTRSLQDPCSNCPAGTFCDNNRNQICSPCPPNSFSSAGGQRTCDICRQC
KGVFRTRKECSSTSNAECDCTPGFHCLGAGCSMCEQDCKQGQELTKKGCKDCCFGTFNDQKRGICRPW
TNCSLDGKSVLVNGTKERDVVCGPSPADLSPGASSVTPPAPAREPGHSPQIISFFLALTSTALLFLLF
FLTLRFSVVKRGRKKLLYIFKQPFMRPVQTTQEEDGCSCRFPEEEEGGCEL

In certain embodiments, the 4-1BB polypeptide comprises the amino acid sequence set forth in SEQ ID NO: 42. In certain embodiments, the 4-1BB polypeptide consists of the amino acid sequence set forth in SEQ ID NO: 42. SEQ ID NO: 42 is provided below.

[SEQ ID NO: 42]
KRGRKKLLYIFKQPFMRPVQTTQEEDGCSCRFPEEEEGGCEL

An exemplary nucleotide sequence encoding the amino acid sequence of SEQ ID NO: 42 is set forth in SEQ ID NO: 132, which is provided below.

[SEQ ID NO: 132]
AAACGGGGCAGAAAGAAACTCCTGTATATATTCAAACAACCATTTATGA
GACCAGTACAAACTACTCAAGAGGAAGATGGCTGTAGCTGCCGATTTCC
AGAAGAAGAAGAAGGAGGATGTGAACTGTGA

In certain embodiments, the 4-1BB polypeptide retains at least about 80%, at least about 85%, at least about 90%, at least about 95%, or at least about 100% of the primary function of a native 4-1BB polypeptide. Non-limiting examples of the primary functions of the intracellular domain of 4-1BB include providing co-stimulatory signaling for the activation and proliferation of an immunoresponsive cell (e.g., a T cell), and interacting and activating downstream adaptors (e.g., TRAFs).

In certain embodiments, the co-stimulatory molecule polypeptide is a CD28 polypeptide. In certain embodiments, the CD28 polypeptide is a human CD28 polypeptide. In certain embodiments, the CD28 polypeptide comprises or consists of an amino acid sequence that is at least about 85%, about 90%, about 95%, about 96%, about 97%, about 98%, about 99%, or about 100% identical or homologous to the amino acid sequence having a UniProt Reference No: P10747 (SEQ ID NO: 43) or a fragment thereof, and/or may optionally comprise up to one or up to two or up to three conservative amino acid substitutions. In certain embodiments, the CD28 polypeptide comprises or consists of an amino acid sequence that is a consecutive portion of SEQ ID NO: 43, which is at least about 20, or at least about 30, at least about 40, at least about 50, at least about 100, at least about 150, at least about 180, at least about 200, and up to about 220 amino acids in length. In certain embodiments, the CD28 polypeptide comprises or consists of the amino acid sequence of amino acids 1 to 220, 19 to 220, 153 to 220, or 180 to 220 of SEQ ID NO: 43. In certain embodiments, the CD28 polypeptide comprises or consists of the amino acid sequence of amino acids 180 to 220 of SEQ ID NO: 43. SEQ ID NO: 43 is provided below.

[SEQ ID NO: 43]
MLRLLLALNLFPSIQVTGNKILVKQSPMLVAYDNAVNLSCKYSYNLFSREFRASLHKGLDSAVEVCVV
YGNYSQQLQVYSKTGFNCDGKLGNESVTFYLQNLYVNQTDIYFCKIEVMYPPPYLDNEKSNGTIIHVK
GKHLCPSPLFPGPSKPFWVLVVVGGVLACYSLLVTVAFIIFWVRSKRSRLLHSDYMNMTPRRPGPTRK
HYQPYAPPRDFAAYRS

In certain embodiments, the CD28 polypeptide comprises the amino acid sequence set forth in SEQ ID NO: 44. In certain embodiments, the CD28 polypeptide consists of the amino acid sequence set forth in SEQ ID NO: 44. SEQ ID NO: 44 is provided below.

[SEQ ID NO: 44]
RSKRSRLLHSDYMNMTPRRPGPTRKHYQPYAPPRDFAAYR

In certain embodiments, the CD28 polypeptide retains at least about 80%, at least about 85%, at least about 90%, at least about 95%, or at least about 100% of the primary function of a native CD28 polypeptide. Non-limiting examples of the primary functions of the intracellular domain of CD28 include providing co-stimulatory signaling for the activation and proliferation of an immunoresponsive cell (e.g., a T cell), and interacting with protein adaptors (e.g., PI3K, GRB2, and LCK).

In certain embodiments, the co-stimulatory molecule polypeptide is an OX40 polypeptide. In certain embodiments, the OX40 polypeptide is a human OX40 polypeptide. In certain embodiments, the OX40 polypeptide comprises or consists of an amino acid sequence that is at least about 85%, about 90%, about 95%, about 96%, about 97%, about 98%, about 99%, or about 100% identical or homologous to the amino acid sequence having a UniProt Reference No: P43489 (SEQ ID NO: 45) or a fragment thereof, and/or may optionally comprise up to one or up to two or up to three conservative amino acid substitutions. In certain embodiments, the OX40 polypeptide comprises or consists of an amino acid sequence that is a consecutive portion of SEQ ID NO: 45, which is at least about 20, or at least about 30, or at least about 40, or at least about 50, and up to about 277 amino acids in length. In certain embodiments, the OX40 polypeptide comprises or consists of the amino acid sequence of amino acids 1 to 277, 29 to 277, 215 to 277, or 236 to 277 of SEQ ID NO: 45. In certain embodiments, the OX40 polypeptide comprises or consists of the amino acid sequence of amino acids 236 to 277 of SEQ ID NO: 45. SEQ ID NO: 45 is provided below.

[SEQ ID NO: 45]
MCVGARRLGRGPCAALLLLGLGLSTVTGLHCVGDTYPSNDRCCHECRPGNGMVSRCSRSQNTVCRPCG
PGFYNDVVSSKPCKPCTWCNLRSGSERKQLCTATQDTVCRCRAGTQPLDSYKPGVDCAPCPPGHFSPG
DNQACKPWTNCTLAGKHTLQPASNSSDAICEDRDPPATQPQETQGPPARPITVQPTEAWPRTSQGPST
RPVEVPGGRAVAAILGLGLVLGLLGPLAILLALYLLRRDQRLPPDAHKPPGGGSFRTPIQEEQADAHS
TLAKI

In certain embodiments, the co-stimulatory molecule polypeptide is an ICOS polypeptide. In certain embodiments, the ICOS polypeptide is a human ICOS polypeptide. In certain embodiments, the ICOS polypeptide comprises or consists of an amino acid sequence that is at least about 85%, about 90%, about 95%, about 96%, about 97%, about 98%, about 99%, or about 100% identical or homologous to the amino acid sequence having a UniProt Reference No: Q9Y6W8 (SEQ ID NO: 46) or a fragment thereof, and/or may optionally comprise up to one or up to two or up to three conservative amino acid substitutions. In certain embodiments, the ICOS polypeptide comprises or consists of an amino acid sequence that is a consecutive portion of SEQ ID NO: 46, which is at least about 20, or at least about 30, or at least about 40, or at least about 50, and up to about 199 amino acids in length. In certain embodiments, the ICOS polypeptide comprises or consists of the amino acid sequence of amino acids 1 to 199, 21 to 199, 141 to 199, or 162 to 199 of SEQ ID NO: 46. In certain embodiments, the ICOS polypeptide comprises or consists of the amino acid sequence of amino acids 162 to 199 of SE ID NO: 46. SE ID NO: 46 is provided below.

[SEQ ID NO: 46]
MKSGLWYFFLFCLRIKVLTGEINGSANYEMFIFHNGGVQILCKYPDIVQQFKMQLLKGGQILCDLTKT
KGSGNTVSIKSLKFCHSQLSNNSVSFFLYNLDHSHANYYFCNLSIFDPPPFKVTLTGGYLHIYESQLC
CQLKFWLPIGCAAFVVVCILGCILICWLTKKKYSSSVHDPNGEYMFMRAVNTAKKSRLTDVTL

In certain embodiments, the co-stimulatory molecule polypeptide is a DAP-10 polypeptide. In certain embodiments, the DAP-10 polypeptide is a human DAP-10 polypeptide. In certain embodiments, the DAP-10 polypeptide comprises or consists of an amino acid sequence that is at least about 85%, about 90%, about 95%, about 96%, about 97%, about 98%, about 99%, or about 100% identical or homologous to the amino acid sequence having a UniProt Reference No: Q9UBK5 (SEQ ID NO: 47) or a fragment thereof, and/or may optionally comprise up to one or up to two or up to three conservative amino acid substitutions. In certain embodiments, the DAP-10 polypeptide comprises or consists of an amino acid sequence that is a consecutive portion of SEQ ID NO: 47, which is at least about 20, or at least about 30, or at least about 40, or at least about 50, and up to about 93 amino acids in length. In certain embodiments, the DAP-10 polypeptide comprises or consists of the amino acid sequence of amino acids 1 to 93, 19 to 93, 49 to 93, or 70 to 93 of SEQ ID NO: 47. In certain embodiments, the DAP-10 polypeptide comprises or consists of the amino acid sequence of amino acids 70 to 93 of SEQ ID NO: 47. SEQ ID NO: 47 is provided below.

[SEQ ID NO: 47]
MIHLGHILFLLLLPVAAAQTTPGERSSLPAFYPGTSGSCSGCGSLSLPL
LAGLVAADAVASLLIVGAVFLCARPRRSPAQEDGKVYINMPGRG

In certain embodiments, the co-stimulatory molecule polypeptide is a CD27 polypeptide. In certain embodiments, the CD27 polypeptide is a human CD27 polypeptide. In certain embodiments, the CD27 polypeptide comprises or consists of an amino acid sequence that is at least about 85%, about 90%, about 95%, about 96%, about 97%, about 98%, about 99%, or about 100% identical or homologous to the amino acid sequence having a UniProt Reference No: P26842 (SEQ ID NO: 48) or a fragment thereof, and/or may optionally comprise up to one or up to two or up to three conservative amino acid substitutions. In certain embodiments, the CD27 polypeptide comprises or consists of an amino acid sequence that is a consecutive portion of SEQ ID NO: 48, which is at least about 20, or at least about 30, or at least about 40, or at least about 50, and up to about 260 amino acids in length. In certain embodiments, the CD27 polypeptide comprises or consists of the amino acid sequence of amino acids 1 to 260, 192 to 260, or 213 to 260 of SEQ ID NO: 48. In certain embodiments, the CD27 polypeptide comprises or consists of the amino acid sequence of amino acids 213 to 260 of SEQ ID NO: 48. SEQ ID NO: 48 is provided below.

[SEQ ID NO: 48]
MARPHPWWLCVLGTLVGLSATPAPKSCPERHYWAQGKLCCQMCEPGTFLVKDCDQHRKAAQCDPCIPG
VSFSPDHHTRPHCESCRHCNSGLLVRNCTITANAECACRNGWQCRDKECTECDPLPNPSLTARSSQAL
SPHPQPTHLPYVSEMLEARTAGHMQTLADFRQLPARTLSTHWPPQRSLCSSDFIRILVIFSGMFLVFT
LAGALFLHQRRKYRSNKGESPVEPAEPCHYSCPREEEGSTIPIQEDYRKPEPACSP

In certain embodiments, the co-stimulatory molecule polypeptide is a CD40 polypeptide. In certain embodiments, the CD40 polypeptide is a human CD40 polypeptide. In certain embodiments, the CD40 polypeptide comprises or consists of an amino acid sequence that is at least about 85%, about 90%, about 95%, about 96%, about 97%, about 98%, about 99%, or about 100% identical or homologous to the amino acid sequence having a UniProt Reference No: P25942 (SEQ ID NO: 49) or a fragment thereof, and/or may optionally comprise up to one or up to two or up to three conservative amino acid substitutions. In certain embodiments, the CD40 polypeptide comprises or consists of an amino acid sequence that is a consecutive portion of SEQ ID NO: 49, which is at least about 20, or at least about 30, or at least about 40, or at least about 50, and up to about 277 amino acids in length. In certain embodiments, the CD40 polypeptide comprises or consists of the amino acid sequence of amino acids 1 to 277, 194 to 277, or 216 to 277 of SEQ ID NO: 49. In certain embodiments, the CD40 polypeptide comprises or consists of the amino acid sequence of amino acids 216 to 277 of SEQ ID NO: 49. SEQ ID NO: 49 is provided below.

[SEQ ID NO: 49]
MVRLPLQCVLWGCLLTAVHPEPPTACREKQYLINSQCCSLCQPGQKLVSDCTEFTETECLPCGESEFL
DTWNRETHCHQHKYCDPNLGLRVQQKGTSETDTICTCEEGWHCTSEACESCVLHRSCSPGFGVKQIAT
GVSDTICEPCPVGFFSNVSSAFEKCHPWTSCETKDLVVQQAGTNKTDVVCGPQDRLRALVVIPIIFGI
LFAILLVLVFIKKVAKKPTNKAPHPKQEPQEINFPDDLPGSNTAAPVQETLHGCQPVTQEDGKESRIS
VQERQ

In certain embodiments, the co-stimulatory molecule polypeptide is a CD2 polypeptide. In certain embodiments, the CD2 polypeptide is a human CD2 polypeptide. In certain embodiments, the CD2 polypeptide comprises or consists of an amino acid sequence that is at least about 85%, about 90%, about 95%, about 96%, about 97%, about 98%, about 99%, or about 100% identical or homologous to the amino acid sequence having a UniProt Reference No: P06729 (SEQ ID NO: 50) or a fragment thereof, and/or may optionally comprise up to one or up to two or up to three conservative amino acid substitutions. In certain embodiments, the CD2 polypeptide comprises or consists of an amino acid sequence that is a consecutive portion of SEQ ID NO: 50, which is at least about 20, or at least about 30, or at least about 40, or at least about 50, and up to about 351 amino acids in length. In certain embodiments, the CD2 polypeptide comprises or consists of the amino acid sequence of amino acids 1 to 351, 210 to 351, or 236 to 351 of SEQ ID NO: 50. In certain embodiments, the CD2 polypeptide comprises or consists of the amino acid sequence of amino acids 236 to 351 of SEQ ID NO: 50. SEQ ID NO: 50 is provided below.

[SEQ ID NO: 50]
MSFPCKFVASFLLIFNVSSKGAVSKEITNALETWGALGQDINLDIPSFQMSDDIDDIKWEKTSDKKKI
AQFRKEKETFKEKDTYKLFKNGTLKIKHLKTDDQDIYKVSIYDTKGKNVLEKIFDLKIQERVSKPKIS
WTCINTTLTCEVMNGTDPELNLYQDGKHLKLSQRVITHKWTTSLSAKFKCTAGNKVSKESSVEPVSCP
EKGLDIYLIIGICGGGSLLMVFVALLVFYITKRKKORSRRNDEELETRAHRVATEERGRKPHQIPAST
PQNPATSQHPPPPPGHRSQAPSHRPPPPGHRVQHQPOKRPPAPSGTQVHQQKGPPLPRPRVQPKPPHG
AAENSLSPSSN

In certain embodiments, the co-stimulatory molecule polypeptide is an NKG2D polypeptide. In certain embodiments, the NKG2D polypeptide is a human NKG2D polypeptide. In certain embodiments, the NKG2D polypeptide comprises or consists of an amino acid sequence that is at least about 85%, about 90%, about 95%, about 96%, about 97%, about 98%, about 99%, or about 100% identical or homologous to the amino acid sequence having a UniProt Reference No: P26718 (SEQ ID NO: 51) or a fragment thereof, and/or may optionally comprise up to one or up to two or up to three conservative amino acid substitutions. In certain embodiments, the NKG2D polypeptide comprises or consists of an amino acid sequence that is a consecutive portion of SEQ ID NO: 51, which is at least about 20, or at least about 30, or at least about 40, or at least about 50, and up to about 216 amino acids in length. In certain embodiments, the NKG2D polypeptide comprises or consists of the amino acid sequence of amino acids 1 to 51, 1 to 72, or 1 to 216 of SEQ ID NO: 51. In certain embodiments, the NKG2D polypeptide comprises or consists of the amino acid sequence of amino acids 1 to 51 of SEQ ID NO: 51. SEQ ID NO: 51 is provided below.

[SEQ ID NO: 51]
MGWIRGRRSRHSWEMSEFHNYNLDLKKSDFSTRWQKQRCPVVKSKCRENASPFFFCCFIAVAMGIRFI
IMVTIWSAVFLNSLFNQEVQIPLTESYCGPCPKNWICYKNNCYQFFDESKNWYESQASCMSQNASLLK
VYSKEDQDLLKLVKSYHWMGLVHIPTNGSWQWEDGSILSPNLLTIIEMQKGDCALYASSEKGYIENCS
TPNTYICMQRTV

In certain embodiments, the intracellular domain comprises a second co-stimulatory molecule polypeptide. In certain embodiments, the intracellular domain comprises a third co-stimulatory molecule polypeptide. In certain embodiments, the intracellular domain comprises a fourth co-stimulatory molecule polypeptide. In certain embodiments, the intracellular domain comprises a fifth co-stimulatory molecule polypeptide. In certain embodiments, the first, second, third, fourth, and fifth co-stimulatory molecule polypeptides can be the same or different from each other.

In certain embodiments, the intracellular domain of the presently disclosed fusion protein comprises a 4-1BB polypeptide. In certain embodiments, the intracellular domain of the presently disclosed fusion protein comprises a 4-1BB polypeptide and a CD28 polypeptide.

### 2.2.2. Hinge Spacer Region of the Fusion Polypeptides

In certain embodiments, the intracellular domain of the fusion polypeptide further comprises a hinge/spacer region that links the transmembrane domain to the intracellular domain. In certain embodiments, the hinge/spacer region of the intracellular domain can comprise a 4-1BBL polypeptide, an OX40L polypeptide, a CD70 polypeptide, a GITRL polypeptide, a CD40L polypeptide, a CD80 polypeptide, a CD86 polypeptide, an ICOS ligand (ICOSLG (also known as “CD275”) polypeptide, or a combination thereof.

In certain embodiments, the intracellular domain of the fusion polypeptide further comprises a hinge/spacer region comprising or consisting of a CD80 polypeptide. In certain embodiments, the intracellular domain of the fusion polypeptide further comprises a hinge/spacer region of a CD80 polypeptide comprising or consisting of amino acids 264 to 267 of SEQ ID NO: 32. An exemplary nucleotide sequence encoding the amino acid sequence comprising or consisting of amino acids 264 to 267 of SEQ ID NO: 32 is set forth in SEQ ID NO: 131, which is provided below.

[SEQ ID NO: 131]
ACCTACTGCTTT

In certain embodiments, the co-stimulatory ligand polypeptide and the hinge/spacer region derive from the same molecule. In certain embodiments, the co-stimulatory ligand polypeptide and the hinge/spacer region derive from different molecules.

### 2.3. Transmembrane Domain of the Fusion Polypeptides

In certain embodiments, the fusion polypeptide further comprises a transmembrane domain comprising a co-stimulatory ligand polypeptide (e.g., described in Section 2.1.2). In certain embodiments, the co-stimulatory ligand polypeptide of the transmembrane domain can be selected from the group consisting of tumor necrosis factor (TNF) family members, immunoglobulin (Ig) superfamily members, and combinations thereof. In certain embodiments, the TNF family member can be selected from the group consisting of a 4-1BBL polypeptide, an OX40L polypeptide, a CD70 polypeptide, a GITRL polypeptide, a CD40L polypeptide, and combinations thereof. In certain embodiments, the Ig superfamily member can be selected from the group consisting of a CD80 polypeptide, a CD86 polypeptide, an ICOS ligand (ICOSLG (also known as “CD275”) polypeptide, and combinations thereof. In certain embodiments, the co-stimulatory ligand is a CD80 polypeptide. In certain embodiments, the CD80 polypeptide is a human CD80 polypeptide.

In certain embodiments, the transmembrane domain comprises a CD80 polypeptide comprising or consisting of an amino acid sequence that is a consecutive portion of SEQ ID NO: 32, which is at least about 20, or at least about 30, or at least about 40, at least about 50, at least about 100, at least about 150, at least about 200, at least about 208, at least about 242, at least about 250, and up to about 288 amino acids in length. In certain embodiments, transmembrane domain comprises a CD80 polypeptide comprising or consisting of the amino acid sequence of amino acids 1 to 288, 1 to 242, 1 to 263, 35 to 288, 35 to 263, or 243 to 263 of SEQ ID NO: 32. In certain embodiments, transmembrane domain comprises a CD80 polypeptide comprising or consisting of the amino acid sequence of amino acids 243 to 263 of SEQ ID NO: 32. In certain embodiments, the transmembrane domain comprises a CD80 polypeptide comprising the amino acid sequence set forth in SEQ ID NO: 52. In certain embodiments, the transmembrane domain comprises a CD80 polypeptide consisting of the amino acid sequence set forth in SEQ ID NO: 52. SEQ ID NO: 52 is provided below.

[SEQ ID NO: 52]
LLPSWAITLISVNGIFVICCL

An exemplary nucleotide sequence encoding the amino acid sequence of SEQ ID NO: 52 is set forth in SEQ ID NO: 130, which is provided below.

[SEQ ID NO: 130]
CTGCTCCCATCCTGGGCCATTACCTTAATCTCAGTAAATGGAATTTTTG
TGATATGCTGCCTG

In certain embodiments, the co-stimulatory ligand polypeptide and the transmembrane domain derive from the same molecule. In certain embodiments, the co-stimulatory ligand polypeptide and the transmembrane domain derive from different molecules.

### 2.4. Cytokine Receptors

In certain embodiments, the fusion polypeptide further comprises a cytokine receptor. In certain embodiments, the fusion polypeptide further comprises a signaling domain of a cytokine receptor. The cytokine receptor or the signaling domain of the cytokine receptor can be positioned at the N-terminus or C-terminus of the fusion polypeptide. In certain embodiments, the cytokine receptor or the signaling domain of the cytokine receptor is positioned at the C-terminus of the fusion polypeptide. In certain embodiments, the cytokine receptor or the signaling domain of the cytokine receptor is positioned at the C-terminus of the co-stimulatory ligand (e.g., CD80, 4-1BBL, OX40L, CD70, GITRL, CD40L, CD86, or ICOSLG). Non-limiting examples of cytokine receptors include CD121a, CDw121b, IL-18Ra, IL18Rb, CD122, CD25, CD132, CD124, CD213a13, CD127, IL-9R, IL15Ra, CDw125, CDw131, CD126, CD130, IL11Ra, CD114, CD212, CD4, CDw217, CD118, and CDw119.

### 2.5. JAK-STAT Signaling Domains

In certain embodiments, the fusion polypeptide further comprises a polypeptide of a JAK-STAT signaling domain (e.g., derived from Jak1, Jak3 Stat1, Stat5a, Stat5b, Stat3, or Stat6). Signal transducer and activators of transcription (STAT) polypeptides are transcription factors that induce expression and regulation of certain target genes involved in the metabolic pathways (e.g., activation of immune cells). In certain embodiments, the JAK-STAT signaling domain can be positioned at the C-terminus of the fusion polypeptide. In certain embodiments, the JAK-STAT signaling domain can be positioned at the intracellular domain of the fusion polypeptide.

In certain embodiments, the JAK-STAT signaling domain is a STAT3 signaling domain. In certain embodiments, the STAT3 signaling domain comprises or consists of the amino acid sequence set forth in SEQ ID NO: 142, which is provided below.

[SEQ ID NO: 142]
YXXQ

In certain embodiments, the amino acid residue X of the STAT3 signaling domain set forth in SEQ ID NO: 142 can be any amino acid. In certain embodiments, the amino acid residue X of the STAT3 signaling domain set forth in SEQ ID NO: 142 is selected from the group consisting of leucine, arginine, histidine, phenylalanine, lysine, proline, methionine, valine, glutamine, threonine, and aspartate. In certain embodiments, the STAT3 signaling domain comprises or consists of the amino acid sequence set forth in SEQ ID NOs: 143-154, which are provided below.

[SEQ ID NO: 143]
YRHQ
[SEQ ID NO: 144]
YLRQ
[SEQ ID NO: 145]
YHNQ
[SEQ ID NO: 146]
YFKQ
[SEQ ID NO: 147]
YLPQ
[SEQ ID NO: 148]
YMPQ
[SEQ ID NO: 149]
YVLQ
[SEQ ID NO: 150]
YQPQ
[SEQ ID NO: 151]
YKPQ
[SEQ ID NO: 152]
YRPQ
[SEQ ID NO: 153]
YTHQ
[SEQ ID NO: 154]
YLKQ

In certain embodiments, the JAK-STAT signaling domain is a STAT5 signaling domain. In certain embodiments, the STAT5 signaling domain comprises or consists of the amino acid sequence set forth in SEQ ID NOs: 155-157, which are provided below.

[SEQ ID NO: 155]
YXXL
[SEQ ID NO: 156]
YFFF
[SEQ ID NO: 157]
YCTF

### 2.6. T Cell Signaling Molecules

In certain embodiments, the fusion polypeptide further comprises a T cell signaling molecule. T cell signaling molecules are molecules (e.g., polypeptides) that are physiologically involved and regulate the T cell receptor signal transduction pathways. In certain embodiments, the T cell signaling molecule can be a LAT polypeptide or a functional fragment thereof, a LCK polypeptide or a functional fragment thereof, an AKT polypeptide or a functional fragment thereof, or a SLP-76 polypeptide or a functional fragment thereof.

In certain embodiments, the T cell signaling molecule can be a LAT polypeptide or a functional fragment thereof. In certain embodiments, the LAT polypeptide is a human LAT polypeptide. In certain embodiments, the LAT polypeptide comprises or consists of an amino acid sequence that is at least about 85%, about 90%, about 95%, about 96%, about 97%, about 98%, about 99%, or about 100% identical or homologous to the amino acid sequence having a UniProt Reference No: 043561 (SEQ ID NO: 159) or a fragment thereof, and/or may optionally comprise up to one or up to two or up to three conservative amino acid substitutions. In certain embodiments, the LAT polypeptide comprises or consists of an amino acid sequence that is a consecutive portion of SEQ ID NO: 159, which is at least about 20, at least about 30, at least about 40, at least about 50, at least about 100, at least about 150, at least about 200, at least about 250, and up to about 262 amino acids in length. SEQ ID NO: 159 is provided below.

[SEQ ID NO: 159]
MEEAILVPCVLGLLLLPILAMLMALCVHCHRLPGSYDSTSSDSLYPRGIQFKRPHTVAPWPPAYPPVT
SYPPLSQPDLLPIPRSPQPLGGSHRTPSSRRDSDGANSVASYENEGASGIRGAQAGWGVWGPSWTRLT
PVSLPPEPACEDADEDEDDYHNPGYLVVLPDSTPATSTAAPSAPALSTPGIRDSAFSMESIDDYVNVP
ESGESAEASLDGSREYVNVSQELHPGAAKTEPAALSSQEAEEVEEEGAPDYENLQELN

In certain embodiments, the T cell signaling molecule can be a LCK polypeptide or a functional fragment thereof. In certain embodiments, the LCK polypeptide is a human LCK polypeptide. In certain embodiments, the LCK polypeptide comprises or consists of an amino acid sequence that is at least about 85%, about 90%, about 95%, about 96%, about 97%, about 98%, about 99%, or about 100% identical or homologous to the amino acid sequence having a UniProt Reference No: P06239 (SEQ ID NO: 160) or a fragment thereof, and/or may optionally comprise up to one or up to two or up to three conservative amino acid substitutions. In certain embodiments, the LCK polypeptide comprises or consists of an amino acid sequence that is a consecutive portion of SEQ ID NO: 160, which is at least about 20, at least about 30, at least about 40, at least about 50, at least about 100, at least about 150, at least about 200, at least about 300, at least about 400, at least about 500, and up to about 509 amino acids in length. SEQ ID NO: 160 is provided below.

[SEQ ID NO: 160]
MGCGCSSHPEDDWMENIDVCENCHYPIVPLDGKGTLLIRNGSEVRDPLVTYEGSNPPASPLQDNLVIA
LHSYEPSHDGDLGFEKGEQLRILEQSGEWWKAQSLTTGQEGFIPFNFVAKANSLEPEPWFFKNLSRKD
AERQLLAPGNTHGSFLIRESESTAGSFSLSVRDFDQNQGEVVKHYKIRNLDNGGFYISPRITFPGLHE
LVRHYTNASDGLCTRLSRPCQTQKPQKPWWEDEWEVPRETLKLVERLGAGQFGEVWMGYYNGHTKVAV
KSLKQGSMSPDAFLAEANLMKQLQHQRLVRLYAVVTQEPIYIITEYMENGSLVDFLKTPSGIKLTINK
LLDMAAQIAEGMAFIEERNYIHRDLRAANILVSDTLSCKIADFGLARLIEDNEYTAREGAKFPIKWTA
PEAINYGTFTIKSDVWSFGILLTEIVTHGRIPYPGMTNPEVIQNLERGYRMVRPDNCPEELYQLMRLC
WKERPEDRPTFDYLRSVLEDFFTATEGQYQPQP

In certain embodiments, the T cell signaling molecule can be an AKT polypeptide or a functional fragment thereof. In certain embodiments, the AKT polypeptide is a human AKT polypeptide. In certain embodiments, the AKT polypeptide comprises or consists of an amino acid sequence that is at least about 85%, about 90%, about 95%, about 96%, about 97%, about 98%, about 99%, or about 100% identical or homologous to the amino acid sequence having a UniProt Reference No: P31749 (SEQ ID NO: 161) or a fragment thereof, and/or may optionally comprise up to one or up to two or up to three conservative amino acid substitutions. In certain embodiments, the AKT polypeptide comprises or consists of an amino acid sequence that is a consecutive portion of SEQ ID NO: 161, which is at least about 20, at least about 30, at least about 40, at least about 50, at least about 100, at least about 150, at least about 200, at least about 300, at least about 400, at least about 450, and up to about 480 amino acids in length. SEQ ID NO: 161 is provided below.

[SEQ ID NO: 161]
MSDVAIVKEGWLHKRGEYIKTWRPRYFLLKNDGTFIGYKERPQDVDQREAPLNNFSVAQCQLMKTERP
RPNTFIIRCLQWTTVIERTFHVETPEEREEWTTAIQTVADGLKKQEEEEMDFRSGSPSDNSGAEEMEV
SLAKPKHRVTMNEFEYLKLLGKGTFGKVILVKEKATGRYYAMKILKKEVIVAKDEVAHTLTENRVLQN
SRHPFLTALKYSFQTHDRLCFVMEYANGGELFFHLSRERVFSEDRARFYGAEIVSALDYLHSEKNVVY
RDLKLENLMLDKDGHIKITDFGLCKEGIKDGATMKTFCGTPEYLAPEVLEDNDYGRAVDWWGLGVVMY
EMMCGRLPFYNQDHEKLFELILMEEIRFPRTLGPEAKSLLSGLLKKDPKQRLGGGSEDAKEIMQHRFF
AGIVWQHVYEKKLSPPFKPQVTSETDTRYFDEEFTAQMITITPPDQDDSMECVDSERRPHFPQFSYSA
SGTA

In certain embodiments, the T cell signaling molecule can be a SLP-76 polypeptide or a functional fragment thereof. In certain embodiments, the SLP-76 polypeptide is a human SLP-76 polypeptide. In certain embodiments, the SLP-76 polypeptide comprises or consists of an amino acid sequence that is at least about 85%, about 90%, about 95%, about 96%, about 97%, about 98%, about 99%, or about 100% identical or homologous to the amino acid sequence having a UniProt Reference No: Q13094 (SEQ ID NO: 162) or a fragment thereof, and/or may optionally comprise up to one or up to two or up to three conservative amino acid substitutions. In certain embodiments, the SLP-76 polypeptide comprises or consists of an amino acid sequence that is a consecutive portion of SEQ ID NO: 162, which is at least about 20, at least about 30, at least about 40, at least about 50, at least about 100, at least about 150, at least about 200, at least about 300, at least about 400, at least about 500, and up to about 533 amino acids in length. SEQ ID NO: 162 is provided below.

[SEQ ID NO: 162]
MALRNVPFRSEVLGWDPDSLADYFKKLNYKDCEKAVKKYHIDGARFLNLTENDIQKFPKLRVPILSKL
SQEINKNEERRSIFTRKPQVPRFPEETESHEEDNGGWSSFEEDDYESPNDDQDGEDDGDYESPNEEEE
APVEDDADYEPPPSNDEEALQNSILPAKPFPNSNSMYIDRPPSGKTPQQPPVPPQRPMAALPPPPAGR
NHSPLPPPQTNHEEPSRSRNHKTAKLPAPSIDRSTKPPLDRSLAPFDREPFTLGKKPPFSDKPSIPAG
RSLGEHLPKIQKPPLPPTTERHERSSPLPGKKPPVPKHGWGPDRRENDEDDVHQRPLPQPALLPMSSN
TFPSRSTKPSPMNPLPSSHMPGAFSESNSSFPQSASLPPYFSQGPSNRPPIRAEGRNFPLPLPNKPRP
PSPAEEENSLNEEWYVSYITRPEAEAALRKINQDGTFLVRDSSKKTTTNPYVLMVLYKDKVYNIQIRY
QKESQVYLLGTGLRGKEDFLSVSDIIDYFRKMPLLLIDGKNRGSRYQCTLTHAAGYP

### 2.7. Exemplary Fusion Polypeptides

In certain embodiments, the presently disclosed fusion polypeptide comprises an extracellular domain comprising an antigen-binding fragment and a co-stimulatory ligand polypeptide, and an intracellular domain comprising a first co-stimulatory molecule polypeptide. In certain embodiments, the antigen-binding fragment binds to PD-L1. In certain embodiments, the antigen-binding fragment comprises a CDR1 comprising the amino acid sequence set forth in SEQ ID NO: 27, a CDR2 comprising the amino acid sequence set forth in SEQ ID NO: 28, and a CDR3 comprising the amino acid sequence set forth in SEQ ID NO: 29. In certain embodiments, the antigen-binding fragment comprises the amino acid sequence set forth in SEQ ID NO: 30. In certain embodiments, the co-stimulatory ligand polypeptide is a CD80 polypeptide. In certain embodiments, the CD80 polypeptide comprises the amino acid sequence set forth in SEQ ID NO: 33. In certain embodiments, the co-stimulatory molecule polypeptide is a 4-1BB polypeptide. In certain embodiments, the 4-1BB polypeptide comprises the amino acid sequence set forth in SEQ ID NO: 42.

In certain embodiments, the presently disclosed fusion polypeptide comprises an extracellular domain comprising an antigen-binding fragment and a co-stimulatory ligand polypeptide, and an intracellular domain comprising a first co-stimulatory molecule polypeptide. In certain embodiments, the antigen-binding fragment binds to PD-L1. In certain embodiments, the co-stimulatory ligand polypeptide is a CD80 polypeptide. In certain embodiments, the co-stimulatory molecule polypeptide is a 4-1BB polypeptide. In certain embodiments, the fusion polypeptide comprises or consists of the amino acid sequence set forth in SEQ ID NO: 53, which is provided below.

[SEQ ID NO: 53]
MGHTRRQGTSPSKCPYLNFFQLLVLAGLSHFCSGQVQLQESGGGLVQPGGSLRLSCAASGKMSSRRCM
AWFRQAPGKERERVAKLLTTSGSTYLADSVKGRFTISQNNAKSTVYLQMNSLKPEDTAMYYCAADSFE
DPTCTLVTSSGAFQYWGQGTQVTVSSGGGGSGGGGSGGGGSGGGGSVIHVTKEVKEVATLSCGHNVSV
EELAQTRIYWQKEKKMVLTMMSGDMNIWPEYKNRTIFDITNNLSIVILALRPSDEGTYECVVLKYEKD
AFKREHLAEVTLSVKADFPTPSISDFEIPTSNIRRIICSTSGGFPEPHLSWLENGEELNAINTTVSQD
PETELYAVSSKLDFNMTTNHSFMCLIKYGHLRVNQTFNWNTTKQEHFPDNLLPSWAITLISVNGIFVI
CCLTYCFKRGRKKLLYIFKQPFMRPVQTTQEEDGCCRFPEEEEGGCEL

An exemplary nucleotide sequence encoding the amino acid sequence of SEQ ID NO: 53 is set forth in SEQ ID NO: 54, which is provided below.

[SEQ ID NO: 54]
ATGGGACACACACGGAGGCAAGGAACCTCCCCATCCAAATGTCCTTACTTGAATTTTTTCCAACTGCT
TGTACTCGCAGGACTCAGCCACTTCTGTAGCGGACAGGTCCAACTGCAAGAGTCTGGTGGGGGTCTTG
TCCAACCTGGGGGCTCCCTGAGGCTCTCATGTGCAGCTAGTGGCAAAATGAGCAGTAGGAGGTGCATG
GCCTGGTTCCGGCAGGCACCCGGTAAGGAAAGAGAACGAGTAGCTAAACTTCTGACCACTTCAGGCTC
AACCTACCTCGCTGACAGTGTAAAAGGACGATTCACCATATCACAAAATAATGCTAAATCCACTGTGT
ACCTCCAAATGAATAGTCTGAAGCCTGAGGACACCGCCATGTATTACTGTGCCGCAGACTCATTTGAA
GATCCCACCTGCACACTCGTGACCAGCAGCGGGGCCTTCCAATACTGGGGTCAAGGCACCCAAGTGAC
AGTCTCCAGTGGAGGAGGGGGATCAGGAGGCGGTGGATCTGGTGGGGGTGGTTCAGGAGGTGGCGGAT
CTGTTATCCACGTGACCAAGGAAGTGAAAGAAGTGGCAACGCTGTCCTGTGGTCACAATGTTTCTGTT
GAAGAGCTGGCACAAACTCGCATCTACTGGCAAAAGGAGAAGAAAATGGTGCTGACTATGATGTCTGG
GGACATGAATATATGGCCCGAGTACAAGAACCGGACCATCTTTGATATCACTAATAACCTCTCCATTG
TGATCCTGGCTCTGCGCCCATCTGACGAGGGCACATACGAGTGTGTTGTTCTGAAGTATGAAAAAGAC
GCTTTCAAGCGGGAACACCTGGCTGAAGTGACGTTATCAGTCAAAGCTGACTTCCCTACACCTAGTAT
ATCTGACTTTGAAATTCCAACTTCTAATATTAGAAGGATAATTTGCTCAACCTCTGGAGGTTTTCCAG
AGCCTCACCTCTCCTGGTTGGAAAATGGAGAAGAATTAAATGCCATCAACACAACAGTTTCCCAAGAT
CCTGAAACTGAGCTCTATGCTGTTAGCAGCAAACTGGATTTCAATATGACAACCAACCACAGCTTCAT
GTGTCTCATCAAGTATGGACATTTAAGAGTGAATCAGACCTTCAACTGGAATACAACCAAGCAAGAGC
ATTTTCCTGATAACCTGCTCCCATCCTGGGCCATTACCTTAATCTCAGTAAATGGAATTTTTGTGATA
TGCTGCCTGACCTACTGCTTTAAACGGGGCAGAAAGAAACTCCTGTATATATTCAAACAACCATTTAT
GAGACCAGTACAAACTACTCAAGAGGAAGATGGCTGTAGCTGCCGATTTCCAGAAGAAGAAGAAGGAG
GATGTGAACTG

In certain embodiments, the presently disclosed fusion polypeptide comprises an extracellular domain comprising an antigen-binding fragment and a co-stimulatory ligand polypeptide, and an intracellular domain comprising a first co-stimulatory molecule polypeptide. In certain embodiments, the antigen-binding fragment binds to PD-L1. In certain embodiments, the antigen-binding fragment comprises a V_{H} comprising a CDR1 comprising the amino acid sequence set forth in SEQ ID NO: 16, a CDR2 comprising the amino acid sequence set forth in SEQ ID NO: 17, a CDR3 comprising the amino acid sequence set forth in SEQ ID NO: 18; and a V_{L} comprising a CDR1 comprising the amino acid sequence set forth in SEQ ID NO: 19, a CDR2 comprising the amino acid sequence set forth in SEQ ID NO: 20, and a CDR3 comprising the amino acid sequence set forth in SEQ ID NO: 21. In certain embodiments, the antigen-binding fragment comprises a V_{H} comprising the amino acid sequence set forth in SEQ ID NO: 22 and a V_{L} comprising the amino acid sequence set forth in SEQ ID NO: 23. In certain embodiments, the co-stimulatory ligand polypeptide is a CD80 polypeptide. In certain embodiments, the CD80 polypeptide comprises the amino acid sequence set forth in SEQ ID NO: 33. In certain embodiments, the co-stimulatory molecule polypeptide is a 4-1BB polypeptide. In certain embodiments, the 4-1BB polypeptide comprises the amino acid sequence set forth in SEQ ID NO: 42.

In certain embodiments, the presently disclosed fusion polypeptide comprises an extracellular domain comprising an antigen-binding fragment and a co-stimulatory ligand polypeptide, and an intracellular domain comprising a first co-stimulatory molecule polypeptide. In certain embodiments, the antigen-binding fragment binds to PD-L1. In certain embodiments, the co-stimulatory ligand polypeptide is a CD80 polypeptide. In certain embodiments, the co-stimulatory molecule polypeptide is a 4-1BB polypeptide. In certain embodiments, the fusion polypeptide comprises or consists of the amino acid sequence set forth in SEQ ID NO: 55, which is provided below.

[SEQ ID NO: 55]
MALPVTALLLPLALLLHAEVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRHWPGGFDYWGQGTLVTVSS
GGGGSGGGGSGGGGSDIQMTQSPSSLSASVGDRVTITCRASQDVSTAVAWYQQKPGKAPKLLIYSASF
LYSGVPSRFSGSGSGTDFTLTISSLQPEDFATYYCQQYLYHPATFGQGTKVEIKRTVGGGGSGGGGSG
GGGSGGGGSVIHVTKEVKEVATLSCGHNVSVEELAQTRIYWQKEKKMVLTMMSGDMNIWPEYKNRTIF
DITNNLSIVILALRPSDEGTYECVVLKYEKDAFKREHLAEVTLSVKADFPTPSISDFEIPTSNIRRII
CSTSGGFPEPHLSWLENGEELNAINTTVSQDPETELYAVSSKLDFNMTTNHSFMCLIKYGHLRVNQTF
NWNTTKQEHFPDNLLPSWAITLISVNGIFVICCLTYCFKRGRKKLLYIFKQPFMRPVQTTQEEDGCSC
RFPEEEEGGCEL

An exemplary nucleotide sequence encoding the amino acid sequence of SEQ ID NO: 55 is set forth in SEQ ID NO: 56, which is provided below.

[SEQ ID NO: 54]
ATGGCTCTCCCAGTGACTGCCCTACTGCTTCCCCTAGCGCTTCTCCTGCATGCAGAGGTGCAGCTGGT
GGAGTCTGGGGGTGGCCTGGTGCAGCCTGGGGGCTCACTGAGGCTTTCCTGCGCCGCTTCTGGCTTTA
CATTCAGTGACAGCTGGATTCACTGGGTGAGGCAGGCTCCTGGAAAGGGTCTTGAGTGGGTTGCATGG
ATTTCTCCTTACGGTGGTAGCACTTACTACGCTGACAGCGTGAAGGGTCGATTCACAATCTCCGCAGA
CACCTCCAAGAACACAGCCTACCTGCAGATGAACAGCCTACGGGCCGAGGACACCGCAGTCTATTACT
GTGCAAGAAGACACTGGCCTGGTGGATTTGACTACTGGGGTCAAGGGACCCTGGTCACCGTCTCCTCA
GGTGGAGGTGGATCAGGTGGAGGTGGATCTGGTGGAGGTGGATCTGACATTCAGATGACCCAGTCTCC
ATCATCCCTGTCCGCATCAGTAGGAGACAGGGTCACCATCACCTGCCGGGCCAGTCAGGATGTGTCAA
CTGCTGTAGCCTGGTATCAACAGAAACCAGGAAAGGCCCCTAAACTGCTGATTTACTCGGCCAGCTTT
CTGTACAGTGGAGTCCCTAGTCGCTTCAGCGGCAGTGGATCTGGGACAGATTTCACTCTCACCATCTC
TTCCCTGCAGCCTGAGGACTTCGCAACCTATTACTGTCAACAATATCTGTACCACCCGGCCACGTTCG
GACAGGGGACCAAGGTGGAGATCAAACGGACCGTGGGAGGCGGGGGTAGCGGAGGCGGCGGATCTGGT
GGGGGTGGTTCAGGAGGTGGCGGATCTGTTATCCACGTGACCAAGGAAGTGAAAGAAGTGGCAACGCT
GTCCTGTGGTCACAATGTTTCTGTTGAAGAGCTGGCACAAACTCGCATCTACTGGCAAAAGGAGAAGA
AAATGGTGCTGACTATGATGTCTGGGGACATGAATATATGGCCCGAGTACAAGAACCGGACCATCTTT
GATATCACTAATAACCTCTCCATTGTGATCCTGGCTCTGCGCCCATCTGACGAGGGCACATACGAGTG
TGTTGTTCTGAAGTATGAAAAAGACGCTTTCAAGCGGGAACACCTGGCTGAAGTGACGTTATCAGTCA
AAGCTGACTTCCCTACACCTAGTATATCTGACTTTGAAATTCCAACTTCTAATATTAGAAGGATAATT
TGCTCAACCTCTGGAGGTTTTCCAGAGCCTCACCTCTCCTGGTTGGAAAATGGAGAAGAATTAAATGC
CATCAACACAACAGTTTCCCAAGATCCTGAAACTGAGCTCTATGCTGTTAGCAGCAAACTGGATTTCA
ATATGACAACCAACCACAGCTTCATGTGTCTCATCAAGTATGGACATTTAAGAGTGAATCAGACCTTC
AACTGGAATACAACCAAGCAAGAGCATTTTCCTGATAACCTGCTCCCATCCTGGGCCATTACCTTAAT
CTCAGTAAATGGAATTTTTGTGATATGCTGCCTGACCTACTGCTTTAAACGGGGCAGAAAGAAACTCC
TGTATATATTCAAACAACCATTTATGAGACCAGTACAAACTACTCAAGAGGAAGATGGCTGTAGCTGC
CGATTTCCAGAAGAAGAAGAAGGAGGATGTGAACTG

### 2.8. Activities of Fusion Polypeptides

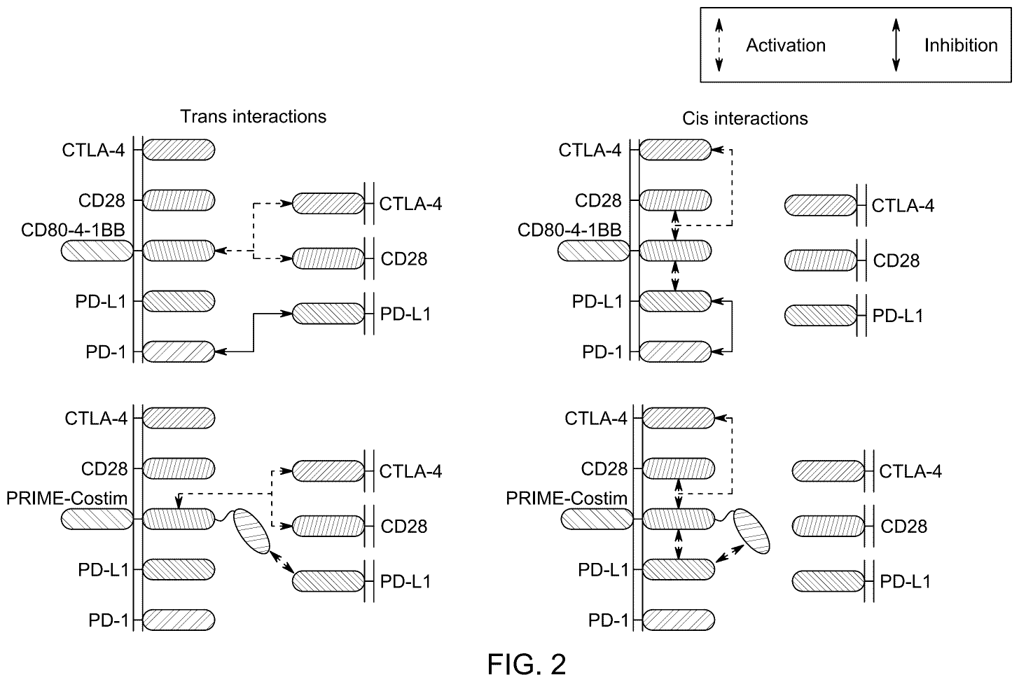

The presently disclosed fusion polypeptide is capable of stimulating a cell comprising an antigen-recognizing receptor (e.g., a CAR, a TCR, or a TCR like molecule). In certain embodiments, the fusion polypeptide provides stimulation to T cells in cis and/or in trans as detailed in FIG. 2.

In certain embodiments, the antigen-binding fragment of the presently disclosed fusion polypeptide binds to an immune checkpoint molecule. In certain embodiments, the antigen-binding fragment of the presently disclosed fusion polypeptide binds to PD-L1. By binding to PD-L1, the antigen-binding fragment can disrupt the interaction between PD-1 and PD-L1 in cis (e.g., when PD-1 and PD-L1 are expressed by the same cell) or in trans (e.g., when PD-1 and PD-L1 are expressed by the same cell). Similarly, different antigen-binding fragments targeting other immune checkpoint molecules will function as immune checkpoint inhibitors.

In certain embodiments, the co-stimulatory ligand polypeptide of the presently disclosed fusion polypeptide is CD80. CD80 can interact with CD28, CTLA-4, and PD-L1. CD80 is the ligand of CD28, the main co-stimulatory molecule expressed by T cells. Upon activation, T cells express CTLA-4 and PD-L1, which negatively regulate T-cell receptor (“TCR”) and CD28 downstream signaling. CTLA-4 functions as a CD28 competitor by interacting with CD80 (affinity of the interaction of CTLA-4/CD80 is usually higher than CD80/CD28). PD-L1, the receptor of PDL1, functions as a T cell inhibitor by de-phosphorylation of TCR and CD28 downstream signalosome. Similar to CTLA-4, PD-L1 can interact with CD80 under certain conditions.

As illustrated in FIG. 2, the presently disclosed fusion polypeptides (e.g., including an extracellular domain comprising a CD80 polypeptide) can ligate three different receptors: CD28, CTLA-4, and PD-L1. By doing so, these ligations provide (i) signaling through the intracellular domain of the co-stimulatory molecule (e.g., 4-1BB, CD28, OX40, ICOS, DAP-10, CD27, CD40, NKG2D, or CD2) and (ii) competes with CTLA4/CD80 and PD-L1/PD-1 ligation in cis and in trans, thereby shifting inhibitory signaling to activation signaling to T cells. Constitutive expression of CD80 (e.g., at a high level) on T cells provides a strong co-stimulation upon interaction with endogenous CD28. By interacting with CTLA-4 and PD-L1, the fusion polypeptide disclosed herein can compete with endogenous CD80/CTLA4 and PD-1/PD-L1 (e.g., expressed at a low level) to afford positive signaling into T cells. CTLA-4 is expressed on engineered and bystander T cells. PD-L1 is expressed on different cell types (mainly antigen-presenting cells and tumor cells) and can be found on engineered T cells as well. Replacing the intracellular domain of CD80 with a co-stimulatory derived signaling domain (e.g., including an extracellular domain comprising a CD80 polypeptide) shifts CD80 signaling from negative to positive.

The presently disclosed fusion polypeptide (e.g., a fusion polypeptide comprising an extracellular domain and a transmembrane domain of CD80) provides stimulation of T cells in cis and trans via engagement with CD28 molecule as well as delivering signaling via the intracellular domain of one or more co-stimulatory molecules (e.g., 4-1BB, and/or CD28). Moreover, the presently disclosed fusion polypeptide can engage with CTLA-4 and PD-L1, two inhibitory molecules that are known to be up-regulated in the tumor microenvironment. These interactions shift T cell inhibition into T cell activation. Thus, the presently disclosed fusion polypeptide (e.g., including an extracellular domain comprising a CD80 polypeptide) affords co-stimulation into T cells and benefits positively from T cell inhibitory receptors expressed in a hostile tumor microenvironment.

### 2.9. Regulatability of the Fusion Polypeptides

In certain embodiments, the fusion polypeptide can be regulated, e.g., is regulatable. A regulator can regulate or modulate the expression and/or activity of the fusion polypeptide. With the regulator, the expression and/or activity of the fusion polypeptide can be switched on, or switched off. The fusion polypeptide can be regulated by controlling the levels of its expression (i.e., constitutive high, constitutive low, or inducible) or by administration of exogenous molecules (e.g., antibodies, Ig fusion proteins). These approaches serve the purpose of switching on and off the function of the fusion polypeptide.

Non-limiting examples of regulators include promoters (e.g., inducible promoters) that are capable of controlling the expression of the fusion polypeptide, molecules that are capable of regulating or modulating the expression and/or activity of the co-stimulatory ligand comprised in the fusion polypeptide, and molecules that are capable of regulating or modulating the expression and/or activity of the co-stimulatory molecule comprised in the fusion polypeptide.

Molecules that are capable of regulating or modulating the expression and/or activity of the co-stimulatory ligand comprised in the fusion polypeptide include antibodies binding to the co-stimulatory ligand, and fusion proteins binding to the co-stimulatory ligand and modulating expression and/or activity of the co-stimulatory ligand. In certain embodiments, the regulator is an antibody that binds to the co-stimulatory ligand.

Molecules that are capable of regulating or modulating the expression and/or activity of the co-stimulatory molecule comprised in the fusion polypeptide include antibodies binding to the co-stimulatory molecule, and fusion proteins binding to the co-stimulatory molecule and modulating expression and/or activity of the co-stimulatory molecule. In certain embodiments, the regulator is an antibody that binds to the co-stimulatory molecule.

In certain embodiments, the fusion polypeptide comprises an extracellular domain comprising a CD80 polypeptide, and the regulator is an anti-CD80 antibody. In certain embodiments, the fusion polypeptide comprises an extracellular domain comprising a CD80 polypeptide, and the regulator is a fusion protein that binds to CD80 and modulates the activity of CD80. In certain embodiments, the regulator comprises a CTLA4 fragment that binds to CD80. In certain embodiments, the CTLA4 fragment is abatacept and belatacept.

### 2.10. Nucleic Acid Molecules Encoding the Fusion Polypeptides

The presently disclosed subject matter further provides nucleic acid molecules encoding the fusion polypeptides disclosed herein. In addition, the presently disclosed subject matter provides vectors comprising the nucleic acid molecules described herein. The vectors can be viral vectors or non-viral vectors. In certain embodiments, the vector is a viral vector. In certain embodiments, the viral vector is a retroviral vector, e.g., a gamma-retroviral vector, or a lentiviral vector.

### 2.11. Delivery of the Fusion Polypeptides

In certain embodiments, the fusion polypeptide or a polynucleotide encoding the same is delivered to the cell by a viral method. In certain embodiments, the viral method comprises a viral vector. In certain embodiments, the viral vector is a retroviral vector (e.g., a gamma-retroviral vector or a lentiviral vector). Other viral vectors include adenoviral vectors, adeno-associated viral vectors, vaccinia viruses, bovine papilloma viruses, and herpes viruses (e.g., such as Epstein-Barr Virus).

In certain embodiments, the fusion polypeptide or a polynucleotide encoding the same is delivered to the cell by a non-viral method. Any targeted genome editing methods can also be used to deliver the fusion polypeptide to the cell. In certain embodiments, the fusion polypeptide is delivered to the cell by a method comprising homologous recombination, a Zinc finger nuclease, a meganuclease, a Transcription activator-like effector nuclease (TALEN), a Clustered regularly-interspaced short palindromic repeats (CRISPR) system, or a combination thereof. In certain embodiments, a CRISPR system is used to deliver the fusion polypeptide to the cell.

In certain embodiments, the cell is a T cell, and the fusion polypeptide or a polynucleotide encoding the same is integrated at a locus within the genome of the T cell. Non-limiting examples of loci include a TRAC locus, a TRBC locus, a TRDC locus, and a TRGC locus. In certain embodiments, the locus is a TRAC locus or a TRBC locus. In certain embodiments, the cell is a T cell, and the fusion polypeptide is integrated at a TRAC locus. Methods of targeting a CAR to a site within the genome of T cells are disclosed in WO2017180989 and Eyquem et al., Nature. (2017 Mar. 2); 543(7643): 113-117, both of which are incorporated by reference in their entirety. In certain embodiments, the cell further comprises a gene disruption of a TRBC locus. In certain embodiments, the gene disruption of a TRBC locus results in the knockout of the TRBC locus.

Additionally or alternatively, the fusion polypeptide or a polynucleotide encoding the same is integrated at a genomic safe harbor within the genome of the T cell. Further information on genomic safe harbors and on methods for identifying the same can be found in International Patent Publications No. 2021/055592 and No. 2021/055616, the contents of each of which are incorporated by reference in their entirety.

### 3. CELLS

The presently disclosed subject matter provides cells comprising a fusion polypeptide disclosed herein. In certain embodiments, the fusion protein is capable of promoting an anti-tumor effect of the cell.

In certain embodiments, the cell is selected from the group consisting of cells of lymphoid lineage and cells of myeloid lineage. In certain embodiments, the cell is an immunoresponsive cell. In certain embodiments, the immunoresponsive cell is a cell of lymphoid lineage.

In certain embodiments, the cell is a cell of the lymphoid lineage. Cells of the lymphoid lineage can provide production of antibodies, regulation of cellular immune system, detection of foreign agents in the blood, detection of cells foreign to the host, and the like. Non-limiting examples of cells of the lymphoid lineage include T cells, Natural Killer (NK) cells, B cells, dendritic cells, stem cells from which lymphoid cells may be differentiated. In certain embodiments, the stem cell is a pluripotent stem cell (e.g., embryonic stem cell).

In certain embodiments, the cell is a T cell. T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), two types of effector memory T cells: e.g., T_{EM} cells and T_{EMRA} cells, Regulatory T cells (also known as suppressor T cells), tumor-infiltrating lymphocyte (TIL), virus-specific T cells (VST), Natural Killer T cells, Mucosal associated invariant T cells, and γδ T cells. Cytotoxic T cells (CTL or killer T cells) are a subset of T lymphocytes capable of inducing the death of infected somatic or tumor cells. A patient's own T cells may be genetically modified to target specific antigens through the introduction of an antigen-recognizing receptor, e.g., a CAR or a TCR. The T cell can be a CD4⁺ T cell or a CD8⁺ T cell. In certain embodiments, the T cell is a CD4⁺ T cell. In certain embodiments, the T cell is a CD8⁺ T cell. In certain embodiments, the CD8⁺ T cell is CD4 independent. In certain embodiments, the T cell is derived from an induced pluripotent stem cell (iPSC). In certain embodiments, the T cell is a CD8⁺ T cell that is CD4 independent, and the CD8⁺ T cell is derived from an iPSC.

In certain embodiments, the T cell is a tumor-infiltrating lymphocyte (TIL). TILs are lymphocytes that have left the bloodstream and migrated towards a tumor. TILs can be found in the tumor stroma and within the tumor itself.

In certain embodiments, the T cell is a virus-specific T cell (VST). VSTs are lymphocytes that express T cells receptors capable of recognizing viral antigens when expressed in the context of MHC Class I or MHC Class II complexes. In certain embodiments, the VSTs encompassed by the presently disclosed subject matter recognize CMV antigens. In certain embodiments, the VSTs encompassed by the presently disclosed subject matter recognize EBV antigens.

In certain embodiments, the T cell is a CD62L⁺ T cell. In certain embodiments, the T cell is a CD45RA⁺ T cell. In certain embodiments, the T cell is a CD62L⁺/CD45RA⁺ T cell.

In certain embodiments, the cell is an NK cell. Natural Killer (NK) cells can be lymphocytes that are part of cell-mediated immunity and act during the innate immune response. NK cells do not require prior activation in order to perform their cytotoxic effect on target cells.

Types of human lymphocytes of the presently disclosed subject matter include, without limitation, peripheral donor lymphocytes, e.g., those disclosed in Sadelain, M., et al. 2003 Nat Rev Cancer 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the α and β heterodimer), in Panelli, M. C., et al. 2000 J Immunol 164:495-504; Panelli, M. C., et al. 2000 J Immunol 164:4382-4392 (disclosing lymphocyte cultures derived from tumor infiltrating lymphocytes (TILs) in tumor biopsies), and in Dupont, J., et al. 2005 Cancer Res 65:5417-5427; Papanicolaou, G. A., et al. 2003 Blood 102:2498-2505 (disclosing selectively in vitro-expanded antigen-specific peripheral blood leukocytes employing artificial antigen-presenting cells (AAPCs) or pulsed dendritic cells).

In certain embodiments, the cell (e.g., T cell) is autologous. In certain embodiments, the cell (e.g., T cell) is non-autologous. In certain embodiments, the cell (e.g., T cell) is allogeneic. In certain embodiments, the cell (e.g., T cell) is derived in vitro from an engineered progenitor or stem cell.

In certain embodiments, the cell is a cell of the myeloid lineage. Non-limiting examples of cells of the myeloid lineage include monocytes, macrophages, neutrophils, basophils, eosinophils, erythrocytes, megakaryocytes, and stem cells from which myeloid cells may be differentiated.

In certain embodiments, the stem cell is a pluripotent stem cell (e.g., an embryonic stem cell or an induced pluripotent stem cell).

In certain embodiments, the cell further comprises an antigen-recognizing receptor. In certain embodiments, the antigen-recognizing receptor is capable of activating the cell. The cells can be transduced with an antigen-recognizing receptor and a fusion polypeptide such that the cells co-express the antigen-recognizing receptor and the fusion polypeptide.

### 3.1. Antigen-Recognizing Receptor

The antigen-recognizing receptor targets an antigen. The antigen can be a tumor antigen or a pathogen antigen.

### 3.1.1. Antigen

In certain embodiments, the antigen is a tumor antigen. In certain embodiments, the antigen is an antigen with low cell density. In certain embodiments, the tumor antigen is an antigen with low tumor density.

Any tumor antigen (antigenic peptide) can be used in the tumor-related embodiments described herein. Sources of antigen include, but are not limited to, cancer proteins. The antigen can be expressed as a peptide or as an intact protein or a portion thereof. The intact protein or portion thereof can be native or mutagenized. Non-limiting examples of tumor antigens include CD19, CD70, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell (e.g. a cell surface antigen), ANO9, AQP2, ASIC3, ASPRV1, ATP6VOA4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD276, CD30, CD300LF, CD312, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBPlB, FLRT1, folate receptor-a, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Ra2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, LlCAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSCI, MART1, GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Proteinase3 (PR1), PSD2, PTPRJ, RDH16, receptor tyrosine-protein kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycoprotein 72 (TAG-72), Tyrosinase, vascular endothelial growth factor R2 (VEGF-R2), VLA-4, Wilms tumor protein (WT-1), WNT4, WT1, and ZDHHC11.

In certain embodiments, the antigen is selected from the group consisting of CD312, CLEC12A, CD33, CD123, IL1RAP, SIGLEC-6, GRP78, TIM3, CD70, CD20, CD22, CD19, GPRC5D, SLAMF7, BCMA, CD276, and CAIX. In certain embodiments, the antigen is CD312. In certain embodiments, the antigen is CD276. In certain embodiments, the antigen is CD19.

In certain embodiments, the antigen is a pathogen antigen. In certain embodiments, the pathogen antigen is an antigen of a virus or a bacteria. Non-limiting examples of viruses include, Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Naira viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses). Non-limiting examples of bacteria include Pasteurella, Staphylococci, Streptococcus, Escherichia coli, Pseudomonas species, and Salmonella species. Specific examples of infectious bacteria include but are not limited to, Helicobacter pylori, Borrelia burgdorferi, Legionella, Legionella pneumophilia, Mycobacteria sps (e.g. M. tuberculosis, M. avium, M. intracellulare, M. kansaii, M. gordonae, M leprae), Staphylococcus aureus, Staphylococcus epidermidis, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Campylobacter jejuni, Enterococcus sp., Haemophilus influenzae, Bacillus antracis, Corynebacterium diphtheriae, Corynebacterium sp., Erysipelothrix rhusiopathiae, Clostridium spp., Clostridium perfringens, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, Rickettsia, and Actinomyces israelli. Mycoplasma, Pseudomonas aeruginosa, Pseudomonas fluorescens, Corynobacteria diphtheriae, Bartonella henselae, Bartonella quintana, Coxiella burnetii, chlamydia, shigella, Yersinia enterocolitica, Yersinia pseudotuberculosis, Listeria monocytogenes, Mycoplasma spp., Vibrio cholerae, Borrelia, Francisella, Brucella melitensis, Proteus mirabilis, and Proteus.

In certain embodiments, the pathogen antigen is a viral antigen present in Cytomegalovirus (CMV), a viral antigen present in Epstein Barr Virus (EBV), a viral antigen present in Human Immunodeficiency Virus (HIV), or a viral antigen present in influenza virus.

### 3.1.2. Chimeric Antigen Receptors (CARs)

In certain embodiments, the antigen-recognizing receptor is a chimeric antigen receptor (CAR). CARs are engineered receptors, which graft or confer a specificity of interest onto an immune effector cell. CARs can be used to graft the specificity of a monoclonal antibody onto a T cell; with transfer of their coding sequence facilitated by retroviral vectors.

There are three generations of CARs. “First generation” CARs are typically composed of an extracellular antigen-binding domain (e.g., an scFv) that binds to a target antigen, and an intracellular signaling domain. In certain embodiments, the CAR further comprises a transmembrane domain. “First generation” CARs can provide de novo antigen recognition and cause activation of both CD4+ and CD8⁺ T cells through their CD3ζ chain signaling domain in a single fusion molecule, independent of HLA-mediated antigen presentation. “Second generation” CARs include a signaling domain of a co-stimulatory molecule (e.g., CD28, 4-1BB, ICOS, OX40, CD27, CD40, NKG2D, DAP-10, CD2, CD150, CD226) to the intracellular signaling domain of the CAR to provide co-stimulation signals to the cell (e.g., T cell or NK cell). “Second generation” CARs comprise those that provide both co-stimulation (e.g., CD28 or 4-1BB) and activation (CD3ζ). “Third generation” CARs comprise those that provide multiple co-stimulation (e.g., CD28 and 4-1BB) and activation (CD3ζ).

In certain embodiments, the antigen-recognizing receptor is a CAR comprising an extracellular antigen-binding domain that binds to an antigen (e.g., one disclosed in Section 3.1.1) and an intracellular signaling domain. In certain embodiments, the CAR further comprises a transmembrane domain. In certain embodiments, the CAR further comprises a hinger/spacer region.

### 3.1.2.1. Extracellular Antigen-Binding Domain

In certain embodiments, the extracellular antigen-binding domain of the CAR (for example, an scFv) binds to the antigen with a dissociation constant (K_{D}) of about 5×10⁻⁷ M or less, about 1×10⁻⁷ M or less, about 5×10⁻⁸ M or less, about 1×10⁻⁸ M or less, about 5×10⁻⁹ M or less, or about 1×10⁻⁹M or less, or about 1×10⁻¹⁰ M or less. In certain embodiments, the extracellular antigen-binding domain of the CAR (for example, an scFv) binds to the antigen with a K_{D} of about 1×10⁻⁸M or less.

Binding of the extracellular antigen-binding domain (for example, in an scFv) can be confirmed by, for example, enzyme-linked immunosorbent assay (ELISA), radioimmunoassay (RIA), FACS analysis, bioassay (e.g., growth inhibition), or Western Blot assay. Each of these assays generally detects the presence of protein-antibody complexes of particular interest by employing a labeled reagent (e.g., an antibody, or an scFv) specific to the complex of interest. For example, the scFv can be radioactively labeled and used in a radioimmunoassay (RIA) (see, for example, Weintraub, B., Principles of Radioimmunoassays, Seventh Training Course on Radioligand Assay Techniques, The Endocrine Society, March 1986, which is incorporated by reference herein). The radioactive isotope can be detected by such means as the use of a γ counter or a scintillation counter or autoradiography. In certain embodiments, the extracellular antigen-binding domain of the CAR is labeled with a fluorescent marker. Non-limiting examples of fluorescent markers include green fluorescent protein (GFP), blue fluorescent protein (e.g., EBFP, EBFP2, Azurite, and mKalama1), cyan fluorescent protein (e.g., ECFP, Cerulean, and CyPet), and yellow fluorescent protein (e.g., YFP, Citrine, Venus, and YPet).

The extracellular antigen-binding domain can comprise or be an scFv, a Fab (which is optionally crosslinked), or an F(ab)₂. In certain embodiments, any of the foregoing molecules may be comprised in a chimeric protein with a heterologous sequence to form the extracellular antigen-binding domain. In certain embodiments, the extracellular antigen-binding domain comprises or is an scFv. In certain embodiments, the scFv is a human scFv. In certain embodiments, the scFv is a humanized scFv. In certain embodiments, the scFv is a murine scFv.

### 3.1.2.1.1. Exemplary Extracellular Antigen-Binding Domains

In certain embodiments, the antigen-recognizing receptor is a CAR comprising an extracellular antigen-binding domain that binds to CD19. In certain embodiments, the extracellular antigen-binding domain of the CAR comprises or consists of the amino acid sequence set forth in SEQ ID NO: 67 and specifically binds to CD19, e.g., a human CD19 polypeptide. SEQ ID NO: 67 is provided in Table 3 below.

In certain embodiments, the extracellular antigen-binding domain of the CAR comprises a heavy chain variable region (V_{H}) comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 59 or a conservative modification thereof. In certain embodiments, the extracellular antigen-binding domain of the CAR comprises a VH comprising a CDR1 comprising the amino acid sequence set forth in SEQ ID NO: 57, a CDR2 comprising the amino acid sequence set forth in SEQ ID NO: 58, and a CDR3 comprising the amino acid sequence set forth in SEQ ID NO: 59. SEQ ID NOs: 57-59 are provided in Table 3 below.

In certain embodiments, the extracellular antigen-binding domain of the CAR comprises a light chain variable region (V_{L}) comprising a CDR1 comprising the amino acid sequence set forth in SEQ ID NO: 60 or a conservative modification thereof, a CDR2 comprising the amino acid sequence set forth in SEQ ID NO: 61 or a conservative modification thereof, and a CDR3 comprising the amino acid sequence set forth in SEQ ID NO: 62 or a conservative modification thereof. In certain embodiments, the extracellular antigen-binding domain of the CAR comprises a V_{L} comprising a CDR1 comprising the amino acid sequence set forth in SEQ ID NO: 60, a CDR2 comprising the amino acid sequence set forth in SEQ ID NO: 61, and a CDR3 comprising the amino acid sequence set forth in SEQ ID NO: 62. SEQ ID NOs: 60-62 are provided in Table 3 below.

In certain embodiments, the extracellular antigen-binding domain of the CAR comprises a V_{H} comprising a CDR1 comprising the amino acid sequence set forth in SEQ ID NO: 57 or a conservative modification thereof, a CDR2 comprising the amino acid sequence set forth in SEQ ID NO: 58 or a conservative modification thereof, and a CDR3 comprising the amino acid sequence set forth in SEQ ID NO: 59, a conservative modification thereof, a V_{L} comprising a CDR1 comprising the amino acid sequence set forth in SEQ ID NO: 60 or a conservative modification thereof, a CDR2 comprising the amino acid sequence set forth in SEQ ID NO: 61 or a conservative modification thereof, and a CDR3 comprising the amino acid sequence set forth in SEQ ID NO: 62 or a conservative modification thereof. In certain embodiments, the extracellular antigen-binding domain of the CAR comprises a V_{H} comprising a CDR1 comprising amino acids having the sequence set forth in SEQ ID NO: 57, a CDR2 comprising the amino acid sequence set forth in SEQ ID NO: 58, and a CDR3 comprising the amino acid sequence set forth in SEQ ID NO: 59; and a V_{L} comprising a CDR1 comprising the amino acid sequence set forth in SEQ ID NO: 60, a CDR2 comprising the amino acid sequence set forth in SEQ ID NO: 61, and a CDR3 comprising the amino acid sequence set forth in SEQ ID NO: 62.

In certain embodiments, the extracellular antigen-binding domain of the CAR comprises a V_{H} comprising an amino acid sequence that is at least about 80% (e.g., at least about 85%, at least about 90%, or at least about 95%) homologous or identical to the amino acid sequence set forth in SEQ ID NO: 63 or SEQ ID NO: 64. For example, the extracellular antigen-binding domain of the antigen-recognizing receptor comprises a VH comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 63 or SEQ ID NO: 64. In certain embodiments, the extracellular antigen-binding domain of the CAR comprises a V_{H} comprising the amino acid sequence set forth in SEQ ID NO: 63 or SEQ ID NO: 64. SEQ ID NO: 63 and SEQ ID NO: 64 are provided in Table 3 below.

In certain embodiments, the extracellular antigen-binding domain of the CAR comprises a V_{L} comprising an amino acid sequence that is at least about 80% (e.g., at least about 85%, at least about 90%, or at least about 95%) homologous or identical to the amino acid sequence set forth in SEQ ID NO: 65 or SEQ ID NO: 66. For example, the extracellular antigen-binding domain of the CAR comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 65 or SEQ ID NO: 66. In certain embodiments, the extracellular antigen-binding domain of the CAR comprises a V_{L} comprising the amino acid sequence set forth in SEQ ID NO: 65 or SEQ ID NO: 66. SEQ ID NO: 65 and SEQ ID NO: 66 are provided in Table 3 below.

In certain embodiments, the extracellular antigen-binding domain of the CAR comprises a V_{H} comprising an amino acid sequence that is at least about 80% (e.g., at least about 85%, at least about 90%, or at least about 95%) homologous or identical to the amino acid sequence set forth in SEQ ID NO: 63 or SEQ ID NO: 64, and a V_{L} comprising an amino acid sequence that is at least about 80% (e.g., at least about 85%, at least about 90%, or at least about 95%) homologous or identical to the amino acid sequence set forth in SEQ ID NO: 65 or SEQ ID NO: 66.

In certain embodiments, the extracellular antigen-binding domain of the CAR comprises a V_{H} comprising the amino acid sequence set forth in SEQ ID NO: 63 or SEQ ID NO: 64. In certain embodiments, the extracellular antigen-binding domain of the CAR comprises a V_{L} comprising the amino acid sequence set forth in SEQ ID NO: 65 or SEQ ID NO: 66. In certain embodiments, the extracellular antigen-binding domain of the CAR comprises V_{H} comprising the amino acid sequence set forth in SEQ ID NO: 63 or SEQ ID NO: 64 and a V_{L} comprising the amino acid sequence set forth in SEQ ID NO: 65 or SEQ ID NO: 66.

In certain embodiments, the extracellular antigen-binding domain of the CAR is an scFv that comprises or consists of the amino acid sequence set forth in SEQ ID NO: 67. SEQ ID NOs: 57-67 are provided in the following Table 3. In certain embodiments, the V_{H} and V_{L} are linked via a linker. In certain embodiments, the linker comprises the amino acid sequence set forth in SEQ ID NO: 1, SEQ ID NO: 2, SEQ ID NO: 3, SEQ ID NO: 4, SEQ ID NO: 5, SEQ ID NO: 6, or SEQ ID NO: 26.

In certain embodiments, the CDRs regions/sequences disclosed herein are delineated using the Kabat system (Swindells et al., J Mol Biol. 2017 Feb. 3; 429(3):356-364).

TABLE 3
ANTI-HUMAN CD19 SCFV (SJ25C1)

| CDRS | 1 | 2 | 3 |
|---|---|---|---|
| V_{H} | GYAFSS | YPGDGD | KTISSVVDF |
| | [SEQ ID NO: 57] | [SEQ ID NO: 58] | [SEQ ID NO: 59] |
| V_{L} | KASQNVGTNVA | SATYRN | QQYNRYPYT |
| | [SEQ ID NO: 60] | [SEQ ID NO: 61] | [SEQ ID NO: 62] |

| | |
|---|---|
| FULL V_{H} | EVKLQQSGAELVRPGSSVKISCKASGYAFSSYWMNWVKQRPGQGLEWIGQIYPGDG |
| | DTNYNGKFKGQATLTADKSSSTAYMQLSGLTSEDSAVYFCARKTISSVVDFYFDYW |
| | GQGTTVTVSS [SEQ ID NO: 63] |
| FULL V_{H} | EVKLQQSGAELVRPGSSVKISCKASGYAFSSYWMNWVKQRPGQGLEWIGQIYPGDG |
| V2 | DTNYNGKFKGQATLTADKSSSTAYMQLSGLTSEDSAVYFCARKTISSVVDFYFDYW |
| | GQGTTVTV [SEQ ID NO: 64] |
| FULL V_{L} | DIELTQSPKFMSTSVGDRVSVTCKASQNVGTNVAWYQQKPGQSPKPLIYSATYRNS |
| | GVPDRFTGSGSGTDFTLTITNVQSKDLADYFCQQYNRYPYTSGGGTKLEIKR [SEQ |
| | ID NO: 65] |
| FULL V_{L} | DIELTQSPKFMSTSVGDRVSVTCKASQNVGTNVAWYQQKPGQSPKPLIYSATYRNS |
| V2 | GVPDRFTGSGSGTDFTLTITNVQSKDLADYFCQQYNRYPYTSGGGTKLEI [SEQ |
| | ID NO: 66] |
| SCFV | MALPVTALLLPLALLLHAEVKLQQSGAELVRPGSSVKISCKASGYAFSSYWMNWVK |
| | QRPGQGLEWIGQIYPGDGDTNYNGKFKGQATLTADKSSSTAYMQLSGLTSEDSAVY |
| | FCARKTISSVVDFYFDYWGQGTTVTVSSGGGGSGGGGSGGGGSDIELTQSPKFMST |
| | SVGDRVSVTCKASQNVGTNVAWYQQKPGQSPKPLIYSATYRNSGVPDRFTGSGSGT |
| | DFTLTITNVQSKDLADYFCQQYNRYPYTSGGGTKLEIKR [SEQ ID NO: 67] |

In certain embodiments, the antigen-recognizing receptor is a CAR comprising an extracellular antigen-binding domain that binds to CD70. In certain embodiments, the extracellular antigen-binding domain of the CAR comprises or consists of the amino acid sequence set forth in SEQ ID NO: 141 and specifically binds to CD70, e.g., a human CD70 polypeptide. SEQ ID NO: 141 is provided in Table 4 below.

In certain embodiments, the extracellular antigen-binding domain of the CAR comprises a heavy chain variable region (V_{H}) comprising a CDR1 comprising the amino acid sequence set forth in SEQ ID NO: 133 or a conservative modification thereof, a CDR2 comprising the amino acid sequence set forth in SEQ ID NO: 134 or a conservative modification thereof, and a CDR3 comprising the amino acid sequence set forth in SEQ ID NO: 135 or a conservative modification thereof. In certain embodiments, the extracellular antigen-binding domain of the CAR comprises a V_{H} comprising a CDR1 comprising the amino acid sequence set forth in SEQ ID NO: 133, a CDR2 comprising the amino acid sequence set forth in SEQ ID NO: 134, and a CDR3 comprising the amino acid sequence set forth in SEQ ID NO: 135. SEQ ID NOs: 133-135 are provided in Table 4 below.

In certain embodiments, the extracellular antigen-binding domain of the CAR comprises a light chain variable region (V_{L}) comprising a CDR1 comprising the amino acid sequence set forth in SEQ ID NO: 136 or a conservative modification thereof, a CDR2 comprising the amino acid sequence set forth in SEQ ID NO: 137 or a conservative modification thereof, and a CDR3 comprising the amino acid sequence set forth in SEQ ID NO: 138 or a conservative modification thereof. In certain embodiments, the extracellular antigen-binding domain of the CAR comprises a V_{L} comprising a CDR1 comprising the amino acid sequence set forth in SEQ ID NO: 136, a CDR2 comprising the amino acid sequence set forth in SEQ ID NO: 137, and a CDR3 comprising the amino acid sequence set forth in SEQ ID NO: 138. SEQ ID NOs: 136-138 are provided in Table 4 below.

In certain embodiments, the extracellular antigen-binding domain of the CAR comprises a V_{H} comprising a CDR1 comprising the amino acid sequence set forth in SEQ ID NO: 133 or a conservative modification thereof, a CDR2 comprising the amino acid sequence set forth in SEQ ID NO: 134 or a conservative modification thereof, and a CDR3 comprising the amino acid sequence set forth in SEQ ID NO: 135, a conservative modification thereof, a V_{L} comprising a CDR1 comprising the amino acid sequence set forth in SEQ ID NO: 136 or a conservative modification thereof, a CDR2 comprising the amino acid sequence set forth in SEQ ID NO: 137 or a conservative modification thereof, and a CDR3 comprising the amino acid sequence set forth in SEQ ID NO: 138 or a conservative modification thereof. In certain embodiments, the extracellular antigen-binding domain of the CAR comprises a V_{H} comprising a CDR1 comprising amino acids having the sequence set forth in SEQ ID NO: 133, a CDR2 comprising the amino acid sequence set forth in SEQ ID NO: 134, and a CDR3 comprising the amino acid sequence set forth in SEQ ID NO: 135; and a V_{L} comprising a CDR1 comprising the amino acid sequence set forth in SEQ ID NO: 136, a CDR2 comprising the amino acid sequence set forth in SEQ ID NO: 137, and a CDR3 comprising the amino acid sequence set forth in SEQ ID NO: 138.

In certain embodiments, the extracellular antigen-binding domain of the CAR comprises a V_{H} comprising an amino acid sequence that is at least about 80% (e.g., at least about 85%, at least about 90%, or at least about 95%) homologous or identical to the amino acid sequence set forth in SEQ ID NO: 139. For example, the extracellular antigen-binding domain of the antigen-recognizing receptor comprises a VH comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 139. In certain embodiments, the extracellular antigen-binding domain of the CAR comprises a V_{H} comprising the amino acid sequence set forth in SEQ ID NO: 139. SEQ ID NO: 139 is provided in Table 4 below.

In certain embodiments, the extracellular antigen-binding domain of the CAR comprises a V_{L} comprising an amino acid sequence that is at least about 80% (e.g., at least about 85%, at least about 90%, or at least about 95%) homologous or identical to the amino acid sequence set forth in SEQ ID NO: 140. For example, the extracellular antigen-binding domain of the CAR comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 140. In certain embodiments, the extracellular antigen-binding domain of the CAR comprises a V_{L} comprising the amino acid sequence set forth in SEQ ID NO: 140. SEQ ID NO: 140 is provided in Table 4 below.

In certain embodiments, the extracellular antigen-binding domain of the CAR comprises a VH comprising an amino acid sequence that is at least about 80% (e.g., at least about 85%, at least about 90%, or at least about 95%) homologous or identical to the amino acid sequence set forth in SEQ ID NO: 139, and a V_{L} comprising an amino acid sequence that is at least about 80% (e.g., at least about 85%, at least about 90%, or at least about 95%) homologous or identical to the amino acid sequence set forth in SEQ ID NO: 140.

In certain embodiments, the extracellular antigen-binding domain of the CAR comprises a V_{H} comprising the amino acid sequence set forth in SEQ ID NO: 139. In certain embodiments, the extracellular antigen-binding domain of the CAR comprises a V_{L} comprising the amino acid sequence set forth in SEQ ID NO: 140. In certain embodiments, the extracellular antigen-binding domain of the CAR comprises V_{H} comprising the amino acid sequence set forth in SEQ ID NO: 139 and a V_{L} comprising the amino acid sequence set forth in SEQ ID NO: 140.

In certain embodiments, the extracellular antigen-binding domain of the CAR is an scFv that comprises or consists of the amino acid sequence set forth in SEQ ID NO: 141. SEQ ID NOs: 133-141 are provided in the following Table 4. In certain embodiments, the V_{H} and V_{L} are linked via a linker. In certain embodiments, the linker comprises the amino acid sequence set forth in SEQ ID NO: 1, SEQ ID NO: 2, SEQ ID NO: 3, SEQ ID NO: 4, SEQ ID NO: 5, SEQ ID NO: 6, or SEQ ID NO: 26.

In certain embodiments, the extracellular antigen-binding domain of the CAR comprises a CDR1, a CDR2, and a CDR3 of a V_{H} sequence of an anti-CD70 antibody disclosed in International Patent Publication No. WO 2007/038637, which is incorporated by reference in its entirety. In certain embodiments, the extracellular antigen-binding domain of the CAR comprises a CDR1, a CDR2, and a CDR3 of the V_{H} sequence of the anti-CD70 antibody 2H5 disclosed in International Patent Publication No. WO 2007/038637. In certain embodiments, the extracellular antigen-binding domain of the CAR comprises a V_{H} sequence of an anti-CD70 antibody disclosed in International Patent Publication No. WO 2007/038637.

In certain embodiments, the extracellular antigen-binding domain of the CAR comprises a CDR1, a CDR2, and a CDR3 of a V_{L} sequence of an anti-CD70 antibody disclosed in International Patent Publication No. WO 2007/038637. In certain embodiments, the extracellular antigen-binding domain of the CAR comprises a CDR1, a CDR2, and a CDR3 of the V_{L} sequence of the anti-CD70 antibody 2H5 disclosed in International Patent Publication No. WO 2007/038637. In certain embodiments, the extracellular antigen-binding domain of the CAR comprises a V_{L} sequence of an anti-CD70 antibody disclosed in International Patent Publication No. WO 2007/038637.

In certain embodiments, the extracellular antigen-binding domain of the CAR comprises an antigen-binding fragment of an anti-CD70 antibody disclosed in International Patent Publication No. WO 2007/038637. In certain embodiments, the extracellular antigen-binding domain of the CAR comprises an antigen-binding fragment of the anti-CD70 antibody 2H5 disclosed in International Patent Publication No. WO 2007/038637.

In certain embodiments, the extracellular antigen-binding domain of the CAR comprises an antigen-binding fragment of an anti-CD70 antibody disclosed in International Patent Publication No. WO 2007/038637. In certain embodiments, the extracellular antigen-binding domain of the CAR comprises an antigen-binding fragment of the anti-CD70 antibody 2H5 disclosed in International Patent Publication No. WO 2007/038637.

In certain embodiments, the CDRs regions/sequences disclosed herein are delineated using the PyIgClassify system (Adolf-Bryfogle et al., Nucleic acids research 43.D1 (2015): D432-D438).

TABLE 4

| CDRs | 1 | 2 | 3 |
|---|---|---|---|
| V_{H} | AASGFTFSSYIMH | VISYDGRNKY | ARDTDGYDFDY |
| | [SEQ ID NO: 133] | [SEQ ID NO: 134] | [SEQ ID NO: 135] |
| V_{L} | RASQSVSSYLA | YDASNRAT | QQRTNWPLT |
| | [SEQ ID NO: 136] | [SEQ ID NO: 137] | [SEQ ID NO: 138] |

| | |
|---|---|
| Full V_{H} | QVQLVESGGGVVQPGRSLRLSCAASGFTFSSYIMHWVRQAPGKGLEWVAVISYDGRNKYYAD |
| | SVKGRFTISRDNSKNTLYLQMNSLRAEDTAVYYCARDTDGYDFDYWGQGTLVTVSS [SEQ |
| | ID NO: 139] |
| Full V_{L} | EIVLTQSPATLSLSPGERATLSCRASQSVSSYLAWYQQKPGQAPRLLIYDASNRATGIPARF |
| | SGSGSGTDFTLTISSLEPEDFAVYYCQQRTNWPLTFGGGTKVEIK [SEQ ID NO: 140] |
| scFv | QVQLVESGGGVVQPGRSLRLSCAASGFTFSSYIMHWVRQAPGKGLEWVAVISYDGRNKYYAD |
| | SVKGRFTISRDNSKNTLYLQMNSLRAEDTAVYYCARDTDGYDFDYWGQGTLVTVSSGGGGSG |
| | GGGSGGGGSEIVLTQSPATLSLSPGERATLSCRASQSVSSYLAWYQQKPGQAPRLLIYDASN |
| | RATGIPARFSGSGSGTDFTLTISSLEPEDFAVYYCQQRTNWPLTFGGGTKVEIK [SEQ ID |
| | NO: 141] |

The V_{H} and/or V_{L} amino acid sequences having at least about 80%, at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homology or identity to a specific sequence (e.g., SEQ ID NOs: 63, 64, 65, 66, 139, and 140) may contain substitutions (e.g., conservative substitutions), insertions, or deletions relative to the specified sequence(s), but retain the ability to bind to a target antigen (e.g., CD19, CD70). In certain embodiments, a total of 1 to 10 amino acids are substituted, inserted, and/or deleted in a specific sequence (e.g., SEQ ID NOs: 63, 64, 65, 66, 139, and 140). In certain embodiments, substitutions, insertions, or deletions occur in regions outside the CDRs (e.g., in the FRs) of the extracellular antigen-binding domain. In certain embodiments, the extracellular antigen-binding domain of the CAR comprises V_{H} and/or V_{L} sequence selected from SEQ ID NOs: 63, 64, 65, 66, 139, and 140, including post-translational modifications of that sequence (SEQ ID NO: 63, 64, 65, 66, 139, and 140).

In addition, the extracellular antigen-binding domain of the CAR can comprise a leader or a signal peptide that directs the nascent protein into the endoplasmic reticulum. Signal peptide or leader can be essential if the CAR is to be glycosylated and anchored in the cell membrane. The signal sequence or leader can be a peptide sequence (about 5, about 10, about 15, about 20, about 25, or about 30 amino acids long) present at the N-terminus of newly synthesized proteins that directs their entry to the secretory pathway. In certain embodiments, the signal peptide is covalently joined to the 5′ terminus (N-terminus) of the extracellular antigen-binding domain of the CAR. In certain embodiments, the signal peptide comprises a CD8 polypeptide, e.g., the CAR comprises a truncated CD8 signal peptide. In certain embodiments, the signal peptide comprises or consists of the amino acid sequence set forth in SEQ ID NO: 12.

### 3.1.2.2. Transmembrane Domain and Hinge Spacer Region

In certain embodiments, the antigen-recognizing receptor is a CAR that comprises a transmembrane domain. Different transmembrane domains result in different receptor stability. After antigen recognition, receptors cluster, and a signal is transmitted to the cell. In accordance with the presently disclosed subject matter, the transmembrane domain of the antigen-recognizing receptor can comprise a native or modified transmembrane domain of a CD8 polypeptide, a CD28 polypeptide, a CD3ζ polypeptide, a CD40 polypeptide, a 4-1BB polypeptide, an OX40 polypeptide, a CD84 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, an NKG2D polypeptide, a synthetic polypeptide (not based on a protein associated with the immune response), or a combination thereof.

In certain embodiments, the transmembrane domain of the CAR comprises a CD28 polypeptide (e.g., the transmembrane domain of CD28 or a portion thereof). In certain embodiments, the transmembrane domain of the CAR comprises a transmembrane domain of human CD28 or a portion thereof. In certain embodiments, the CD28 polypeptide comprises or consists of an amino acid sequence that is a consecutive portion of the amino acid sequence having an NCBI Reference No: NP_006130 (SEQ ID NO: 43), which is at least about 20, or at least about 25, or at least about 30, and/or up to about 220 amino acids in length. In certain embodiments, the CD28 polypeptide comprises or consists of an amino acid sequence of amino acids 1 to 220, 1 to 50, 50 to 100, 100 to 150, 114 to 220, 150 to 200, 153 to 179, or 200 to 220 of SEQ ID NO: 43. In certain embodiments, the transmembrane domain of the CAR comprises a CD28 polypeptide that comprises or consists of amino acids 153 to 179 of SEQ ID NO: 43.

In certain embodiments, the antigen-recognizing receptor is a CAR that further comprises a hinge/spacer region that links the extracellular antigen-binding domain to the transmembrane domain. The hinge/spacer region can be flexible enough to allow the antigen binding domain to orient in different directions to facilitate antigen recognition. In certain embodiments, the hinge/spacer region of the CAR can comprise a native or modified hinge region of a CD8 polypeptide, a CD28 polypeptide, a CD3ζ polypeptide, a CD40 polypeptide, a 4-1BB polypeptide, an OX40 polypeptide, a CD84 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, an NKG2D polypeptide, a synthetic polypeptide (not based on a protein associated with the immune response), or a combination thereof. The hinge/spacer region can be the hinge region from IgG1, the CH₂CH₃ region of immunoglobulin and portions of CD3, a portion of a CD28 polypeptide (e.g., a portion of SEQ ID NO: 43), a portion of a CD8 polypeptide, or a synthetic spacer sequence.

In certain embodiments, the antigen-recognizing receptor is a CAR that further comprises a hinge/spacer region comprising a native or modified hinge region of a CD28 polypeptide. In certain embodiments, the hinge/spacer region of the antigen-recognizing receptor (e.g., a CAR) comprises a CD28 polypeptide comprising or consisting of amino acids 114 to 152 of SEQ ID NO: 43.

In certain embodiments, the hinge/spacer region is positioned between the extracellular antigen-binding domain and the transmembrane domain. In certain embodiments, the hinge/spacer region comprises a CD8 polypeptide, a CD28 polypeptide, a CD3ζ polypeptide, a CD4 polypeptide, a 4-1BB polypeptide, an OX40 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, an NKG2D polypeptide, a synthetic polypeptide (not based on a protein associated with the immune response), or a combination thereof. In certain embodiments, the transmembrane domain comprises a CD8 polypeptide, a CD28 polypeptide, a CD3ζ polypeptide, a CD4 polypeptide, a 4-1BB polypeptide, an OX40 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, an NKG2D polypeptide, a synthetic polypeptide (not based on a protein associated with the immune response), or a combination thereof.

In certain embodiments, the transmembrane domain and the hinge/spacer region are derived from the same molecule. In certain embodiments, the transmembrane domain and the hinge/spacer region are derived from different molecules. In certain embodiments, the hinge/spacer region comprises a CD28 polypeptide and the transmembrane domain comprises a CD28 polypeptide. In certain embodiments, the hinge/spacer region comprises a CD28 polypeptide and the transmembrane domain comprises a CD28 polypeptide. In certain embodiments, the hinge/spacer region comprises a CD84 polypeptide and the transmembrane domain comprises a CD84 polypeptide. In certain embodiments, the hinge/spacer region comprises a CD166 polypeptide and the transmembrane domain comprises a CD166 polypeptide. In certain embodiments, the hinge/spacer region comprises a CD8a polypeptide and the transmembrane domain comprises a CD8a polypeptide. In certain embodiments, the hinge/spacer region comprises a CD8b polypeptide and the transmembrane domain comprises a CD8b polypeptide. In certain embodiments, the hinge/spacer region comprises a CD28 polypeptide and the transmembrane domain comprises an ICOS polypeptide.

### 3.1.2.3. Intracellular Signaling Domain

In certain embodiments, the antigen-recognizing receptor is a CAR that comprises an intracellular signaling domain. In certain embodiments, the intracellular signaling domain of the CAR comprises a CD3ζ polypeptide. CD3ζ can activate or stimulate a cell (e.g., a cell of the lymphoid lineage, e.g., a T-cell). Wild type (“native”) CD3ζ comprises three functional immunoreceptor tyrosine-based activation motifs (ITAMs), and three functional basic-rich stretch (BRS) regions (BRS1, BRS2, and BRS3). CD3ζ transmits an activation signal to the cell (e.g., a cell of the lymphoid lineage, e.g., a T-cell) after the antigen is bound. The intracellular signaling domain of the CD3ζ-chain is the primary transmitter of signals from endogenous TCRs.

In certain embodiments, the intracellular signaling domain of the CAR comprises a native CD3ζ. In certain embodiments, the native CD3ζ comprises or consists of an amino acid sequence that is at least about 85%, about 90%, about 95%, about 96%, about 97%, about 98%, about 99%, or about 100% identical or homologous to the amino acid sequence having an NCBI Reference No: NP_932170 (SEQ ID NO: 68) or a fragment thereof, and/or may optionally comprise up to one or up to two or up to three conservative amino acid substitutions. In certain embodiments, the CD3ζ polypeptide comprises or consists of an amino acid sequence that is a consecutive portion of SEQ ID NO: 68, which is at least about 20, or at least about 30, or at least about 40, or at least about 50, and up to about 164 amino acids in length. In certain embodiments, the native CD3ζ comprises or consists of the amino acid sequence of amino acids 1 to 164, 1 to 50, 50 to 100, 52 to 164, 100 to 150, or 150 to 164 of SEQ ID NO: 68. In certain embodiments, the intracellular signaling domain of the CAR comprises a native CD3ζ comprising or consisting of the amino acid sequence of amino acids 52 to 164 of SEQ ID NO: 68. SEQ ID NO: 68 is provided below:

[SEQ ID NO: 68]
MKWKALFTAAILQAQLPITEAQSFGLLDPKLCYLLDGILFIYGVILTAL
FLRVKFSRSADAPAYQQGQNQLYNELNLGRREEYDVLDKRRGRDPEMGG
KPQRRKNPQEGLYNELQKDKMAEAYSEIGMKGERRRGKGHDGLYQGLST
ATKDTYDALHMQALPPR

In certain embodiments, the native CD3ζ comprises or consists of an amino acid sequence that is at least about 85%, about 90%, about 95%, about 96%, about 97%, about 98%, about 99%, or about 100% identical or homologous to the amino acid sequence set forth in SEQ ID NO: 69. SEQ ID NO: 69 is provided below:

[SEQ ID NO: 69]
RVKESRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR

In certain embodiments, the intracellular signaling domain of the CAR comprises a modified CD3ζ polypeptide. In certain embodiments, the modified CD3ζ polypeptide comprises one, two, or three ITAMs. In certain embodiments, the modified CD3ζ polypeptide comprises a native ITAM1. In certain embodiments, the native ITAM1 comprises or consists of the amino acid sequence set forth in SEQ ID NO: 70.

[SEQ ID NO: 70]
QNQLYNELNLGRREEYDVLDKR

An exemplary nucleic acid sequence encoding the amino acid sequence of SEQ ID NO: 70 is set forth in SEQ ID NO: 71, which is provided below.

[SEQ ID NO: 71]
CAGAACCAGCTCTATAACGAGCTCAATCTAGGACGAAGAGAGGAGTACG
ATGTTTTGGACAAGAGA

In certain embodiments, the modified CD3ζ polypeptide comprises an ITAM1 variant comprising one or more loss-of-function mutations. In certain embodiments, the ITAM1 variant comprises or consists of two loss-of-function mutations. In certain embodiments, each 35 of the one or more (e.g., two) loss of function mutations comprises a mutation of a tyrosine residue in ITAM1. In certain embodiments, the ITAM1 variant consists of two loss-of-function mutations. In certain embodiments, the ITAM1 variant comprises or consists of the amino acid sequence set forth in SEQ ID NO: 72, which is provided below.

[SEQ ID NO: 72]
QNQLENELNLGRREEFDVLDKR

An exemplary nucleic acid sequence encoding the amino acid sequence of SEQ ID NO: 72 is set forth in SEQ ID NO: 73, which is provided below.

[SEQ ID NO: 73]
CAGAACCAGCTCTTTAACGAGCTCAATCTAGGACGAAGAGAGGAGTTCG
ATGTTTTGGACAAGAGA

In certain embodiments, the modified CD3ζ polypeptide comprises a native ITAM2. In certain embodiments, the native ITAM2 comprises or consists of the amino acid sequence set forth in SEQ ID NO: 74, which is provided below.

[SEQ ID NO: 74]
QEGLYNELQKDKMAEAYSEIGMK

An exemplary nucleic acid sequence encoding the amino acid sequence of SEQ ID NO: 74 is set forth in SEQ ID NO: 75, which is provided below.

[SEQ ID NO: 75]
CAGGAAGGCCTGTACAATGAACTGCAGAAAGATAAGATGGCGGAGGCCT
ACAGTGAGATTGGGATGAAA

In certain embodiments, the modified CD3ζ polypeptide comprises an ITAM2 variant. In certain embodiments, the ITAM2 variant comprises or consists of one or more loss-of-function mutations. In certain embodiments, the ITAM2 variant comprises or consists of two loss-of-function mutations. In certain embodiments, each of the one or more (e.g., two) loss of function mutations comprises a mutation of a tyrosine residue in ITAM2. In certain embodiments, the ITAM1 variant consists of two loss-of-function mutations. In certain embodiments, the ITAM2 variant comprises or consists of the amino acid sequence set forth in SEQ ID NO 76, which is provided below

[SEQ ID NO: 76]
QEGLFNELQKDKMAEAFSEIGMK

An exemplary nucleic acid sequence encoding the amino acid sequence of SEQ ID NO: 76 is set forth in SEQ ID NO: 77, which is provided below.

[SEQ ID NO: 77]
CAGGAAGGCCTGTTCAATGAACTGCAGAAAGATAAGATGGCGGAGGCCT
TCAGTGAGATTGGGATGAAA

In certain embodiments, the modified CD3ζ polypeptide comprises a native ITAM3. In certain embodiments, the native ITAM3 comprises or consists of the amino acid sequence set forth in SEQ ID NO: 78, which is provided below.

[SEQ ID NO: 78]
HDGLYQGLSTATKDTYDALHMQ

An exemplary nucleic acid sequence encoding the amino acid sequence of SEQ ID NO: 78 is set forth in SEQ ID NO: 79, which is provided below.

[SEQ ID NO: 79]
CACGATGGCCTTTACCAGGGTCTCAGTACAGCCACCAAGGACACCTACG
ACGCCCTTCACATGCAG

In certain embodiments, the modified CD3ζ polypeptide comprises an ITAM3 variant. In certain embodiments, the ITAM3 variant comprises or consists of two loss-of-function mutations. In certain embodiments, each of the one or more (e.g., two) loss of function mutations comprises a mutation of a tyrosine residue in ITAM3. In certain embodiments, the ITAM3 variant comprises or consists of two loss-of-function mutations. In certain embodiments, the ITAM3 variant comprises or consists of the amino acid sequence set forth in SEQ ID NO: 80, which is provided below.

[SEQ ID NO: 80]
HDGLFQGLSTATKDTEDALHMQ

An exemplary nucleic acid sequence encoding the amino acid sequence of SEQ ID NO: 80 is set forth in SEQ ID NO: 81, which is provided below.

[SEQ ID NO: 81]
CACGATGGCCTTTTCCAGGGGCTCAGTACAGCCACCAAGGACACCTTCG
ACGCCCTTCACATGCAG

Various modified CD3ζ polypeptides and CARs comprising modified CD3ζ polypeptides are disclosed in International Patent Application Publication No. WO2019/133969, which is incorporated by reference hereby in its entirety.

In certain embodiments, the intracellular signaling domain of the CAR comprises a modified CD3ζ polypeptide comprising a native ITAM1, an ITAM2 variant comprising or consisting of one or more (e.g., two) loss-of-function mutations, and an ITAM3 variant comprising or consisting of one or more (e.g., two) loss-of-function mutations. In certain embodiments, the intracellular signaling domain of the CAR comprises a modified CD3ζ polypeptide comprising a native ITAM1, an ITAM2 variant consisting of two loss-of-function mutations, and an ITAM3 variant consisting of two loss-of-function mutations. In certain embodiments, the intracellular signaling domain of the CAR comprises a modified CD3ζ polypeptide comprising a native ITAM1 consisting of the amino acid sequence set forth in SEQ ID NO: 70, an ITAM2 variant consisting of the amino acid sequence set forth in SEQ ID NO: 76, and an ITAM3 variant consisting of the amino acid sequence set forth in SEQ ID NO: 80. In certain embodiments, the CAR is designated as “1XX”. In certain embodiments, the modified CD3ζ polypeptide comprises or consists of the amino acid sequence set forth in SEQ ID NO: 82. SEQ ID NO: 82 is provided below:

[SEQ ID NO: 82]
RVKFSRSADAPAYQQGQNQLYNELNLGRREEYDVLDKRRGRDPEMGGKPR
RKNPQEGLFNELQKDKMAEAFSEIGMKGERRRGKGHDGLFQGLSTATKDT
EDALHMQALPPR

In certain embodiments, the intracellular signaling domain of the CAR comprises a modified CD3ζ polypeptide comprising or consisting of an amino acid sequence that is at least about 80%, at least about 85%, at least about 90%, at least about 95%, at least about 96%, at least about 97%, at least about 98%, or at least about 99%, at least about 100% identical to SEQ ID NO: 82 or a fragment thereof, and/or may optionally comprise up to one or up to two or up to three conservative amino acid substitutions.

An exemplary nucleic acid sequence encoding the amino acid sequence of SEQ ID NO: 82 is set forth in SEQ ID NO: 83, which is provided below.

[SEQ ID NO: 83]
AGAGTGAAGTTCAGCAGGAGCGCAGACGCCCCCGCGTACCAGCAGGGCCA
GAACCAGCTCTATAACGAGCTCAATCTAGGACGAAGAGAGGAGTACGATG
TTTTGGACAAGAGACGTGGCCGGGACCCTGAGATGGGGGGAAAGCCGAGA
AGGAAGAACCCTCAGGAAGGCCTGTTCAATGAACTGCAGAAAGATAAGAT
GGCGGAGGCCTTCAGTGAGATTGGGATGAAAGGCGAGCGCCGGAGGGGCA
AGGGGCACGATGGCCTTTTCCAGGGGCTCAGTACAGCCACCAAGGACACC
TTCGACGCCCTTCACATGCAGGCCCTGCCCCCTCGC

In certain embodiments, the intracellular signaling domain of the CAR further comprises at least one co-stimulatory signaling region. In certain embodiments, the at least one co-stimulatory region comprises a co-stimulatory molecule or a portion thereof. In certain embodiments, the at least one co-stimulatory region comprises at least an intracellular domain of at least one co-stimulatory molecule or a portion thereof. Non-limiting examples of costimulatory molecules include CD28, 4-1BB, OX40, CD27, CD40, CD154, CD97, CD11a/CD18, ICOS, DAP-10, CD2, CD150, CD226, and NKG2D.

In certain embodiments, the intracellular signaling domain of the CAR comprises a co-stimulatory signaling region that comprises a CD28 polypeptide, e.g., an intracellular domain of CD28 or a portion thereof. In certain embodiments, the intracellular signaling domain of the CAR comprises a co-stimulatory signaling region that comprises an intracellular domain of human CD28 or a portion thereof.

In certain embodiments, the CD28 polypeptide comprised in the co-stimulatory signaling region of the antigen-recognizing receptor comprises or consists of an amino acid sequence that is at least about 80%, at least about 85%, at least about 90%, at least about 95%, at least about 96%, at least about 97%, at least about 98%, or at least about 99%, at least about 100% identical or homologous to the amino acid sequence set forth in SEQ ID NO: 43 or a fragment thereof, and/or may optionally comprise up to one or up to two or up to three conservative amino acid substitutions. In certain embodiments, the CD28 polypeptide comprised in the co-stimulatory signaling region of the CAR comprises or consists of an amino acid sequence that is a consecutive portion of SEQ ID NO: 7, which is at least about 20, or at least about 30, or at least about 40, or at least about 50, and up to about 220 amino acids in length. Alternatively or additionally, in certain embodiments, the CD28 polypeptide comprised in the co-stimulatory signaling region of the CAR comprises or consists of amino acids 1 to 220, 1 to 50, 50 to 100, 100 to 150, 114 to 220, 150 to 200, 180 to 220, or 200 to 220 of SEQ ID NO: 43. In certain embodiments, the intracellular signaling domain of the CAR comprises a co-stimulatory signaling region that comprises a CD28 polypeptide comprising or consisting of amino acids 180 to 220 of SEQ ID NO: 43.

An exemplary nucleic acid sequence encoding the amino acid sequence of amino acids 180 to 220 of SEQ ID NO: 43 is set forth in SEQ ID NO: 84, which is provided below.

[SEQ ID NO: 84]
AGGAGTAAGAGGAGCAGGCTCCTGCACAGTGACTACATGAACATGACTCC
CCGCCGCCCCGGGCCCACCCGCAAGCATTACCAGCCCTATGCCCCACCAC
GCGACTTCGCAGCCTATCGCTCC

In certain embodiments, the intracellular signaling domain of the antigen-recognizing receptor comprises a co-stimulatory signaling region that comprises an intracellular domain of mouse CD28 or a portion thereof. In certain embodiments, the CD28 polypeptide comprised in the co-stimulatory signaling region comprises or consists of an amino acid sequence that is at least about 80%, at least about 85%, at least about 90%, at least about 95%, at least about 96%, at least about 97%, at least about 98%, or at least about 99%, at least about 100% identical or homologous to the amino acid sequence having an NCBI Reference No: NP_031668.3 (or SEQ ID NO: 85) or a fragment thereof, and/or may optionally comprise up to one or up to two or up to three conservative amino acid substitutions. In certain embodiments, the CD28 polypeptide comprised in the co-stimulatory signaling region of the CAR comprises or consists of an amino acid sequence that is a consecutive portion of SEQ ID NO: 85, which is at least about 20, or at least about 30, or at least about 40, or at least about 50, and up to 218 amino acids in length. In certain embodiments, the CD28 polypeptide comprised in the co-stimulatory signaling region of the CAR comprises or consists of the amino acid sequence of amino acids 1 to 218, 1 to 50, 50 to 100, 100 to 150, 150 to 218, 178 to 218, or 200 to 218 of SEQ ID NO: 85. In certain embodiments, the co-stimulatory signaling region of the CAR comprises a CD28 polypeptide that comprises or consists of amino acids 178 to 218 of SEQ ID NO: 85. SEQ ID NO: 85 is provided below.

[SEQ ID NO: 85]
MTLRLLFLALNFFSVQVTENKILVKQSPLLVVDSNEVSLSCRYSYNLLAK
EFRASLYKGVNSDVEVCVGNGNFTYQPQFRSNAEENCDGDEDNETVTFRL
WNLHVNHTDIYFCKIEFMYPPPYLDNERSNGTIIHIKEKHLCHTQSSPKL
FWALVVVAGVLFCYGLLVTVALCVIWTNSRRNRLLQSDYMNMTPRRPGLT
RKPYQPYAPARDFAAYRP

In certain embodiments, the intracellular signaling domain of the CAR comprises a co-stimulatory signaling region that comprises a 4-1BB polypeptide, e.g., an intracellular domain of 4-1BB or a portion thereof. In certain embodiments, the co-stimulatory signaling region comprises an intracellular domain of human 4-1BB or a portion thereof. In certain embodiments, the 4-1BB comprised in the co-stimulatory signaling region of the CAR comprises or consists of an amino acid sequence that is at least about 80%, at least about 85%, at least about 90%, at least about 95%, at least about 96%, at least about 97%, at least about 98%, or at least about 99%, at least about 100% identical or homologous to the sequence having an NCBI Ref No.: NP_001552 (SEQ ID NO: 41 or SEQ ID NO: 42) or a fragment thereof, and/or may optionally comprise up to one or up to two or up to three conservative amino acid substitutions. In certain embodiments, the 4-1BB comprised in the co-stimulatory signaling region of the CAR comprises or consists of an amino acid sequence that is a consecutive portion of SEQ ID NO: 41 or SEQ ID NO: 42, which is at least about 20, or at least about 30, or at least about 40, or at least about 50, and/or up to about 50, up to about 60, up to about 70, up to about 80, up to about 90, up to about 100, up to about 200, or up to about 255 amino acids in length. In certain embodiments, the co-stimulatory signaling region of the CAR comprises a 4-1BB polypeptide that comprises or consists of the amino acid sequence of amino acids 1 to 255, 1 to 50, 50 to 100, 100 to 150, 150 to 200, or 200 to 255 of SEQ ID NO: 41 or SEQ ID NO: 42. In certain embodiments, the co-stimulatory signaling region of the CAR comprises a 4-1BB polypeptide comprising or consisting of the amino acid sequence of amino acids 214 to 255 of SEQ ID NO: 41, or SEQ ID NO: 42.

In certain embodiments, the intracellular signaling domain of the CAR comprises two co-stimulatory signaling regions, wherein the first co-stimulatory signaling region comprises an intracellular domain of a first co-stimulatory molecule or a portion thereof, and the second co-stimulatory signaling region comprises an intracellular domain of a second co-stimulatory molecule or a portion thereof. The first and second co-stimulatory molecules are independently selected from the group consisting of CD28, 4-1BB, OX40, CD27, CD40, CD154, CD97, CD11a/CD18, ICOS, DAP-10, CD2, CD150, CD226, and NKG2D. In certain embodiments, the intracellular signaling domain of the CAR comprises two co-stimulatory signaling regions, wherein the first co-stimulatory signaling region comprises an intracellular domain of CD28 or a portion thereof and the second co-stimulatory signaling region comprises an intracellular domain of 4-1BB or a portion thereof.

In certain embodiments, the antigen-recognizing receptor is a CAR that comprises i) an extracellular antigen-binding domain, ii) a transmembrane domain comprising a CD28 polypeptide (e.g., human CD28 polypeptide, e.g., a transmembrane domain of CD28 (e.g., human CD28) or a portion thereof), iii) a hinge/spacer region derived from a CD28 polypeptide (e.g., a human CD28 polypeptide), iv) an intracellular signaling domain comprising a) a native CD3ζ polypeptide, and b) a co-stimulatory signaling region comprising a CD28 polypeptide (e.g., a human CD28 polypeptide, e.g., an intracellular domain of CD28 (e.g., human CD28) of a portion thereof). In certain embodiments, the transmembrane domain comprises a CD28 polypeptide comprising or consisting of amino acids 153 to 179 of SEQ ID NO: 43. In certain embodiments, the hinge/spacer region comprises a CD28 polypeptide comprising or consisting of amino acids 114 to 152 of SEQ ID NO: 43. In certain embodiments, the intracellular signaling domain comprises a native CD3ζ polypeptide comprising or consisting of the amino acid sequence set forth in SEQ ID NO: 69, and a co-stimulatory signaling region comprising a CD28 polypeptide that comprises or consists of 180 to 220 of SEQ ID NO: 43. In certain embodiments, the CAR is designated as “28z”. In certain embodiments, the CAR (e.g., 28z) is encoded by a nucleotide sequence that is at least about 80%, at least about 85%, at least about 90%, at least about 95%, at least about 96%, at least about 97%, at least about 98%, or at least about 99%, at least about 100% identical to the nucleotide sequence set forth in SEQ ID NO: 86. In certain embodiments, the CAR (e.g., 28z) comprises the nucleotide sequence set forth in SEQ ID NO: 86. SEQ ID NO: 86 is provided below.

[SEQ ID NO: 86]
ATTGAAGTTATGTATCCTCCTCCTTACCTAGACAATGAGAAGAGCAATGG
AACCATTATCCATGTGAAAGGGAAACACCTTTGTCCAAGTCCCCTATTTC
CCGGACCTTCTAAGCCCTTTTGGGTGCTGGTGGTGGTTGGTGGAGTCCTG
GCTTGCTATAGCTTGCTAGTAACAGTGGCCTTTATTATTTTCTGGGTGAG
GAGTAAGAGGAGCAGGCTCCTGCACAGTGACTACATGAACATGACTCCCC
GCCGCCCCGGGCCCACCCGCAAGCATTACCAGCCCTATGCCCCACCACGC
GACTTCGCAGCCTATCGCTCCAGAGTGAAGTTCAGCAGGAGCGCAGACGC
CCCCGCGTACCAGCAGGGCCAGAACCAGCTCTATAACGAGCTCAATCTAG
GACGAAGAGAGGAGTACGATGTTTTGGACAAGAGACGTGGCCGGGACCCT
GAGATGGGGGGAAAGCCGAGAAGGAAGAACCCTCAGGAAGGCCTGTACAA
TGAACTGCAGAAAGATAAGATGGCGGAGGCCTACAGTGAGATTGGGATGA
AAGGCGAGCGCCGGAGGGGCAAGGGGCACGATGGCCTTTACCAGGGTCTC
AGTACAGCCACCAAGGACACCTACGACGCCCTTCACATGCAGGCCCTGCC
CCCTCGC

In certain embodiments, the antigen-recognizing receptor is a CAR that comprises i) an extracellular antigen-binding domain, ii) a transmembrane domain comprising a CD28 polypeptide (e.g., human CD28 polypeptide, e.g., a transmembrane domain of CD28 (e.g., human CD28) or a portion thereof), iii) a hinge/spacer region derived from a CD28 polypeptide (e.g., a human CD28 polypeptide), iv) an intracellular signaling domain comprising a) a modified CD3ζ polypeptide (e.g., a modified human CD3ζ polypeptide) comprising a native ITAM1, an ITAM2 variant consisting of two loss-of-function mutations, and an ITAM3 variant consisting of two loss-of-function mutations, and b) a co-stimulatory signaling region comprising a CD28 polypeptide (e.g., a human CD28 polypeptide, e.g., an intracellular domain of CD28 (e.g., human CD28) of a portion thereof). In certain embodiments, the transmembrane domain comprises a CD28 polypeptide comprising or consisting of amino acids 153 to 179 of SEQ ID NO: 43. In certain embodiments, the hinge/spacer region comprises a CD28 polypeptide comprising or consisting of amino acids 114 to 152 of SEQ ID NO: 43. In certain embodiments, the intracellular signaling domain comprises a modified CD3ζ polypeptide comprising or consisting of the amino acid sequence set forth in SEQ ID NO: 82, and a co-stimulatory signaling region comprising a CD28 polypeptide that comprises or consists of 180 to 220 of SEQ ID NO: 43. In certain embodiments, the CAR is designated as “28z1xx”. In certain embodiments, the CAR (e.g., 28z1xx) is encoded by a nucleotide sequence that is at least about 80%, at least about 85%, at least about 90%, at least about 95%, at least about 96%, at least about 97%, at least about 98%, or at least about 99%, at least about 100% identical to the nucleotide sequence set forth in SEQ ID NO: 87 or SEQ ID NO: 88. In certain embodiments, the CAR (e.g., 28z1xx) comprises the nucleotide sequence set forth in SEQ ID NO: 87 or SEQ ID NO: 88. SEQ ID NO: 87 and SEQ ID NO: 88 are provided below.

[SEQ ID NO: 87]
ATTGAAGTTATGTATCCTCCTCCTTACCTAGACAATGAGAAGAGCAATGG
AACCATTATCCATGTGAAAGGGAAACACCTTTGTCCAAGTCCCCTATTTC
CCGGACCTTCTAAGCCCTTTTGGGTGCTGGTGGTGGTTGGTGGAGTCCTG
GCTTGCTATAGCTTGCTAGTAACAGTGGCCTTTATTATTTTCTGGGTGAG
GAGTAAGAGGAGCAGGCTCCTGCACAGTGACTACATGAACATGACTCCCC
GCCGCCCCGGGCCCACCCGCAAGCATTACCAGCCCTATGCCCCACCACGC
GACTTCGCAGCCTATCGCTCCAGAGTGAAGTTCAGCAGGAGCGCAGACGC
CCCCGCGTACCAGCAGGGCCAGAACCAGCTCTATAACGAGCTCAATCTAG
GACGAAGAGAGGAGTACGATGTTTTGGACAAGAGACGTGGCCGGGACCCT
GAGATGGGGGGAAAGCCGAGAAGGAAGAACCCTCAGGAAGGCCTGTTCAA
TGAACTGCAGAAAGATAAGATGGCGGAGGCCTTCAGTGAGATTGGGATGA
AAGGCGAGCGCCGGAGGGGCAAGGGGCACGATGGCCTTTTCCAGGGTCTC
AGTACAGCCACCAAGGACACCTTCGACGCCCTTCACATGCAGGCCCTGCC
CCCTCGC
[SEQ ID NO: 88]
ATTGAAGTTATGTATCCTCCTCCTTACCTAGACAATGAGAAGAGCAATGG
AACCATTATCCATGTGAAAGGGAAACACCTTTGTCCAAGTCCCCTATTTC
CCGGACCTTCTAAGCCCTTTTGGGTGCTGGTGGTGGTTGGTGGAGTCCTG
GCTTGCTATAGCTTGCTAGTAACAGTGGCCTTTATTATTTTCTGGGTGAG
GAGTAAGAGGAGCAGGCTCCTGCACAGTGACTACATGAACATGACTCCCC
GCCGCCCCGGGCCCACCCGCAAGCATTACCAGCCCTATGCCCCACCACGC
GACTTCGCAGCCTATCGCTCCAGAGTGAAGTTCAGCAGGAGCGCAGACGC
CCCCGCGTACCAGCAGGGCCAGAACCAGCTCTATAACGAGCTCAATCTAG
GACGAAGAGAGGAGTACGATGTTTTGGACAAGAGACGTGGCCGGGACCCT
GAGATGGGGGGAAAGCCGAGAAGGAAGAACCCTCAGGAAGGCCTGTTCAA
TGAACTGCAGAAAGATAAGATGGCGGAGGCCTTCAGTGAGATTGGGATGA
AAGGCGAGCGCCGGAGGGGCAAGGGGCACGATGGCCTTTTCCAGGGTCTC
AGTACAGCCACCAAGGACACCTTCGACGCCCTTCACATGCAGGCCCTGCC
CCCTCGC

In certain embodiments, the antigen-recognizing receptor is a CAR that comprises i) an extracellular antigen-binding domain, ii) a transmembrane domain, iii) a hinge/spacer region, iv) an intracellular signaling domain comprising a) a native CD3ζ polypeptide, and b) a co-stimulatory signaling region comprising a 4-1BB polypeptide (e.g., a human 4-1BB polypeptide, e.g., an intracellular domain of 4-1BB (e.g., human 4-1BB) of a portion thereof). In certain embodiments, the intracellular signaling domain comprises a native CD3ζ polypeptide comprising or consisting of the amino acid sequence set forth in SEQ ID NO: 69, and a co-stimulatory signaling region comprising a 4-1BB polypeptide that comprises or consists of amino acids 214 to 255 of SEQ ID NO: 41, or SEQ ID NO: 42. In certain embodiments, the CAR is designated as “BBz”. In certain embodiments, the CAR (e.g., BBz) is encoded by a nucleotide sequence that is at least about 80%, at least about 85%, at least about 90%, at least about 95%, at least about 96%, at least about 97%, at least about 98%, or at least about 99%, at least about 100% identical to the nucleotide sequence set forth in SEQ ID NO: 89 or SEQ ID NO: 90. In certain embodiments, the CAR (e.g., BBz) comprises the nucleotide sequence set forth in SEQ ID NO: 89 or SEQ ID NO: 90. SEQ ID NO: 89 and SEQ ID NO: 90 are provided below.

[SEQ ID NO: 89]
CCCACCACGACGCCAGCGCCGCGACCACCAACCCCGGCGCCCACGATCGC
GTCGCAGCCCCTGTCCCTGCGCCCAGAGGCGTGCCGGCCAGCGGCGGGGG
GCGCAGTGCACACGAGGGGGCTGGACTTCGCCTGTGATATCTACATCTGG
GCGCCCCTGGCCGGGACTTGTGGGGTCCTTCTCCTGTCACTGGTTATCAC
CCTTTACTGCAACAAACGGGGCAGAAAGAAGCTCCTGTATATATTCAAAC
AACCATTTATGAGACCAGTACAAACTACTCAAGAGGAAGATGGCTGTAGC
TGCCGATTTCCAGAAGAAGAAGAAGGAGGATGTGAACTGAGAGTGAAGTT
CAGCAGGAGCGCAGACGCCCCCGCGTACCAGCAGGGCCAGAACCAGCTCT
ATAACGAGCTCAATCTAGGACGAAGAGAGGAGTACGATGTTTTGGACAAG
AGACGTGGCCGGGACCCTGAGATGGGGGGAAAGCCGAGAAGGAAGAACCC
TCAGGAAGGCCTGTACAATGAACTGCAGAAAGATAAGATGGCGGAGGCCT
ACAGTGAGATTGGGATGAAAGGCGAGCGCCGGAGGGGCAAGGGGCACGAT
GGCCTTTACCAGGGTCTCAGTACAGCCACCAAGGACACCTACGACGCCCT
TCACATGCAGGCCCTGCCCCCTCGC
[SEQ ID NO: 90]
CCCACCACGACGCCAGCGCCGCGACCACCAACCCCGGCGCCCACGATCGC
GTCGCAGCCCCTGTCCCTGCGCCCAGAGGCGTGCCGGCCAGCGGCGGGGG
GCGCAGTGCACACGAGGGGGCTGGACTTCGCCTGTGATATCTACATCTGG
GCGCCCCTGGCCGGGACTTGTGGGGTCCTTCTCCTGTCACTGGTTATCAC
CCTTTACTGCAACAAACGGGGCAGAAAGAAGCTCCTGTATATATTCAAAC
AACCATTTATGAGACCAGTACAAACTACTCAAGAGGAAGATGGCTGTAGC
TGCCGATTTCCAGAAGAAGAAGAAGGAGGATGTGAACTGAGAGTGAAGTT
TAGCCGGAGTGCCGATGCTCCAGCCTATCAGCAAGGACAAAACCAACTTT
ACAACGAACTTAATCTCGGTAGGCGCGAGGAATACGATGTGCTTGATAAA
CGCCGGGGTCGCGATCCCGAAATGGGCGGGAAACCTCGGCGCAAGAATCC
TCAAGAAGGACTCTATAATGAGTTGCAGAAGGACAAGATGGCTGAGGCAT
ATAGTGAGATCGGTATGAAGGGAGAACGGAGAAGGGGGAAAGGGCATGAT
GGATTGTACCAAGGACTTAGCACAGCTACTAAAGATACATATGACGCCCT
GCACATGCAAGCATTGCCTCCACGC

### 3.1.3. TCR-Like Fusion Molecules

In certain embodiments, the antigen-recognizing receptor is a TCR-like fusion molecule. Non-limiting examples of TCR fusion molecules include HLA-Independent TCR-based Chimeric Antigen Receptor (also known as “HIT”, e.g., those disclosed in International Patent Application No. PCT/US19/017525, which is incorporated by reference in its entirety), and T cell receptor fusion constructs (TRuCs) (e.g., those disclosed in Baeuerle et al., “Synthetic TRuC receptors engaging the complete T cell receptor for potent anti-tumor response,” Nature Communications volume 10, Article number: 2087 (2019), which is incorporated by reference in its entirety).

In certain embodiments, the TCR-like fusion molecule is a recombinant T cell receptor (TCR). In certain embodiments, the recombinant TCR comprises at least one antigen-binding chain. In certain embodiments, the antigen-binding domain of the recombinant TCR comprises a ligand for a cell-surface receptor, a receptor for a cell-surface ligand, an antigen-binding portion of an antibody or a fragment thereof, or an antigen-binding portion of a TCR. In certain embodiments, the recombinant TCR comprises two antigen binding chains, i.e., a first antigen binding chain and a second antigen binding chain. In certain embodiments, the first and second antigen-binding chains each comprise a constant domain. In certain embodiments, the recombinant TCR binds to an antigen (e.g., one disclosed in Section 3.1) in an HLA-independent manner. Thus, in certain embodiments, the recombinant TCR is an HLA-independent (or non-HLA restricted) TCR (referred to as “HIT”).

In certain embodiments, the first antigen-binding chain comprises an antigen-binding fragment of a heavy chain variable region (V_{H}) of an antibody. In certain embodiments, the second antigen-binding chain comprises an antigen-binding fragment of a light chain variable region (V_{L}) of an antibody. In certain embodiments, the first antigen-binding chain comprises an antigen-binding fragment of a V_{H} of an antibody, and the second antigen-binding chain comprises an antigen-binding fragment of a V_{L} of the antibody.

In certain embodiments, the constant domain comprises a TCR constant region selected from the group consisting of a native or modified TRAC polypeptide, a native or modified TRBC polypeptide, a native or modified TRDC polypeptide, a native or modified TRGC polypeptide, and any variants or functional fragments thereof. In certain embodiments, the constant domain comprises a native or modified TRAC polypeptide. In certain embodiments, the constant domain comprises a native or modified TRBC polypeptide. In certain embodiments, the first antigen-binding chain comprises a TRAC polypeptide, and the second antigen-binding chain comprises a TRBC polypeptide. In certain embodiments, the first antigen-binding chain comprises a TRBC polypeptide, and the second antigen-binding chain comprises a TRAC polypeptide.

In certain embodiments, the first antigen-binding chain comprises a V_{H} of an antibody and a TRAC polypeptide, and the second antigen-binding chain comprises a V_{L} of an antibody and a TRBC polypeptide.

In certain embodiments, the first antigen-binding chain comprises a V_{H} of an antibody and a TRBC polypeptide, and the second antigen-binding chain comprises a V_{L} of an antibody and a TRAC polypeptide.

In certain embodiments, at least one of the TRAC polypeptide and the TRBC polypeptide is endogenous. In certain embodiments, the TRAC polypeptide is endogenous. In certain embodiments, the TRBC polypeptide is endogenous. In certain embodiments, both the TRAC polypeptide and the TRBC polypeptide are endogenous.

In certain embodiments, the antigen binding chain is capable of associating with a CD3ζ polypeptide. In certain embodiments, the antigen binding chain, upon binding to an antigen, is capable of activating the CD3ζ polypeptide associated with the antigen binding chain. In certain embodiments, the activation of the CD3ζ polypeptide is capable of activating an immunoresponsive cell. In certain embodiments, the TCR-like fusion molecule is capable of integrating with a CD3 complex and providing HLA-independent antigen recognition. In certain embodiments, the TCR-like fusion molecule replaces an endogenous TCR in a CD3/TCR complex.

In certain embodiments, the first and second antigen binding chains bind to an antigen with a dissociation constant (K_{D}) of about 2×10⁻⁷ M or less. In certain embodiments, the first and second antigen binding chains bind to an antigen with a high binding affinity. In certain embodiments, the K_{D} is about 2×10⁻⁷ M or less, about 1×10⁻⁷ M or less, about 9×10⁻⁸ M or less, about 1×10⁻⁸ M or less, about 9×10⁻⁹ M or less, about 5×10⁻⁹ M or less, about 4×10⁻⁹ M or less, about 3×10⁻⁹ or less, about 2×10⁻⁹ M or less, or about 1×10⁻⁹ M or less. In certain embodiments, the K_{D} is about 1×10⁻⁸ M or less. In certain embodiments, the K_{D} is about 3×10⁻⁹ M or less. In certain embodiments, the K_{D} is about 5×10⁻⁹ M or less. In certain embodiments, the K_{D} is from about 1×10⁻⁹ M to about 1×10⁻⁸ M. In certain embodiments, the K_{D} is from about 1.5×10⁻⁹ M to about 1×10⁻⁸ M. In certain embodiments, the K_{D} is from about 5×10⁻⁹ M to about 1×10⁻⁸ M.

In certain embodiments, the constant domain comprises a TCR constant region, e.g., T cell receptor alpha constant region (TRAC), T cell receptor beta constant region (TRBC, e.g., TRBC1 or TRBC2), T cell receptor gamma constant region (TRGC, e.g., TRGC1 or TRGC2), T cell receptor delta constant region (TRDC) or any variants or functional fragments thereof.

In certain embodiments, the first antigen binding chain or the second antigen binding chain comprises a constant domain that comprises a native or modified TRAC polypeptide. In certain embodiments, the TRAC polypeptide comprises an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 91 or a fragment thereof, and/or may optionally comprise up to one or up to two or up to three conservative amino acid substitutions. In certain embodiments, the TRAC polypeptide comprises or consists of the amino acid sequence set forth in SEQ ID NO: 91. SEQ ID NO: 91 is provided below.

[SEQ ID NO: 91]
IQNPDPAVYQLRDSKSSDKSVCLFTDFDSQTNVSQSKDSDVYITDKTVLD
MRSMDFKSNSAVAWSNKSDEACANAFNNSIIPEDTFFPSPESSCDVKLVE
KSFETDTNLNFQNLSVIGFRILLLKVAGENLLMTLRLWSS

An exemplary nucleotide sequence encoding the amino acid sequence of SEQ ID NO: 91 is set forth in SEQ ID NO: 92, which is provided below.

[SEQ ID NO: 92]
ATCCAGAACCCTGACCCTGCCGTGTACCAGCTGAGAGACTCTAAATCCAG
TGACAAGTCTGTCTGCCTATTCACCGATTTTGATTCTCAAACAAATGTGT
CACAAAGTAAGGATTCTGATGTGTATATCACAGACAAAACTGTGCTAGAC
ATGAGGTCTATGGACTTCAAGAGCAACAGTGCTGTGGCCTGGAGCAACAA
ATCTGACTTTGCATGTGCAAACGCCTTCAACAACAGCATTATTCCAGAAG
ACACCTTCTTCCCCAGCCCAGAAAGTTCCTGTGATGTCAAGCTGGTCGAG
AAAAGCTTTGAAACAGATACGAACCTAAACTTTCAAAACCTGTCAGTGAT
TGGGTTCCGAATCCTCCTCCTGAAAGTGGCCGGGTTTAATCTGCTCATGA
CGCTGCGGCTGTGGTCCAGC

In certain embodiments, the TRAC polypeptide comprises an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 93 or a fragment thereof, and/or may optionally comprise up to one or up to two or up to three conservative amino acid substitutions. In certain embodiments, the TRAC polypeptide comprises or consists of the amino acid sequence set forth in SEQ ID NO: 93. SEQ ID NO: 93 is provided below.

[SEQ ID NO: 93]
IPNIQNPDPAVYQLRDSKSSDKSVCLFTDFDSQTNVSQSKDSDVYITDKT
VLDMRSMDFKSNSAVAWSNKSDFACANAENNSIIPEDTFFPSPESSCDVK
LVEKSFETDTNLNFQNLSVIGFRILLLKVAGENLLMTLRLWSS

An exemplary nucleotide sequence encoding the amino acid sequence of SEQ ID NO: 93 is set forth in SEQ ID NO: 94, which is provided below.

[SEQ ID NO: 94]
ATTCCCAATATCCAGAACCCTGACCCTGCCGTGTACCAGCTGAGAGACTC
TAAATCCAGTGACAAGTCTGTCTGCCTATTCACCGATTTTGATTCTCAAA
CAAATGTGTCACAAAGTAAGGATTCTGATGTGTATATCACAGACAAAACT
GTGCTAGACATGAGGTCTATGGACTTCAAGAGCAACAGTGCTGTGGCCTG
GAGCAACAAATCTGACTTTGCATGTGCAAACGCCTTCAACAACAGCATTA
TTCCAGAAGACACCTTCTTCCCCAGCCCAGAAAGTTCCTGTGATGTCAAG
CTGGTCGAGAAAAGCTTTGAAACAGATACGAACCTAAACTTTCAAAACCT
GTCAGTGATTGGGTTCCGAATCCTCCTCCTGAAAGTGGCCGGGTTTAATC
TGCTCATGACGCTGCGGCTGTGGTCCAGC

In certain embodiments, the TRAC polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence encoded by a transcript expressed by the gene of NCBI Genbank ID: 28755, NG_001332.3, range 925603 to 930229 (SEQ ID NO: 95) or a fragment thereof, and/or may optionally comprise up to one or up to two or up to three conservative amino acid substitutions. In certain embodiments, the TRAC polypeptide comprises or consists of the amino acid sequence encoded by the nucleotide sequence of SEQ ID NO: 95. SEQ ID NO: 95 is provided below.

[SEQ ID NO: 95]
ATATCCAGAACCCTGACCCTGCCGTGTACCAGCTGAGAGACTCTAAATCC
AGTGACAAGTCTGTCTGCCTATTCACCGATTTTGATTCTCAAACAAATGT
GTCACAAAGTAAGGATTCTGATGTGTATATCACAGACAAAACTGTGCTAG
ACATGAGGTCTATGGACTTCAAGAGCAACAGTGCTGTGGCCTGGAGCAAC
AAATCTGACTTTGCATGTGCAAACGCCTTCAACAACAGCATTATTCCAGA
AGACACCTTCTTCCCCAGCCCAGGTAAGGGCAGCTTTGGTGCCTTCGCAG
GCTGTTTCCTTGCTTCAGGAATGGCCAGGTTCTGCCCAGAGCTCTGGTCA
ATGATGTCTAAAACTCCTCTGATTGGTGGTCTCGGCCTTATCCATTGCCA
CCAAAACCCTCTTTTTACTAAGAAACAGTGAGCCTTGTTCTGGCAGTCCA
GAGAATGACACGGGAAAAAAGCAGATGAAGAGAAGGTGGCAGGAGAGGGC
ACGTGGCCCAGCCTCAGTCTCTCCAACTGAGTTCCTGCCTGCCTGCCTTT
GCTCAGACTGTTTGCCCCTTACTGCTCTTCTAGGCCTCATTCTAAGCCCC
TTCTCCAAGTTGCCTCTCCTTATTTCTCCCTGTCTGCCAAAAAATCTTTC
CCAGCTCACTAAGTCAGTCTCACGCAGTCACTCATTAACCCACCAATCAC
TGATTGTGCCGGCACATGAATGCACCAGGTGTTGAAGTGGAGGAATTAAA
AAGTCAGATGAGGGGTGTGCCCAGAGGAAGCACCATTCTAGTTGGGGGAG
CCCATCTGTCAGCTGGGAAAAGTCCAAATAACTTCAGATTGGAATGTGTT
TTAACTCAGGGTTGAGAAAACAGCTACCTTCAGGACAAAAGTCAGGGAAG
GGCTCTCTGAAGAAATGCTACTTGAAGATACCAGCCCTACCAAGGGCAGG
GAGAGGACCCTATAGAGGCCTGGGACAGGAGCTCAATGAGAAAGGAGAAG
AGCAGCAGGCATGAGTTGAATGAAGGAGGCAGGGCCGGGTCACAGGGCCT
TCTAGGCCATGAGAGGGTAGACAGTATTCTAAGGACGCCAGAAAGCTGTT
GATCGGCTTCAAGCAGGGGAGGGACACCTAATTTGCTTTTCTTTTTTTTT
TTTTTTTTTTTTTTTTTTTTTGAGATGGAGTTTTGCTCTTGTTGCCCAGG
CTGGAGTGCAATGGTGCATCTTGGCTCACTGCAACCTCCGCCTCCCAGGT
TCAAGTGATTCTCCTGCCTCAGCCTCCCGAGTAGCTGAGATTACAGGCAC
CCGCCACCATGCCTGGCTAATTTTTTGTATTTTTAGTAGAGACAGGGTTT
CACTATGTTGGCCAGGCTGGTCTCGAACTCCTGACCTCAGGTGATCCACC
CGCTTCAGCCTCCCAAAGTGCTGGGATTACAGGCGTGAGCCACCACACCC
GGCCTGCTTTTCTTAAAGATCAATCTGAGTGCTGTACGGAGAGTGGGTTG
TAAGCCAAGAGTAGAAGCAGAAAGGGAGCAGTTGCAGCAGAGAGATGATG
GAGGCCTGGGCAGGGTGGTGGCAGGGAGGTAACCAACACCATTCAGGTTT
CAAAGGTAGAACCATGCAGGGATGAGAAAGCAAAGAGGGGATCAAGGAAG
GCAGCTGGATTTTGGCCTGAGCAGCTGAGTCAATGATAGTGCCGTTTACT
AAGAAGAAACCAAGGAAAAAATTTGGGGTGCAGGGATCAAAACTTTTTGG
AACATATGAAAGTACGTGTTTATACTCTTTATGGCCCTTGTCACTATGTA
TGCCTCGCTGCCTCCATTGGACTCTAGAATGAAGCCAGGCAAGAGCAGGG
TCTATGTGTGATGGCACATGTGGCCAGGGTCATGCAACATGTACTTTGTA
CAAACAGTGTATATTGAGTAAATAGAAATGGTGTCCAGGAGCCGAGGTAT
CGGTCCTGCCAGGGCCAGGGGCTCTCCCTAGCAGGTGCTCATATGCTGTA
AGTTCCCTCCAGATCTCTCCACAAGGAGGCATGGAAAGGCTGTAGTTGTT
CACCTGCCCAAGAACTAGGAGGTCTGGGGTGGGAGAGTCAGCCTGCTCTG
GATGCTGAAAGAATGTCTGTTTTTCCTTTTAGAAAGTTCCTGTGATGTCA
AGCTGGTCGAGAAAAGCTTTGAAACAGGTAAGACAGGGGTCTAGCCTGGG
TTTGCACAGGATTGCGGAAGTGATGAACCCGCAATAACCCTGCCTGGATG
AGGGAGTGGGAAGAAATTAGTAGATGTGGGAATGAATGATGAGGAATGGA
AACAGCGGTTCAAGACCTGCCCAGAGCTGGGTGGGGTCTCTCCTGAATCC
CTCTCACCATCTCTGACTTTCCATTCTAAGCACTTTGAGGATGAGTTTCT
AGCTTCAATAGACCAAGGACTCTCTCCTAGGCCTCTGTATTCCTTTCAAC
AGCTCCACTGTCAAGAGAGCCAGAGAGAGCTTCTGGGTGGCCCAGCTGTG
AAATTTCTGAGTCCCTTAGGGATAGCCCTAAACGAACCAGATCATCCTGA
GGACAGCCAAGAGGTTTTGCCTTCTTTCAAGACAAGCAACAGTACTCACA
TAGGCTGTGGGCAATGGTCCTGTCTCTCAAGAATCCCCTGCCACTCCTCA
CACCCACCCTGGGCCCATATTCATTTCCATTTGAGTTGTTCTTATTGAGT
CATCCTTCCTGTGGTAGCGGAACTCACTAAGGGGCCCATCTGGACCCGAG
GTATTGTGATGATAAATTCTGAGCACCTACCCCATCCCCAGAAGGGCTCA
GAAATAAAATAAGAGCCAAGTCTAGTCGGTGTTTCCTGTCTTGAAACACA
ATACTGTTGGCCCTGGAAGAATGCACAGAATCTGTTTGTAAGGGGATATG
CACAGAAGCTGCAAGGGACAGGAGGTGCAGGAGCTGCAGGCCTCCCCCAC
CCAGCCTGCTCTGCCTTGGGGAAAACCGTGGGTGTGTCCTGCAGGCCATG
CAGGCCTGGGACATGCAAGCCCATAACCGCTGTGGCCTCTTGGTTTTACA
GATACGAACCTAAACTTTCAAAACCTGTCAGTGATTGGGTTCCGAATCCT
CCTCCTGAAAGTGGCCGGGTTTAATCTGCTCATGACGCTGCGGCTGTGGT
CCAGCTGAGGTGAGGGGCCTTGAAGCTGGGAGTGGGGTTTAGGGACGCGG
GTCTCTGGGTGCATCCTAAGCTCTGAGAGCAAACCTCCCTGCAGGGTCTT
GCTTTTAAGTCCAAAGCCTGAGCCCACCAAACTCTCCTACTTCTTCCTGT
TACAAATTCCTCTTGTGCAATAATAATGGCCTGAAACGCTGTAAAATATC
CTCATTTCAGCCGCCTCAGTTGCACTTCTCCCCTATGAGGTAGGAAGAAC
AGTTGTTTAGAAACGAAGAAACTGAGGCCCCACAGCTAATGAGTGGAGGA
AGAGAGACACTTGTGTACACCACATGCCTTGTGTTGTACTTCTCTCACCG
TGTAACCTCCTCATGTCCTCTCTCCCCAGTACGGCTCTCTTAGCTCAGTA
GAAAGAAGACATTACACTCATATTACACCCCAATCCTGGCTAGAGTCTCC
GCACCCTCCTCCCCCAGGGTCCCCAGTCGTCTTGCTGACAACTGCATCCT
GTTCCATCACCATCAAAAAAAAACTCCAGGCTGGGTGCGGGGGCTCACAC
CTGTAATCCCAGCACTTTGGGAGGCAGAGGCAGGAGGAGCACAGGAGCTG
GAGACCAGCCTGGGCAACACAGGGAGACCCCGCCTCTACAAAAAGTGAAA
AAATTAACCAGGTGTGGTGCTGCACACCTGTAGTCCCAGCTACTTAAGAG
GCTGAGATGGGAGGATCGCTTGAGCCCTGGAATGTTGAGGCTACAATGAG
CTGTGATTGCGTCACTGCACTCCAGCCTGGAAGACAAAGCAAGATCCTGT
CTCAAATAATAAAAAAAATAAGAACTCCAGGGTACATTTGCTCCTAGAAC
TCTACCACATAGCCCCAAACAGAGCCATCACCATCACATCCCTAACAGTC
CTGGGTCTTCCTCAGTGTCCAGCCTGACTTCTGTTCTTCCTCATTCCAGA
TCTGCAAGATTGTAAGACAGCCTGTGCTCCCTCGCTCCTTCCTCTGCATT
GCCCCTCTTCTCCCTCTCCAAACAGAGGGAACTCTCCTACCCCCAAGGAG
GTGAAAGCTGCTACCACCTCTGTGCCCCCCCGGCAATGCCACCAACTGGA
TCCTACCCGAATTTATGATTAAGATTGCTGAAGAGCTGCCAAACACTGCT
GCCACCCCCTCTGTTCCCTTATTGCTGCTTGTCACTGCCTGACATTCACG
GCAGAGGCAAGGCTGCTGCAGCCTCCCCTGGCTGTGCACATTCCCTCCTG
CTCCCCAGAGACTGCCTCCGCCATCCCACAGATGATGGATCTTCAGTGGG
TTCTCTTGGGCTCTAGGTCCTGCAGAATGTTGTGAGGGGTTTATTTTTTT
TTAATAGTGTTCATAAAGAAATACATAGTATTCTTCTTCTCAAGACGTGG
GGGGAAATTATCTCATTATCGAGGCCCTGCTATGCTGTGTATCTGGGCGT
GTTGTATGTCCTGCTGCCGATGCCTTC

In certain embodiments, the first antigen binding chain or the second antigen binding chain comprises a constant domain comprising a native or modified TRBC polypeptide. In certain embodiments, the TRBC polypeptide is a TRBC2 polypeptide. In certain embodiments, the TRBC2 polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 96 or a fragment thereof, and/or may optionally comprise up to one or up to two or up to three conservative amino acid substitutions. In certain embodiments, the TRBC2 polypeptide comprises or consists of the amino acid sequence set forth in SEQ ID NO: 96. SEQ ID NO: 96 is provided below.

[SEQ ID NO: 96]
DLKNVEPPEVAVFEPSEAEISHTQKATLVCLATGFYPDHVELSWWVNGKE
VHSGVSTDPQPLKEQPALNDSRYCLSSRLRVSATFWQNPRNHFRCQVQFY
GLSENDEWTQDRAKPVTQIVSAEAWGRADCGETSESYQQGVLSATILYEI
LLGKATLYAVLVSALVLMAMVKRKDSRG

An exemplary nucleotide sequence encoding the amino acid sequence of SEQ ID NO: 96 is set forth in SEQ ID NO: 97, which is provided below.

[SEQ ID NO: 97]
GATCTGAAAAACGTGTTCCCTCCTGAAGTGGCTGTCTTTGAACCATCCGA
GGCCGAGATTTCCCATACCCAGAAAGCAACTCTGGTCTGTCTGGCCACTG
GATTCTACCCCGATCACGTGGAACTGTCTTGGTGGGTGAACGGCAAGGAA
GTCCATTCCGGAGTCTCTACCGACCCTCAGCCCCTCAAGGAGCAGCCTGC
TCTCAACGATTCTCGGTACTGCCTGTCATCTCGACTGAGAGTGTCTGCCA
CCTTCTGGCAGAACCCTAGAAACCACTTTCGGTGTCAGGTCCAGTTTTAC
GGCCTGAGCGAGAACGATGAGTGGACACAGGATAGAGCCAAACCTGTGAC
ACAGATTGTGAGCGCCGAGGCTTGGGGACGAGCCGATTGTGGCTTCACAT
CCGAGTCTTACCAGCAGGGAGTGCTGTCTGCTACAATCCTCTACGAAATT
CTCCTGGGGAAGGCCACCCTGTACGCTGTCCTCGTGTCTGCTCTGGTGCT
CATGGCTATGGTCAAACGAAAGGACTCTAGAGGC

In certain embodiments, the TRBC polypeptide is a TRBC2 polypeptide. In certain embodiments, the TRBC2 polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 98 or a fragment thereof, and/or may optionally comprise up to one or up to two or up to three conservative amino acid substitutions. In certain embodiments, the TRBC2 polypeptide comprises or consists of the amino acid sequence set forth in SEQ ID NO: 98. SEQ ID NO: 98 is provided below.

[SEQ ID NO: 98]
LEDLKNVEPPEVAVFEPSEAEISHTQKATLVCLATGFYPDHVELSWWVNG
KEVHSGVSTDPQPLKEQPALNDSRYCLSSRLRVSATFWQNPRNHERCQVQ
FYGLSENDEWTQDRAKPVTQIVSAEAWGRADCGETSESYQQGVLSATILY
EILLGKATLYAVLVSALVLMAMVKRKDSRG

An exemplary nucleotide sequence encoding the amino acid sequence of SEQ ID NO: 98 is set forth in SEQ ID NO: 99, which is provided below.

[SEQ ID NO: 99]
CTGGAGGATCTGAAAAACGTGTTCCCTCCTGAAGTGGCTGTCTTT
GAACCATCCGAGGCCGAGATTTCCCATACCCAGAAAGCAACTCTG
GTCTGTCTGGCCACTGGATTCTACCCCGATCACGTGGAACTGTCT
TGGTGGGTGAACGGCAAGGAAGTCCATTCCGGAGTCTCTACCGAC
CCTCAGCCCCTCAAGGAGCAGCCTGCTCTCAACGATTCTCGGTAC
TGCCTGTCATCTCGACTGAGAGTGTCTGCCACCTTCTGGCAGAAC
CCTAGAAACCACTTTCGGTGTCAGGTCCAGTTTTACGGCCTGAGC
GAGAACGATGAGTGGACACAGGATAGAGCCAAACCTGTGACACAG
ATTGTGAGCGCCGAGGCTTGGGGACGAGCCGATTGTGGCTTCACA
TCCGAGTCTTACCAGCAGGGAGTGCTGTCTGCTACAATCCTCTAC
GAAATTCTCCTGGGGAAGGCCACCCTGTACGCTGTCCTCGTGTCT
GCTCTGGTGCTCATGGCTATGGTCAAACGAAAGGACTCTAGAGGC

In certain embodiments, the TRBC polypeptide is a TRBC1 polypeptide. In certain embodiments, the TRBC1 polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 100 or a fragment 10 thereof, and/or may optionally comprise up to one or up to two or up to three conservative amino acid substitutions. In certain embodiments, the TRBC1 polypeptide comprises or consists of the amino acid sequence set forth in SEQ ID NO: 100. SEQ ID NO: 100 is provided below.

[SEQ ID NO: 100]
LNKVFPPEVAVFEPSEAEISHTQKATLVCLATGFFPDHVELSWWV
NGKEVHSGVSTDPQPLKEQPALNDSRYCLSSRLRVSATFWQNPRN
HFRCQVQFYGLSENDEWTQDRAKPVTQIVSAEAWGRADCGFTSVS
YQQGVLSATILYEILLGKATLYAVLVSALVLMAMVKRKDF

In certain embodiments, the TRBC1 polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 101 or a fragment thereof, and/or may optionally comprise up to one or up to two or up to three conservative amino acid substitutions. In certain embodiments, the TRBC1 polypeptide comprises or consists of the amino acid sequence set forth in SEQ ID NO: 101. SEQ ID NO: 101 is provided below.

[SEQ ID NO: 101]
DLNKVFPPEVAVFEPSEAEISHTQKATLVCLATGFFPDHVELSWW
VNGKEVHSGVSTDPQPLKEQPALNDSRYCLSSRLRVSATFWQNPR
NHFRCQVQFYGLSENDEWTQDRAKPVTQIVSAEAWGRADCGETSV
SYQQGVLSATILYEILLGKATLYAVLVSALVLMAMVKRKDF

An exemplary nucleotide sequence encoding the amino acid sequence of SEQ ID NO: 101 is set forth in SEQ ID NO: 102, which is provided below.

[SEQ ID NO: 102]
GACCTGAACAAGGTGTTCCCACCCGAGGTCGCTGTGTTTGAGCCA
TCAGAAGCAGAGATCTCCCACACCCAAAAGGCCACACTGGTGTGC
CTGGCCACAGGCTTCTTCCCCGACCACGTGGAGCTGAGCTGGTGG
GTGAATGGGAAGGAGGTGCACAGTGGGGTCAGCACAGACCCGCAG
CCCCTCAAGGAGCAGCCCGCCCTCAATGACTCCAGATACTGCCTG
AGCAGCCGCCTGAGGGTCTCGGCCACCTTCTGGCAGAACCCCCGC
AACCACTTCCGCTGTCAAGTCCAGTTCTACGGGCTCTCGGAGAAT
GACGAGTGGACCCAGGATAGGGCCAAACCCGTCACCCAGATCGTC
AGCGCCGAGGCCTGGGGTAGAGCAGACTGTGGCTTTACCTCGGTG
TCCTACCAGCAAGGGGTCCTGTCTGCCACCATCCTCTATGAGATC
CTGCTAGGGAAGGCCACCCTGTATGCTGTGCTGGTCAGCGCCCTT
GTGTTGATGGCCATGGTCAAGAGAAAGGATTTC

In certain embodiments, the TRBC polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence encoded by a transcript expressed by a gene of NCBI Genbank ID: 28639, NG_001333.2, range 645749 to 647196 (TRBC1, SEQ ID NO: 103), NCBI Genbank ID: 28638, NG_001333.2 range 655095 to 656583 (TRBC2, SEQ ID NO: 104) or a fragment thereof, and/or may optionally comprise up to one or up to two or up to three conservative amino acid substitutions. In certain embodiments, the TRBC polypeptide comprises or consists of the amino acid sequence encoded by the nucleotide sequence of SEQ ID NO: 103. In certain embodiments, the TRBC polypeptide comprises or consists of the amino acid sequence encoded by the nucleotide sequence of SEQ ID NO: 104. SEQ ID NO: 103 and 104 are provided below.

[SEQ ID NO: 103]
AGGACCTGAACAAGGTGTTCCCACCCGAGGTCGCTGTGTTTGAGC
CATCAGAAGCAGAGATCTCCCACACCCAAAAGGCCACACTGGTGT
GCCTGGCCACAGGCTTCTTCCCCGACCACGTGGAGCTGAGCTGGT
GGGTGAATGGGAAGGAGGTGCACAGTGGGGTCAGCACAGACCCGC
AGCCCCTCAAGGAGCAGCCCGCCCTCAATGACTCCAGATACTGCC
TGAGCAGCCGCCTGAGGGTCTCGGCCACCTTCTGGCAGAACCCCC
GCAACCACTTCCGCTGTCAAGTCCAGTTCTACGGGCTCTCGGAGA
ATGACGAGTGGACCCAGGATAGGGCCAAACCCGTCACCCAGATCG
TCAGCGCCGAGGCCTGGGGTAGAGCAGGTGAGTGGGGCCTGGGGA
GATGCCTGGAGGAGATTAGGTGAGACCAGCTACCAGGGAAAATGG
AAAGATCCAGGTAGCAGACAAGACTAGATCCAAAAAGAAAGGAAC
CAGCGCACACCATGAAGGAGAATTGGGCACCTGTGGTTCATTCTT
CTCCCAGATTCTCAGCCCAACAGAGCCAAGCAGCTGGGTCCCCTT
TCTATGTGGCCTGTGTAACTCTCATCTGGGTGGTGCCCCCCATCC
CCCTCAGTGCTGCCACATGCCATGGATTGCAAGGACAATGTGGCT
GACATCTGCATGGCAGAAGAAAGGAGGTGCTGGGCTGTCAGAGGA
AGCTGGTCTGGGCCTGGGAGTCTGTGCCAACTGCAAATCTGACTT
TACTTTTAATTGCCTATGAAAATAAGGTCTCTCATTTATTTTCCT
CTCCCTGCTTTCTTTCAGACTGTGGCTTTACCTCGGGTAAGTAAG
CCCTTCCTTTTCCTCTCCCTCTCTCATGGTTCTTGACCTAGAACC
AAGGCATGAAGAACTCACAGACACTGGAGGGTGGAGGGTGGGAGA
GACCAGAGCTACCTGTGCACAGGTACCCACCTGTCCTTCCTCCGT
GCCAACAGTGTCCTACCAGCAAGGGGTCCTGTCTGCCACCATCCT
CTATGAGATCCTGCTAGGGAAGGCCACCCTGTATGCTGTGCTGGT
CAGCGCCCTTGTGTTGATGGCCATGGTAAGCAGGAGGGCAGGATG
GGGCCAGCAGGCTGGAGGTGACACACTGACACCAAGCACCCAGAA
GTATAGAGTCCCTGCCAGGATTGGAGCTGGGCAGTAGGGAGGGAA
GAGATTTCATTCAGGTGCCTCAGAAGATAACTTGCACCTCTGTAG
GATCACAGTGGAAGGGTCATGCTGGGAAGGAGAAGCTGGAGTCAC
CAGAAAACCCAATGGATGTTGTGATGAGCCTTACTATTTGTGTGG
TCAATGGGCCCTACTACTTTCTCTCAATCCTCACAACTCCTGGCT
CTTAATAACCCCCAAAACTTTCTCTTCTGCAGGTCAAGAGAAAGG
ATTTCTGA
[SEQ ID NO: 104]
AGGACCTGAAAAACGTGTTCCCACCCGAGGTCGCTGTGTTTGAGC
CATCAGAAGCAGAGATCTCCCACACCCAAAAGGCCACACTGGTAT
GCCTGGCCACAGGCTTCTACCCCGACCACGTGGAGCTGAGCTGGT
GGGTGAATGGGAAGGAGGTGCACAGTGGGGTCAGCACAGACCCGC
AGCCCCTCAAGGAGCAGCCCGCCCTCAATGACTCCAGATACTGCC
TGAGCAGCCGCCTGAGGGTCTCGGCCACCTTCTGGCAGAACCCCC
GCAACCACTTCCGCTGTCAAGTCCAGTTCTACGGGCTCTCGGAGA
ATGACGAGTGGACCCAGGATAGGGCCAAACCCGTCACCCAGATCG
TCAGCGCCGAGGCCTGGGGTAGAGCAGGTGAGTGGGGCCTGGGGA
GATGCCTGGAGGAGATTAGGTGAGACCAGCTACCAGGGAAAATGG
AAAGATCCAGGTAGCGGACAAGACTAGATCCAGAAGAAAGCCAGA
GTGGACAAGGTGGGATGATCAAGGTTCACAGGGTCAGCAAAGCAC
GGTGTGCACTTCCCCCACCAAGAAGCATAGAGGCTGAATGGAGCA
CCTCAAGCTCATTCTTCCTTCAGATCCTGACACCTTAGAGCTAAG
CTTTCAAGTCTCCCTGAGGACCAGCCATACAGCTCAGCATCTGAG
TGGTGTGCATCCCATTCTCTTCTGGGGTCCTGGTTTCCTAAGATC
ATAGTGACCACTTCGCTGGCACTGGAGCAGCATGAGGGAGACAGA
ACCAGGGCTATCAAAGGAGGCTGACTTTGTACTATCTGATATGCA
TGTGTTTGTGGCCTGTGAGTCTGTGATGTAAGGCTCAATGTCCTT
ACAAAGCAGCATTCTCTCATCCATTTTTCTTCCCCTGTTTTCTTT
CAGACTGTGGCTTCACCTCCGGTAAGTGAGTCTCTCCTTTTTCTC
TCTATCTTTCGCCGTCTCTGCTCTCGAACCAGGGCATGGAGAATC
CACGGACACAGGGGCGTGAGGGAGGCCAGAGCCACCTGTGCACAG
GTGCCTACATGCTCTGTTCTTGTCAACAGAGTCTTACCAGCAAGG
GGTCCTGTCTGCCACCATCCTCTATGAGATCTTGCTAGGGAAGGC
CACCTTGTATGCCGTGCTGGTCAGTGCCCTCGTGCTGATGGCCAT
GGTAAGGAGGAGGGTGGGATAGGGCAGATGATGGGGGCAGGGGAT
GGAACATCACACATGGGCATAAAGGAATCTCAGAGCCAGAGCACA
GCCTAATATATCCTATCACCTCAATGAAACCATAATGAAGCCAGA
CTGGGGAGAAAATGCAGGGAATATCACAGAATGCATCATGGGAGG
ATGGAGACAACCAGCGAGCCCTACTCAAATTAGGCCTCAGAGCCC
GCCTCCCCTGCCCTACTCCTGCTGTGCCATAGCCCCTGAAACCCT
GAAAATGTTCTCTCTTCCACAGGTCAAGAGAAAGGATTCCAGAGG
CTAG

In certain embodiments, the first antigen binding chain or the second antigen binding chain comprises a constant domain comprising a native or modified TRGC polypeptide. In certain embodiments, the TRGC polypeptide is a native or modified TRGC1 polypeptide. In certain embodiments, the TRGC1 polypeptide comprises or consists of an amino acid sequence that is at least about 85%, about 90%, about 95%, about 96%, about 97%, about 98%, about 99% or 100% homologous or identical to the amino acid sequence set forth in SEQ ID NO: 105, which is provided below. In certain embodiments, the TRGC1 polypeptide comprises or consists of the amino acid sequence set forth in SEQ ID NO: 105.

[SEQ ID NO: 105]
DKQLDADVSPKPTIFLPSIAETKLQKAGTYLCLLEKFFPDVIKIH
WQEKKSNTILGSQEGNTMKTNDTYMKFSWLTVPEKSLDKEHRCIV
RHENNKNGVDQEIIFPPIKTDVITMDPKDNCSKDANDTLLLQLTN
TSAYYMYLLLLLKSVVYFAIITCCLLRRTAFCCNGEKS

In certain embodiments, the TRGC polypeptide is a native or modified TRGC2 polypeptide. In certain embodiments, the TRGC2 polypeptide comprises or consists of an amino acid sequence that is at least about 85%, about 90%, about 95%, about 96%, about 97%, about 98%, about 99% or 100% homologous or identical to the amino acid sequence set forth in SEQ ID NO: 106, which is provided below. In certain embodiments, the TRGC2 polypeptide comprises or consists of the amino acid sequence set forth in SEQ ID NO: 106.

[SEQ ID NO: 106]
DKQLDADVSPKPTIFLPSIAETKLQKAGTYLCLLEKFFPDIIKIH
WQEKKSNTILGSQEGNTMKINDTYMKFSWLTVPEESLDKEHRCIV
RHENNKNGIDQEIIFPPIKTDVTTVDPKYNYSKDANDVITMDPKD
NWSKDANDTLLLQLTNTSAYYTYLLLLLKSVVYFAIITCCLLRRT
AFCCNGEKS

In certain embodiments, the TRGC polypeptide comprises or consists of an amino acid sequence that is at least about 85%, about 90%, about 95%, about 96%, about 97%, about 98%, about 99%, or 100% homologous or identical to the amino acid sequence encoded by a transcript expressed by a gene of NCBI Genbank ID: 6966, NG_001336.2, range 108270 to 113860 (TRGC1, SEQ ID NO: 107), NCBI Genbank ID: 6967, NG_001336.2, range 124376 to 133924 (TRGC2, SEQ ID NO: 108) or a fragment thereof, and/or may optionally comprise up to one or up to two or up to three conservative amino acid substitutions. In certain embodiments, the TRGC polypeptide comprises or consists of the amino acid sequence encoded by the nucleotide sequence of SEQ ID NO: 107. In certain embodiments, the TRGC polypeptide comprises or consists of the amino acid sequence encoded by the nucleotide sequence of SEQ ID NO: 108. SEQ ID NO: 107 and 108 are provided below.

[SEQ ID NO: 107]
ATAAACAACTTGATGCAGATGTTTCCCCCAAGCCCACTATTTTTCTTCCTTCAATTGCTGAAACAAAG
CTCCAGAAGGCTGGAACATACCTTTGTCTTCTTGAGAAATTTTTCCCTGATGTTATTAAGATACATTG
GCAAGAAAAGAAGAGCAACACGATTCTGGGATCCCAGGAGGGGAACACCATGAAGACTAACGACACAT
ACATGAAATTTAGCTGGTTAACGGTGCCAGAAAAGTCACTGGACAAAGAACACAGATGTATCGTCAGA
CATGAGAATAATAAAAACGGAGTTGATCAAGAAATTATCTTTCCTCCAATAAAGACAGGTATGTGTTT
ACGCATATCATCTGTCAGAACACTTCTTTGAAAGTGAATGCTGCATTTTTTCCTTTCAGTATTAATGA
AAAACAAACATAAATCTTTCTTAAATATTGTTACATTTAATGGTAGCATAAATGCCCTGCTACTTTTC
TATAGAATTAAAATGGTATAGGTTTTGGAGAAAACAAAATTGAAAAAGTTACTGAAGGTTTGTCAGCC
TCAGCTCCATTATCCAAAATAAGAAAGTCACGTGCTGGTTTTTAGGGTTGTTAGATGGATTAAAGAAA
CAACATACACAGAAGCATCTAGCAACGTGACACGTGGTAAACGCTCAAAAAGTGTTCTCCCTTCTTTT
GATGACTTTACTTGATCAGGAAATAACATATATATGTCTTTCAGGAATGTTCTGCCCAAGCAGGAGAG
TCACTCACCTCAATCTTGCTACCCACAAAGTTTAACCTAAAAACAACGGGTTCATTGTTGACAAAATG
ATGTTTATCTGTTGTTGACAGAATGATGTTTATCTAAAAACAGTTCCAATTTTCTATTTCCTTTGCTG
AGACACAAAGGGGAGGCAAATGTGCAAAGCTTGAGGGTAGTCTTACCACTGTGCTTAAGTGTTCTGAT
TTTTCTAGTGATCAGGGCAAAATAAAAAGTATAGTAAGTTCCAAGGCAGTGAATATTATACAGGAGAG
AAGTTACAGTTTTATAATGTGTTTTCCTTTACACTAAATTCTAAAAGTAAAAAGTCTTTTTTTTTTTT
TGACAGAGTTTCACTCTTGTTGCCCAAGCAGGTGTGCTATGGTATGATCTCAGCTCACTGCAACCTCC
ACCTCCCGGGTTCAAGTGATTCTCTTACTTCAGCCTCCCGACAGGCTGGGATTGCAGGCGCCTGCCAC
CACACCTGGCTAATTTTTGTGTTTTTAGTAGAGATGGGGTTTCACCATGTTGGCCAGGCTGGTCTCAA
ATTCCTGACCTCAAGTGATCCATCCACCTCGGCCTCCAAGTGCTGGGATTATGGGCGTCAGCCACTGT
GCCCAGCCTAAAAGTAAAATGTCTTTCATGAGCTTCCCAAGGCAGCTACGTTAAGGAGGACACTTCTC
TTAATGTCATTCTACAGTAGATTTCTAATGCTCTTTCTTGGAAGTTTGTTTTTCTGAGAAAAGCTAAA
AATATAACATGGAAGTGATCATATTATATAATCAATGAAGTGCTTTTCAAGGAGATAAAACTAATCTG
GTCCACACTTGCAACCAACCTTGATTGAGAGAGAGAGAGAACTCAGGATACACTTGAAGATTTTATTA
TGGGGAACAGTTACTTTATTCTTTTTACCTCAATCAATGCATGGAAATAAGTGATAGTCATTTTCATT
TATCTTTTAATAAATGAAGTCACCATGAGGAAAATAAAAAGACATTGAAAACCCATTAAAGTCAGCCC
TTAAAGATATTTGGACATGCAGACTTGATAACTAACGTTTGCATTCTTGAGACTTACCCAAAACCCAT
ACCTCAAGTCCAAGTTTTTAGAATTCATGAAATAAAGATCTCAGTGAGTGCATAAAATTGCGCACCAG
AATCATATCCGTATAGACAAGAACACATCTACTAGAAAAATAATAAACCAACACACCAATGCAACTGT
GTTTTCTTCTGTTTTAAAGTATGTTGTCTTTGTATGCATGTTTGCTTCTTCCTTTTTTTTTTTAACAT
CACAGATAAATTCAACTCTCACCTCAGGTTTTATTGAGAGAACTGTCAATGTGACTTGGCCTCTGTCT
TTCTAGTCCCAGAAAGAATTGCACTGAAATCTGAGCTCCTGTAATAAAAACAACCATTTGCTGAGAGT
AATTAACATACTGAAAGAGATTTTCTTAGAGTACACAATGGTGACATTATATTGCCTCTTTATAAATA
ACTTTCTATCTATTTCTGTGGATTATTCCTACAAAGTACTTTTCATATGTCCAATTTCTTTTCTTCCC
CTACAACTACTGTCTGAATACTGGCTCTGCTATTTGCTGATATGATTCTCGGCAAGTTGCCTGCACTT
TTTAAACTTTATTTCCTCATTCAGAACATGGGGCCATACATAATACAACTCACTTCAGTGTTATTGGG
GAATTAAACAAAAAATGCATGGGAAGCATTTAACATAGTGCCTGACACAATAATGAGTACTCAGTAGA
TGTTAGCTTTTATTAATATTGTTGTTGTTATGTCCAGAAACACTATACCTCCAGAAAATCATGGGTAC
TTGCTGGGGACATTGGGGATATGCATGATTTGGAAAAGAATGACTGCTTTTTTTGCTTAGATGAGAAA
TTTTTCTAAGCCAGACTCCTTCAAATATGTAAGATTCTGTTGTGGATTCAAGGACTGAAAGAATTCTT
GGCCGAGTGTGGTGGCTTATCCCTGTAATCCCAGCATTTTGTGAGGACAAGGCAGGAAGATTGCTTGA
GTCCAGGAGTTTGAAACCAGCCTGCGCAACATGGCGAAACCCTGTCTCTACAAAAAATACAAACATTA
GCTCGGAGTGAGTGCTGACATGTGCCTGTACTCCCAGCTACTCAGAAGGCTGAGATGGGAGGATCTCA
TGAGCCTGGGGAGTTTGAGGCTTCAGTGAGCCGTGATGACACCGTACTATACTCCACTCCAGCCTGGG
TGACAGTGAGACCCTGCCTCAAAAAACAAACAAACAAACAAACAAAACAAAATTAATCTTTTTGCTGA
TGTCATGTCAGCAGTGTGTGTTGAAGGCTGTAAAGCAGCCATTTGTTCAGTTTATTTTTCCATTGAAC
AAGTATTTATCAAAAACATACTTTGTGGCAGTCACTATGCTAGGAGCTATGAATACAGAAGGAAAAGT
AAATGCTCTTGGATACTACACTCCAGTTGTGATAAAAAAGAAAAAATGTATTCTTCACCAACTTCAAC
ATCTTGATGTGCAAAAACATAATACATGAATTAGATCTACCTAATTACACAGAATTAGACCAATTGTT
TCTGGAATTGTGGGCTCATATTTTTAATAACTGTCCTCCTGCCTCTCTGTCGACAGGTTTTATAAATA
TTCATTTAATTACACACACACACACGAACAATTGACTAGTACTTGCTCTCATTCTTCTAGATGTCATC
ACAATGGATCCCAAAGACAATTGTTCAAAAGATGCAAATGGTAAGCTTTTGTGTTTTTCCCTTCCTCC
TGATCATTTTGTTTTGAACTTCTCTGGCTTGAAAAATCAGGGAATGGATTTTGCTAGGTTGGATGCTG
CAGAATGGACCTAGTGATATTTTAAATTAGTCCCTCATTTTCTAGGAGTTGTATTAACAAACCTAACT
ACTGCTTTGGGGTATGAGATGACTGTAAATTAGAGAGGGTACAGTGGTATAGTGATATGCTTTTAATT
ATTTCAAAAAAAAGATTTTATTCATTCATGTGTCTTTTTTCTTTTTCTTTTCTTTTTTTTTTTTTTTT
GGACAGAGTCTTGCTCTGTCACCCAGGCTGGAGTGCGGTGGCAGTATCTCAGCTCACCACAACCTCCG
CCTCCCGGCTTCAAGTGATTCTCCTGCCTCAGCTTCTCGAGTAGCTGGGACTACAGGCGCGTGCCACC
ATGCCCGGCTAATTTTTGTATTTTTAGTAGAGTTGGGGTTTCACCATGTTGGCCAGGATGGCCTCGAA
TTTGTGACCTCGTGATCTGCCCCCTCGCCCTCCCGAACTGTTGGGATTACAGGCGTGAGTCACTGTGC
CCGGCCTCCTGTCCTGTCTTTTGTTTAATGACTGGGAAAAACATGATACCATGTTGCTTCTCGAGTTG
TTTTGTTTTAGTCTTTGGTCTTTGCTAGTAGCTAATAACACGAACTAGTGTTTATCAAGTGCTTTTTA
CACAGAAGGGCTTGGGCTGTGTTCTGCATTTTCTTGTTTAACCCTCTTAAAACTCCTATAAAATGGTA
CATATTTTTCTCCCAATTTACAGTCCCTTTAAAGCAAATAATTATAAAAATCCCTATACATGTCACAC
AGCTAGATCTGGGATTTCAAATCAGGCCATCAAACAAAGAGTTTATGTACTTAGTAAGTTTTCTGTTC
TTTTTCTACAATAGAGTCAGATAGCAAGAAATTACCAAGCCAGGAACCTGAAACAAAACGGACATCAT
GTGGGGCTGGGTGGGTGCATGGGCTTTGCAGACTGGACTTTCACTCCAGCTCTTTTAATGATTAGGTG
TAAGTGACCTACATTTTGTGAGCAACAGTTTTCTCATCAGCCAACAAAGAATAATTACACCAGATTCA
CAGTTATTGAAGAGATAAAGGCATGAATGTGAGATGTCTGGCATAGGGCATCTCATTTAGCAGACACA
GAATGAGTACTTGTTTCTGGCTTTTTCTCTCTACATATGCACAAAGAATGCGACTAGAAGCATGGGCT
CTAGCCCTGCTCAACTTTCCTCTATTTCCAATACCAAGGGGCTCTGACTTAGGCTGCCACACCAGGCA
AGGAGGGCAGTACCACCTCACTTGACCAAGGGCAGGGAGTCACGGACACATCACTTCTTGAGATCCTT
TTCCACACCAAGGACTGATGTTTCTGGAATTCTCACTTTATGAAGACAAAACATATAAATGGAAATTT
TCTCAGGTAGAGACTCACTCTTGTAGCTCATTGAGTAGGCACTAGTGGTCCACCCCCACTGTCTTTAC
TTATTCCTTGACATCACATATCTCTTGCAAAACCTCAAATAATATTAAATGCAATCACCCAATAATAG
CATAGCCATAATTAGAGGCATTTAGGAAAGACAGGTGAGTGTGCCACAACTACCTAACACATCAGCAA
ATCTGGATTAACCACTTTCTTTGATTTTCCACAATGCAACCTTACTTTTTAATAGTTGGGAATGTTCT
AAGTGAATTTAGCAGAGGTTGTTAATCAACTTGAAAGCTGAATTCTGACTTGTCTGACTCTTGGTGGT
GCTGGTAGCAGTAGATGTTTACTTTTAGGTTTTGGTGGTGGTGGAATATCACTTCAACGTAAATCATC
AGAAATAAGTATTTGTGAACCCCTCTCGCATTAATGTATCTTATTCTGTAAAAAGAACATGTGCAATT
TCTCTTAGATACACTACTGCTGCAGCTCACAAACACCTCTGCATATTACATGTACCTCCTCCTGCTCC
TCAAGAGTGTGGTCTATTTTGCCATCATCACCTGCTGTCTGCTTAGAAGAACGGCTTTCTGCTGCAAT
GGAGAGAAATCATAA
[SEQ ID NO: 108]
ATAAACAACTTGATGCAGATGTTTCCCCCAAGCCCACTATTTTTCTTCCTTCGATTGCTGAAACAAAA
CTCCAGAAGGCTGGAACATACCTTTGTCTTCTTGAGAAATTTTTCCCAGATATTATTAAGATACATTG
GCAAGAAAAGAAGAGCAACACGATTCTGGGATCCCAGGAGGGGAACACCATGAAGACTAACGACACAT
ACATGAAATTTAGCTGGTTAACGGTGCCAGAAGAGTCACTGGACAAAGAACACAGATGTATCGTCAGA
CATGAGAATAATAAAAACGGAATTGATCAAGAAATTATCTTTCCTCCAATAAAGACAGGTATGTGTTT
ACACATATCATCTGTCAGAACACTTCTTTGAAAGTGAATGCTGCATTTTTTCCTTTCAGTATTAATGA
AAAACATAAATCTTTCTTAAAAATTGTTACATTTAATGGTAGCGTAAATGCCCTGCTACTTTTCTATA
GAATTAAAATGGTATAGGTTTTGGAGAAAACAAAATTGAAAAAGTTGCTGAAGGTTTGTCAGCCTCAG
CTCCATTATCCAAAATAAGAAAGTCACGTGCTGGTTTTTAGGGTTGTTAGATGGATTAAAGAAACAAC
ATACACAGAAGCATCTAGCAACGTGACACGTGGTAAACGCTCAAAAAGTGTTCTCCCTTCTTTTGATG
ACTTTACTTGATCAGGAAATAACATATATATGTCTTTCAGGAATGTTCTGCCCAAGCAGGAGAGTCAC
TCACCTCAATCTTGCTACCCACAAAGTTTAACCTAAAAACAACGGGTTCATTGTTGACAAAATAATGT
TTATCTGAAGATAACTGTAGATCATATTTATCTGTAGATAATGTTTATCTGTGGAGTGTGGCTCTACA
AAACATAGAATAGTCTTGGTCACTGCAGTTTTATAGAGGCCTTGGGTTTTTCAGAGTTTCATTTTATA
TATCACCATAAAGTAACATTTCATAATTACAGGTTGGTAAGGCTTACATGTACAAACATTCTTCCATT
TTCCATAATAAATGCATTTCCTGCCATTGGTGAATGCAGCTCAATAAACATTTATTGTACAATTATGA
CACGCCAGGCTTAGTGGAAATGTGGATGAACAGACAAGGATGAGTTACTGTCCTAAGGATGATGCATG
ACAGTGCAGAGAATATACTCTCTTCCTGATCACTCAGGGTCACTCATGATTCATGCGCGAGGTCCCAA
AACAGTGCCTTTGATGCAGATTCTGTACATCTCTAGACGATTGGTCCAAGGGCTGAATGTGCTCTGGC
CCAGTGGTCCAGTCTGTCACTATATGTCAACATCCTGAATATGAACATAACAGTCCAACATCTCAAGA
GTGGGCATGAAAAGGACTCATTTTGTGCTTTTTCCTGTGGTTAACAAGTCCTTTTTAGCCTGGGGGAA
CAAGCATTAACAAAATGTTTGAAGATCTTTGCCACGTACCATTCCAAATTTCTAGGGTAAGTCTTTAG
CTTTTCAGATCCTGAGTTTCTGCAATGATCAAATGTGATTTGGACAGTTGCGTTGACTTTCTCCTGGG
GCTATAATGGAGTGCAAAGGAAACAATGGCAGGGAAAATGCTTGCTTTCAAAATGGTAGCATGGATGT
GTTCATTCGTGTAGTTACTGTATTAGGTATAGCCTTTCCTGAAACTAACTGAAGTGGGGTTATAAAAA
CAGTCCCAATTTTCTATTTCCTTTGCTGAGACACAAAGAGGAGACAAAAGAGCAAAGCTTGAGGGTAG
TTTTACCACTGTGCTTAAGTGTTCTGATTTTTCCAGTGATCAGGGTGAAATAAAAAGCATAGTAAGTT
CCAGGGCAGTGAATACCATACAGGAGACAAGTTACAGTTTTATAATGTGTTTTACTTTACACTAAATT
CTAAAAGTAAAATGTCTTTTTTTTTTTCCGAGACAGAGTTTCACTCTTGTAGCCCAGGCAGGAGTGCT
ATGGTGTGATCTCGGCTCACAGCAACCTCCACCTCCCAGTTTCAAGCGATTCTTCTGCCTCAGCCTCC
CGAGAAGTTGAAATTACAGGTGCCTGGCACCATATCTCGCTAATTATTCTATTTTTAGTAGAGATCGG
GTTTTACCATGTTGGCCAGGCTGGTCTCGAACTCCTGACTTCAAGTGATCCACCCGCCTCAGCCTCCC
AAAGTGCTGGGATTACAGGTGTGAGTCACTGTGCCGGACCTAACAGTAAAATGTCTTTCATGTGCTTC
TCAAGGCAACTACATTAAGGAGGACACATCTCTTAATGTCATTCTACAGTAGATTTCTAATGCTCTTT
CTTGGAAGTTTGTTTTTCTGAGAAGAGCTAAAAATATAATAACATGGAAGTGATCATATTATATAATC
AATGAAGTGCTTTCAAAGGAGATAAAACTAACCTGGTCTGCATTTGCAACCAGCCTTGATTGAGAGAG
AGAGAACTCAGGATACACTTAGAGATTTTATTATGGGGAATAGTTACTTTATTCATTTTACCTCAATC
AATGCATGGAAATAAGTGACAGTCATTTTCATTTATCTTTTAATAAATAAAGTCACCATGAGGAAAAT
GAAAACCCATTAAAGTCAGTCCTTAAAGATATTTGGACATGCAGACATGATAACTAACATTTCCATTC
GTGAGACTTACCCAAAACCTATACCTCAAGTCCATTTCTTAGAATACATGAAATAAAGATCTCAGTGA
GTGTATAAAACTGCACACCAGAATCATATCCGTATAGACAAGAATACATCTACTAGAAAAATATAAAC
CAAAACACCAAGGTGACTCTGTTTTTTTCTGTTTTAAAATATGTTGTCTTTGTATGCATGTTTGCTTC
TTCCTTTTTTTTTTTAAACATCGCAGATAAATTCAACTCTCACCTCAGTTGAGAGAGAACTGTCAATG
TGACTTGGCCTCTCTCTTTCTAGTCCCAGAAAGAATTGCACTGAAATGCTGAGCTCCTGTAATAAAAA
TGACCATTTGCTGAGAGTAATTAACATACTGAAAGAGATTTTCTTAGAATAGTGCACAATGGCCCAAT
GGTGACATTATATTGTCTCTTTATAAATTATTTTCTATCTATTTCTGTGGATTATTTCTACAAAGCAC
TTTTCATATGTCCAATTCCTTTTATTCCCCTACAAGTACTGACTGACTACTGGCTCTGCTGTTCACTG
ATATGACTTTCGGCAAGTTGCCTGCACTTTTTAAACGTTATTTCCTCATTCAGAACATGGGGCCATAC
AAAATACAACTCACTTCAGTGTTATTGGGGAATTAAACAAATAAATGCATGGGAAGCATTTAACATAG
TGCCTGACACAATAATGAGCACTCAGTAGATGTTAGCTTTTATTAATATTGTTGTTGCTATGTCCAGA
AACACTATACCTCCAGAAAATCATGGGTACTTGCTGGGGACGTTGGGGATATGCATGATTTTGAAAGG
AGTGACTGCTCTTTACTGCTCAGATGAGAAATTTTTCTAAGCCAGACTCCTTCAAACATGTAAGATTC
TGTTGTGGATTCTAGGACTGAAAGAATTCTTGGCCGAGTGTGGTGGCTTATCCTGGTAATCTCATCAT
TTGGGAGGACAAGGCAGGAAGATTGCTTGAGCCCAGGAGTTGGAAACAAGCCTGGACAACATGGCGAA
ACCCTGTCTCTACAAAAAATACAAACATTAGCTGGTCATGGGAGTGAGTGCCTGTACTCCCAGCTACT
CAGGAGGCTAAGATAGGAGGATCACCTGAGCCTGGGCAGTTTGAGGTTTCAGTGAGCCGTGATGACAC
CATACTATACTCCACTCCAGCCTGGGTGACAGTGACATCCTGCCTCAAAAAAACCCCCAAAATTATTC
TTTTTGCTGATTTCATGTCAGCAGTGTGTGCTGAAGGCTGTAAAGTAGCCACTTGTTCTGTTTATTTT
TCCATTGAACAAGTATTTATCAAAAACGTACTTTGTGGAAGGCACTGTGCTAGGAACTATGCATACAG
AAGGAAAACCAAATGTTCTTGGATACTACACTCCAGTTGTGATAAAAAAGAAAAAAGTATTCTTCACA
AACTTCAACATTTTGATGTGCAAAAACATAATATATGAATTAGATCTACCTAACTACACAGAATTAGA
CCAATTATTTCTGGGATTATGGGCTCATATTTTTAATAACTGTCCTCCTACCTCTCTGTTGACAGGTT
TTATAAATATTCATTTAATTACACACAGTCACAGACACACTCAGACACACACACATACACACACACAC
ACACCTTGACAAATAATGGGCATGAACAATTGACTGGTACTTGCTCTCATTCTTCTAGATGTCACCAC
AGTGGATCCCAAATACAATTATTCAAAGGATGCAAATGGTAAGTTTTTGTGTTTTTTATTTCCTCCTG
ATCATTTTAAGTTTTGAACTTCTCTGGCTTGAAAAATCAGGGAATGGATTTTGCTAGGTTGGATGCTG
CAGAATGGACCTAATCATATTTTAAATTAGTCCCTCTTTTTCTAGGAGTTGTATTAACAAACCTAACT
ACTGCTTCATGTAAGAGATGACTGTAAATTGAAGGGTACAGTGATATGCTTTCAGTTATTTCAAAAAA
CAGACTTTACTCATCCATGTGTCTTTTTTCTTTTCTTTTTTTTCTTTTTTGAGACGGAGTCTCGCTCT
GTTGAACAGGCTGGATTGCAGTGACGCGATCTCACCTCACTACAACCTCCGCCTCTGGAGTTCAAGCG
ATTCTCCAGCCTCAGCTTCTCAAGTAGCTGGGACTACAGGCACATGCCACCATGTCCGGGTCATCTTT
GTATTTTTAGCAGAGACCGGGTTTCACTATGTTGGCCAGGCTGGTCTAGAATTCCTGACTTCGTGATC
TGCCCCCTCAGCCCTCCGAAGTGCTGGGATTACAGACGTGAGTCACTGTGCCCGGCCTAACAGTAAAA
TGTCTTTCATGCGCTTCTCAAGGCAACTACGTTAAGGAGGACACTTCTCTTAATGTCATTCTACAGTA
GATTTCTAATGCTCTTTCTTGGAAGTTTGTTTTTCTGAGAAAAGCTAAAAATATAACATGGAAGTGAT
CATATTGTATAATCAATGAAGTGCTTTTCAAGGAGATAAAACTAATCTGGTCCACGTTTGCAACCAAC
CTTGATTGAGAGAGAGAGAGAACTCAGGATACACTTGGAGATTTTATTATGGGGAATAGTTACTTTAT
TCTTTTTTCCTCAATCAATTCATGGAAATAAGTGATAGTCATATTCATTTATCTTTTAATAAATGAAG
TCACCATGAGGAAAATAAAAAGACATTGAAAACCCATTAAAGTTAGCCCTTAAAGATATTTGGACATG
CAGACTTGATAACTAACGTTTGCATTCTTGAGACTTACCCAAAACCCATACCTCAAGTCCATGTTTTT
AGAATTCATGAAATAAAGATCTCAGTGAGTGCATAAAATTGCGCACCAGAATCATATCCGTATAGACA
AGAACACATCTACTAGAAAAATAATAAACCAACACACCAATGCAACTGTGTTTTCTTCTGTTTTAAAA
TATGTTGTCTTTGTATGCATGTTTGCTTCTTCCTTTTTTTTTTTTAACATCACAGATAAATTCAACTC
TCACCTCAGGTTTTATTGAGAGAACTGTCAATGTGACTTGGCCTCTGTCTTTCTAGTCCCAGAAAGAA
TCGCACTGAAATGCTGAGCTCCTGTAATAAAAATGACCATTTGCTGAGAGTAATTAACATACTGAAAG
AGATTTTCTTAGAGTACACAATGGTGACATTATATTGTCTCTTTATAAATAACTTTCTATCTATTTCT
GTGGATTATTCCTACAAAGTACTTTTCATATGTCCAGTTTCTTTTCTTCCCCTACAACTACCGTCTGA
ATACTGGCTCTGCTATTTGCTGATATGATTCTCGGCAAGTTGCCTGCACTTTTTAAACTTTATTTCCT
CATTCAGAACATGGGGCCATGTAATACTCATGTACGTGAGTATTACGTAATAATGCTCACTTAAGTGT
TACTGGGGAATTAAACAAAAAAATGCATGGCAAGCATTTAACATAGTGCCTGACACAATAATGAGCAC
TCAGTAGATGTTAGATTTTATTAATATTGTTGTTGTTATGTCCGGAAACACTATACCTCCAGAAAATC
ATGGGTACTTGCTTGGGATGTTGGGGATATGCATGATTTGGAAAGGTATGACTGCTTTTTTCTGCTTA
GATGAGAAATTTTTCTAAGCCAGACTCCTTCAAATATGTAAGATTCTGTTGTGGATTCTAGGACGGAA
AGAATTCTTGGTCAGGTGTGGTTTCTTATCCCTGTAATCCCAGAATTTTGGGAGGACAAGGCAGGAAG
ATTGCTTGAGCCCAGGAGTTTGAAACCAGCCTGGGCAACAAGACGAAACCCTGTCTCTACAAAAGTAC
ATAAATTAGCTTGGCTTGGTGGTGTGTGCCTGTATTACCAGCTATTCGGGAGACTGAGATGGGAGGAT
CTCCTGAACCTGTGAAGTTTGAGGCTTCAGTGAGCCGTGATGACACCATACTATACTCGACTCCAGCC
TGTGCGACAGTGAGACTCTGCGTCAAAAAAAAAACCCCAAAATTATTGTTTTTGCTGATTTCAGGTCA
GCAGTGTGTGCTGAAGGGTGTAAAGTAGCCACTTGATCAGTTTATTTTTCCACTGAACAAGTATTTAT
CAAAAACATACTTTGTGGTCTGTTTTTGATAAATAAAAAGGCACTGTGCTAGGAGCCATGAATACAGA
AGGAAAACCAAATGTTCTTGGATACTACACTCCAGTTGTGATAAAAAAGAAAAATGTATTCTTCACGA
ACTTCAACATTTTGATATGCAAAAACATAGTATATAAATTAGATCTACCTGATTACGTAGAATCAGAC
CAATTATTTCTGGAATTGAGGGCTCATATTTTTAATAACTGTCCTCCTGCCTCTCTGTTGACAGGTTT
TATAAATATTCATTTAATTACACACACACACACACACACCTTGACAAATAATGGACATGAACAATTGA
CTAGTACTTGCTCTCATTCTTCTAGATGTCATCACAATGGATCCCAAAGACAATTGGTCAAAAGATGC
AAATGGTAAGCTTTTGTGTTTTTCCTTTCCTCCTGATCATTTTAAGTTTTGAACTTCTCTGGCTTGAA
AAATCAGGGAATGGGCCGGGTGCGGTGGCTCACGCCTGTAATCCCAGCACTTTGGGAGGCCGAGGCGG
GCGGATCACGAGGTCAGGAGATCGAGACCATCCCGGCTAAAACGGTGAAACCCCGTCTCTACTAAAAA
TACAAAAAATTAGCCGGGCTTAGTGGCGGGCGCCTGTAGTCCCAGCTACTTGGGAGGCTGAGGCAGGA
GAATGGCGTGAACCCGGGAGGCGGAGCTTGCAGTGAGCCGAGATTGCGCCACTGCACTCCACTCCAGC
CTGGGCGACAGAGCGAGACTCCGTCTCAAAAAAAAAAAAAAAAAAAAAAAAAGAAAAATCAGGGAATG
GATTTTGCTAGGTTGGATGCTGCAGAATGGACCTAGTGATATTTTAAATTAGTCCCTCTTTTTCTAGG
AGTTGTATTAACAAACCTAACTACTGCTTCGGGTATGAGATGACTGTAAATTAGAGGGTACAGTGATA
TGCTTTCAGTTATTTCAAAAAACAGACTTTATTCATCCGTCTGTCTTTTTTTTTTTTTTTTTTTTTTT
TTTTTGAGACGGAGGAGTCTCACTCTATCACCCAGGCTGGAGTGCAGTGGCGCGATCTCGGCTCACCA
TAACCTCCGCCTTACTGGTTCAAGCGATTCTCCAGCCTCAGCTTCTCAAGTAGCTGGGACTACAGGTG
CACACCACCATACCTGGCTAATTTTTGTATTTTTAATAGAGATGGGGTTTCACCACGCTGGCCAGGAT
GGTCTTGAATTCTTGACCTCGTGATCTGCCCCCTCGGGCTCCCAAACTTCTGGGATTATAGGCGTGAG
CCACTGTGCCCGGCCTTCTGTCTTTTGTTATAATGACTGGGGAAAACATGATACCATGTTGCTTCTTG
AGTTGTTTTGTTTTAGTCTTTGGTCTTTGCTAGTAGCTAATAACACGAACTAGTGTTTATCAAGTGCT
TTTTACACAGAAGGGCTTGTTCTGCATTTTCTAGTTTAATCATCTTAATACTCCTATAAAGTAGTACA
ATATATTTTCTCCCATTTTACAGTCCCTTTAAAGTAAATAACTATAAAAATCCCTTATACATGTCACA
CAGCTAGGTCTGGCATTTCAAATCAGGACATCAAACAAAGAATTCGTGCAGTTACTAAGTCCTCTATT
TTTTCTACAATAGAAAAAATAGCAAGAATTACAGATAGCAAGACATTACAAGGCAGGAATCTGAAACG
AAAGGGACATAATGTGGGGCTGGGTGGGTGCATGAGCTTTGCAGACTAGACTTTCATTCCAGCTCTTT
TAATGATTAGGTGTAAGTGACCTACATTTTGTGAGTAACAGTTTTCTCATCAGCCAACTAAGAATAAT
TACACCAGATTCACAGTTATTGAAGAGATAAGGGCATGAATGTGAGATGTCTGGCGTAGGGTATCTCA
TTTAGCAGACACAGAATGAATACTTGTTTCTGGCTTTTTCTCTCTACATATGCACAAAGAATGTGACT
AGAAGCATTGGCTCTAGCCCTGCTCAACTTTCCTCTATTTCCAATACCAAGGGGCTCTGACTTAGGCT
GCCACACCAGGCAAGGAGGGGCAGTACCACCTCACTTGACCAAGGGCAGGGAGTCACGGACACATCAC
TTCCTGAGATCCTTTTCCACACCAAGGACTGATGTTTCTGGAATTCTCACTTTATGAAGACAAAACAT
ATAAATGGAAATTTCTGCAGGAAGAGACTCACTCTTGTAGCTCATTGAGTAGGCACTAGTGGTCCACC
CCCACTGTCTTTACTTATTCCTTGACATCACATATCTCTTGTAAAACCTCAAATAATGTTAAATGCAA
TCACCCAATAATAGCATAGCCATAATTAGAGGCATTTAGGAAAGACAGGTGAGTGTGCCACAACTACC
TAACACATCAGCAAATCTGGATTAACCACTTTCTTTGATTTTCCACAATGCAACCTTACTTTTTAATA
GTTGGGAATGTTCTAAGTGAATTTAGCAGAGGTTGTTAATCAACTTGAAAGCTGAATTCTGACTTGTC
TGACTCTTGGTGGTGCTGGTAGCAGTAGATGTTTACTTTTAGGTTTTGGTGGTGGTGGAATATCACTT
CAACGTAAATCATCAGAAATAAGTATTTGTGAACCCCTCTCGCATTAATATATCTTATTCTGTAAAAA
GAACATGTGCAATTTCTCTTAGATACACTACTGCTGCAGCTCACAAACACCTCTGCATATTACACGTA
CCTCCTCCTGCTCCTCAAGAGTGTGGTCTATTTTGCCATCATCACCTGCTGTCTGCTTAGAAGAACGG
CTTTCTGCTGCAATGGAGAGAAATCATAA

In certain embodiments, the first antigen binding chain or the second antigen binding chain comprises a constant domain comprising a native or modified TRDC polypeptide. In certain embodiments, the TRDC polypeptide comprises or consists of an amino acid sequence that is at least about 85%, about 90%, about 95%, about 96%, about 97%, about 98%, about 99%, or 100% homologous or identical to the amino acid sequence set forth in SEQ ID NO: 109, which is provided below. In certain embodiments, the TRDC polypeptide comprises or consists of the amino acid sequence set forth in SEQ ID NO: 109.

[SEQ ID NO: 109]
SQPHTKPSVFVMKNGTNVACLVKEFYPKDIRINLVSSKKITEFDP
AIVISPSGKYNAVKLGKYEDSNSVTCSVQHDNKTVHSTDFEVKTD
STDHVKPKETENTKQPSKSCHKPKAIVHTEKVNMMSLTVLGLRML
EAKTVAVNELLTAKLFFL

In certain embodiments, the TCR-like fusion molecule comprises a hinge/spacer region that links the first antigen binding chain to the constant domain. In certain embodiments, the TCR-like fusion molecule comprises a hinge/spacer region that links the second antigen binding chain to the constant domain. The hinge/spacer region can be flexible enough to allow the antigen binding chain to orient in different directions to facilitate antigen recognition. In certain embodiments, the hinge/spacer region can be the hinge region from IgG1, the CH₂CH₃ region of immunoglobulin and portions of CD3, a portion of a TCRα polypeptide, a portion of a TCRβ polypeptide, a portion of a CD28 polypeptide, a portion of a CD8 polypeptide, or a synthetic spacer sequence. In certain embodiments, the hinge/spacer region comprises a portion of a TCRα polypeptide. In certain embodiments, the hinge/spacer region comprises a portion of the variable region (TRAV), a portion of the diversity region (TRAD), a portion of the joining region (TRAJ), a portion of the constant region (TRAC), or a combination thereof. In certain embodiments, the hinge/spacer region comprises a portion of the TRAJ region and a portion of the TRAC region of the TCRα polypeptide. In certain embodiments, the hinge/spacer region comprises or consists of the amino acid sequence set forth in SEQ ID NO: 110. In certain embodiments, the hinge/spacer region comprises or consists of amino acids 1 to 3 of the sequence set forth in SEQ ID NO: 110. An exemplary nucleic acid sequence encoding the amino acid sequence of SEQ ID NO: 110 is set forth in SEQ ID NO: 111. SEQ ID NO: 110 and 111 are provided below.

[SEQ ID NO: 110]
IPNIQNPDPA
[SEQ ID NO: 111]
ATTCCCAATATCCAGAACCCTGACCCTGCC

In certain embodiments, the hinge/spacer region comprises a portion of a TCRβ polypeptide. In certain embodiments, the hinge/spacer region comprises a portion of the variable region (TRBV), a portion of the diversity region (TRBD), a portion of the joining region (TRBJ), a portion of the constant region (TRBC), or a combination thereof. In certain embodiments, the hinge/spacer region comprises a portion of the TRBJ region and a portion of the TRAC region (C) of the TCRβ polypeptide. In certain embodiments, the hinge/spacer region comprises or consists of the amino acid sequence set forth in SEQ ID NO: 112. In certain embodiments, the hinge/spacer region comprises or consists of amino acids 1 to 2 of the sequence set forth in SEQ ID NO: 112. An exemplary nucleic acid sequence encoding the amino acid sequence of SEQ ID NO: 112 is set forth in SEQ ID NO: 113. SEQ ID NO: 112 and 113 are provided below.

[SEQ ID NO: 112]
LEDLKNVEPPE
[SEQ ID NO: 113]
CTGGAGGATCTGAAAAACGTGTTCCCTCCTGAA

In certain embodiments, the antigen binding chain does not comprise an intracellular domain. In certain embodiments, the antigen binding chain is capable of associating with a CD3ζ polypeptide. In certain embodiments, the antigen binding chain associating with the CD3ζ polypeptide via the constant domain. In certain embodiments, the CD3ζ polypeptide is endogenous. In certain embodiments, the CD3ζ polypeptide is exogenous. In certain embodiments, binding of the antigen binding chain to a target antigen is capable of activating the CD3ζ polypeptide associated to the antigen binding chain. In certain embodiments, the exogenous CD3ζ polypeptide is fused to or integrated with a costimulatory molecule disclosed herein.

In certain embodiments, the TCR-like fusion molecule comprises an antigen binding chain that comprises an intracellular domain. In certain embodiments, the intracellular domain comprises a CD3ζ polypeptide. In certain embodiments, binding of the antigen binding chain to an antigen is capable of activating the CD3ζ polypeptide of the antigen binding chain.

In certain embodiments, the CD3ζ polypeptide comprises or consists of an amino acid sequence that is at least about 85%, about 90%, about 95%, about 96%, about 97%, about 98%, about 99% or about 100% homologous to the amino acid sequence set forth in SEQ ID NO: 68 or a fragment thereof, and/or may optionally comprise up to one or up to two or up to three conservative amino acid substitutions. In certain embodiments, the CD3ζ polypeptide comprises or consists of an amino acid sequence that is a consecutive portion of SEQ ID NO: 68, which is at least about 20, or at least about 30, or at least about 40, or at least about 50, and up to about 164 amino acids in length. In certain embodiments, the CD3ζ comprises or consists of the amino acid sequence of amino acids 1 to 164, 1 to 50, 50 to 100, 52 to 164, 100 to 150, or 150 to 164 of SEQ ID NO: 68. In certain embodiments, the CD3ζ polypeptide comprises or consists of amino acids 52 to 164 of SEQ ID NO: 68.

In certain embodiments, the CD3ζ polypeptide comprises or consists of an amino acid sequence that is at least about 85%, about 90%, about 95%, about 96%, about 97%, about 98%, about 99% or about 100% homologous or identical to SEQ ID NO: 69 or a fragment thereof, and/or may optionally comprise up to one or up to two or up to three conservative amino acid substitutions. In certain embodiments, the CD3ζ polypeptide comprises or consists of the amino acid sequence set forth in SEQ ID NO: 69.

In certain embodiments, the TCR-like fusion molecule comprises an antigen binding chain that comprises an intracellular domain, wherein the intracellular domain comprises a co-stimulatory signaling region. In certain embodiments, the intracellular domain comprises a co-stimulatory signaling region and a CD3 polypeptide. In certain embodiments, the intracellular domain comprises a co-stimulatory signaling region and does not comprise a CD3ζ polypeptide. In certain embodiments, the co-stimulatory signaling region comprises at least an intracellular domain of a co-stimulatory molecule disclosed herein.

In certain embodiments, the TCR-like fusion molecule is capable of associating with a CD3 complex (also known as “T-cell co-receptor”). In certain embodiments, the TCR-like fusion molecule and the CD3 complex form an antigen recognizing receptor complex similar to a native TCR/CD3 complex. In certain embodiments, the CD3 complex is endogenous. In certain embodiments, the CD3 complex is exogenous. In certain embodiments, the TCR-like fusion molecule replaces a native and/or an endogenous TCR in the CD3/TCR complex. In certain embodiments, the CD3 complex comprises a CD3γ chain, a CD3δ chain, and two CD3ε chains.

In certain embodiments, the CD3γ chain comprises or consists of an amino acid sequence that is at least about 85%, about 90%, about 95%, about 96%, about 97%, about 98%, about 99% or about 100% homologous or identical to the amino acid sequence having a NCBI reference number: NP_000064.1 (SEQ ID NO: 114) or a fragment thereof, and/or may optionally comprise up to one or up to two or up to three conservative amino acid substitutions. SEQ ID NO: 114 is provided below.

[SEQ ID NO: 114]
MEQGKGLAVLILAIILLQGTLAQSIKGNHLVKVYDYQEDGSVLLT
CDAEAKNITWEKDGKMIGELTEDKKKWNLGSNAKDPRGMYQCKGS
QNKSKPLQVYYRMCQNCIELNAATISGELFAEIVSIFVLAVGVYF
IAGQDGVRQSRASDKQTLLPNDQLYQPLKDREDDQYSHLQGNQLR
RN

In certain embodiments, the CD3δ chain comprises or consists of an amino acid sequence that is at least about 85%, about 90%, about 95%, about 96%, about 97%, about 98%, about 99% or about 100% homologous or identical to the amino acid sequence having a NCBI reference numbers: NP_000723.1 (SEQ ID NO: 115) or a fragment thereof, or the amino acid sequence having a NCBI reference numbers: NP_001035741.1 (SEQ ID NO: 116) or a fragment thereof, and/or may optionally comprise up to one or up to two or up to three conservative amino acid substitutions. SEQ ID NO: 115 and 116 are provided below.

[SEQ ID NO: 115]
MEHSTFLSGLVLATLLSQVSPEKIPIEELEDRVFVNCNTSITWVE
GTVGTLLSDITRLDLGKRILDPRGIYRCNGTDIYKDKESTVQVHY
RMCQSCVELDPATVAGIIVTDVIATLLLALGVFCFAGHETGRLSG
AADTQALLRNDQVYQPLRDRDDAQYSHLGGNWARNK
[SEQ ID NO: 116]
MEHSTFLSGLVLATLLSQVSPEKIPIEELEDRVFVNCNTSITWVE
GTVGTLLSDITRLDLGKRILDPRGIYRCNGTDIYKDKESTVQVHY
RTADTQALLRNDQVYQPLRDRDDAQYSHLGGNWARNK

In certain embodiments, the CD3ε chain comprises or consists of an amino acid sequence that is at least about 85%, about 90%, about 95%, about 96%, about 97%, about 98%, about 99% or about 100% homologous or identical to the amino acid sequence having a NCBI reference number: NP_000724.1 (SEQ ID NO: 117) or a fragment thereof, and/or may optionally comprise up to one or up to two or up to three conservative amino acid substitutions. SEQ ID NO: 117 is provided below.

[SEQ ID NO: 117]
MQSGTHWRVLGLCLLSVGVWGQDGNEEMGGITQTPYKVSISGTTV
ILTCPQYPGSEILWQHNDKNIGGDEDDKNIGSDEDHLSLKEFSEL
EQSGYYVCYPRGSKPEDANFYLYLRARVCENCMEMDVMSVATIVI
VDICITGGLLLLVYYWSKNRKAKAKPVTRGAGAGGRQRGQNKERP
PPVPNPDYEPIRKGQRDLYSGLNQRRI

In certain embodiments, the TCR-like fusion molecule exhibits a greater antigen sensitivity than a CAR targeting the same antigen. In certain embodiments, the TCR-like fusion molecule is capable of inducing an immune response when binding to an antigen that has a low cell density. In certain embodiments, the TCR-like fusion molecule is capable of inducing an immune response when binding to an antigen that has a low tumor density.

In certain embodiments, the antigen-recognizing receptor is a TCR-like fusion molecule that comprises a first antigen binding chain comprising a V_{H} of an antibody and a constant domain comprising a TRBC polypeptide; and a second antigen binding chain comprising a V_{L} of an antibody and a constant domain comprising a TRAC polypeptide. In certain embodiments, the first antigen binding chain is designated as “V_{H}-TRBC chain”. In certain embodiments, the second antigen binding chain is designated as “V_{L}-TRAC chain”. In certain embodiments, the first antigen binding chain comprises a hinge region between the V_{H} and the TRBC polypeptide. In certain embodiments, the hinge region comprises or consists of the amino acid sequence set forth in SEQ ID NO: 110 or SEQ ID NO: 112. In certain embodiments, the second antigen binding chain comprises a hinge region between the V_{L} and the TRAC polypeptide. In certain embodiments, the hinge region comprises or consists of the amino acid sequence set forth in SEQ ID NO: 110 or SEQ ID NO: 112.

In certain embodiments, the antigen-recognizing receptor is a TCR-like fusion molecule that comprises a first antigen binding chain comprising a V_{H} of an antibody and a constant domain comprising a TRAC polypeptide; and a second antigen binding chain comprising a V_{L} of an antibody and a constant domain comprising a TRBC polypeptide. In certain embodiments, the first antigen binding chain is designated as “VH-TRAC chain”. In certain embodiments, the second antigen binding chain is designated as “V_{L}-TRBC chain”. In certain embodiments, the first antigen binding chain comprises a hinge region between the V_{H} and the TRAC polypeptide. In certain embodiments, the second antigen binding chain comprises a hinge region between the V_{L} and the TRBC polypeptide. In certain embodiments, the first antigen binding chain and the second antigen binding chain bind to an antigen.

In certain embodiments, the antigen-recognizing receptor is a TCR-like fusion molecule that comprises a first antigen binding chain comprising a VH of an antibody and a constant domain comprising a TRBC polypeptide; and a second antigen binding chain comprising a V_{L} of an antibody and a constant domain comprising a TRAC polypeptide. In certain embodiments, the first antigen binding chain is designated as “V_{H}-TRBC chain”. In certain embodiments, the second antigen binding chain is designated as “V_{L}-TRAC chain”. In certain embodiments, the first antigen binding chain comprises a hinge region between the V_{H} and the TRAC polypeptide. In certain embodiments, the second antigen binding chain comprises a hinge region between the V_{L} and the TRBC polypeptide. In certain embodiments, the first antigen binding chain and the second antigen binding chain bind to an antigen.

### 3.1.3.1. Exemplary TCR-Like Fusion Molecules

In certain embodiments, the antigen-recognizing receptor is a TCR-like fusion molecule that binds to CD19 (e.g., human CD19) and comprises two antigen binding chains, e.g., a first antigen binding chain that comprises a V_{H} and a TRBC polypeptide (“V_{H}-TRBC chain”) and a second antigen binding chain that comprises a V_{L} and a TRBC polypeptide (“V_{L}-TRAC chain”), which are capable of dimerizing and binding to CD19. In certain embodiments, the VH comprises a CDR1 comprising the amino acid sequence set forth in SEQ ID NO: 118, a CDR2 comprising the amino acid sequence set forth in SEQ ID NO: 119, and a CDR3 comprising the amino acid sequence set forth in SEQ ID NO: 120. In certain embodiments, the V_{H} comprises the amino acid sequence set forth in SEQ ID NO: 124. In certain embodiments, the V_{L} comprises a CDR1 comprising the amino acid sequence set forth in SEQ ID NO: 121, a CDR2 comprising the amino acid sequence set forth in SEQ ID NO: 122, and a CDR3 comprising the amino acid sequence set forth in SEQ ID NO: 123. In certain embodiments, the V_{L} comprises the amino acid sequence set forth in SEQ ID NO: 126. In certain embodiments, the TRAC polypeptide comprises the amino acid sequence set forth in SEQ ID NO: 93. In certain embodiments, the TRBC polypeptide is a TRBC2 polypeptide. In certain embodiments, the TRBC2 polypeptide comprises the amino acid sequence set forth in SEQ ID NO: 98. SEQ ID NO: 118-127 are provided in Table 5 below.

In certain embodiments, the CDRs regions/sequences disclosed herein are delineated using the Kabat system (Swindells et al., J Mol Biol. 2017 Feb. 3; 429(3):356-364).

TABLE 5

| CDRS | 1 | 2 | 3 |
|---|---|---|---|
| V_{H} | SYWMN | QIYPGDGDTNYNGKFKG | KTISSVVDFYFDY |
| | [SEQ ID NO: 118] | [SEQ ID NO: 119] | [SEQ ID NO: 120] |
| V_{L} | KASQNVGTNVA | SATYRNS | QQYNRYPYT |
| | [SEQ ID NO: 121] | [SEQ ID NO: 122] | [SEQ ID NO: 123] |

| | |
|---|---|
| FULL V_{H} | MALPVTALLLPLALLLHAEVKLQQSGAELVRPGSSVKISCKASGYAFSSYWMNWVK |
| | QRPGQGLEWIGQIYPGDGDTNYNGKFKGQATLTADKSSSTAYMQLSGLTSEDSAVY |
| | FCARKTISSVVDFYFDYWGQGTTVTV |
| | [SEQ ID NO: 124] |
| FULL V_{H}- | ATGGCTCTCCCAGTGACTGCCCTACTGCTTCCCCTAGCGCTTCTCCTGCATGCAGA |
| DNA | GGTGAAGCTGCAGCAGTCTGGGGCTGAGCTGGTGAGGCCTGGGTCCTCAGTGAAGA |
| | TTTCCTGCAAGGCTTCTGGCTATGCATTCAGTAGCTACTGGATGAACTGGGTGAAG |
| | CAGAGGCCTGGACAGGGTCTTGAGTGGATTGGACAGATTTATCCTGGAGATGGTGA |
| | TACTAACTACAATGGAAAGTTCAAGGGTCAAGCCACACTGACTGCAGACAAATCCT |
| | CCAGCACAGCCTACATGCAGCTCAGCGGCCTAACATCTGAGGACTCTGCGGTCTAT |
| | TTCTGTGCAAGAAAGACCATTAGTTCGGTAGTAGATTTCTACTTTGACTACTGGGG |
| | CCAAGGGACCACGGTCACCGTC |
| | [SEQ ID NO: 125] |
| FULL V_{L} | MALPVTALLLPLALLLHADIELTQSPKFMSTSVGDRVSVTCKASQNVGTNVAWYQQ |
| | KPGQSPKPLIYSATYRNSGVPDRFTGSGSGTDFTLTITNVQSKDLADYFCQQYNRY |
| | PYTSGGGTKLEI |
| | [SEQ ID NO: 126] |
| FULL V_{L}- | ATGGCACTGCCCGTGACCGCTCTGCTTCTCCCACTAGCTCTGCTTCTCCACGCAGA |
| DNA | CATTGAGCTCACCCAGTCTCCAAAATTCATGTCCACATCAGTAGGAGACAGGGTCA |
| | GCGTCACCTGCAAGGCCAGTCAGAATGTGGGTACTAATGTAGCCTGGTATCAACAG |
| | AAACCAGGACAATCTCCTAAACCACTGATTTACTCGGCAACCTACCGGAACAGTGG |
| | AGTCCCTGATCGCTTCACAGGCAGTGGATCTGGGACAGATTTCACTCTCACCATCA |
| | CTAACGTGCAGTCTAAAGACTTGGCAGACTATTTCTGTCAACAATATAACAGGTAT |
| | CCGTACACGTCCGGAGGGGGGACCAAGCTGGAGATC [SEQ ID NO: 127] |

In certain embodiments, the antigen-recognizing receptor is a TCR-like fusion molecule that binds to CD70 (e.g., human CD70) and comprises two antigen binding chains, e.g., a first antigen binding chain that comprises a V_{H} and a TRBC polypeptide (“V_{H}-TRBC chain”) and a second antigen binding chain that comprises a V_{L} and a TRBC polypeptide (“V_{L}-TRAC chain”), which are capable of dimerizing and binding to CD70. In certain embodiments, the V_{H} comprises a CDR1 comprising the amino acid sequence set forth in SEQ ID NO: 133, a CDR2 comprising the amino acid sequence set forth in SEQ ID NO: 134, and a CDR3 comprising the amino acid sequence set forth in SEQ ID NO: 135. In certain embodiments, the V_{H} comprises the amino acid sequence set forth in SEQ ID NO: 139. In certain embodiments, the V_{L} comprises a CDR1 comprising the amino acid sequence set forth in SEQ ID NO: 136, a CDR2 comprising the amino acid sequence set forth in SEQ ID NO: 137, and a CDR3 comprising the amino acid sequence set forth in SEQ ID NO: 138. In certain embodiments, the V_{L} comprises the amino acid sequence set forth in SEQ ID NO: 140. In certain embodiments, the TRAC polypeptide comprises the amino acid sequence set forth in SEQ ID NO: 93. In certain embodiments, the TRBC polypeptide is a TRBC2 polypeptide. In certain embodiments, the TRBC2 polypeptide comprises the amino acid sequence set forth in SEQ ID NO: 98. SEQ ID NO: 133-140 are provided in Table 4.

In certain embodiments, the antigen-recognizing receptor is a TCR-like fusion molecule that binds to CD70 (e.g., human CD70) and comprises two antigen binding chains, e.g., a first antigen binding chain that comprises a VH and a TRBC polypeptide (“V_{H}-TRBC chain”) and a second antigen binding chain that comprises a V_{L} and a TRBC polypeptide (“V_{L}-TRAC chain”), which are capable of dimerizing and binding to CD70. In certain embodiments, the VH comprises a CDR1, a CDR2, and a CDR3 of a VH sequence of an anti-CD70 antibody disclosed in International Patent Publication No. WO 2007/038637, which is incorporated by reference in its entirety. In certain embodiments, the V_{H} comprises a CDR1, a CDR2, and a CDR3 of a V_{H} sequence of an anti-CD70 antibody 2H5 disclosed in International Patent Publication No. WO 2007/038637. In certain embodiments, the V_{L} comprises a CDR1, a CDR2, and a CDR3 of a V_{H} sequence of an anti-CD70 antibody disclosed in International Patent Publication No. WO 2007/038637. In certain embodiments, the V_{L} comprises a CDR1, a CDR2, and a CDR3 of a V_{H} sequence of an anti-CD70 antibody 2H5 disclosed in International Patent Publication No. WO 2007/038637.

Various TCR-like fusion molecules are disclosed in International Patent Application Publication No. WO2019/133969, which is incorporated by reference hereby in its entirety.

### 3.1.4. T Cell Receptors (TCRs)

In certain embodiments, the antigen-recognizing receptor is a T cell receptor (TCR). A TCR is a disulfide-linked heterodimeric protein consisting of two variable chains expressed as part of a complex with the invariant CD3 chain molecules. A TCR is found on the surface of T cells, and is responsible for recognizing antigens as peptides bound to major histocompatibility complex (MHC) molecules. In certain embodiments, a TCR comprises an alpha chain and a beta chain (encoded by TRA and TRB, respectively). In certain embodiments, a TCR comprises a gamma chain and a delta chain (encoded by TRG and TRD, respectively).

Each chain of a TCR is composed of two extracellular domains: Variable (V) region and a Constant (C) region. The Constant region is proximal to the cell membrane, followed by a transmembrane region and a short cytoplasmic tail. The variable region binds to the peptide/MHC complex. The variable domain of both chains each has three complementarity determining regions (CDRs).

In certain embodiments, a TCR can form a receptor complex with three dimeric signaling modules CD3δ/ε, CD3γ/ε and CD247 ζ/ζ or ζ/η. When a TCR complex engages with its antigen and MHC (peptide/MHC), the T cell expressing the TCR complex is activated.

In certain embodiments, the TCR is an endogenous TCR. In certain embodiments, the TCR is naturally occurring TCR.

In certain embodiments, the TCR is an exogenous TCR. In certain embodiments, the TCR is a recombinant TCR. In certain embodiments, the TCR is a non-naturally occurring TCR. In certain embodiments, the non-naturally occurring TCR differs from any naturally occurring TCR by at least one amino acid residue. In certain embodiments, the non-naturally occurring TCR differs from any naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues. In certain embodiments, the non-naturally occurring TCR is modified from a naturally occurring TCR by at least one amino acid residue. In certain embodiments, the non-naturally occurring TCR is modified from a naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues.

### 3.2. Chimeric Receptors

In certain embodiments, the cells comprising the fusion polypeptide and the antigen-recognizing receptor further comprise a chimeric receptor. In certain embodiments, the chimeric receptor can be a chimeric ligand receptor or a CCR.

### 3.2.1. Chimeric Ligand Receptors

In certain embodiments, the chimeric receptor is a chimeric ligand receptor that comprises a ligand or a portion thereof that binds to the antigen. In certain embodiments, the chimeric ligand receptor further comprises a transmembrane domain and an intracellular signaling domain.

In certain embodiments, the transmembrane domain is fused to the ligand or portion thereof. In certain embodiments, the transmembrane domain is fused to the intracellular signaling domain. In certain embodiments, the transmembrane domain is positioned between the ligand or portion thereof and the intracellular signaling domain. In certain embodiments the transmembrane domain of the chimeric ligand receptor is a transmembrane domain disclosed in Section 3.1.2.2. In certain embodiments, the intracellular signaling domain of the chimeric ligand receptor comprises a CD3ζ polypeptide (e.g., as disclosed in Section 3.1.2.3).

Additional information on the presently disclosed chimeric ligand receptor can be found in Sauer et al., Blood (2021) 138 (4): 318-330, the content of which is incorporated by reference in its entirety.

### 3.2.2. CCRs

In certain embodiments, the chimeric receptor is a CCR. The term “chimeric co-stimulating receptor” or “CCR” refers to a chimeric receptor that binds to an antigen and provides a co-stimulatory signal, but does not provide a T-cell activation signal to a cell comprising the CCR. Various CCRs are described in US20020018783 the contents of which are incorporated by reference in their entireties. CCRs mimic co-stimulatory signals, but unlike, CARs, do not provide a T-cell activation signal. In certain embodiments, the CCR lacks a CD3ζ polypeptide.

CCRs provide co-stimulation signal (e.g., a CD28-like signal or 4-1BB-like signal), in the absence of the natural co-stimulatory ligand on the antigen-presenting cell. A combinatorial antigen recognition, i.e., use of a CCR in combination with a CAR, can augment T-cell reactivity against the dual-antigen expressing T cells, thereby improving selective tumor targeting. Kloss et al., describe a strategy that integrates combinatorial antigen recognition, split signaling, and, critically, balanced strength of T-cell activation and co-stimulation to generate T cells that eliminate target cells that express a combination of antigens while sparing cells that express each antigen individually (Kloss et al., Nature Biotechnology (2013); 31(1):71-75, the content of which is incorporated by reference in its entirety). With this approach, T-cell activation requires CAR-mediated recognition of one antigen, whereas co-stimulation is independently mediated by a CCR specific for a second antigen. To achieve tumor selectivity, the combinatorial antigen recognition approach diminishes the efficiency of T-cell activation to a level where it is ineffective without rescue provided by simultaneous CCR recognition of the second antigen.

In certain embodiments, the CCR comprises an extracellular antigen-binding domain that binds to a second antigen and an intracellular domain that is capable of delivering a costimulatory signal to the cell but does not alone deliver an activation signal to the cell. In certain embodiments, the CCR further comprises a transmembrane domain. In certain embodiments, the intracellular domain of the CCR comprises at least an intracellular domain of a co-stimulatory molecule or a portion thereof. In certain embodiments, the co-stimulatory molecule is selected from the group consisting of CD28, 4-1BB, OX40, CD27, CD40, CD154, CD97, CD11a/CD18, ICOS, DAP-10, CD2, CD150, CD226, and NKG2D.

In certain embodiments, the CCR comprises an intracellular domain of CD28 or a portion thereof. In certain embodiments, the CCR comprises an intracellular domain of 4-1BB or a portion thereof. In certain embodiments, the CCR comprises an intracellular domain of CD28 or a portion thereof, and an intracellular domain of 4-1BB or a portion thereof.

In certain embodiments, the second antigen is selected so that expression of both the first antigen and the second antigen is restricted to the targeted cells (e.g., cancerous tissue or cancerous cells, LSCs, or AML HSPCs). Similar to a CAR, the extracellular antigen-binding domain can be an scFv, a Fab, an F(ab)₂, or a chimeric protein with a heterologous sequence to form the extracellular antigen-binding domain.

In certain embodiments, the cell comprises a first antigen-recognizing receptor (e.g., a CAR, a TCR, or a TCR-like fusion molecule) and a CCR. In certain embodiments, a cell comprising a first antigen-recognizing receptor (e.g., a CAR, a TCR, or a TCR-like fusion molecule) and a CCR exhibits a greater degree of cytolytic activity against cells that are positive for both the first antigen and the second antigen as compared to against cells that are singly positive for the first antigen. In certain embodiments, the cell comprising the first antigen-recognizing receptor and the CCR exhibits substantially no or negligible cytolytic activity against cells that are singly positive for the first antigen.

In certain embodiments, the first antigen recognizing receptor binds to the first antigen with a low binding affinity, e.g., a dissociation constant (K_{D}) of about 1×10⁻⁸ M or more, about 5×10⁻⁸ M or more, about 1×10⁻⁷ M or more, about 5×10⁻⁷ M or more, or about 1×10⁻⁶ M or more, or from about 1×10⁻⁸ M to about 1×10⁻⁶ M. In certain embodiments, the antigen recognizing receptor (e.g., a CAR, a TCR, or a TCR-like fusion molecule) binds to the antigen with a low binding avidity. In certain embodiments, the antigen recognizing receptor (e.g., a CAR, a TCR, or a TCR-like fusion molecule) binds to the antigen at an epitope of low accessibility. In certain embodiments, the antigen recognizing receptor (e.g., a CAR, a TCR, or a TCR-like fusion molecule) binds to the antigen with a binding affinity that is lower compared to the binding affinity with which the CCR binds to the second antigen. In certain embodiments, the CCR binds to the third antigen with a binding affinity K_{D} of from about 1×10⁻⁹ M to about 1×10⁻⁷ M, e.g., about 1×10⁻⁷ M or less, about 1×10⁻⁸ M or less, or about 1×10⁻⁹ M or less.

### 3.3. Delivery of the Antigen-Recognizing Receptor and Chimeric Receptors

In certain embodiments, the antigen-recognizing receptor and/or the chimeric receptor, or a polynucleotide encoding the same, can be delivered to the cell by a viral method. In certain embodiments, the viral method comprises a viral vector. In certain embodiments, the viral vector is a retroviral vector (e.g., a gamma-retroviral vector or a lentiviral vector). Other viral vectors include adenoviral vectors, adeno-associated viral vectors, vaccinia viruses, bovine papilloma viruses, and herpes viruses (e.g., such as Epstein-Barr Virus).

In certain embodiments, the antigen-recognizing receptor and/or the chimeric receptor, or a polynucleotide encoding the same, can be delivered to the cell by a non-viral method. Any targeted genome editing methods can also be used to deliver the second antigen-recognizing receptor to the cell. In certain embodiments, the antigen-recognizing receptor and/or the chimeric receptor is delivered to the cell by a method comprising homologous recombination, a Zinc finger nuclease, a meganuclease, a Transcription activator-like effector nuclease (TALEN), a Clustered regularly-interspaced short palindromic repeats (CRISPR) system, or a combination thereof. In certain embodiments, a CRISPR system is used to deliver the second antigen-recognizing receptor to the cell.

In certain embodiments, the cell is a T cell, and the antigen-recognizing receptor, or a polynucleotide encoding the same, is integrated at a locus within the genome of the T cell. Non-limiting examples of loci include a TRAC locus, a TRBC locus, a TRDC locus, and a TRGC locus. In certain embodiments, the locus is a TRAC locus or a TRBC locus. In certain embodiments, the cell is a T cell, and the antigen-recognizing receptor is integrated at a TRAC locus.

Additionally or alternatively, the antigen-recognizing receptor and/or the chimeric receptor, or a polynucleotide encoding the same, can be integrated at a genomic safe harbor within the genome of the T cell. Further information on genomic safe harbors and on methods for identifying the same can be found in International Patent Publications No. 2021/055592 and No. 2021/055616, the contents of each of which are incorporated by reference in their entirety.

### 3.4. Gene Disruptions

In certain embodiments, a presently disclosed cell comprising a presently disclosed fusion polypeptide further comprises gene disruption of a TRAC locus. In certain embodiments, the gene disruption of the TRAC locus results in a non-functional TCR. In certain embodiments, the gene disruption of the TRAC locus results in the knockout of the TCR gene expression.

Non-limiting examples of gene disruptions include substitutions, deletions, insertions, or combinations thereof. In certain embodiments, the mutation comprises a missense mutation, a nonsense mutation, or a combination thereof. In certain embodiments, the deletion comprises a non-frameshift deletion, a frameshift deletion, or a combination thereof. In certain embodiments, the insertion comprises a non-frameshift insertion, a frameshift insertion, or a combination thereof.

In certain embodiments, the TRAC locus is a human TRAC locus. The gene disruption of the TRAC locus can be generated by any suitable gene editing methods. In certain embodiments, the gene disruption of the TRAC locus (e.g., knockout of the TRAC locus) is generated using a viral method. In certain embodiments, the viral method comprises a viral vector. In certain embodiments, the viral vector is a retroviral vector (e.g., a gamma-retroviral vector or a lentiviral vector). Other viral vectors include adenoviral vectors, adena-associated viral vectors, vaccinia viruses, bovine papilloma viruses, and herpes viruses (e.g., such as Epstein-Barr Virus).

In certain embodiments, the gene disruption of the TRAC locus (e.g., knockout of the TRAC locus) is generated using a non-viral method. Non-viral approaches can also be employed for genetic modification of a cell. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g. Zinc finger nucleases, meganucleases, or TALE nucleases, CRISPR). Transient expression may be obtained by RNA electroporation.

Any targeted genome editing methods can also be used to generate the gene disruption of the TRAC locus. In certain embodiments, the gene disruption of the TRAC locus is generated by a method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

In certain embodiments, a CRISPR system is used to generate the gene disruption of the TRAC locus.

Clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. The crRNA needs to be designed for each application as this is the sequence that Cas9 uses to identify and directly bind to the target DNA in a cell. The repair template carrying CAR expression cassette need also be designed for each application, as it must overlap with the sequences on either side of the cut and code for the insertion sequence. Multiple crRNA's and the tracrRNA can be packaged together to form a single-guide RNA (sgRNA). This sgRNA can be joined together with the Cas9 gene and made into a plasmid in order to be transfected into cells. In certain embodiments, the CRISPR system comprises base editors. In certain embodiments, the CRISPR system comprises transposases/recombinases. In certain embodiments, the CRISPR system comprises prime editors. In certain embodiments, the CRISPR system comprises an epigenetic modulator. In certain embodiments, the CRISPR system comprises is a CRISPRoff system. Additional details on the CRISPR systems of the presently disclosed subject matter can be found in Anzalone et al., Nature biotechnology 38.7 (2020): 824-844 and in Nunez et al., Cell 184.9 (2021): 2503-2519, the contents of each of which are incorporated by reference in their entireties.

In certain embodiments, the TRAC locus is disrupted using a gRNA molecule to knockout expression of TRAC. The gRNA molecule can target a coding sequence of a TRAC gene (e.g., a human TRAC gene) or a non-coding sequence of a TRAC gene (e.g., a human TRAC gene). In certain embodiments, the gRNA molecule targets a coding sequence of a TRAC gene (e.g., a human TRAC gene). In certain embodiments, the gRNA molecule targets a target sequence within a human TRAC gene.

In certain embodiments, zinc-finger nucleases are used to generate the gene disruption of the TRAC locus. A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of basepairs. The most common method to generate new zinc-finger domain is to combine smaller zinc-finger “modules” of known specificity. The most common cleavage domain in ZFNs is the non-specific cleavage domain from the type IIs restriction endonuclease FokI. Using the endogenous homologous recombination (HR) machinery and a homologous DNA template carrying a desired expression cassette, ZFNs can be used to insert the desired expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome.

In certain embodiments, a TALEN system is used to generate the gene disruption of the TRAC locus. Transcription activator-like effector nucleases (TALEN) are restriction enzymes that can be engineered to cut specific sequences of DNA. TALEN system operates on almost the same principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome. cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

In certain embodiments, the gene disruption of the TRAC locus can be a disruption of the coding region of the TRAC locus and/or a disruption of the non-coding region of the TRAC locus. In certain embodiments, the gene disruption of the TRAC locus comprises a disruption of the coding region of the TRAC locus. In certain embodiments, the gene disruption of the TRAC locus comprises an insertion at the coding region of the TRAC locus. Human TRAC protein comprises 4 exons: exon 1, exon 2, exon 3, and exon 4. In certain embodiments, the coding region of the TRAC locus comprises exon 1, exon 2, exon 3, and exon 4. In certain embodiments, the gene disruption of the TRAC locus comprises a disruption at one or more of exon 1 through exon 4 of the TRAC locus. In certain embodiments, the gene disruption of the TRAC locus comprises a disruption at exon 1 of the TRAC locus. In certain embodiments, the gene disruption of the TRAC locus comprises an insertion at exon 1 of the TRAC locus.

The presently disclosed cells further comprise a gene disruption of a CD70 locus. The gene disruption of the CD70 locus can result in a non-functional CD70 protein or a knockout of the CD70 gene expression. In certain embodiments, the gene disruption of the CD70 locus results in knockout of the CD70 gene expression. Non-limiting examples of gene disruptions include substitutions, deletions, insertions, or combinations thereof. In certain embodiments, the mutation comprises a missense mutation, a nonsense mutation, or a combination thereof. In certain embodiments, the deletion comprises a non-frameshift deletion, a frameshift deletion, or a combination thereof. In certain embodiments, the insertion comprises a non-frameshift insertion, a frameshift insertion, or a combination thereof.

In certain embodiments, the CD70 locus is a human CD70 locus. The gene disruption of the CD70 locus can be generated by any suitable gene editing methods. In certain embodiments, the gene disruption of the CD70 locus (e.g., knockout of the CD70 locus) is generated using a viral method. In certain embodiments, the viral method comprises a viral vector. In certain embodiments, the viral vector is a retroviral vector (e.g., a gamma-retroviral vector or a lentiviral vector). Other viral vectors include adenoviral vectors, adena-associated viral vectors, vaccinia viruses, bovine papilloma viruses, and herpes viruses (e.g., such as Epstein-Barr Virus).

In certain embodiments, the gene disruption of the CD70 locus (e.g., knockout of the CD70 locus) is generated using a non-viral method.

Any targeted genome editing methods can also be used to generate the gene disruption of the CD70 locus. In certain embodiments, the gene disruption of the CD70 locus is generated by a method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

In certain embodiments, the gene disruption of the CD70 locus can be a disruption of the coding region of the CD70 locus and/or a disruption of the non-coding region of the CD70 locus. In certain embodiments, the gene disruption of the CD70 locus comprises a disruption of the coding region of the CD70 locus. In certain embodiments, the gene disruption of the CD70 locus comprises an insertion at the coding region of the CD70 locus. Human CD70 protein comprises three exons: exon 1, exon 2, and exon 3. In certain embodiments, the gene disruption of the CD70 locus comprises a disruption at one or more of exon 1, exon 2, and exon 3 of the CD70 locus. In certain embodiments, the gene disruption of the CD70 locus comprises a disruption at exon 1 of the CD70 locus. In certain embodiments, the gene disruption of the CD70 locus comprises an insertion at exon 1 of the CD70 locus.

### 4. NUCLEIC ACIDS AND VECTORS

The presently disclosed subject matter provides nucleic acids comprising a polynucleotide encoding a fusion polypeptide disclosed herein (e.g., disclosed in Section 2). In certain embodiments, the nucleic acids further comprise a second polynucleotide encoding an antigen-recognizing receptor and/or a chimeric receptor disclosed herein (e.g., disclosed in Sections 3.1 and 3.2, respectively). Also provided are cells comprising such nucleic acids. In certain embodiments, the nucleic acid further comprises a promoter that is operably linked to the fusion polypeptide disclosed herein. In certain embodiments, the nucleic acid further comprises a second promoter that is operably linked to the antigen-recognizing receptor and/or chimeric receptor.

In certain embodiments, one or both of the first and second promoters are endogenous or exogenous.

In certain embodiments, the exogenous promoter is selected from an elongation factor (EF)-1 promoter, a CMV promoter, a SV40 promoter, a PGK promoter, and a metallothionein promoter. In certain embodiments, one or both of the first and second promoters are inducible promoters. In certain embodiments, the inducible promoter is selected from an NFAT transcriptional response element (TRE) promoter, a CD69 promoter, a CD25 promoter, and an IL-2 promoter.

In certain embodiments, the polynucleotide is integrated at a locus within the genome of the T cell, e.g., a TRAC locus, a TRBC locus, a TRDC locus, or a TRGC locus. In certain embodiments, the locus is a TRAC locus. In certain embodiments, the expression of the fusion polypeptide is under the control of an endogenous promoter. In certain embodiments, the expression of the fusion polypeptide and the antigen-recognizing receptor is under the control of an endogenous promoter. Non-limiting examples of endogenous promoters include an endogenous TRAC promoter, an endogenous TRBC promoter, an endogenous TRDC promoter, and an endogenous TRGC promoter. In certain embodiments, the endogenous promoter is an endogenous TRAC promoter.

In certain embodiments, the nucleic acid composition is a vector. In certain embodiments, the vector is a retroviral vector (e.g., a gamma-retroviral vector or a lentiviral vector). In certain embodiments, the vector is viral vectors selected from the group consisting of adenoviral vectors, adena-associated viral vectors, vaccinia viruses, bovine papilloma viruses, and herpes viruses (e.g., such as Epstein-Barr Virus).

Additionally, the nucleic acids can be administered to subjects or and/delivered into cells by art-known methods or as described herein. Genetic modification of a cell (e.g., a T cell or an NK cell) can be accomplished by transducing a substantially homogeneous cell composition with a recombinant DNA construct. In certain embodiments, a retroviral vector (either gamma-retroviral or lentiviral) is employed for the introduction of the nucleic acid compositions into the cell. For example, the first polynucleotide and the second polynucleotide can be cloned into a retroviral vector and expression can be driven from its endogenous promoter, from the retroviral long terminal repeat, or from a promoter specific for a target cell type of interest. Non-viral vectors may be used as well.

In certain embodiments, when the nucleic acid includes multiple polynucleotides, the first polynucleotide and the second polynucleotide can be constructed in a single, multicistronic expression cassette, in multiple expression cassettes of a single vector, or multiple vectors. Examples of elements that create polycistronic expression cassette include, but is not limited to, various viral and non-viral Internal Ribosome Entry Sites (IRES, e.g., FGF-1 IRES, FGF-2 IRES, VEGF IRES, IGF-II RES, NF-κB IRES, RUNX1 IRES, p53 IRES, hepatitis A IRES, hepatitis C IRES, pestivirus IRES, aphthovirus IRES, picornavirus IRES, poliovirus IRES, and encephalomyocarditis virus IRES) and cleavable linkers (e.g., 2A peptides, e.g., P2A, T2A, E2A and F2A peptides). Combinations of retroviral vectors and an appropriate packaging line are also suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD 114, or GALV envelope and any other known in the art.

Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Other transducing viral vectors can be used to modify a cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adena-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for genetic modification of a cell. For example, a nucleic acid molecule can be delivered into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Transient expression may be obtained by RNA electroporation.

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but are not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

In certain embodiments, the delivery methods include the use of colloids. As used herein, the term “colloid” refers to systems in which there are two or more phases, with one phase (e.g., the dispersed phase) distributed in the other phase (e.g., the continuous phase). Moreover, at least one of the phases has small dimensions (in the range of about 10⁻⁹ to about 10⁻⁶ m). Non-limiting examples of colloids encompassed by the presently disclosed subject matter include macromolecule complexes, nanocapsules, microspheres, beads, and lipid-based systems (e.g., micelles, liposomes, and lipid nanoparticles).

In certain embodiments, the delivery methods include the use of liposomes. The term “liposome,” as used herein, refers to single- or multi-layered spherical lipid bilayer structures produced from lipids dissolved in organic solvents and then dispersed in aqueous media. Experimentally and therapeutically used for delivering an active pharmaceutical ingredient (e.g., nucleic acid compositions disclosed herein) to cells, liposomes fuse with cell membranes so the contents are transferred into the cytoplasm.

In certain embodiments, the delivery methods include the use of lipid nanoparticles. As used herein, the term “lipid nanoparticle” refers to a particle having at least one dimension in the order of nanometers (e.g., from about 1 nm to about 1,000 nm) and including at least one lipid. In certain embodiments, the lipid nanoparticles can include an active pharmaceutical ingredient (e.g., nucleic acid compositions disclosed herein) for delivering to cells. The morphology of the lipid nanoparticles can be different from liposomes. While liposomes are characterized by a lipid bilayer surrounding a hydrophilic core, lipid nanoparticles have an electron-dense core where cationic lipids and/or ionizable lipids are organized into inverted micelles around an active pharmaceutical ingredient (e.g., nucleic acid compositions disclosed herein). Additional information on the morphology and properties of lipid nanoparticles and liposomes can be found in Wilczewska, et al., Pharmacological reports 64, no. 5 (2012): 1020-1037; Eygeris et al., Accounts of Chemical Research 55, no. 1 (2021): 2-12; Zhang et al., Chemical Reviews 121, no. 20 (2021): 12181-12277; and Fan et al., Journal of pharmaceutical and biomedical analysis 192 (2021): 113642.

In certain embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm.

In certain embodiments, the lipid nanoparticles can include a cationic lipid or an ionizable lipid. The term “cationic lipid” refers to lipids including a head group with permanent positive charges. Non-limiting examples of cationic lipids encompassed by the presently disclosed subject matter include 1,2-di-O-octadecenyl-3-trimethylammonium-propane (DOTMA), 1,2-dioleoyl-3-trimethylammonium-propane (DOTAP), 2,3-dioleyloxy-N-[2-(sperminecarboxamido)ethyl]-N,N-dimethyl-1-propanaminium trifluoroacetate (DOSPA), and ethylphosphatidylcholine (ePC).

As used herein, the term “ionizable lipid” refers to lipids that are protonated at low pH and are neutral at physiological pH. The pH-sensitivity of ionizable lipids is particularly beneficial for delivery in vivo (e.g., delivery of nucleic acid compositions disclosed herein), because neutral lipids have less interactions with the anionic membranes of blood cells and, thus, improve the biocompatibility of the lipid nanoparticles. Once trapped in endosomes, ionizable lipids are protonated and promote membrane destabilization to allow the endosomal escape of the nanoparticles. Non-limiting example of ionizable lipids encompassed by the presently disclosed subject matter include tetrakis(8-methylnonyl) 3,3′,3″,3′″-(((methylazanediyl) bis(propane-3,1 diyl))bis (azanetriyl))tetrapropionate; decyl (2-(dioctylammonio)ethyl) phosphate; ((4-hydroxybutyl)azanediyl)bis(hexane-6,1-diyl)bis(2-hexyldecanoate); bis(2-(dodecyldisulfanyl)ethyl) 3,3′-((3-methyl-9-oxo-10-oxa-13,14-dithia-3,6-diazahexacosyl)azanediyl)dipropionate; 1,1′-((2-(4-(2-((2-(bis(2-hydroxydodecyl)amino)ethyl) (2-hydroxydodecyl)amino)ethyl) piperazin-1-yl)ethyl)azanediyl) bis(dodecan-2-ol); cKK-E12, 3,6-bis(4-(bis(2-hydroxydodecyl)amino)butyl)piperazine-2,5-dione; (6Z,9Z,28Z,31Z)-heptatriaconta-6,9,28,31-tetraen-19-yl 4-(dimethylamino) butanoate; hexa(octan-3-yl) 9,9′,9″,9′″,9″″,9′″″-((((benzene-1,3,5-tricarbonyl)yris(azanediyl)) tris (propane-3,1-diyl)) tris(azanetriyl))hexanonanoate; heptadecan-9-yl 8-((2-hydroxyethyl)(6-oxo-6-(undecyloxy)hexyl)amino) octanoate; and (((3,6-dioxopiperazine-2,5-diyl)bis(butane-4, 1-diyl))bis(azanetriyl))tetrakis(ethane-2,1-diyl) (9Z,9′Z,9″Z,9′″Z,12Z,12′Z,12″Z,12′″Z)-tetrakis (octadeca-9,12-dienoate).

Additionally, in certain embodiments, the lipid nanoparticles can include other lipids. For example, but without any limitation, the lipid nanoparticles of the presently disclosed subject matter can include phospholipids, cholesterol, polyethylene glycol (PEG)-functionalized lipids (PEG-lipids). These lipids can improve certain properties of the lipid nanoparticles (e.g., stability, biodistribution, etc.). For example, cholesterol enhances the stability of the lipid nanoparticles by modulating their integrity and rigidity. Non-limiting examples of other lipids present in lipid nanoparticles include cholesterol, DC-cholesterol, 0-sitosterol, BHEM-cholesterol, ALC-0159, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoylphosphatidylethanolamine (DSPE), 16-O-monomethyl PE, 16-O-dimethyl PE, 18-1-trans PE, 1-stearioyl-2-oleoyl-phosphatidyethanol amine (SOPE), and 1,2-dielaidoyl-sn-glycero-3-phophoethanolamine (transDOPE).

In certain embodiments, the lipid nanoparticles can include a targeting moiety that binds to a ligand. The use of the targeting moieties allows selective delivery of an active pharmaceutical ingredient (e.g., nucleic acid compositions disclosed herein) to target cells expressing the ligand (e.g., T cells). In certain embodiments, the targeting moiety can be an antibody or antigen-binding fragment thereof that binds to a cell surface receptor. For example, but without any limitation, the targeting domain is an antibody or antigen-binding fragment thereof that binds to a receptor expressed on the surface of a T cell (e.g., CD3, CD4, CD8, CD16, CD40L, CD95, FasL, CTLA-4, OX40, GITR, LAG3, ICOS, and PD-1).

In certain embodiments, the delivery methods are in vivo delivery methods. In certain embodiments, the delivery methods are ex vivo delivery methods.

### 5. FORMULATIONS AND ADMINISTRATION

The presently disclosed subject matter provides compositions comprising presently disclosed cells (e.g., disclosed in Section 3). Additionally, the presently disclosed subject matter provides compositions comprising presently disclosed nucleic acids (e.g., disclosed in Section 4). In certain embodiments, the compositions are pharmaceutical compositions that further comprise a pharmaceutically acceptable excipient.

Compositions comprising the presently disclosed cells and/or nucleic acids can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate-buffered saline, polyol (for example, glycerol, propylene glycol, liquid polyethylene glycol, and the like), and suitable mixtures thereof.

Compositions comprising the presently disclosed cells and/or nucleic acids can be provided systemically or directly to a subject for inducing and/or enhancing an immune response to an antigen and/or treating and/or preventing a neoplasm. In certain embodiments, the presently disclosed cells, nucleic acids, or compositions comprising thereof are directly injected into an organ of interest (e.g., an organ affected by a neoplasm). Alternatively, the presently disclosed cells, nucleic acids, or compositions comprising thereof are provided indirectly to the organ of interest, for example, by administration into the circulatory system (e.g., the tumor vasculature). Expansion and differentiation agents can be provided prior to, during, or after administration of the cells or compositions to increase the production of cells in vitro or in vivo.

The number of cells to be administered can vary for the subject being treated. In certain embodiments, between about 10⁴ and about 10¹⁰, between about 10⁴ and about 10⁷, between about 10⁵ and about 10⁷, between about 10⁵ and about 10⁹, or between about 10⁶ and about 10⁸ of the presently disclosed cells are administered to a subject. In certain embodiments, between about 10⁵ and about 10⁷ of the presently disclosed cells are administered to a subject. More effective cells may be administered in even smaller numbers. Usually, at least about 1×10⁵ cells will be administered, eventually reaching about 1×10¹⁰ or more. In certain embodiments, at least about 1×10⁵, about 5×10⁵, about 1×10⁶, about 5×10⁶, about 1×10⁷, about 5×10⁷, about 1×10⁸, or about 5×10⁸ of the presently disclosed cells are administered to a subject. In certain embodiments, about 1×10⁵ of the presently disclosed cells are administered to a subject. In certain embodiments, about 5×10⁵ of the presently disclosed cells are administered to a subject. In certain embodiments, about 1×10⁶ of the presently disclosed cells are administered to a subject. The precise determination of what would be considered an effective dose can be based on factors individual to each subject, including the size, age, sex, weight, and condition of the particular subject. Dosages can be readily ascertained by those skilled in the art from this disclosure and the knowledge in the art.

The presently disclosed cells and compositions can be administered by any method known in the art including, but not limited to, intravenous administration, subcutaneous administration, intranodal administration, intratumoral administration, intrathecal administration, intrapleural administration, intraosseous administration, intraperitoneal administration, pleural administration, and direct administration to the subject. The presently disclosed cells can be administered in any physiologically acceptable vehicle, normally intravascularly, although they may also be introduced into bone or other convenient sites where the cells may find an appropriate site for regeneration and differentiation (e.g., thymus). The cells can be introduced by injection, catheter, or the like.

Compositions comprising the presently disclosed cells and/or nucleic acids can be provided systemically or directly to a subject for inducing and/or enhancing an immune response to an antigen and/or treating and/or preventing a neoplasm (e.g., cancer), pathogen infection, or infectious disease. In certain embodiments, the presently disclosed cells, nucleic acids, or compositions thereof are directly injected into an organ of interest (e.g., an organ affected by a neoplasm). Alternatively, the presently disclosed cells, nucleic acids, or compositions thereof are provided indirectly to the organ of interest, for example, by administration into the circulatory system (e.g., the tumor vasculature). Expansion and differentiation agents can be provided prior to, during, or after administration of the cells, compositions, or nucleic acid compositions to increase production of the cells (e.g., T cells (e.g., CTL cells) or NK cells) in vitro or in vivo.

The presently disclosed compositions can be pharmaceutical compositions comprising the presently disclosed cells or their progenitors and a pharmaceutically acceptable carrier. Administration can be autologous or heterologous. For example, cells, or progenitors can be obtained from one subject and administered to the same subject or a different, compatible subject. Peripheral blood derived cells or their progeny (e.g., in vivo, ex vivo, or in vitro derived) can be administered via localized injection, including catheter administration, systemic injection, localized injection, intravenous injection, or parenteral administration. When administering a therapeutic composition of the presently disclosed subject matter (e.g., a pharmaceutical composition comprising a presently disclosed cell), it can be formulated in a unit dosage injectable form (solution, suspension, emulsion).

### 6. METHODS OF TREATMENT

The presently disclosed subject matter provides various methods of using the presently disclosed cells, nucleic acids, or compositions thereof. The presently disclosed cells, nucleic acids, or compositions thereof can be used in therapy or medicament. For example, the presently disclosed subject matter provides methods for inducing and/or increasing an immune response in a subject in need thereof. The presently disclosed cells and compositions comprising thereof can be used for reducing tumor burden in a subject. The presently disclosed cells and compositions comprising thereof can reduce the number of tumor cells, reduce tumor size, and/or eradicate the tumor in the subject. The presently disclosed cells, nucleic acids, or compositions thereof can be used for treating and/or preventing a tumor (or neoplasm) in a subject. The presently disclosed cells, nucleic acids, or compositions thereof can be used for prolonging the survival of a subject suffering from a tumor. In certain embodiments, the tumor is cancer. The presently disclosed cells, nucleic acids, or compositions thereof can also be used for treating and/or preventing a pathogen infection or other infectious disease in a subject, such as an immunocompromised human subject. The presently disclosed cells, nucleic acids, or compositions thereof can also be used for treating and/or preventing an autoimmune disease in a subject. In certain embodiments, each of the above-noted methods comprises administering the presently disclosed cells, nucleic acids, or compositions thereof (e.g., a pharmaceutical composition comprising the cells or a pharmaceutical composition comprising the nucleic acids) to achieve the desired effect, e.g., palliation of an existing condition or prevention of recurrence. For treatment, the amount administered is an amount effective in producing the desired effect. An effective amount can be provided in one or a series of administrations. An effective amount can be provided in a bolus or by continuous perfusion.

Non-limiting examples of tumors (or neoplasms) include blood cancers (e.g. leukemias, lymphomas, and myelomas), ovarian cancer, breast cancer, bladder cancer, brain cancer, colon cancer, intestinal cancer, liver cancer, lung cancer, pancreatic cancer, prostate cancer, skin cancer, stomach cancer, glioblastoma, throat cancer, melanoma, neuroblastoma, adenocarcinoma, glioma, soft tissue sarcoma, and various carcinomas (including prostate and small cell lung cancer). Suitable carcinomas further include any known in the field of oncology, including, but not limited to, astrocytoma, fibrosarcoma, myxosarcoma, liposarcoma, oligodendroglioma, ependymoma, medulloblastoma, primitive neural ectodermal tumor (PNET), chondrosarcoma, osteogenic sarcoma, pancreatic ductal adenocarcinoma, small and large cell lung adenocarcinomas, chordoma, angiosarcoma, endotheliosarcoma, squamous cell carcinoma, bronchoalveolar carcinoma, epithelial adenocarcinoma, and liver metastases thereof, lymphangiosarcoma, lymphangioendotheliosarcoma, hepatoma, cholangiocarcinoma, synovioma, mesothelioma, Ewing's tumor, rhabdomyosarcoma, colon carcinoma, basal cell carcinoma, sweat gland carcinoma, papillary carcinoma, sebaceous gland carcinoma, papillary adenocarcinoma, cystadenocarcinoma, medullary carcinoma, bronchogenic carcinoma, renal cell carcinoma, bile duct carcinoma, choriocarcinoma, seminoma, embryonal carcinoma, Wilms' tumor, testicular tumor, medulloblastoma, craniopharyngioma, ependymoma, pinealoma, hemangioblastoma, acoustic neuroma, oligodendroglioma, meningioma, neuroblastoma, retinoblastoma, leukemia, multiple myeloma, Waldenstrom's macroglobulinemia, and heavy chain disease, breast tumors such as ductal and lobular adenocarcinoma, squamous and adenocarcinomas of the uterine cervix, uterine and ovarian epithelial carcinomas, prostatic adenocarcinomas, transitional squamous cell carcinoma of the bladder, B and T cell lymphomas (nodular and diffuse) plasmacytoma, acute and chronic leukemias, malignant melanoma, soft tissue sarcomas and leiomyosarcomas. In certain embodiments, the neoplasm is cancer. In certain embodiments, the neoplasm is selected from the group consisting of blood cancers (e.g. leukemias, lymphomas, and myelomas), ovarian cancer, prostate cancer, breast cancer, bladder cancer, brain cancer, colon cancer, intestinal cancer, liver cancer, lung cancer, pancreatic cancer, prostate cancer, skin cancer, stomach cancer, glioblastoma, and throat cancer. In certain embodiments, the presently disclosed cells, nucleic acids, or compositions thereof can be used for treating and/or preventing blood cancers (e.g., leukemias, lymphomas, and myelomas) or ovarian cancer, which are not amenable to conventional therapeutic interventions.

In certain embodiments, the tumor and/or neoplasm is a solid tumor. Non-limiting examples of solid tumors include renal cell carcinoma, non-small-cell lung cancer, lung adenocarcinoma, lung squamous cell carcinoma, lung neuroendocrine carcinoma, small-cell lung cancer, pancreatic cancer, breast cancer, astrocytoma, glioblastoma, laryngeal/pharyngeal carcinoma, EBV-associated nasopharyngeal carcinoma, and ovarian carcinoma.

In certain embodiments, the tumor and/or neoplasm is a blood cancer. Non-limiting examples of blood cancer include multiple myeloma, leukemia, and lymphomas. Non-limiting examples of leukemia include acute myeloid leukemia (AML), chronic myeloid leukemia (CML), acute lymphocytic leukemia (ALL), chronic lymphocytic leukemia (CLL), acute promyelocytic leukemia (APL), mixed-phenotype acute leukemia (MLL), hairy cell leukemia, and B cell prolymphocytic leukemia. The lymphoma can be Hodgkin's lymphoma or non-Hodgkin's lymphoma. In certain embodiments, the lymphoma is non-Hodgkin's lymphoma, including B-cell non-Hodgkin's lymphoma and T-cell non-Hodgkin's lymphoma.

In certain embodiments, the tumor and/or neoplasm is a B cell malignancy. Non-limiting examples of B cell malignancy include B cell non-Hodgkin lymphomas (NHL), B cell Hodgkin's lymphomas, B cell acute lymphocytic leukemia (ALL), B cell chronic lymphocytic leukemia (CLL), multiple myeloma (MM), CLL with Richter's transformation, and CNS lymphoma.

In certain embodiments, the tumor and/or neoplasm is a B cell-related neoplasm. Non-limiting examples of B cell-related neoplasm include chronic lymphocytic leukemia/small lymphocytic lymphoma (CLL/SLL), B-cell prolymphocytic leukemia, splenic marginal zone lymphoma, hairy cell leukemia, splenic B-cell lymphoma/leukemia (unclassifiable), splenic diffuse red pulp small B-cell lymphoma, lymphoplasmacytic lymphoma, Waldenstrom macroglobulinemia, monoclonal gammopathy of undetermined significance (MGUS, IgM), heavy-chain diseases (μ, γ, α), MGUS (IgG/A), plasma cell myeloma, solitary plasmacytoma of bone, extraosseous plasmacytoma, monoclonal immunoglobulin deposition diseases, extranodal marginal zone lymphoma of mucosa-associated lymphoid tissue (MALT lymphoma), nodal marginal zone lymphoma, pediatric nodal marginal zone lymphoma, follicular lymphoma, in situ follicular neoplasia, duodenal-type follicular lymphoma, pediatric-type follicular lymphoma, large B-cell lymphoma with IRF4 rearrangement, primary cutaneous follicle center cell lymphoma, mantle cell lymphoma, in situ mantle cell neoplasia, diffuse large B-cell lymphoma (DLBCL) (not otherwise specified (NOS)), germinal center B-cell type, activated B-cell type, T-cell/histiocyte-rich large B-cell lymphoma, primary DLBCL of the central nervous system (CNS), primary cutaneous DLBCL (leg type), Epstein-Barr virus (EBV)-positive DLBCL (NOS), EBV-positive mucocutaneous ulcer, DLBCL associated with chronic inflammation, lymphomatoid granulomatosis, primary mediastinal (thymic) large B-cell lymphoma, intravascular large B-cell lymphoma, anaplastic lymphoma kinase (ALK)-positive large B-cell lymphoma, plasmablastic lymphoma, primary effusion lymphoma, human herpesvirus 8 (HHV-8)-associated DLBCL (NOS), Burkitt lymphoma, Burkitt-like lymphoma with 11q aberration, high-grade B-cell lymphoma with MYC and BLC2 and/or BCL6 rearrangements, high-grade B-cell lymphoma (NOS), and B-cell lymphoma (unclassifiable).

In certain embodiments, the tumor and/or neoplasm is a myeloid disorder. Non-limiting examples of myeloid disorders include myelodysplastic syndromes, myeloproliferative neoplasms, chronic myelomonocytic leukemia, acute myeloid leukemia (AML), blastic plasmacytoid dendritic cell neoplasm, acute myeloblastic leukemia, acute promyelocytic leukemia, acute myelomonocytic leukemia, chronic myelocytic leukemia, and polycythemia vera. In certain embodiments, the myeloid disorder is acute myeloid leukemia (AML).

The presently disclosed subject matter provides methods for treating and/or preventing a viral infection in a subject. The method can comprise administering an effective amount of the presently disclosed cells, nucleic acids, or compositions thereof to a subject having a viral infection. Non-limiting examples of viral infections include those caused by cytomegalovirus (CMV), Epstein-Barr virus (EBV), hepatitis A, B, C, D, E, F or G, human immunodeficiency virus (HIV), adenovirus, BK polyomavirus, coronavirus, coxsackievirus, poliovirus, herpes simplex type 1, herpes simplex type 2, human cytomegalovirus, human herpesvirus type 8, varicella-zoster virus, influenza virus, measles virus, mumps virus, parainfluenza virus, respiratory syncytial virus, papillomavirus, rabies virus, and Rubella virus. Other viral targets include Paramyxoviridae (e.g., pneumovirus, morbillivirus, metapneumovirus, respirovirus or rubulavirus), Adenoviridae (e.g., adenovirus), Arenaviridae (e.g., arenavirus such as lymphocytic choriomeningitis virus), Arteriviridae (e.g., porcine respiratory and reproductive syndrome virus or equine arteritis virus), Bunyaviridae (e.g., phlebovirus or hantavirus), Caliciviridae (e.g., Norwalk virus), Coronaviridae (e.g., coronavirus or torovirus), Filoviridae (e.g., Ebola-like viruses), Flaviviridae (e.g., hepacivirus or flavivirus), Herpesviridae (e.g., simplexvirus, varicellovirus, cytomegalovirus, roseolovirus, or lymphocryptovirus), Orthomyxoviridae (e.g., influenza virus or thogotovirus), Parvoviridae (e.g., parvovirus), Picomaviridae (e.g., enterovirus or hepatovirus), Poxviridae (e.g., orthopoxvirus, avipoxvirus, or leporipoxvirus), Retroviridae (e.g., lentivirus or spumavirus), Reoviridae (e.g., rotavirus), Rhabdoviridae (e.g., lyssavirus, novirhabdovirus, or vesiculovirus), and Togaviridae (e.g., alphavirus or rubivirus). In certain embodiments, the viral infections include human respiratory coronavirus, influenza viruses A-C, hepatitis viruses A to G, and herpes simplex viruses 1-9. In certain embodiments, the subject has an immunodeficiency.

The presently disclosed subject matter provides methods for treating and/or preventing a bacterial infection in a subject. The method can comprise administering an effective amount of the presently disclosed cells, nucleic acids, or compositions thereof to a subject having a bacterial infection. Bacterial infections include, but are not limited to, Mycobacteria, Rickettsia, Mycoplasma, Neisseria meningitides, Neisseria gonorrheoeae, Legionella, Vibrio cholerae, Streptococci, Staphylococcus aureus, Staphylococcus epidermidis, Pseudomonas aeruginosa, Corynobacteria diphtheriae, Clostridium spp., enterotoxigenic Escherichia coli, Bacillus anthracis, Rickettsia, Bartonella henselae, Bartonella quintana, Coxiella burnetii, chlamydia, Mycobacterium leprae, Salmonella, shigella, Yersinia enterocolitica, Yersinia pseudotuberculosis; Legionella pneumophila; Mycobacterium tuberculosis; Listeria monocytogenes; Mycoplasma spp., Pseudomonas fluorescens, Vibrio cholerae, Haemophilus influenzae, Bacillus anthracis, Treponema pallidum, Leptospira, Borrelia, Corynebacterium diphtheriae, Francisella, Brucella melitensis, Campylobacter jejuni, Enterobacter, Proteus mirabilis, Proteus, and Klebsiella pneumoniae.

The presently disclosed subject matter provides methods for treating and/or preventing an autoimmune disease in a subject. The method can comprise administering an effective amount of the presently disclosed cells, nucleic acids, or compositions thereof to a subject having an autoimmune disease.

The presently disclosed subject matter provides methods for treating and/or preventing an inflammatory disease in a subject. The method can comprise administering an effective amount of the presently disclosed cells, nucleic acids, or compositions thereof to a subject having an infectious disease.

Non-limiting examples of autoimmune diseases and inflammatory diseases or conditions thereof include arthritis, e.g., rheumatoid arthritis (RA), Type I diabetes, systemic lupus erythematosus (SLE), inflammatory bowel disease, ulcerative colitis, psoriasis, psoriatic arthritis, scleroderma, autoimmune thyroid disease, Grave's disease, Crohn's disease, multiple sclerosis, systemic sclerosis, asthma, organ transplant rejection, a disease or condition associated with transplant, Takayasu arteritis, giant-cell arteritis, Kawasaki disease, polyarteritis nodosa, Behcet's syndrome, Wegener's granulomatosis, ANCA-vasculitides, Churg-Strauss syndrome, microscopic polyangiitis, vasculitis of connective tissue diseases, Hennoch-Schonlein purpura, cryoglobulinemic vasculitis, cutaneous leukocytoclastic angiitis, Sarcoidosis, Cogan's syndrome, Wiskott-Aldrich Syndrome, primary angiitis of the CNS, thromboangiitis obliterans, paraneoplastic arteritis, myelodysplastic syndrome, erythema elevatum diutinum, amyloidosis, autoimmune myositis, Guillain-Barre Syndrome, histiocytosis, atopic dermatitis, pulmonary fibrosis, glomerulonephritis, Whipple's disease, Still's disease, Sjogren's syndrome, osteomyelofibrosis, chronic inflammatory demyelinating polyneuropathy, Kimura's disease, systemic sclerosis, chronic periaortitis, chronic prostatitis, idiopathic pulmonary fibrosis, chronic granulomatous disease, idiopathic, bleomycin-induced lung inflammation, cytarabine-induced lung inflammation, autoimmune thrombocytopenia, autoimmune neutropenia, autoimmune hemolytic anemia, autoimmune lymphocytopenia, chronic autoimmune thyroiditis, autoimmune hepatitis, Hashimoto's thyroiditis, atopic thyroiditis, Graves disease, autoimmune polyglandular syndrome, autoimmune Addison syndrome, and/or myasthenia gravis. In accordance with the presently disclosed subject matter, the above-described various methods can comprise administering to the subject a checkpoint immune blockade agent.

The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

Further modification can be introduced to the presently disclosed cells to avert or minimize the risks of immunological complications (known as “malignant T-cell transformation”), e.g., graft versus-host disease (GvHD), or when healthy tissues express the same target antigens as the tumor cells, leading to outcomes similar to GvHD. A potential solution to this problem is engineering a suicide gene into the presently disclosed cells. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. In certain embodiments, the suicide gene is an EGFRt polypeptide. The EGFRt polypeptide can enable T-cell elimination by administering anti-EGFR monoclonal antibody (e.g., cetuximab). EGFRt can be covalently joined to the upstream of the antigen-recognizing receptor. The suicide gene can be included within the vector comprising nucleic acids encoding a presently disclosed antigen-recognizing receptor. In this way, administration of a prodrug designed to activate the suicide gene (e.g., a prodrug (e.g., AP1903 that can activate iCasp-9) during malignant T-cell transformation (e.g., GVHD) triggers apoptosis in the suicide gene-activated cells expressing the presently disclosed antigen-recognizing receptor. The incorporation of a suicide gene into a presently disclosed antigen-recognizing receptor gives an added level of safety with the ability to eliminate the majority of receptor-expressing cells within a very short time period. A presently disclosed cell incorporated with a suicide gene can be pre-emptively eliminated at a given timepoint post the cell infusion, or eradicated at the earliest signs of toxicity.

### 7. KITS

The presently disclosed subject matter provides kits for inducing and/or enhancing an immune response and/or treating and/or preventing a neoplasm or a pathogen infection (e.g., an autoimmune disease or an infectious disease) in a subject. In certain embodiments, the kit comprises an effective amount of presently disclosed cells, nucleic acids, or compositions thereof. In certain embodiments, the kit comprises a sterile container; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In certain non-limiting embodiments, the kit includes a nucleic acid molecule encoding the fusion polypeptide disclosed herein. In certain non-limiting embodiments, the kit further includes a nucleic acid molecule encoding an antigen-recognizing receptor disclosed herein (e.g., a CAR, a TCR, or a TCR-like fusion molecule) directed toward an antigen of interest in expressible form, which may optionally be comprised in the same or different vectors.

If desired, the cells, nucleic acids, or compositions thereof are provided together with instructions for administering the cells, nucleic acids, or compositions thereof to a subject having or at risk of developing a tumor (e.g., a cancer) or a pathogen infection (e.g., an infectious disease), or immune disorder (e.g., an autoimmune disease). The instructions generally include information about the use of the cells, nucleic acids, or compositions thereof for the treatment and/or prevention of a neoplasm, or a pathogen infection (e.g., an infectious disease), or an immune disorder (e.g., an autoimmune disease). In certain embodiments, the instructions include at least one of the following: description of the therapeutic agent; dosage schedule and administration for treatment or prevention of a neoplasm, pathogen infection (e.g., an infectious disease), or immune disorder (e.g., an autoimmune disease) or symptoms thereof, precautions; warnings; indications; counter-indications; over-dosage information; adverse reactions; animal pharmacology; clinical studies; and/or references. The instructions may be printed directly on the container (when present), or as a label applied to the container, or as a separate sheet, pamphlet, card, or folder supplied in or with the container.

### 8. EXEMPLARY EMBODIMENTS

Embodiment 1. A fusion polypeptide comprising an extracellular domain comprising an antigen-binding fragment and a co-stimulatory ligand polypeptide, and an intracellular domain comprising a first co-stimulatory molecule polypeptide.

Embodiment 2. The fusion polypeptide of embodiment 1, wherein the antigen-binding fragment binds to an immune checkpoint molecule.

Embodiment 3. The fusion polypeptide of embodiment 2, wherein the immune checkpoint molecule is selected from the group consisting of PD-L1, PD-L2, VISTA, B7-H3, B7-H4, B7-H7, herpesvirus entry mediator (HVEM), CD155, CD112, CD200, galectin 9 (GAL9), TIGIT, BTLA, LAG-3, TIM-3, and 2B4 (CD224).

Embodiment 4. The fusion polypeptide of any one of embodiments 1-3, wherein the antigen-binding fragment binds to PD-L1.

Embodiment 5. The fusion polypeptide of embodiment 1, wherein the antigen-binding fragment is a Fab, a Fab′, a F(ab′)2, a variable fragment (Fv), a single chain variable region (scFv), a nanobody, a microantibody, an affibody molecule, an affilin, an affimer, an affitin, an alphabody, an anticalin protein, an avimer, a DARPin (designed ankyrin repeat proteins), or an aptamer.

Embodiment 6. The fusion polypeptide of embodiment 5, wherein the antigen-binding fragment is a nanobody.

Embodiment 7. The fusion polypeptide of embodiment 6, wherein the nanobody comprises a CDR1 comprising the amino acid sequence set forth in SEQ ID NO: 27 or a conservative modification thereof, a CDR2 comprising the amino acid sequence set forth in SEQ ID NO: 28 or a conservative modification thereof, and CDR3 comprising the amino acid sequence set forth in SEQ ID NO: 29 or a conservative modification thereof.

Embodiment 8. The fusion polypeptide of embodiment 7, wherein the nanobody comprises a CDR1 comprising the amino acid sequence set forth in SEQ ID NO: 27, a CDR2 comprising the amino acid sequence set forth in SEQ ID NO: 28, and CDR3 comprising the amino acid sequence set forth in SEQ ID NO: 29.

Embodiment 9. The fusion polypeptide of embodiment 6, wherein the nanobody comprises or consists of an amino acid sequence that is at least about 80% identical to the amino acid sequence set forth in SEQ ID NO: 30.

Embodiment 10. The fusion polypeptide of embodiment 9, wherein the nanobody comprises the amino acid sequence set forth in SEQ ID NO: 30.

Embodiment 11. The fusion polypeptide of embodiment 5, wherein the antigen-binding fragment is an scFv.

Embodiment 12. The fusion polypeptide of embodiment 11, wherein the scFv comprises a heavy chain variable region (VH) comprising a CDR1 comprising the amino acid sequence set forth in SEQ ID NO: 16 or a conservative modification thereof, a CDR2 comprising the amino acid sequence set forth in SEQ ID NO: 17 or a conservative modification thereof, and a CDR3 comprising the amino acid sequence set forth in SEQ ID NO: 18 or a conservative modification thereof, and a light chain variable region (VL) comprising a CDR1 comprising the amino acid sequence set forth in SEQ ID NO: 19 or a conservative modification thereof, a CDR2 comprising the amino acid sequence set forth in SEQ ID NO: 20 or a conservative modification thereof, and a CDR3 comprising the amino acid sequence set forth in SEQ ID NO: 21 or a conservative modification thereof.

Embodiment 13. The fusion polypeptide of embodiment 12, wherein the VH comprises a CDR1 comprising the amino acid sequence set forth in SEQ ID NO: 16, a CDR2 comprising the amino acid sequence set forth in SEQ ID NO: 17, and a CDR3 comprising the amino acid sequence set forth in SEQ ID NO: 18; and the VL comprises a CDR1 comprising the amino acid sequence set forth in SEQ ID NO: 19, a CDR2 comprising the amino acid sequence set forth in SEQ ID NO: 20, and a CDR3 comprising the amino acid sequence set forth in SEQ ID NO: 21.

Embodiment 14. The fusion polypeptide of embodiment 11, wherein the scFv comprises a heavy chain variable region (VH) that is at least about 80% identical to the amino acid sequence set forth in SEQ ID NO: 22, and a light chain variable region (VL) that is at least about 80% identical to the amino acid sequence set forth in SEQ ID NO: 23.

Embodiment 15. The fusion polypeptide of embodiment 14, wherein the VH comprises the amino acid sequence set forth in SEQ ID NO: 22, and the VL comprises the amino acid sequence set forth in SEQ ID NO: 23.

Embodiment 16. The fusion polypeptide of embodiment 11, wherein the scFv comprises or consists of an amino acid sequence that is at least about 80% identical to the amino acid sequence set forth in SEQ ID NO: 24.

Embodiment 17. The fusion polypeptide of embodiment 16, wherein the scFv comprises the amino acid sequence set forth in SEQ ID NO: 24.

Embodiment 18. The fusion polypeptide of any one of embodiments 1-17, wherein the co-stimulatory ligand polypeptide is selected from the group consisting of a tumor necrosis factor (TNF) family member, an immunoglobulin (Ig) superfamily member, and combinations thereof.

Embodiment 19. The fusion polypeptide of embodiment 18, wherein the TNF family member is selected from the group consisting of 4-1BBL, OX40L, CD70, GITRL, CD40L, and combinations thereof.

Embodiment 20. The fusion polypeptide of embodiment 18, wherein the Ig superfamily member is selected from the group consisting of CD80, CD86, ICOSLG, and combinations thereof.

Embodiment 21. The fusion polypeptide of any one of embodiments 1-20, wherein the co-stimulatory ligand polypeptide is a CD80 polypeptide.

Embodiment 22. The fusion polypeptide of embodiment 21, wherein the CD80 polypeptide comprises or consists of an amino acid sequence that is at least about 80% identical to the amino acid sequence set forth in SEQ ID NO: 32, or a functional fragment thereof.

Embodiment 23. The fusion polypeptide of embodiment 21 or 22, wherein the CD80 polypeptide comprises or consists of an amino acid sequence that is at least about 80% identical to the amino acid sequence set forth in SEQ ID NO: 33.

Embodiment 24. The fusion polypeptide of any one of embodiments 21-23, wherein the CD80 polypeptide comprises or consists of the amino acid sequence set forth in SEQ ID NO: 33.

Embodiment 25. The fusion polypeptide of any one of embodiments 1-24, wherein the first co-stimulatory molecule polypeptide is selected from the group consisting of CD28, 4-1BB, OX40, ICOS, DAP-10, CD27, CD40, NKG2D, CD2, and combinations thereof.

Embodiment 26. The fusion polypeptide of any one of embodiments 1-25, wherein the first co-stimulatory molecule polypeptide is a 4-1BB polypeptide.

Embodiment 27. The fusion polypeptide of embodiment 26, wherein the 4-1BB polypeptide comprises or consists of an amino acid sequence that is at least about 80% identical to the amino acid sequence set forth in SEQ ID NO: 41, or a functional fragment thereof.

Embodiment 28. The fusion polypeptide of embodiment 26 or 27, wherein the 4-1BB polypeptide comprises or consists of an amino acid sequence that is at least about 80% identical to the amino acid sequence set forth in SEQ ID NO: 42.

Embodiment 29. The fusion polypeptide of any one of embodiments 26-28, wherein the 4-1BB polypeptide comprises the amino acid sequence set forth in SEQ ID NO: 42.

Embodiment 30. The fusion polypeptide of any one of embodiments 1-29, wherein the intracellular domain further comprises a second co-stimulatory molecule polypeptide.

Embodiment 31. The fusion polypeptide of embodiment 30, wherein the second co-stimulatory molecule polypeptide is selected from the group consisting of CD28, 4-1BB, OX40, ICOS, DAP-10, CD27, CD40, NKG2D, CD2, and combinations thereof.

Embodiment 32. The fusion polypeptide of embodiment 30 or 31, wherein the second co-stimulatory molecule polypeptide is a CD28 polypeptide.

Embodiment 33. The fusion polypeptide of embodiment 32, wherein the CD28 polypeptide comprises or consists of an amino acid sequence that is at least about 80% identical to the amino acid sequence set forth in SEQ ID NO: 43, or a functional fragment thereof.

Embodiment 34. The fusion polypeptide of embodiment 32 or 33, wherein the CD28 polypeptide comprises or consists of an amino acid sequence that is at least about 80% identical to the amino acid sequence set forth in SEQ ID NO: 44.

Embodiment 35. The fusion polypeptide of any one of embodiments 32-34, wherein the CD28 polypeptide comprises the amino acid sequence set forth in SEQ ID NO: 44.

Embodiment 36. The fusion polypeptide of any one of embodiments 1-35, wherein the intracellular domain further comprises a spacer region.

Embodiment 37. The fusion polypeptide of embodiment 36, wherein the spacer region comprises a CD80 polypeptide.

Embodiment 38. The fusion polypeptide of embodiment 36 or 37, wherein the spacer region comprises a CD80 polypeptide comprising or consisting of amino acids 264 to 267 of SEQ ID NO: 32.

Embodiment 39. The fusion polypeptide of any one of embodiments 1-38, further comprising a transmembrane domain.

Embodiment 40. The fusion polypeptide of embodiment 39, wherein the transmembrane domain comprises a CD80 polypeptide.

Embodiment 41. The fusion polypeptide of embodiment 39 or 40, wherein the transmembrane domain comprises a CD80 polypeptide comprising or consisting of amino acids 243 to 263 of SEQ ID NO: 32.

Embodiment 42. The fusion polypeptide of embodiment 39 or 40, wherein the transmembrane domain comprises a CD80 polypeptide comprising or consisting of SEQ ID NO: 52.

Embodiment 43. The fusion polypeptide of any one of embodiments 1-42, wherein the antigen-binding fragment binds to PD-L1, the co-stimulatory ligand polypeptide is selected from the group consisting of 4-1BBL, OX40L, CD70, GITRL, CD40L, CD80, CD86, ICOSLG, and the first co-stimulatory molecule polypeptide is selected from the group consisting of CD28, 4-1BB, OX40, ICOS, DAP-10, CD27, CD40, NKG2D, and CD2.

Embodiment 44. The fusion polypeptide of any one of embodiments 1-43, wherein the antigen-binding fragment binds to PD-L1, the co-stimulatory ligand polypeptide is a CD80 polypeptide, and the first co-stimulatory molecule polypeptide is selected from the group consisting of CD28, 4-1BB, OX40, ICOS, DAP-10, CD27, CD40, NKG2D, and CD2.

Embodiment 45. The fusion polypeptide of any one of embodiments 1-44, wherein the antigen-binding fragment binds to PD-L1, the co-stimulatory ligand polypeptide is a CD80 polypeptide, and the first co-stimulatory molecule polypeptide is a 4-1BB polypeptide.

Embodiment 46. The fusion polypeptide of any one of embodiments 1-45, wherein the fusion polypeptide comprises or consists of an amino acid sequence that is at least about 80% identical to the amino acid sequence set forth in SEQ ID NO: 53.

Embodiment 47. The fusion polypeptide of embodiment 46, wherein the fusion polypeptide comprises the amino acid sequence set forth in SEQ ID NO: 53.

Embodiment 48. The fusion polypeptide of any one of embodiments 1-45, wherein the fusion polypeptide comprises or consists of an amino acid sequence that is at least about 80% identical to the amino acid sequence set forth in SEQ ID NO: 55.

Embodiment 49. The fusion polypeptide of embodiment 48, wherein the fusion polypeptide comprises the amino acid sequence set forth in SEQ ID NO: 55.

Embodiment 50. The fusion polypeptide of any one of embodiments 1-45, wherein the antigen-binding fragment binds to PD-L1, the co-stimulatory ligand is CD80, the first co-stimulatory molecule is 4-1BB, and the second co-stimulatory molecule is CD28.

Embodiment 51. The fusion polypeptide of any one of embodiments 1-50 further comprising a signal peptide.

Embodiment 52. The fusion polypeptide of embodiment 51, wherein the signal peptide is selected from the group consisting of a human IL-2 signal sequence, a mouse IL-2 signal sequence, a human kappa leader sequence, a mouse kappa leader sequence, a human CD8 leader sequence, a truncated human CD8 signal peptide, a human albumin signal sequence, and a human prolactin signal sequence.

Embodiment 53. The fusion polypeptide of any one of embodiments 1-52, further comprising a signaling domain of a cytokine receptor.

Embodiment 54. The fusion polypeptide of embodiment 53, wherein the cytokine receptor is selected from the group consisting of CD121a, CDw121b, IL-18Ra, IL18Rb, CD122, CD25, CD132, CD124, CD213a13, CD127, IL-9R, IL15Ra, CDw125, CDw131, CD126, CD130, IL11Ra, CD114, CD212, CD4, CDw217, CD118, and CDw119.

Embodiment 55. The fusion polypeptide of any one of embodiment 1-54, further comprising a JAK-STAT signaling domain.

Embodiment 56. The fusion polypeptide of embodiment 55, wherein the JAK-STAT signaling domain is a STAT3 signaling domain or a STAT5 signaling domain.

Embodiment 57. The fusion polypeptide of any one of embodiment 1-56, further comprising a T cell signaling molecule.

Embodiment 58. The fusion polypeptide of any one of embodiments 1-57, wherein the fusion polypeptide is capable of stimulating a cell comprising an antigen-recognizing receptor.

Embodiment 59. The fusion polypeptide of any one of embodiments 1-58, wherein the fusion polypeptide is capable of enhancing the activity of an immunoresponsive cell comprising an antigen-recognizing receptor.

Embodiment 60. The fusion polypeptide of embodiment 59, wherein the activity comprises cytotoxicity, cell proliferation, and cell persistence.

Embodiment 61. The fusion polypeptide of any one of embodiments 58-60, wherein the antigen-recognizing receptor is a chimeric antigen receptor (CAR), a T-Cell Receptor (TCR), or a TCR like fusion molecule.

Embodiment 62. A nucleic acid encoding a fusion polypeptide of any one of embodiments 1-61.

Embodiment 63. A nucleic acid comprising a first polynucleotide encoding a fusion polypeptide of any one of embodiments 1-61, and a second polynucleotide encoding an antigen-recognizing receptor that binds to an antigen.

Embodiment 64. The nucleic acid of embodiment 62 or 63, further comprising a first promoter that is operably linked to the fusion polypeptide.

Embodiment 65. The nucleic acid of embodiment 63 or 64, further comprising a second promoter that is operably linked to the antigen-recognizing receptor.

Embodiment 66. The nucleic acid of embodiment 64 or 65, wherein one or both of the first and second promoters are endogenous or exogenous.

Embodiment 67. The nucleic acid of embodiment 66, wherein the exogenous promoter is selected from the group consisting of an elongation factor (EF)-1 promoter, a CMV promoter, a SV40 promoter, a PGK promoter, and a metallothionein promoter.

Embodiment 68. The nucleic acid of embodiment 64 or 65, wherein one or both of the first and second promoters are inducible promoters.

Embodiment 69. The nucleic acid of embodiment 68, wherein the inducible promoter is selected from the group consisting of a NFAT transcriptional response element (TRE) promoter, a CD69 promoter, a CD25 promoter, and an IL-2 promoter.

Embodiment 70. A vector comprising the nucleic acid of any one of embodiments 62-69.

Embodiment 71. A lipid nanoparticle comprising the nucleic acid of any one of embodiments 62-69.

Embodiment 72. A cell comprising a fusion polypeptide of any one of embodiments 1-61, the nucleic acid of any one of embodiments 62-69, the vector of embodiment 70, or the lipid nanoparticle of embodiment 71.

Embodiment 73. The cell of embodiment 72, further comprising an antigen-recognizing receptor that binds to an antigen.

Embodiment 74. The cell of embodiment 73, wherein the antigen-recognizing receptor is a chimeric antigen receptor (CAR), a T cell receptor (TCR), or a TCR like fusion molecule.

Embodiment 75. The cell of embodiment 74, wherein the antigen is a tumor antigen or a pathogen antigen.

Embodiment 76. The cell of embodiment 75, wherein the antigen is a tumor antigen.

Embodiment 77. The cell of embodiment 74 or 75, wherein the tumor antigen is selected from CD19, carbonic anhydrase IX (CAIX), carcinoembryonic antigen (CEA), CD8, CD7, CD10, CD20, CD22, CD30, CD33, CLL1, CD34, CD38, CD41, CD44, CD49f, CD56, CD74, CD133, CD138, CD123, CD44V6, an antigen of a cytomegalovirus (CMV) infected cell (e.g., a cell surface antigen), epithelial glycoprotein-2 (EGP-2), epithelial glycoprotein-40 (EGP-40), epithelial cell adhesion molecule (EpCAM), receptor tyrosine-protein kinases Erb-B2,3,4 (erb-B2,3,4), folate-binding protein (FBP), fetal acetylcholine receptor (AChR), folate receptor-α, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13RSYMBOL2), SYMBOL-light chain, kinase insert domain receptor (KDR), Lewis Y (LeY), L1 cell adhesion molecule (LlCAM), melanoma antigen family A, 1 (MAGE-A1), Mucin 16 (MUC16), Mucin 1 (MUC1), Mesothelin (MSLN), ERBB2, MAGEA3, p53, MART1, GP100, Proteinase3 (PR1), Tyrosinase, Survivin, hTERT, EphA2, NKG2D ligands, cancer-testis antigen NY-ESO-1, oncofetal antigen (h5T4), prostate stem cell antigen (PSCA), prostate-specific membrane antigen (PSMA), ROR1, tumor-associated glycoprotein 72 (TAG-72), vascular endothelial growth factor R2 (VEGF-R2), Wilms tumor protein (WT-1), BCMA, NKCS1, EGF1R, EGFR-VIII, CD99, CD70, ADGRE2, CCR1, LILRB2, PRAME CCR4, CD5, CD3, TRBC1, TRBC2, TIM-3, Integrin B7, ICAM-1, CD70, Tim3, CLEC12A, and ERBB.

Embodiment 78. The cell of any one of embodiments 75-77, wherein the tumor antigen is CD19.

Embodiment 79. The cell of any one of embodiments 72-78, wherein the antigen-recognizing receptor is exogenous or endogenous.

Embodiment 80. The cell of any one of embodiments 72-79, wherein the antigen-recognizing receptor is recombinantly expressed.

Embodiment 81. The cell of any one of embodiment 72-80, wherein the antigen-recognizing receptor is expressed from a vector.

Embodiment 82. The cell of any one of embodiments 72-81, wherein the cell is a cell of the lymphoid lineage or a cell of the myeloid lineage.

Embodiment 83. The cell of embodiment 82, wherein the cell of the lymphoid lineage is selected from T cells, B cells, Natural Killer (NK) cells, dendritic cells.

Embodiment 84. The cell of any one of embodiments 72-83, wherein the cell is a T cell.

Embodiment 85. The cell of embodiment 84, wherein the T cell is derived from an induced pluripotent stem cell.

Embodiment 86. The cell of embodiment 84 or 85, wherein the T cell is a CD8+ T cell.

Embodiment 87. The cell of embodiment 86, wherein the CD8+ T cell is CD4 independent.

Embodiment 88. The cell of any one of embodiments 83-87, wherein the T cell is selected from the group consisting of a cytotoxic T lymphocyte (CTL), a γδ T cell, a tumor-infiltrating lymphocyte (TIL), a virus-specific T cell (VST), a regulatory T cell, and a Natural Killer T (NKT) cell.

Embodiment 89. The cell of embodiment 88, wherein the T cell is a tumor-infiltrating lymphocyte (TIL).

Embodiment 90. The cell of embodiment 88, wherein the T cell is a virus-specific T cell (VST).

Embodiment 91. The cell of any one of embodiments 72-90, wherein the fusion polypeptide is integrated at a locus within the genome of the T cell.

Embodiment 92. The cell of any one of embodiments 73-91, wherein the fusion polypeptide and the antigen-recognizing receptor are integrated at a locus within the genome of the T cell.

Embodiment 93. The cell of embodiment 91 or 92, wherein the locus is selected from the group consisting of a TRAC locus, a TRBC locus, a TRDC locus, and a TRGC locus.

Embodiment 94. The cell of any one of embodiments 91-93, wherein the locus is a TRAC locus or a TRBC locus.

Embodiment 95. The cell of embodiment 94, wherein the locus is a TRAC locus.

Embodiment 96. The cell of any one of embodiments 73-96, wherein the antigen-recognizing receptor is a chimeric antigen receptor (CAR).

Embodiment 97. The cell of any one of embodiments 73-96, wherein the antigen-recognizing receptor is a TCR like fusion protein.

Embodiment 98. The cell of any one of embodiments 73-97, wherein the expressions of fusion polypeptide is under the control of an endogenous promoter.

Embodiment 99. The cell of any one of embodiments 73-97, wherein the expressions of the fusion polypeptide and antigen-recognizing receptor is under the control of an endogenous promoter.

Embodiment 100. The cell of embodiment 98 or 99, wherein the endogenous promoter is selected from the group consisting of an endogenous TRAC promoter, an endogenous TRBC promoter, an endogenous TRDC promoter, an endogenous TRGC promoter, and a combination thereof.

Embodiment 101. The cell of embodiment 100, wherein the endogenous promoter is a TRAC promoter.

Embodiment 102. The cell of any one of embodiments 72-101, wherein said cell is autologous.

Embodiment 103. The cell of any one of embodiments 72-101, wherein said cell is allogeneic.

Embodiment 104. A composition comprising a nucleic acid of any one of embodiments 62-69.

Embodiment 105. A composition comprising a vector of embodiment 70.

Embodiment 106. A composition comprising a lipid nanoparticle of embodiment 71.

Embodiment 107. A composition comprising a cell of any one of embodiments 72-103.

Embodiment 108. The composition of any one of embodiments 104-107, which is a pharmaceutical composition further comprising a pharmaceutically acceptable excipient.

Embodiment 109. The composition of any one of embodiments 104-108, which is for treating and/or preventing a neoplasm, an autoimmune disease, and/or an infectious disease.

Embodiment 110. The composition of any one of embodiments 104-109, further comprising a regulator that is capable of regulating or modulating expression and/or activity of the fusion polypeptide.

Embodiment 111. The composition of embodiment 110, wherein the regulator is selected from the group consisting of promoters that are capable of controlling the expression of the fusion polypeptide, molecules that are capable of regulating or modulating expression and/or activity of the co-stimulatory ligand, molecules that are capable of regulating or modulating expression and/or activity of the co-stimulatory molecule.

Embodiment 112. The composition of embodiment 111, wherein the molecules that are capable of regulating or modulating expression and/or activity of the co-stimulatory ligand are selected from the group consisting of antibodies that bind to the co-stimulatory ligand, and fusion proteins that bind to the co-stimulatory ligand and regulate or modulate the expression and/or activity of the co-stimulatory ligand.

Embodiment 113. The composition of any one of embodiments 110-112, wherein the regulator is an anti-CD80 antibody and the fusion polypeptide comprises an extracellular domain comprising a CD80 polypeptide and a transmembrane domain comprising a CD80 polypeptide.

Embodiment 114. The composition of any one of embodiments 110-113, wherein the regulator is a fusion protein that binds to CD80 and modulates the activity of CD80 and the fusion polypeptide comprises an extracellular domain comprising a CD80 polypeptide and a transmembrane domain comprising a CD80 polypeptide.

Embodiment 115. The composition of embodiment 114, wherein the fusion protein comprises an CTLA-4 fragment that binds to CD80.

Embodiment 116. The composition of embodiment 115, wherein the CTLA-4 fragment that binds to CD80 is abatacept or belatacept.

Embodiment 117. The composition of embodiment 116, wherein the molecules that are capable of regulating or modulating expression and/or activity of the co-stimulatory molecule are selected from the group consisting of antibodies that bind to the co-stimulatory molecule, and fusion proteins that bind to the co-stimulatory molecule and regulate or modulate the expression and/or activity of the co-stimulatory molecule.

Embodiment 118. A method of reducing tumor burden in a subject, the method comprising administering to the subject an effective amount of the cells of any one of embodiments 72-103, the composition of any one of embodiments 104-117, the nucleic acid of any one of embodiments 62-69, the vector of embodiment 70, or the lipid nanoparticle of embodiment 71.

Embodiment 119. The method of embodiment 118, wherein the method reduces the number of tumor cells, reduces tumor size, and/or eradicates the tumor in the subject.

Embodiment 120. A method of treating a subject having a relapse of a neoplasm, administering to the subject an effective amount of the cells of any one of embodiments 72-103, the composition of any one of embodiments 104-117, the nucleic acid of any one of embodiments 62-69, the vector of embodiment 70, or the lipid nanoparticle of embodiment 71.

Embodiment 121. A method of treating and/or preventing a neoplasm in a subject, administering to the subject an effective amount of the cells of any one of embodiments 72-103, the composition of any one of embodiments 104-117, the nucleic acid of any one of embodiments 62-69, the vector of embodiment 70, or the lipid nanoparticle of embodiment 71.

Embodiment 122. The method of any one of embodiments 118-121, wherein the subject received an immunotherapy prior to said administration of the cells or the composition.

Embodiment 123. The method of any one of embodiments 118-122, wherein the neoplasm or tumor is cancer.

Embodiment 124. The method of any one of embodiments 118-123, wherein the neoplasm or tumor is selected from the group consisting of blood cancers and solid tumors.

Embodiment 125. The method of embodiment 124, wherein the blood cancer is multiple myeloma, myeloid disorder, leukemia, or lymphoma.

Embodiment 126. The method of embodiment 125, wherein the leukemia is acute myeloid leukemia (AML), chronic myeloid leukemia (CMIL), acute lymphocytic leukemia (ALL), chronic lymphocytic leukemia (CLL), acute promyelocytic leukemia (APL), mixed-phenotype acute leukemia (MLL), hairy cell leukemia, or B cell prolymphocytic leukemia.

Embodiment 127. The method of embodiment 125, wherein the lymphoma is Hodgkin's lymphoma, non-Hodgkin's lymphoma, B-cell non-Hodgkin's lymphoma, or T-cell non-Hodgkin's lymphoma.

Embodiment 128. The method of embodiment 124, wherein the solid tumor is selected from the group consisting of renal cell carcinoma, non-small-cell lung cancer, lung adenocarcinoma, lung squamous cell carcinoma, lung neuroendocrine carcinoma, small-cell lung cancer, pancreatic cancer, breast cancer, astrocytoma, glioblastoma, laryngeal/pharyngeal carcinoma, EBV-associated nasopharyngeal carcinoma, and ovarian carcinoma.

Embodiment 129. The method of any one of embodiments 118-128, further comprising administering to the subject a regulator that is capable of regulating or modulating expression, activity of the fusion polypeptide.

Embodiment 130. The method of embodiment 129, wherein the regulator is selected from the group consisting of promoters that are capable of controlling the expression of the fusion polypeptide, molecules that are capable of regulating or modulating expression and/or activity of the co-stimulatory ligand, molecules that are capable of regulating or modulating expression and/or activity of the co-stimulatory molecule.

Embodiment 131. The method of embodiment 129, wherein the molecules that are capable of regulating or modulating expression and/or activity of the co-stimulatory ligand are selected from the group consisting of antibodies that bind to the co-stimulatory ligand, and fusion proteins that bind to the co-stimulatory ligand and regulate or modulate the expression and/or activity of the co-stimulatory ligand.

Embodiment 132. The method of any one of embodiments 129-131, wherein the regulator is an anti-CD80 antibody and the fusion polypeptide comprises an extracellular domain comprising a CD80 polypeptide and a transmembrane domain comprising a CD80 polypeptide.

Embodiment 133. The method of any one of embodiments 129-132, wherein the regulator is a fusion protein that binds to CD80 and modulates the activity of CD80 and the fusion polypeptide comprises an extracellular domain comprising a CD80 polypeptide and a transmembrane domain comprising a CD80 polypeptide.

Embodiment 134. The method of embodiment 133, wherein the fusion protein is a CTLA-4 fragment that binds to CD80.

Embodiment 135. The method of embodiment 134, wherein the CTLA-4 fragment that binds to CD80 is abatacept or belatacept.

Embodiment 136. The method of embodiment 129, wherein the molecules that are capable of regulating or modulating expression, activity of the co-stimulatory molecule are selected from the group consisting of antibodies that bind to the co-stimulatory molecule, fusion proteins that bind to the co-stimulatory molecule and regulate or modulate the expression, activity of the co-stimulatory molecule.

Embodiment 137. The method of any one of embodiments 129-136, wherein the regulator is capable of depleting the cell.

Embodiment 138. A method for producing a cell, the method comprising introducing into a cell the nucleic acid of any one of embodiments 62-69, the vector of embodiment 70, or the lipid nanoparticle of embodiment 71.

Embodiment 139. A kit comprising a cell of any one of embodiments 72-103, a composition of any one of embodiments 104-117, a nucleic acid of any one of embodiments 62-69, a vector of embodiment 70, or a lipid nanoparticle of embodiment 71.

Embodiment 140. The kit of embodiment 139, wherein the kit further comprises written instructions for treating and/or preventing a neoplasm, a pathogen infection, and/or an infectious disease.

Embodiment 141. The cells of any one of embodiments 72-103, the composition of any one of embodiments 104-117, the nucleic acid of any one of embodiments 62-69, the vector of embodiment 70, or the lipid nanoparticle of embodiment 71 for use in reducing tumor burden in a subject, treating a subject having a relapse of a neoplasm, and/or treating and/or preventing a neoplasm in a subject.

Embodiment 142. The cell, the composition, the nucleic acid, the vector, or the lipid nanoparticle for use of embodiment 141, wherein the cell, the composition, the nucleic acid, the vector, or the lipid nanoparticle reduces the number of tumor cells, reduces tumor size, and/or eradicates the tumor in the subject.

Embodiment 143. The cell, the composition, the nucleic acid, the vector, or the lipid nanoparticle for use of embodiment 141 or 142, wherein the subject received an immunotherapy prior to said administration of the cells or the composition.

Embodiment 144. The cell, the composition, the nucleic acid, the vector, or the lipid nanoparticle for use of any one of embodiments 141-143, wherein the neoplasm or tumor is cancer.

Embodiment 145. The cell, the composition, the nucleic acid, the vector, or the lipid nanoparticle for use of any one of embodiments 141-144, wherein the neoplasm or tumor is selected from the group consisting of blood cancers and solid tumors.

Embodiment 146. The cell, the composition, the nucleic acid, the vector, or the lipid nanoparticle for use of embodiment 145, wherein the blood cancer is multiple myeloma, myeloid disorder, leukemia, or lymphoma.

Embodiment 147. The cell, the composition, the nucleic acid, the vector, or the lipid nanoparticle for use of embodiment 146, wherein the leukemia is acute myeloid leukemia (AML), chronic myeloid leukemia (CML), acute lymphocytic leukemia (ALL), chronic lymphocytic leukemia (CLL), acute promyelocytic leukemia (APL), mixed-phenotype acute leukemia (MLL), hairy cell leukemia, or B cell prolymphocytic leukemia.

Embodiment 148. The cell, the composition, the nucleic acid, the vector, or the lipid nanoparticle for use of embodiment 146, wherein the lymphoma is Hodgkin's lymphoma, non-Hodgkin's lymphoma, B-cell non-Hodgkin's lymphoma, or T-cell non-Hodgkin's lymphoma.

Embodiment 149. The cell, the composition, the nucleic acid, the vector, or the lipid nanoparticle for use of embodiment 145, wherein the solid tumor is selected from the group consisting of renal cell carcinoma, non-small-cell lung cancer, lung adenocarcinoma, lung squamous cell carcinoma, lung neuroendocrine carcinoma, small-cell lung cancer, pancreatic cancer, breast cancer, astrocytoma, glioblastoma, laryngeal/pharyngeal carcinoma, EBV-associated nasopharyngeal carcinoma, and ovarian carcinoma.

Embodiment 150. The cell, the composition, the nucleic acid, the vector, or the lipid nanoparticle for use of any one of embodiments 141-150, wherein the subject receives or has received a regulator that is capable of regulating or modulating expression, activity of the fusion polypeptide.

Embodiment 151. The cell, the composition, the nucleic acid, the vector, or the lipid nanoparticle for use of embodiment 150, wherein the regulator is selected from the group consisting of promoters that are capable of controlling the expression of the fusion polypeptide, molecules that are capable of regulating or modulating expression and/or activity of the co-stimulatory ligand, molecules that are capable of regulating or modulating expression and/or activity of the co-stimulatory molecule.

Embodiment 152. The cell, the composition, the nucleic acid, the vector, or the lipid nanoparticle for use of embodiment 151, wherein the molecules that are capable of regulating or modulating expression and/or activity of the co-stimulatory ligand are selected from the group consisting of antibodies that bind to the co-stimulatory ligand, and fusion proteins that bind to the co-stimulatory ligand and regulate or modulate the expression and/or activity of the co-stimulatory ligand.

Embodiment 153. The cell, the composition, the nucleic acid, the vector, or the lipid nanoparticle for use of any one of embodiments 150-152, wherein the regulator is an anti-CD80 antibody and the fusion polypeptide comprises an extracellular domain comprising a CD80 polypeptide and a transmembrane domain comprising a CD80 polypeptide.

Embodiment 154. The cell, the composition, the nucleic acid, the vector, or the lipid nanoparticle for use of any one of embodiments 150-153, wherein the regulator is a fusion protein that binds to CD80 and modulates the activity of CD80 and the fusion polypeptide comprises an extracellular domain comprising a CD80 polypeptide and a transmembrane domain comprising a CD80 polypeptide.

Embodiment 155. The cell, the composition, the nucleic acid, the vector, or the lipid nanoparticle for use of embodiment 154, wherein the fusion protein is a CTLA-4 fragment that binds to CD80.

Embodiment 156. The cell, the composition, the nucleic acid, the vector, or the lipid nanoparticle for use of embodiment 155, wherein the CTLA-4 fragment that binds to CD80 is abatacept or belatacept.

Embodiment 157. The cell, the composition, the nucleic acid, the vector, or the lipid nanoparticle for use of embodiment 151, wherein the molecules that are capable of regulating or modulating expression, activity of the co-stimulatory molecule are selected from the group consisting of antibodies that bind to the co-stimulatory molecule, fusion proteins that bind to the co-stimulatory molecule and regulate or modulate the expression, activity of the co-stimulatory molecule.

Embodiment 158. The cell, the composition, the nucleic acid, the vector, or the lipid nanoparticle for use of any one of embodiments 150-157, wherein the regulator is capable of depleting the cell.

Embodiment 159. Use of the cells of any one of embodiments 72-103, the composition of any one of embodiments 104-117, the nucleic acid of any one of embodiments 62-69, the vector of embodiment 70, or the lipid nanoparticle of embodiment 71 for the manufacturing of a medicament for reducing tumor burden in a subject, treating a subject having a relapse of a neoplasm, and/or treating and/or preventing a neoplasm in a subject.

Embodiment 160. The use of embodiment 159, wherein the cell, the composition, the nucleic acid, the vector, or the lipid nanoparticle reduces the number of tumor cells, reduces tumor size, and/or eradicates the tumor in the subject.

Embodiment 161. The use of embodiment 159 or 160, wherein the subject received an immunotherapy prior to said administration of the cells or the composition.

Embodiment 162. The use of any one of embodiments 159-161, wherein the neoplasm or tumor is cancer.

Embodiment 163. The use of any one of embodiments 159-162, wherein the neoplasm or tumor is selected from the group consisting of blood cancers and solid tumors.

Embodiment 164. The use of embodiment 163, wherein the blood cancer is multiple myeloma, myeloid disorder, leukemia, or lymphoma.

Embodiment 165. The use of embodiment 164, wherein the leukemia is acute myeloid leukemia (AML), chronic myeloid leukemia (CML), acute lymphocytic leukemia (ALL), chronic lymphocytic leukemia (CLL), acute promyelocytic leukemia (APL), mixed-phenotype acute leukemia (MLL), hairy cell leukemia, or B cell prolymphocytic leukemia.

Embodiment 166. The use of embodiment 164, wherein the lymphoma is Hodgkin's lymphoma, non-Hodgkin's lymphoma, B-cell non-Hodgkin's lymphoma, or T-cell non-Hodgkin's lymphoma.

Embodiment 167. The use of embodiment 163, wherein the solid tumor is selected from the group consisting of renal cell carcinoma, non-small-cell lung cancer, lung adenocarcinoma, lung squamous cell carcinoma, lung neuroendocrine carcinoma, small-cell lung cancer, pancreatic cancer, breast cancer, astrocytoma, glioblastoma, laryngeal/pharyngeal carcinoma, EBV-associated nasopharyngeal carcinoma, and ovarian carcinoma.

Embodiment 168. The use of any one of embodiments 159-167, wherein the subject receives or has received a regulator that is capable of regulating or modulating expression, activity of the fusion polypeptide.

Embodiment 169. The use of embodiment 168, wherein the regulator is selected from the group consisting of promoters that are capable of controlling the expression of the fusion polypeptide, molecules that are capable of regulating or modulating expression and/or activity of the co-stimulatory ligand, molecules that are capable of regulating or modulating expression and/or activity of the co-stimulatory molecule.

Embodiment 170. The use of embodiment 169, wherein the molecules that are capable of regulating or modulating expression and/or activity of the co-stimulatory ligand are selected from the group consisting of antibodies that bind to the co-stimulatory ligand, and fusion proteins that bind to the co-stimulatory ligand and regulate or modulate the expression and/or activity of the co-stimulatory ligand.

Embodiment 171. The use of any one of embodiments 168-170, wherein the regulator is an anti-CD80 antibody and the fusion polypeptide comprises an extracellular domain comprising a CD80 polypeptide and a transmembrane domain comprising a CD80 polypeptide.

Embodiment 172. The use of any one of embodiments 168-171, wherein the regulator is a fusion protein that binds to CD80 and modulates the activity of CD80 and the fusion polypeptide comprises an extracellular domain comprising a CD80 polypeptide and a transmembrane domain comprising a CD80 polypeptide.

Embodiment 173. The use of embodiment 172, wherein the fusion protein is a CTLA-4 fragment that binds to CD80.

Embodiment 174. The use of embodiment 173, wherein the CTLA-4 fragment that binds to CD80 is abatacept or belatacept.

Embodiment 175. The use of embodiment 169, wherein the molecules that are capable of regulating or modulating expression, activity of the co-stimulatory molecule are selected from the group consisting of antibodies that bind to the co-stimulatory molecule, fusion proteins that bind to the co-stimulatory molecule and regulate or modulate the expression, activity of the co-stimulatory molecule.

Embodiment 176. The use of any one of embodiments 159-175, wherein the regulator is capable of depleting the cell.

### Examples

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides disclosed herein, and, as such, may be considered in making and practicing the presently disclosed subject matter. Particularly useful techniques for particular embodiments will be discussed in the sections that follow.

The following examples are put forth so as to provide those of ordinary skill in the art with a complete disclosure and description of how to make and use the presently disclosed cells and compositions, and are not intended to limit the scope of what the inventors regard as their invention.

### Example 1—PRIME-Costimulatory Fusion Polypeptide

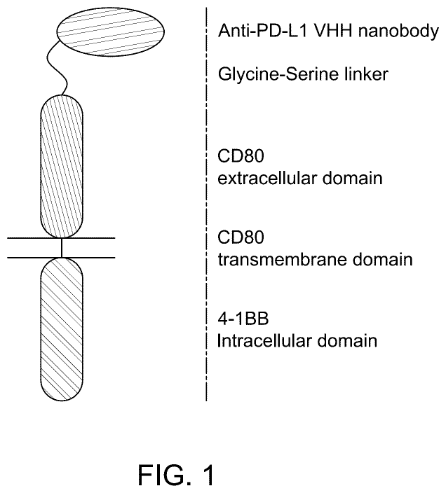

The presently disclosed subject matter generated and designed a novel fusion protein designated as Platform for Rewiring Immune Molecules and Enhance Co-stimulation, hereinafter referred to as “PRIME-costim” or “PRIME-costimulatory fusion polypeptide” (See FIG. 1). The PRIME-costimulatory fusion polypeptide comprises the extracellular domain of the human costimulatory CD80 which has been engineered to bind the tumor-abundant immune checkpoint molecule PD-L1. The presently disclosed PRIME-costimulatory fusion polypeptide targets PD-L1 through a binding domain (i.e., nanobody against human PD-L1) linked to the N terminal of CD80 extracellular domain without compromising CD28:CD80 or CTLA-4:CD80 interactions, while delivering costimulatory signaling through its 4-1BB based intracellular motif. The inventors of the presently disclosed subject matter hypothesized that the PRIME-costimulatory fusion polypeptide function in T cells would surpass other costimulatory molecules (e.g., CD80/4-1BB fusion proteins) not only by combining blockade of key immune checkpoints CTLA-4 or PD-L1, but also providing 4-1BB signaling upon receptors interactions. PRIME-costimulatory fusion polypeptide preserves its dual co-stimulatory feature by providing CD28 and 4-1BB signaling upon CD28:CD80 binding. The multifaceted antigen-independent stimulatory features of PRIME-costimulatory fusion polypeptide combined with OR-gate function (i.e., PRIME-costimulatory fusion polypeptide can function in the presence of any of its ligands) can benefit adoptive cell therapies in an antigen receptor agnostic manner (i.e., CAR, HIT, and TCR). Thus, PRIME-costimulatory fusion polypeptides can enhance tumor control with CAR, TCR, and TILs in hematological malignancies and solid tumors.

The presently disclosed PRIME-costimulatory fusion polypeptide provides significant advantages over CD80/4-1BB fusion proteins. As illustrated in FIG. 2, CD80/4-1BB fusion protein interacts in cis but not in trans with PD-L1, making trans interaction between PD1 and PD-L1 the dominant one over the cis interaction CD80 with PD-L1. In addition, the cis interaction between CD80/4-1BB:PD-L1 interaction does not overcome the inhibitory effect of the interaction between PD-1 and PD-L1. Further, CD80/4-1BB requires very high expression levels on T cell surface in order to provide therapeutical benefits with HIT or TCR receptors. Notably, the presently disclosed PRIME-costimulatory fusion polypeptide interacts in cis and in trans with PD-L1, CD28, and CTLA-4. Importantly, the trans binding to PD-L1 provides significant advantages. For example, it inhibits the interaction between PD-1 and PD-L1, drives co-stimulation downstream of the costimulatory motif and increases avidity binding between T cells and tumor cells. These features further allow the presently disclosed PRIME-costimulatory fusion polypeptide to work at low expression levels (e.g., expression from the TRAC locus).

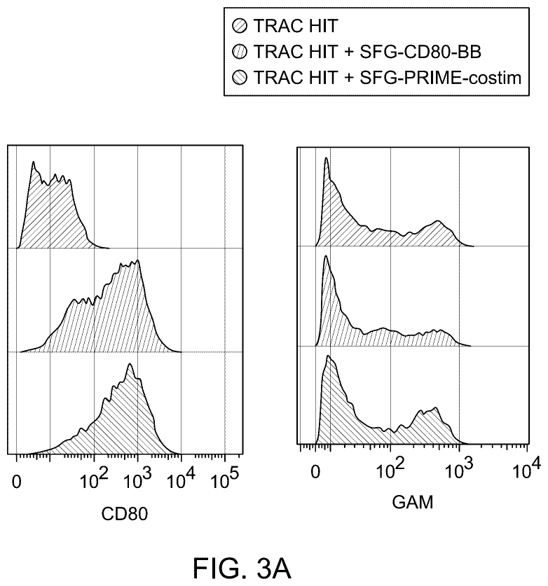

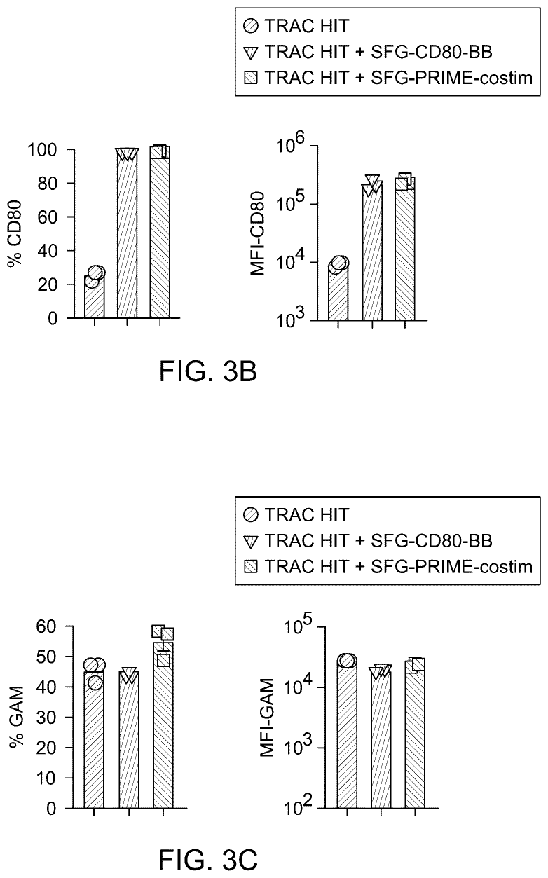

To determine whether the PRIME-costimulatory fusion polypeptide was correctly expressed on the surface of the T cells, T cells were transduced to express the CD80/4-1BB fusion protein (SFG-CD80-BB) or the PRIME-costimulatory fusion polypeptide (SFG-PRIME-costim) along with a HIT receptor inserted into the TRAC locus using the CRISPR-Cas9/AAV6 system (TRAC HIT). As seen in FIGS. 3A-3C, CD80 expression was increased in cells expressing the CD80/4-1BB fusion protein or the PRIME-costimulatory fusion polypeptide at comparable levels. These results demonstrate that the PRIME-costimulatory fusion polypeptide is well expressed on the T cell surface.

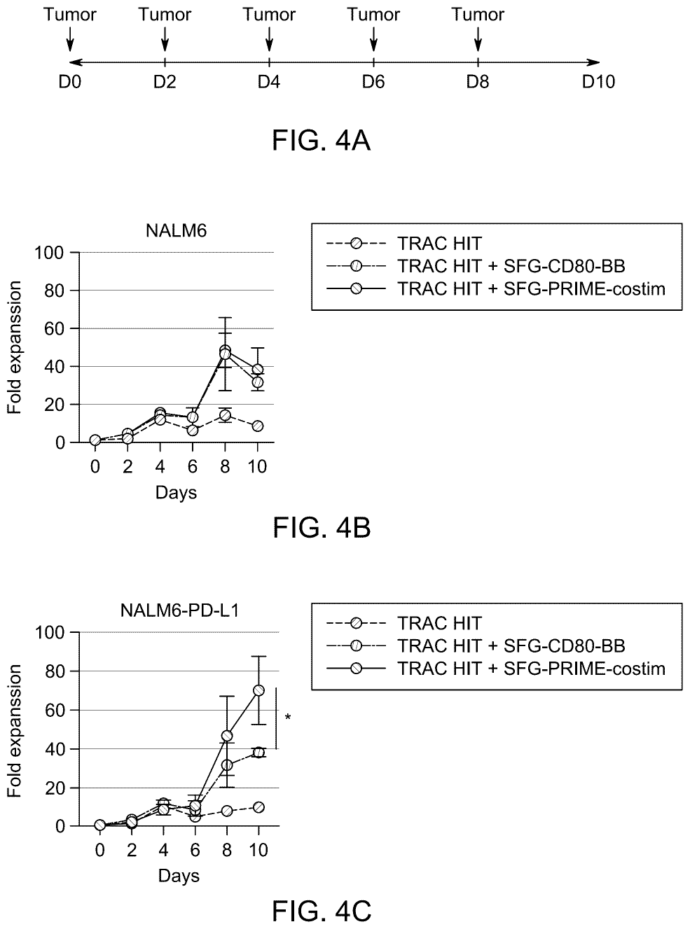

Next, the functional properties of these cells were tested in in vitro settings. In vitro antigen stimulation was performed using NALM6 tumor cells expressing PD-L1 (NALM6-PD-L1) or control (NALM6). See FIGS. 4A-4C. Briefly, T cells were cultured with pre-leukemia tumor cells (NALM6 and NALM6-PD-L1) at a ratio of 2:1. Every 48 hours, half of the T cell volume was discarded, and 10⁶ tumor cells were added for four consecutive rounds (FIG. 4A). After each round of stimulation, cells were counted and the fractions of T cells and tumor cells were determined by flow cytometry. As shown in FIG. 4B, the fold expansion of T cells expressing i) a HIT receptor, and ii) the CD80/4-1BB fusion protein or the PRIME-costimulatory fusion polypeptide cultured with control NALM6 tumor cells showed a similar proliferation increase. However, when T cells expressing the CD80/4-1BB fusion protein or the PRIME-costimulatory fusion polypeptide were cultured with tumor cells expressing PD-L1 (NALM6-PD-L1), the fold expansion of the T cells expressing the PRIME-costimulatory fusion polypeptide was significantly greater compared to the control (TRAC HIT+SFG-CD80-BB) (FIG. 4C).

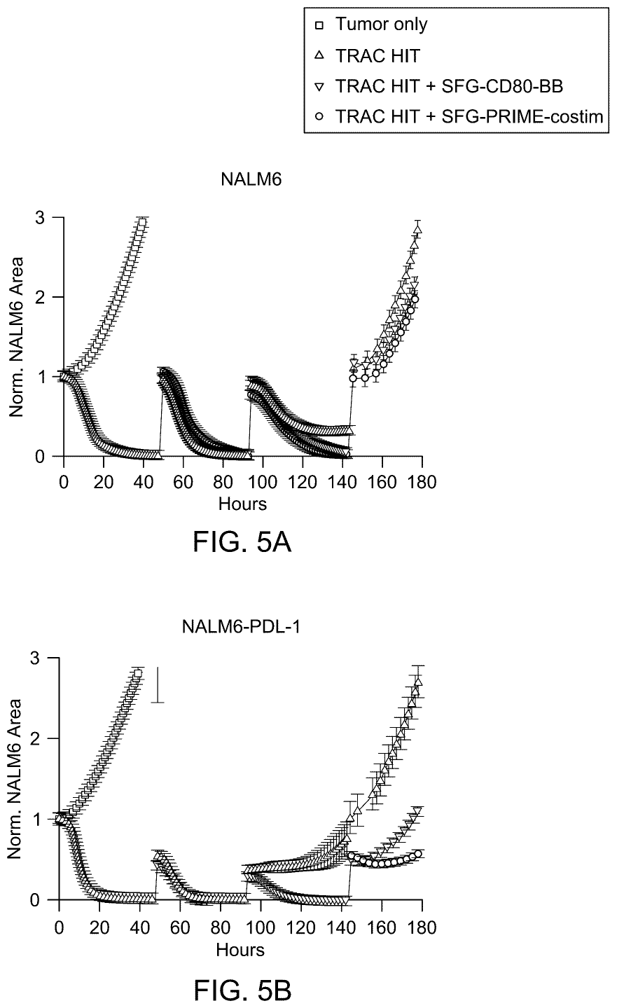

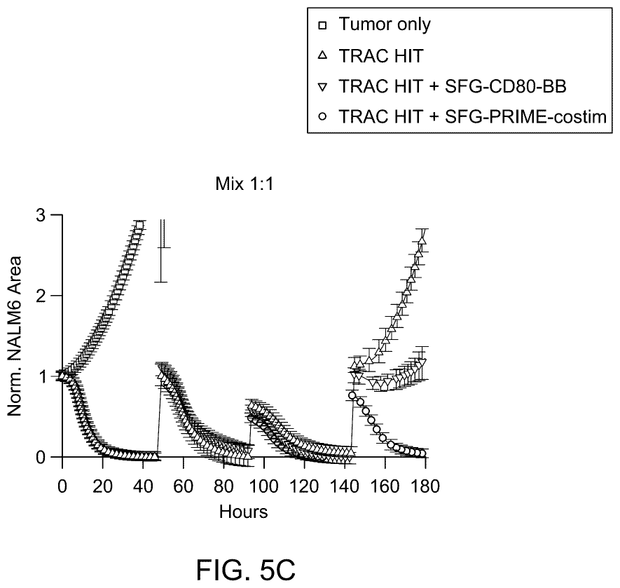

Next, a serial in vitro cytotoxicity assay was performed using NALM6 tumor cells expressing PD-L1 (NALM6-PD-L1), control cells (NALM6), or a mix thereof (NALM6:NALM6-PD-L1). Briefly, T cells expressing the CD80/4-1BB fusion protein or the PRIME-costimulatory fusion polypeptide were cocultured with the tumor cells; a fixed number of tumor cells was added every 48 hours to each well of the coculture, and data were acquired using IncuCyte live cell imaging system to track tumor cells. As shown in FIGS. 5A-5C, T cells expressing the PRIME-costimulatory fusion polypeptide showed a greater reduction in the tumor cell area of tumor cells expressing PD-L1 (NALM6-PD-L1 and NALM6:NALM6-PD-L1) as compared to the controls. All the results shown herein demonstrate that the PRIME-costimulatory fusion polypeptide outperforms the CD80/4-1BB in T cell proliferation and tumor control in vitro in the presence of PD-L1.

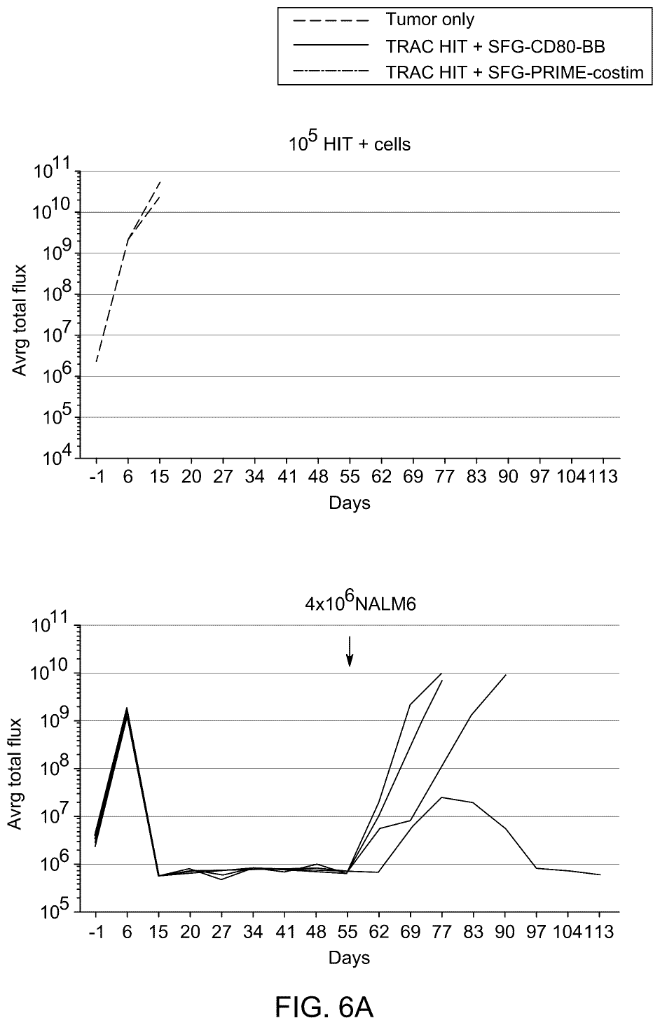

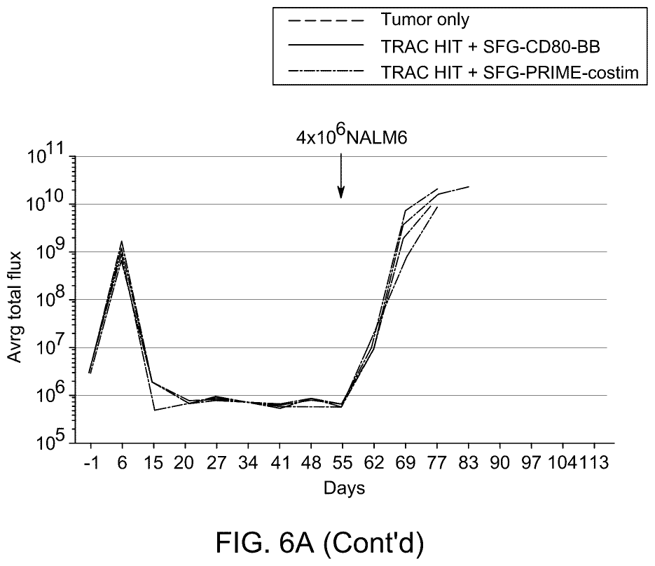

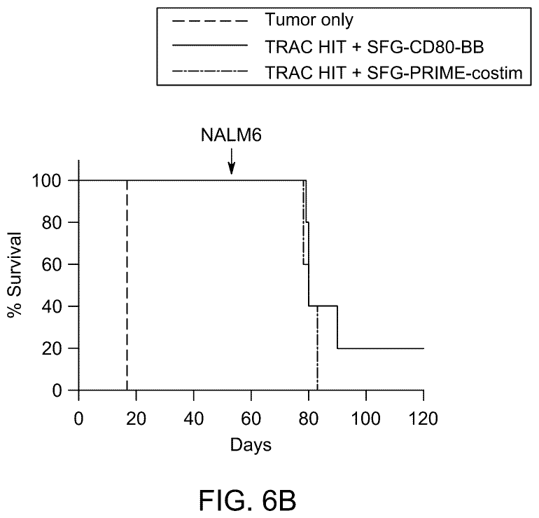

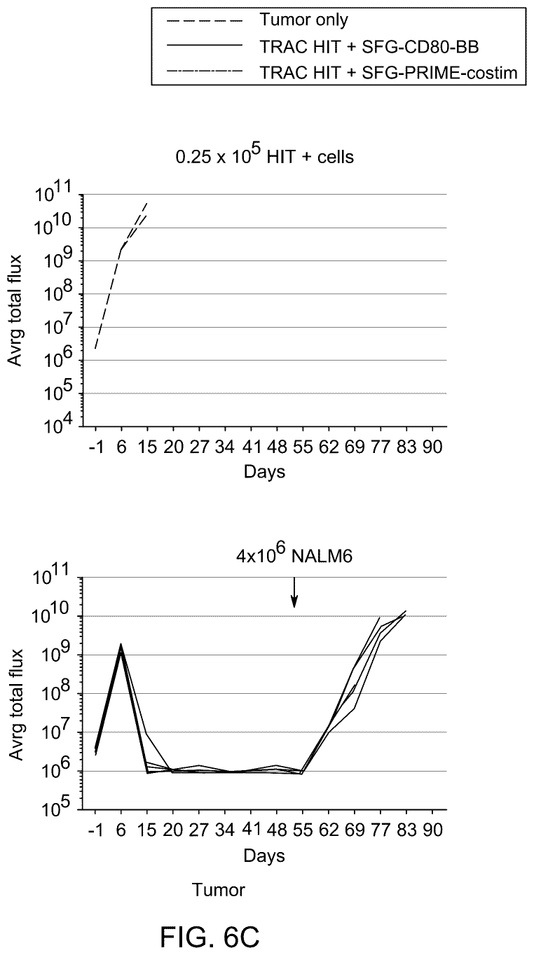

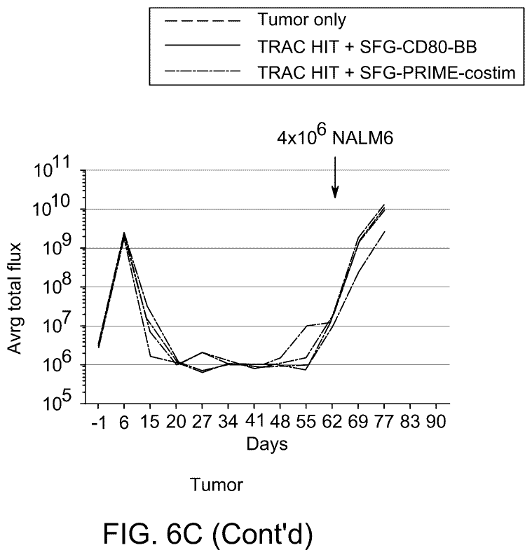

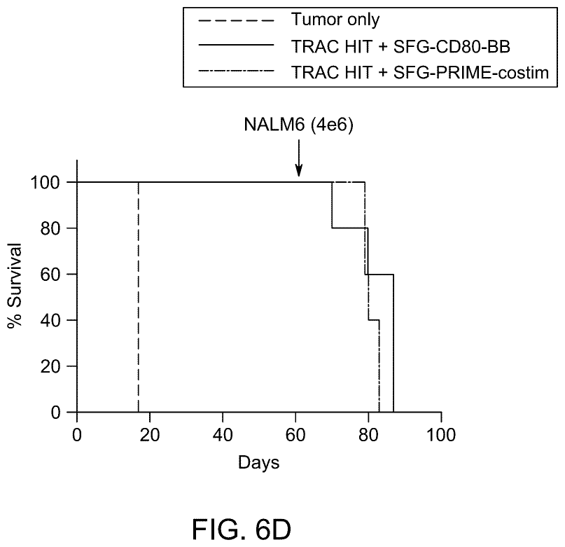

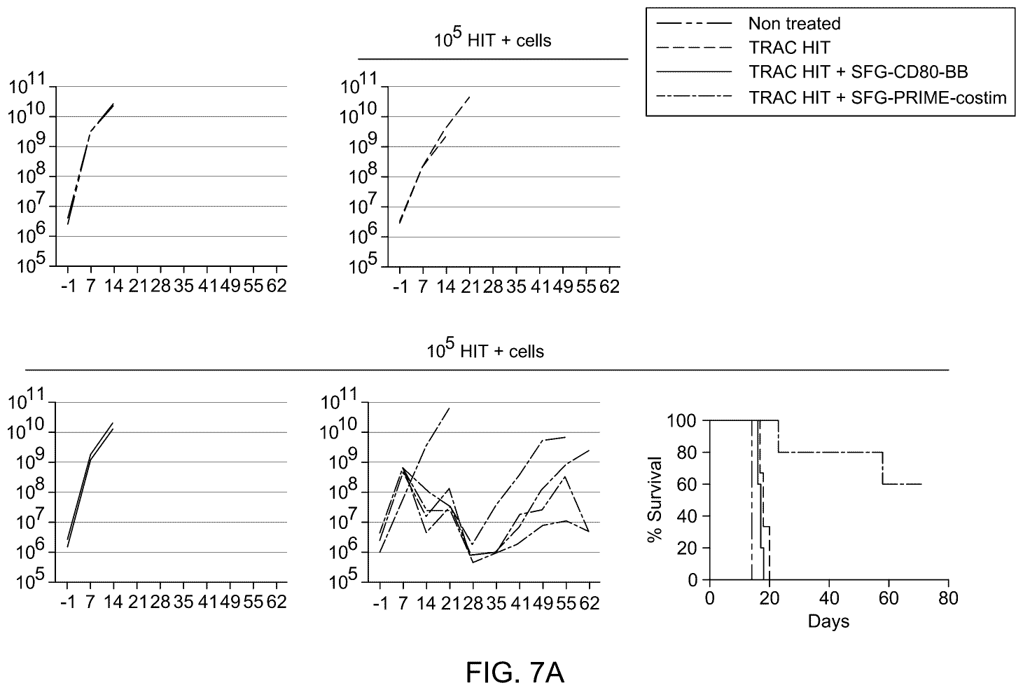

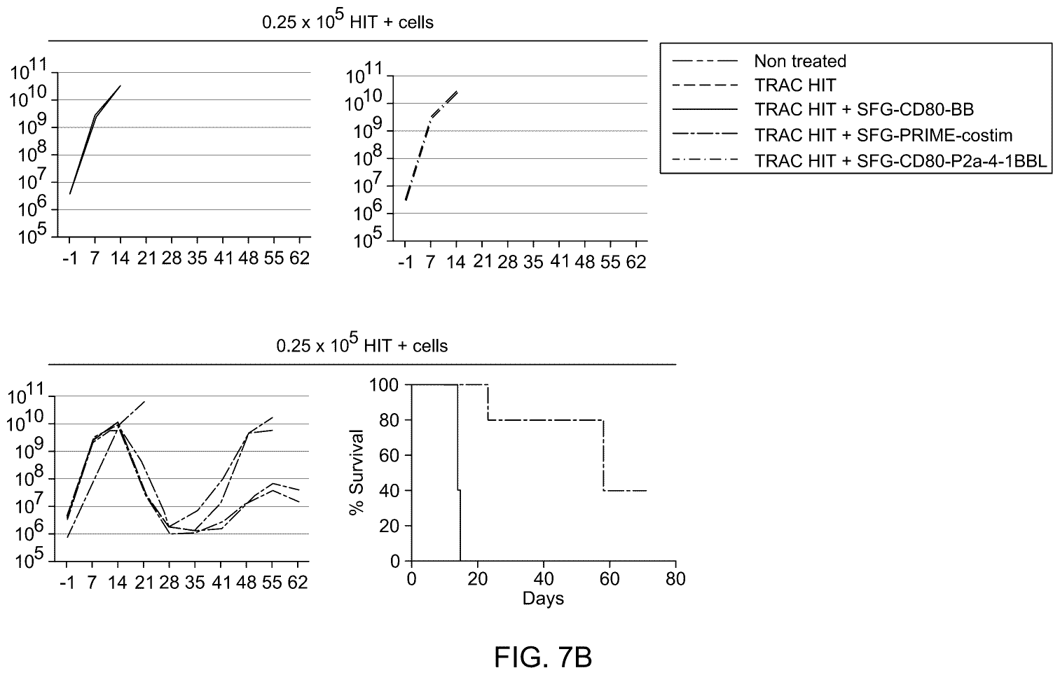

Next, it was determined whether the PRIME-costimulatory fusion polypeptide could control tumors in vivo. To determine the in vivo efficacy of the PRIME-costimulatory fusion polypeptide, mice were injected with 0.5×10⁶ tumor cells not expressing PD-L1 (NALM6-GFP-FFLuc cells) followed by treatment with T cells expressing the CD80/4-1BB fusion protein (TRAC-HIT-CD80-4-1BB) or the PRIME-costimulatory fusion polypeptide (TRAC-HIT-PRIME-costim) 4 days later. Mice were re-challenged with 4×10⁶ tumor cells 59 days after the 1^{st} T cell infusion. As shown in FIGS. 6A-6D both tested T cells showed comparable efficacy in tumor control and survival in presence of PD-L1 negative tumors. Importantly, as shown in FIGS. 7A and 7B, when mice were challenged with tumor cells expressing PD-L1 (NALM6-GFP-FFLuc cells), T cells expressing the PRIME-costimulatory fusion polypeptide (TRAC-HIT+SFG-PRIME-costim) outperformed cells expressing CD80/4-1BB fusion protein (TRAC-HIT+SFG-CD80-BB) or expressing exogenous CD80 and 4-1BBL (TRAC-HIT+SFG-CD80-P2a-4-1BBL). Overall, these data indicate that the PRIME-costimulatory fusion polypeptide has improved tumor control compared to alternative strategies,

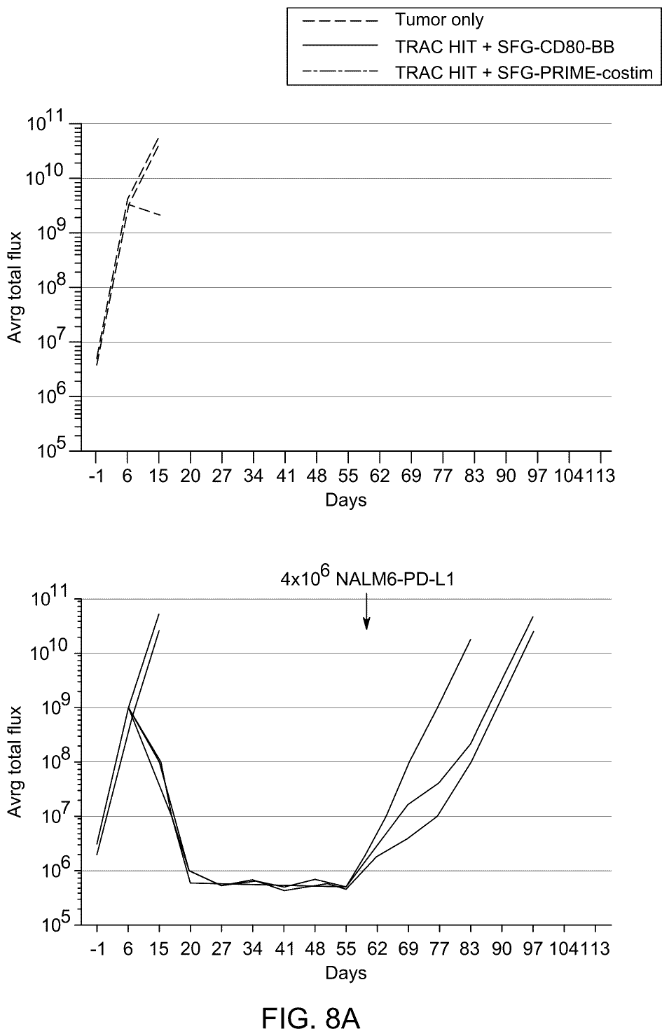

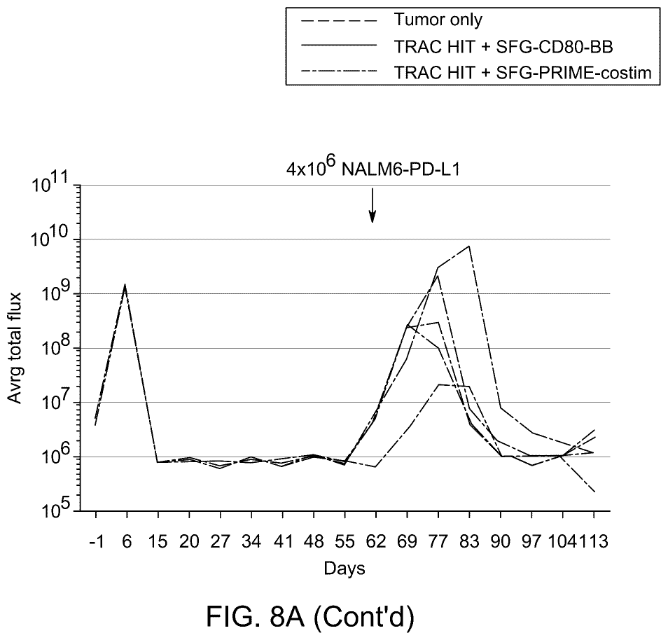

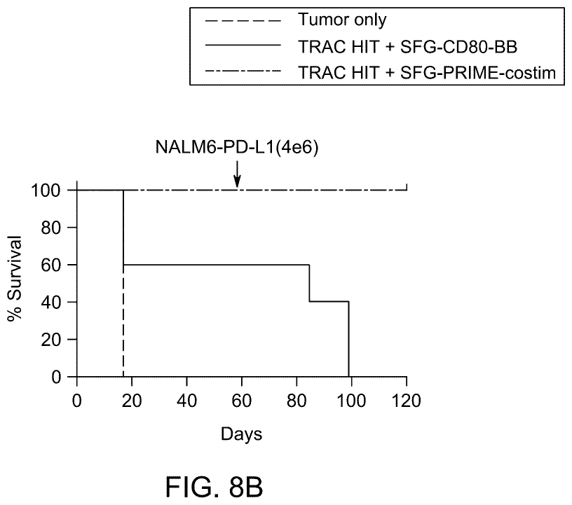

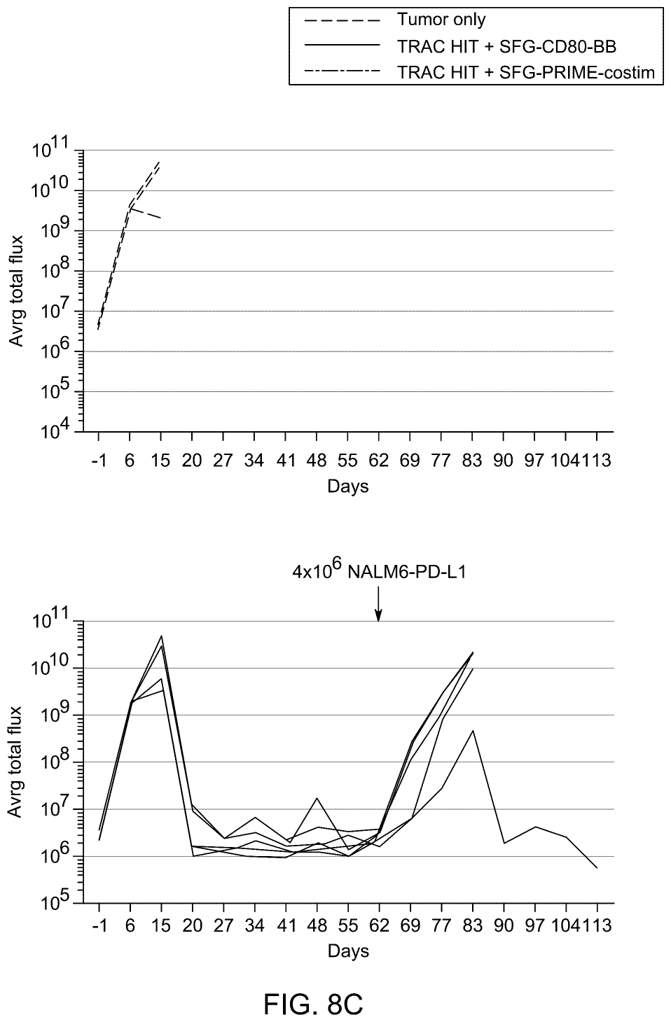

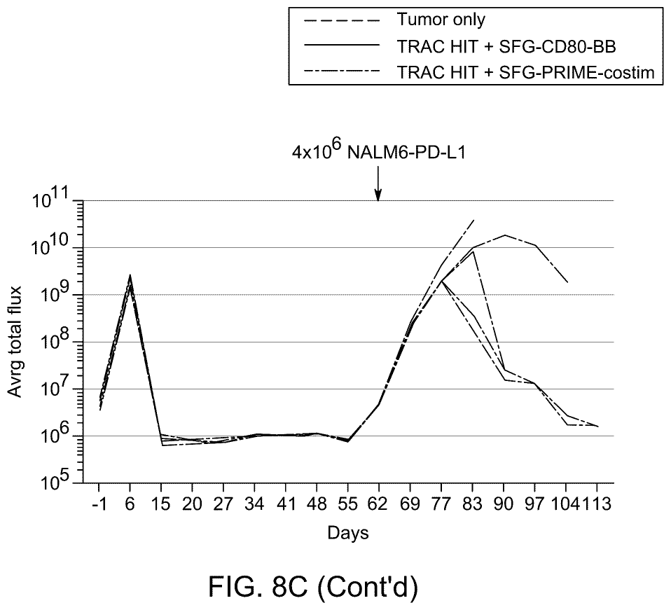

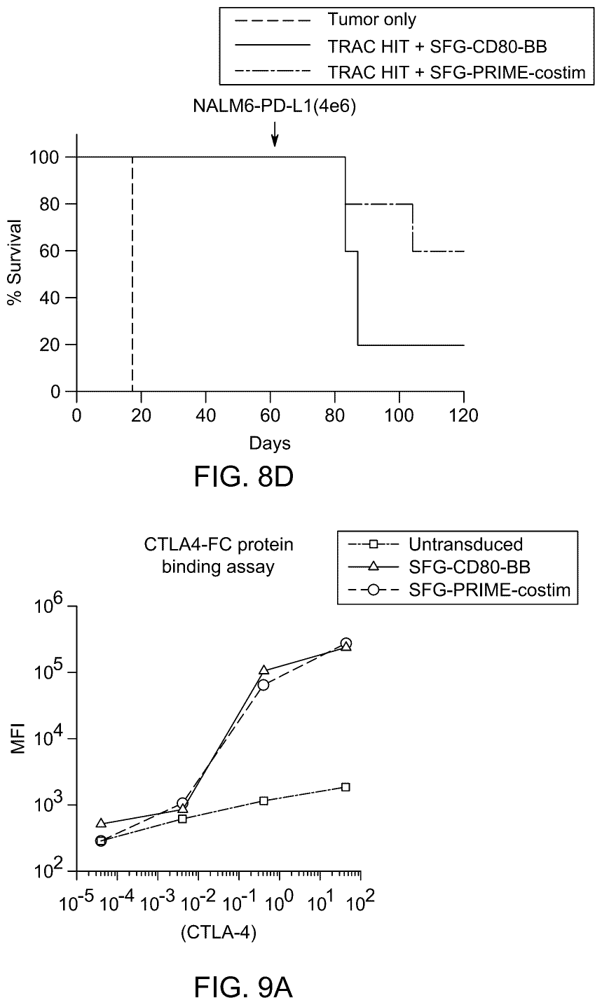

To further confirm the tumor control ability of cells expressing the PRIME-costimulatory fusion polypeptide an additional mouse model was used. Briefly, mice were injected with 0.5×10⁶ tumor cells not expressing PD-L1 (NALM6-GFP-FFLuc cells) followed by treatment with T cells expressing the CD80/4-1BB fusion protein (TRAC-HIT-CD80-4-1BB) or the PRIME-costimulatory fusion polypeptide (TRAC-HIT-PRIME-costim) 4 days later. Mice were then re-challenged with 4×10⁶ tumor cells expressing PD-L1 (NALM6-PD-L1+) 59 days after the first T cell infusion. As shown in FIGS. 8A-8D, the expression of the PRIME-costimulatory fusion polypeptide was sufficient to significantly improve tumor control and increase survival. Notably, such effects were observed even with low doses of T cells (0.25×10⁵ cells).

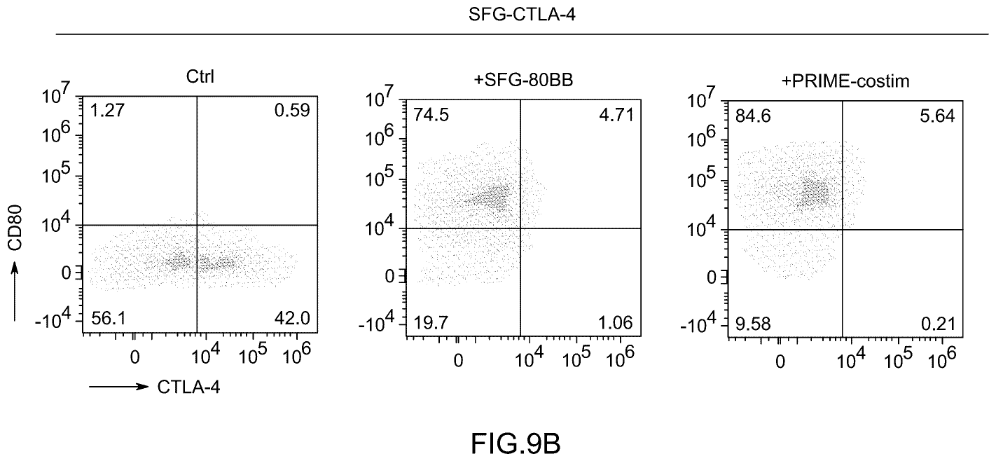

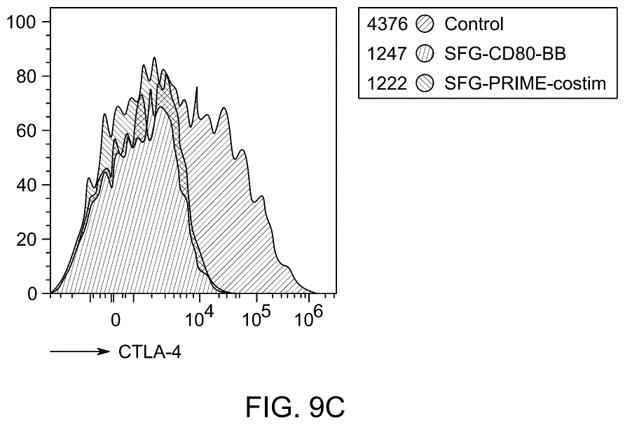

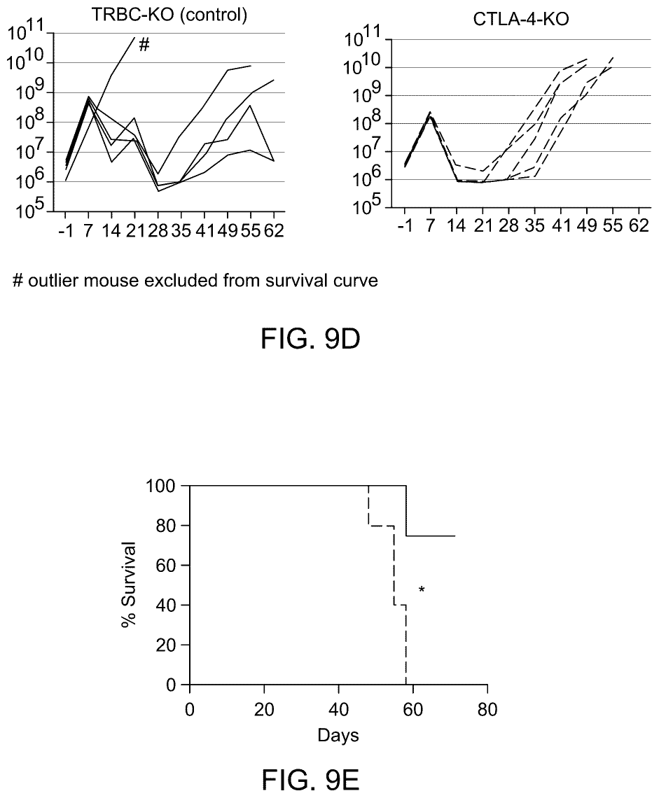

The design of the PRIME-costimulatory fusion polypeptide could theoretically cause steric interference in the ability to bind to any one of the intended ligands. Therefore binding of PRIME-costim to CTLA-4 was investigated. FIG. 9A shows that the PRIME-costimulatory fusion polypeptide did not interfere with the ability of the cells to bind to a CTLA4-Fc fusion protein. Moreover, as shown in FIGS. 9B and 9C, the CD80 polypeptide of the PRIME-costimulatory fusion polypeptide was capable of binding to CTLA4. Next, it was investigated whether the CTLA-4 binding to the PRIME-costimulatory fusion polypeptide is required for enhanced tumor control in vivo. Mice were injected with 0.5×10⁶ tumor cells (NALM6-GFP-FFLuc cells) and 4 days later were injected with T cells expressing PRIME-costimulatory fusion polypeptide in which either TRBC (control) or CTLA-4 was knocked out using CRISPR-Cas9 system. As shown in FIGS. 9D and 9E, knockout of CTLA-4 impaired tumor control and survival as compared to the control.

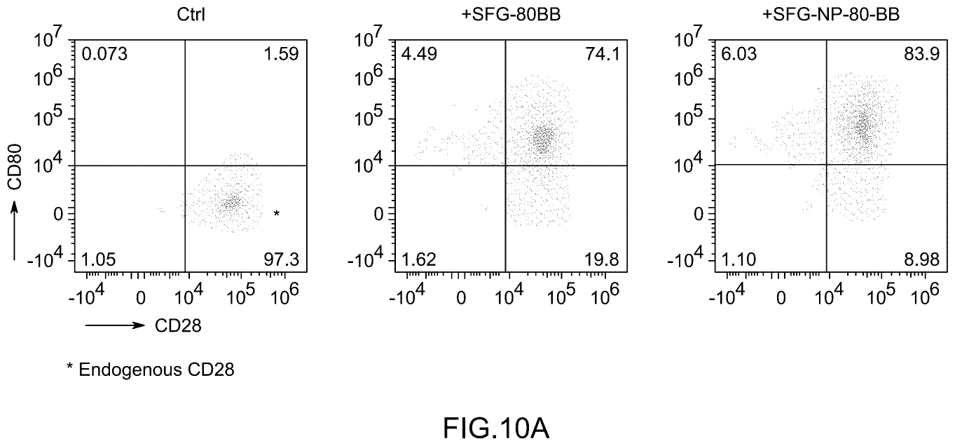

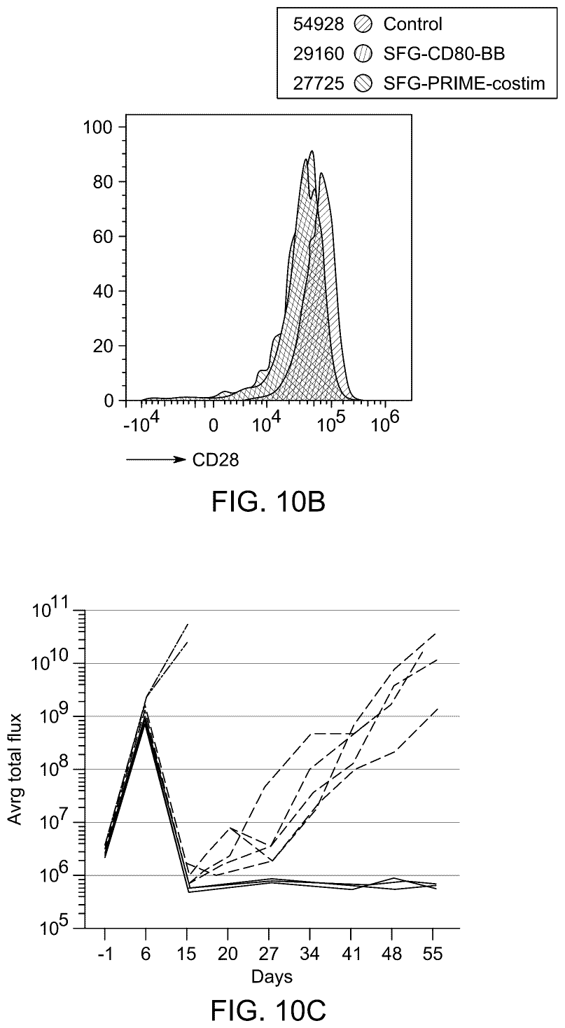

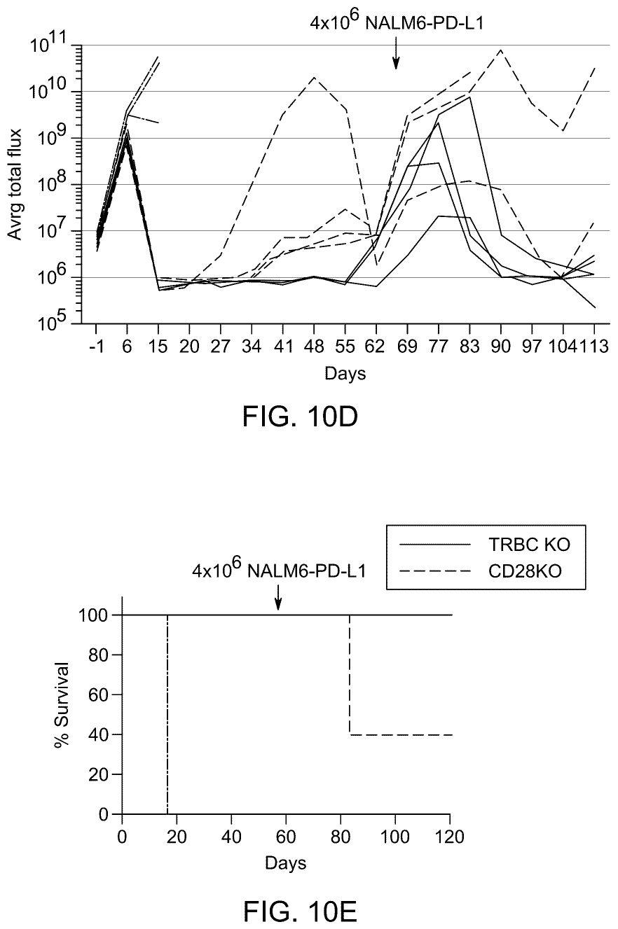

Since CD80 can interact in cis with CD28, the binding of the PRIME-costimulatory fusion polypeptide with endogenous CD28 was studied. Briefly, T cells were transduced with the PRIME-costimulatory fusion polypeptide and titrated with a human CD28-Fc fusion protein. Cells transduced with the PRIME-costimulatory fusion polypeptide bound to CD28 (data not shown). In addition, when assessed by flow cytometry the binding between CD28 and the CD80 polypeptide of the PRIME-costimulatory fusion polypeptide was preserved (FIGS. 10A and 10B). To further determine whether CD28 was required for in vivo tumor control, mice were injected with tumor cells (NALM6-GFP-FFLuc cells) were treated with T cells expressing PRIME-costimulatory fusion polypeptide in which either TRBC (control) or CD28 was knocked out. Notably, as shown in FIGS. 10C-10E, knockout of CD28 impaired tumor control and survival as compared to the controls. These results demonstrate that the interaction (i.e., cis, as depicted in FIG. 2) between the PRIME-costimulatory fusion polypeptide and CTLA-4 or CD28 is preserved and required for the observed enhanced in vivo tumor control.

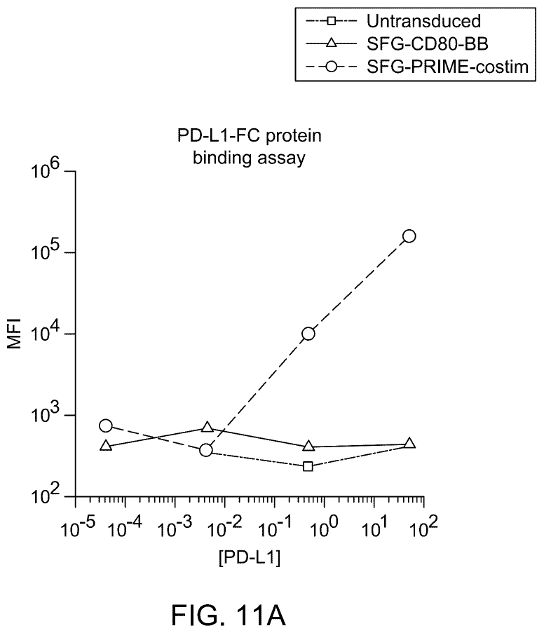

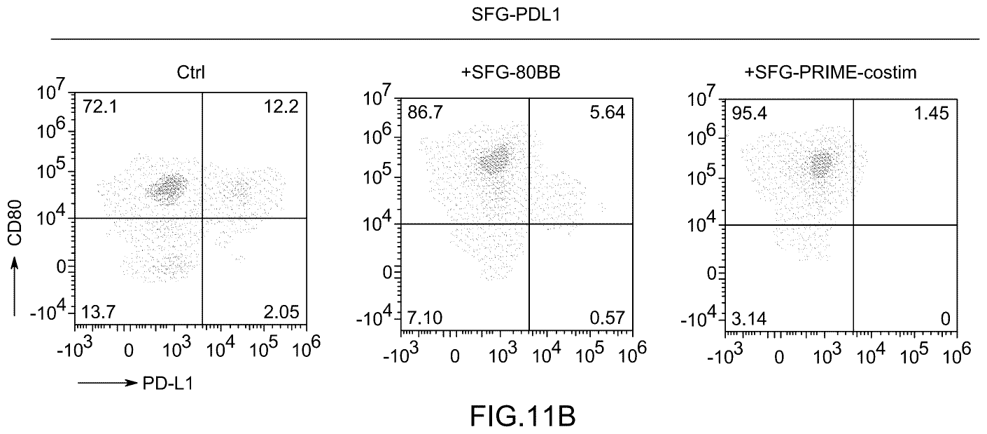

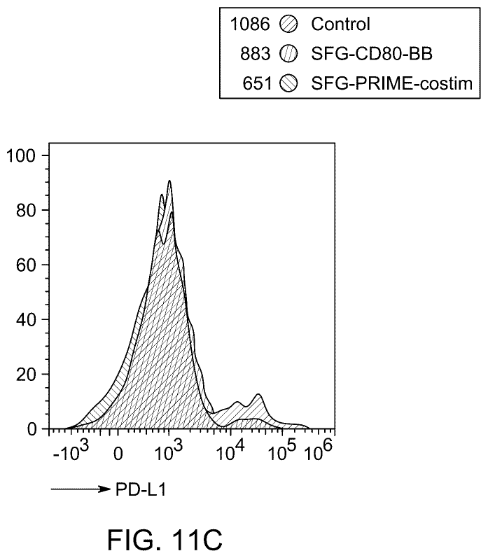

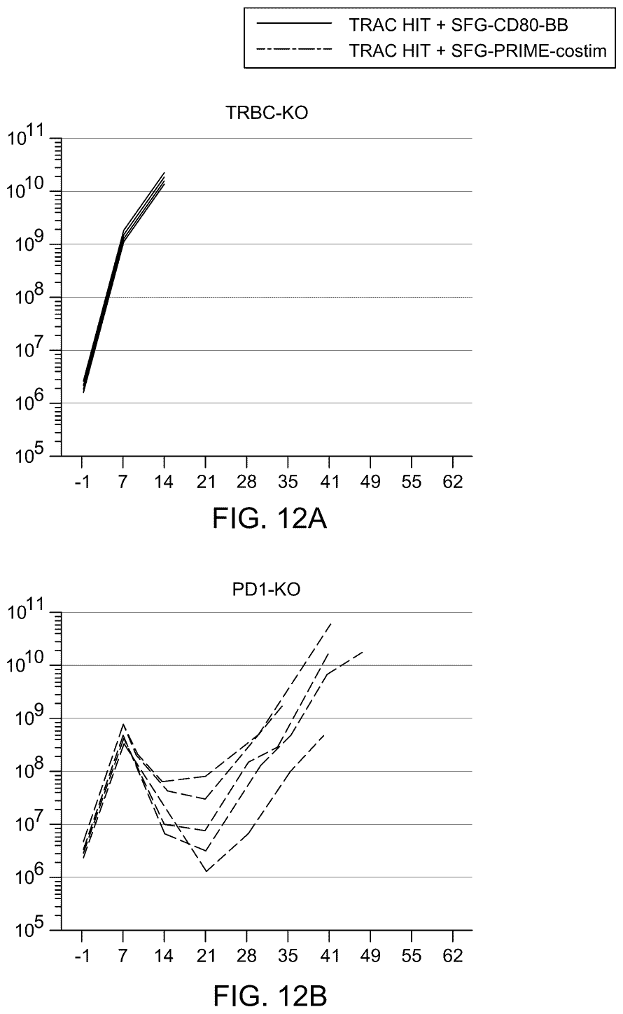

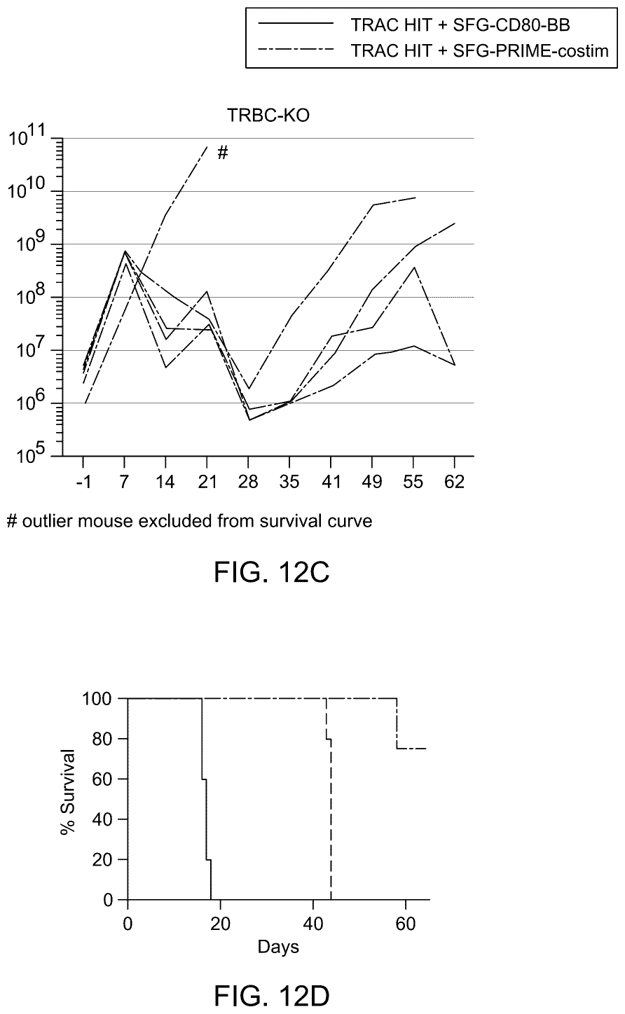

To further test the cis interaction and its effects on the T cells, it was investigated whether the PRIME-costimulatory fusion polypeptide could bind to endogenous PD-L1. Thus, an initial assay using PD-L1-Fc fusion protein was performed. FIG. 11A shows that cells expressing PRIME-costimulatory fusion polypeptides were effectively binding to PD-L1. Further, as shown in flow cytometry analysis in FIGS. 11B and 11C, PD-L1 bound the PRIME-costimulatory fusion polypeptide. Finally, it was determined whether the observed effects of the presently disclosed cells expressing the PRIME-costimulatory fusion polypeptide were associated with the inhibition of the PD-1 signaling. To test this hypothesis, mice challenged with tumors expressing PD-L1 were treated with T cells expressing the PRIME-costimulatory fusion polypeptide or T cells expressing the CD80/4-1BB fusion protein and knockout for the PDCK1 gene. FIGS. 12A-12D show that T cells expressing the PRIME-costimulatory fusion polypeptide outperformed the relative control knockout for PD-1.

In conclusion, the present example provides and illustrates several advantages of the presently disclosed PRIME-costimulatory fusion polypeptide. In addition to being well expressed on the T cell surface, this molecule provides increased T cell proliferation and in vivo tumor control. Importantly, the interactions of this molecule with other important targets (e.g., CTLA-4, CD28, and PD-L1) are required for enhanced tumor control. Thus, the presently disclosed PRIME-costimulatory fusion polypeptide is an important tool that can enhance tumor control in cells expressing an antigen-recognizing receptor (e.g., CAR, TCR, HIT) as well as being adopted for tumor-infiltrating lymphocytes (TILs) in hematological and/or solid tumors.

### Embodiments of the Presently Disclosed Subject Matter

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A fusion polypeptide comprising an extracellular domain comprising an antigen-binding fragment and a co-stimulatory ligand polypeptide, and an intracellular domain comprising a first co-stimulatory molecule polypeptide.

2. The fusion polypeptide of claim 1, wherein the antigen-binding fragment binds to an immune checkpoint molecule.

3. The fusion polypeptide of claim 2, wherein the immune checkpoint molecule is selected from the group consisting of PD-L1, PD-L2, VISTA, B7-H3, B7-H4, B7-H7, herpesvirus entry mediator (HVEM), CD155, CD112, CD200, galectin 9 (GAL9), TIGIT, BTLA, LAG-3, TIM-3, and 2B4 (CD224).

4. The fusion polypeptide of claim 2, wherein the antigen-binding fragment binds to PD-L1.

5. The fusion polypeptide of claim 1, wherein the antigen-binding fragment is a Fab, a Fab′, a F(ab′)2, a variable fragment (Fv), a single chain variable region (scFv), a nanobody, a microantibody, an affibody molecule, an affilin, an affimer, an affitin, an alphabody, an anticalin protein, an avimer, a DARPin (designed ankyrin repeat proteins), or an aptamer.

6. The fusion polypeptide of claim 5, wherein the antigen-binding fragment is a nanobody.

7. The fusion polypeptide of claim 6, wherein the nanobody comprises a CDR1 comprising the amino acid sequence set forth in SEQ ID NO: 27 or a conservative modification thereof, a CDR2 comprising the amino acid sequence set forth in SEQ ID NO: 28 or a conservative modification thereof, and CDR3 comprising the amino acid sequence set forth in SEQ ID NO: 29 or a conservative modification thereof.

8. The fusion polypeptide of claim 7, wherein the nanobody comprises a CDR1 comprising the amino acid sequence set forth in SEQ ID NO: 27, a CDR2 comprising the amino acid sequence set forth in SEQ ID NO: 28, and CDR3 comprising the amino acid sequence set forth in SEQ ID NO: 29.

9. The fusion polypeptide of claim 6, wherein the nanobody comprises or consists of an amino acid sequence that is at least about 80% identical to the amino acid sequence set forth in SEQ ID NO: 30.

10. The fusion polypeptide of claim 9, wherein the nanobody comprises the amino acid sequence set forth in SEQ ID NO: 30.

11. The fusion polypeptide of claim 5, wherein the antigen-binding fragment is an scFv.

12. The fusion polypeptide of claim 11, wherein the scFv comprises a heavy chain variable region (VH) comprising a CDR1 comprising the amino acid sequence set forth in SEQ ID NO: 16 or a conservative modification thereof, a CDR2 comprising the amino acid sequence set forth in SEQ ID NO: 17 or a conservative modification thereof, and a CDR3 comprising the amino acid sequence set forth in SEQ ID NO: 18 or a conservative modification thereof, and a light chain variable region (VL) comprising a CDR1 comprising the amino acid sequence set forth in SEQ ID NO: 19 or a conservative modification thereof, a CDR2 comprising the amino acid sequence set forth in SEQ ID NO: 20 or a conservative modification thereof, and a CDR3 comprising the amino acid sequence set forth in SEQ ID NO: 21 or a conservative modification thereof.

13. The fusion polypeptide of claim 12, wherein the VH comprises a CDR1 comprising the amino acid sequence set forth in SEQ ID NO: 16, a CDR2 comprising the amino acid sequence set forth in SEQ ID NO: 17, and a CDR3 comprising the amino acid sequence set forth in SEQ ID NO: 18; and the VL comprises a CDR1 comprising the amino acid sequence set forth in SEQ ID NO: 19, a CDR2 comprising the amino acid sequence set forth in SEQ ID NO: 20, and a CDR3 comprising the amino acid sequence set forth in SEQ ID NO: 21.

14. The fusion polypeptide of claim 11, wherein the scFv comprises a heavy chain variable region (VH) that is at least about 80% identical to the amino acid sequence set forth in SEQ ID NO: 22, and a light chain variable region (VL) that is at least about 80% identical to the amino acid sequence set forth in SEQ ID NO: 23.

15. The fusion polypeptide of claim 14, wherein the VH comprises the amino acid sequence set forth in SEQ ID NO: 22, and the VL comprises the amino acid sequence set forth in SEQ ID NO: 23.

16. The fusion polypeptide of claim 11, wherein the scFv comprises or consists of an amino acid sequence that is at least about 80% identical to the amino acid sequence set forth in SEQ ID NO: 24.

17. The fusion polypeptide of claim 16, wherein the scFv comprises the amino acid sequence set forth in SEQ ID NO: 24.

18. The fusion polypeptide of claim 1, wherein the co-stimulatory ligand polypeptide is selected from the group consisting of a tumor necrosis factor (TNF) family member, an immunoglobulin (Ig) superfamily member, and combinations thereof.

19. The fusion polypeptide of claim 18, wherein the TNF family member is selected from the group consisting of 4-1BBL, OX40L, CD70, GITRL, CD40L, and combinations thereof.

20. The fusion polypeptide of claim 18, wherein the Ig superfamily member is selected from the group consisting of CD80, CD86, ICOSLG, and combinations thereof.

21. The fusion polypeptide of claim 20, wherein the co-stimulatory ligand polypeptide is a CD80 polypeptide.

22. The fusion polypeptide of claim 21, wherein the CD80 polypeptide comprises or consists of an amino acid sequence that is at least about 80% identical to the amino acid sequence set forth in SEQ ID NO: 32, or a functional fragment thereof.

23. The fusion polypeptide of claim 21, wherein the CD80 polypeptide comprises or consists of an amino acid sequence that is at least about 80% identical to the amino acid sequence set forth in SEQ ID NO: 33.

24. The fusion polypeptide of claim 23, wherein the CD80 polypeptide comprises or consists of the amino acid sequence set forth in SEQ ID NO: 33.

25. The fusion polypeptide of claim 1, wherein the first co-stimulatory molecule polypeptide is selected from the group consisting of CD28, 4-1BB, OX40, ICOS, DAP-10, CD27, CD40, NKG2D, CD2, and combinations thereof.

26. The fusion polypeptide of claim 25, wherein the first co-stimulatory molecule polypeptide is a 4-1BB polypeptide.

27. The fusion polypeptide of claim 26, wherein the 4-1BB polypeptide comprises or consists of an amino acid sequence that is at least about 80% identical to the amino acid sequence set forth in SEQ ID NO: 41, or a functional fragment thereof.

28. The fusion polypeptide of claim 26, wherein the 4-1BB polypeptide comprises or consists of an amino acid sequence that is at least about 80% identical to the amino acid sequence set forth in SEQ ID NO: 42.

29. The fusion polypeptide of claim 28, wherein the 4-1BB polypeptide comprises the amino acid sequence set forth in SEQ ID NO: 42.

30. The fusion polypeptide of claim 1, wherein the intracellular domain further comprises a second co-stimulatory molecule polypeptide.

31. The fusion polypeptide of claim 30, wherein the second co-stimulatory molecule polypeptide is selected from the group consisting of CD28, 4-1BB, OX40, ICOS, DAP-10, CD27, CD40, NKG2D, CD2, and combinations thereof.

32. The fusion polypeptide of claim 31, wherein the second co-stimulatory molecule polypeptide is a CD28 polypeptide.

33. The fusion polypeptide of claim 32, wherein the CD28 polypeptide comprises or consists of an amino acid sequence that is at least about 80% identical to the amino acid sequence set forth in SEQ ID NO: 43, or a functional fragment thereof.

34. The fusion polypeptide of claim 32, wherein the CD28 polypeptide comprises or consists of an amino acid sequence that is at least about 80% identical to the amino acid sequence set forth in SEQ ID NO: 44.

35. The fusion polypeptide of claim 34, wherein the CD28 polypeptide comprises the amino acid sequence set forth in SEQ ID NO: 44.

36. The fusion polypeptide of claim 1, wherein the intracellular domain further comprises a spacer region.

37. The fusion polypeptide of claim 36, wherein the spacer region comprises a CD80 polypeptide.

38. The fusion polypeptide of claim 37, wherein the spacer region comprises a CD80 polypeptide comprising or consisting of amino acids 264 to 267 of SEQ ID NO: 32.

39. The fusion polypeptide of claim 1, further comprising a transmembrane domain.

40. The fusion polypeptide of claim 39, wherein the transmembrane domain comprises a CD80 polypeptide.

41. The fusion polypeptide of claim 40, wherein the transmembrane domain comprises a CD80 polypeptide comprising or consisting of amino acids 243 to 263 of SEQ ID NO: 32.

42. The fusion polypeptide of claim 40, wherein the transmembrane domain comprises a CD80 polypeptide comprising or consisting of SEQ ID NO: 52.

43. The fusion polypeptide of claim 1, wherein the antigen-binding fragment binds to PD-L1, the co-stimulatory ligand polypeptide is selected from the group consisting of 4-1BBL, OX40L, CD70, GITRL, CD40L, CD80, CD86, ICOSLG, and the first co-stimulatory molecule polypeptide is selected from the group consisting of CD28, 4-1BB, OX40, ICOS, DAP-10, CD27, CD40, NKG2D, and CD2.

44. The fusion polypeptide of claim 1, wherein the antigen-binding fragment binds to PD-L1, the co-stimulatory ligand polypeptide is a CD80 polypeptide, and the first co-stimulatory molecule polypeptide is selected from the group consisting of CD28, 4-1BB, OX40, ICOS, DAP-10, CD27, CD40, NKG2D, and CD2.

45. The fusion polypeptide of claim 1, wherein the antigen-binding fragment binds to PD-L1, the co-stimulatory ligand polypeptide is a CD80 polypeptide, and the first co-stimulatory molecule polypeptide is a 4-1BB polypeptide.

46. The fusion polypeptide of claim 1, wherein the fusion polypeptide comprises or consists of an amino acid sequence that is at least about 80% identical to the amino acid sequence set forth in SEQ ID NO: 53.

47. The fusion polypeptide of claim 46, wherein the fusion polypeptide comprises the amino acid sequence set forth in SEQ ID NO: 53.

48. The fusion polypeptide of claim 1, wherein the fusion polypeptide comprises or consists of an amino acid sequence that is at least about 80% identical to the amino acid sequence set forth in SEQ ID NO: 55.

49. The fusion polypeptide of claim 48, wherein the fusion polypeptide comprises the amino acid sequence set forth in SEQ ID NO: 55.

50. The fusion polypeptide of claim 1, wherein the antigen-binding fragment binds to PD-L1, the co-stimulatory ligand is CD80, the first co-stimulatory molecule is 4-1BB, and the second co-stimulatory molecule is CD28.

51. The fusion polypeptide of claim 1 further comprising a signal peptide.

52. The fusion polypeptide of claim 51, wherein the signal peptide is selected from the group consisting of a human IL-2 signal sequence, a mouse IL-2 signal sequence, a human kappa leader sequence, a mouse kappa leader sequence, a human CD8 leader sequence, a truncated human CD8 signal peptide, a human albumin signal sequence, and a human prolactin signal sequence.

53. The fusion polypeptide of claim 1, further comprising a signaling domain of a cytokine receptor.

54. The fusion polypeptide of claim 53, wherein the cytokine receptor is selected from the group consisting of CD121a, CDw121b, IL-18Ra, IL18Rb, CD122, CD25, CD132, CD124, CD213a13, CD127, IL-9R, IL15Ra, CDw125, CDw131, CD126, CD130, IL11Ra, CD114, CD212, CD4, CDw217, CD118, and CDw119.

55. The fusion polypeptide of claim 1, further comprising a JAK-STAT signaling domain.

56. The fusion polypeptide of claim 55, wherein the JAK-STAT signaling domain is a STAT3 signaling domain or a STAT5 signaling domain.

57. The fusion polypeptide of claim 1, further comprising a T cell signaling molecule.

58. The fusion polypeptide of claim 1, wherein the fusion polypeptide is capable of stimulating a cell comprising an antigen-recognizing receptor.

59. The fusion polypeptide of claim 1, wherein the fusion polypeptide is capable of enhancing the activity of an immunoresponsive cell comprising an antigen-recognizing receptor.

60. The fusion polypeptide of claim 59, wherein the activity comprises cytotoxicity, cell proliferation, and cell persistence.

61. The fusion polypeptide of claim 58, wherein the antigen-recognizing receptor is a chimeric antigen receptor (CAR), a T-Cell Receptor (TCR), or a TCR like fusion molecule.

62. A nucleic acid encoding a fusion polypeptide of any one of claims 1-61.

63. A nucleic acid comprising a first polynucleotide encoding a fusion polypeptide of any one of claims 1-61, and a second polynucleotide encoding an antigen-recognizing receptor that binds to an antigen.

64. The nucleic acid of claim 62 or 63, further comprising a first promoter that is operably linked to the fusion polypeptide.

65. The nucleic acid of claim 63 or 64, further comprising a second promoter that is operably linked to the antigen-recognizing receptor.

66. The nucleic acid of claim 64 or 65, wherein one or both of the first and second promoters are endogenous or exogenous.

67. The nucleic acid of claim 66, wherein the exogenous promoter is selected from the group consisting of an elongation factor (EF)-1 promoter, a CMV promoter, a SV40 promoter, a PGK promoter, and a metallothionein promoter.

68. The nucleic acid of claim 64 or 65, wherein one or both of the first and second promoters are inducible promoters.

69. The nucleic acid of claim 68, wherein the inducible promoter is selected from the group consisting of a NFAT transcriptional response element (TRE) promoter, a CD69 promoter, a CD25 promoter, and an IL-2 promoter.

70. A vector comprising the nucleic acid of any one of claims 62-69.

71. A lipid nanoparticle comprising the nucleic acid of any one of claims 62-69.

72. A cell comprising a fusion polypeptide of any one of claims 1-61, the nucleic acid of any one of claims 62-69, the vector of claim 70, or the lipid nanoparticle of claim 71.

73. The cell of claim 72, further comprising an antigen-recognizing receptor that binds to an antigen.

74. The cell of claim 73, wherein the antigen-recognizing receptor is a chimeric antigen receptor (CAR), a T cell receptor (TCR), or a TCR like fusion molecule.

75. The cell of claim 74, wherein the antigen is a tumor antigen or a pathogen antigen.

76. The cell of claim 75, wherein the antigen is a tumor antigen.

77. The cell of claim 74 or 75, wherein the tumor antigen is selected from CD19, carbonic anhydrase IX (CAIX), carcinoembryonic antigen (CEA), CD8, CD7, CD10, CD20, CD22, CD30, CD33, CLL1, CD34, CD38, CD41, CD44, CD49f, CD56, CD74, CD133, CD138, CD123, CD44V6, an antigen of a cytomegalovirus (CMV) infected cell (e.g., a cell surface antigen), epithelial glycoprotein-2 (EGP-2), epithelial glycoprotein-40 (EGP-40), epithelial cell adhesion molecule (EpCAM), receptor tyrosine-protein kinases Erb-B2,3,4 (erb-B2,3,4), folate-binding protein (FBP), fetal acetylcholine receptor (AChR), folate receptor-α, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Ra2), κ-light chain, kinase insert domain receptor (KDR), Lewis Y (LeY), L1 cell adhesion molecule (LlCAM), melanoma antigen family A, 1 (MAGE-A1), Mucin 16 (MUC16), Mucin 1 (MUC1), Mesothelin (MSLN), ERBB2, MAGEA3, p53, MART1, GP100, Proteinase3 (PR1), Tyrosinase, Survivin, hTERT, EphA2, NKG2D ligands, cancer-testis antigen NY-ESO-1, oncofetal antigen (h5T4), prostate stem cell antigen (PSCA), prostate-specific membrane antigen (PSMA), ROR1, tumor-associated glycoprotein 72 (TAG-72), vascular endothelial growth factor R2 (VEGF-R2), Wilms tumor protein (WT-1), BCMA, NKCS1, EGF1R, EGFR-VIII, CD99, CD70, ADGRE2, CCR1, LILRB2, PRAME CCR4, CD5, CD3, TRBC1, TRBC2, TIM-3, Integrin B7, ICAM-1, CD70, Tim3, CLEC12A, and ERBB.

78. The cell of any one of claims 75-77, wherein the tumor antigen is CD19.

79. The cell of any one of claims 72-78, wherein the antigen-recognizing receptor is exogenous or endogenous.

80. The cell of any one of claims 72-79, wherein the antigen-recognizing receptor is recombinantly expressed.

81. The cell of any one of claim 72-80, wherein the antigen-recognizing receptor is expressed from a vector.

82. The cell of any one of claims 72-81, wherein the cell is a cell of the lymphoid lineage or a cell of the myeloid lineage.

83. The cell of claim 82, wherein the cell of the lymphoid lineage is selected from T cells, B cells, Natural Killer (NK) cells, dendritic cells.

84. The cell of any one of claims 72-83, wherein the cell is a T cell.

85. The cell of claim 84, wherein the T cell is derived from an induced pluripotent stem cell.

86. The cell of claim 84 or 85, wherein the T cell is a CD8⁺ T cell.

87. The cell of claim 86, wherein the CD8⁺ T cell is CD4 independent.

88. The cell of any one of claims 83-87, wherein the T cell is selected from the group consisting of a cytotoxic T lymphocyte (CTL), a γδ T cell, a tumor-infiltrating lymphocyte (TIL), a virus-specific T cell (VST), a regulatory T cell, and a Natural Killer T (NKT) cell.

89. The cell of claim 88, wherein the T cell is a tumor-infiltrating lymphocyte (TIL).

90. The cell of claim 88, wherein the T cell is a virus-specific T cell (VST).

91. The cell of any one of claims 72-90, wherein the fusion polypeptide is integrated at a locus within the genome of the T cell.

92. The cell of any one of claims 73-91, wherein the fusion polypeptide and the antigen-recognizing receptor are integrated at a locus within the genome of the T cell.

93. The cell of claim 91 or 92, wherein the locus is selected from the group consisting of a TRAC locus, a TRBC locus, a TRDC locus, and a TRGC locus.

94. The cell of any one of claims 91-93, wherein the locus is a TRAC locus or a TRBC locus.

95. The cell of claim 94, wherein the locus is a TRAC locus.

96. The cell of any one of claims 73-96, wherein the antigen-recognizing receptor is a chimeric antigen receptor (CAR).

97. The cell of any one of claims 73-96, wherein the antigen-recognizing receptor is a TCR like fusion protein.

98. The cell of any one of claims 73-97, wherein the expressions of fusion polypeptide is under the control of an endogenous promoter.

99. The cell of any one of claims 73-97, wherein the expressions of the fusion polypeptide and antigen-recognizing receptor is under the control of an endogenous promoter.

100. The cell of claim 98 or 99, wherein the endogenous promoter is selected from the group consisting of an endogenous TRAC promoter, an endogenous TRBC promoter, an endogenous TRDC promoter, an endogenous TRGC promoter, and a combination thereof.

101. The cell of claim 100, wherein the endogenous promoter is a TRAC promoter.

102. The cell of any one of claims 72-101, wherein said cell is autologous.

103. The cell of any one of claims 72-101, wherein said cell is allogeneic.

104. A composition comprising a nucleic acid of any one of claims 62-69.

105. A composition comprising a vector of claim 70.

106. A composition comprising a lipid nanoparticle of claim 71.

107. A composition comprising a cell of any one of claims 72-103.

108. The composition of any one of claims 104-107, which is a pharmaceutical composition further comprising a pharmaceutically acceptable excipient.

109. The composition of any one of claims 104-108, which is for treating and/or preventing a neoplasm, an autoimmune disease, and/or an infectious disease.

110. The composition of any one of claims 104-109, further comprising a regulator that is capable of regulating or modulating expression and/or activity of the fusion polypeptide.

111. The composition of claim 110, wherein the regulator is selected from the group consisting of promoters that are capable of controlling the expression of the fusion polypeptide, molecules that are capable of regulating or modulating expression and/or activity of the co-stimulatory ligand, molecules that are capable of regulating or modulating expression and/or activity of the co-stimulatory molecule.

112. The composition of claim 111, wherein the molecules that are capable of regulating or modulating expression and/or activity of the co-stimulatory ligand are selected from the group consisting of antibodies that bind to the co-stimulatory ligand, and fusion proteins that bind to the co-stimulatory ligand and regulate or modulate the expression and/or activity of the co-stimulatory ligand.

113. The composition of any one of claims 110-112, wherein the regulator is an anti-CD80 antibody and the fusion polypeptide comprises an extracellular domain comprising a CD80 polypeptide and a transmembrane domain comprising a CD80 polypeptide.

114. The composition of any one of claims 110-113, wherein the regulator is a fusion protein that binds to CD80 and modulates the activity of CD80 and the fusion polypeptide comprises an extracellular domain comprising a CD80 polypeptide and a transmembrane domain comprising a CD80 polypeptide.

115. The composition of claim 114, wherein the fusion protein comprises an CTLA-4 fragment that binds to CD80.

116. The composition of claim 115, wherein the CTLA-4 fragment that binds to CD80 is abatacept or belatacept.

117. The composition of claim 116, wherein the molecules that are capable of regulating or modulating expression and/or activity of the co-stimulatory molecule are selected from the group consisting of antibodies that bind to the co-stimulatory molecule, and fusion proteins that bind to the co-stimulatory molecule and regulate or modulate the expression and/or activity of the co-stimulatory molecule.

118. A method of reducing tumor burden in a subject, the method comprising administering to the subject an effective amount of the cells of any one of claims 72-103, the composition of any one of claims 104-117, the nucleic acid of any one of claims 62-69, the vector of claim 70, or the lipid nanoparticle of claim 71.

119. The method of claim 118, wherein the method reduces the number of tumor cells, reduces tumor size, and/or eradicates the tumor in the subject.

120. A method of treating a subject having a relapse of a neoplasm, administering to the subject an effective amount of the cells of any one of claims 72-103, the composition of any one of claims 104-117, the nucleic acid of any one of claims 62-69, the vector of claim 70, or the lipid nanoparticle of claim 71.

121. A method of treating and/or preventing a neoplasm in a subject, administering to the subject an effective amount of the cells of any one of claims 72-103, the composition of any one of claims 104-117, the nucleic acid of any one of claims 62-69, the vector of claim 70, or the lipid nanoparticle of claim 71.

122. The method of any one of claims 118-121, wherein the subject received an immunotherapy prior to said administration of the cells or the composition.

123. The method of any one of claims 118-122, wherein the neoplasm or tumor is cancer.

124. The method of any one of claims 118-123, wherein the neoplasm or tumor is selected from the group consisting of blood cancers and solid tumors.

125. The method of claim 124, wherein the blood cancer is multiple myeloma, myeloid disorder, leukemia, or lymphoma.

126. The method of claim 125, wherein the leukemia is acute myeloid leukemia (AML), chronic myeloid leukemia (CML), acute lymphocytic leukemia (ALL), chronic lymphocytic leukemia (CLL), acute promyelocytic leukemia (APL), mixed-phenotype acute leukemia (MLL), hairy cell leukemia, or B cell prolymphocytic leukemia.

127. The method of claim 125, wherein the lymphoma is Hodgkin's lymphoma, non-Hodgkin's lymphoma, B-cell non-Hodgkin's lymphoma, or T-cell non-Hodgkin's lymphoma.

128. The method of claim 124, wherein the solid tumor is selected from the group consisting of renal cell carcinoma, non-small-cell lung cancer, lung adenocarcinoma, lung squamous cell carcinoma, lung neuroendocrine carcinoma, small-cell lung cancer, pancreatic cancer, breast cancer, astrocytoma, glioblastoma, laryngeal/pharyngeal carcinoma, EBV-associated nasopharyngeal carcinoma, and ovarian carcinoma.

129. The method of any one of claims 118-128, further comprising administering to the subject a regulator that is capable of regulating or modulating expression, activity of the fusion polypeptide.

130. The method of claim 129, wherein the regulator is selected from the group consisting of promoters that are capable of controlling the expression of the fusion polypeptide, molecules that are capable of regulating or modulating expression and/or activity of the co-stimulatory ligand, molecules that are capable of regulating or modulating expression and/or activity of the co-stimulatory molecule.

131. The method of claim 129, wherein the molecules that are capable of regulating or modulating expression and/or activity of the co-stimulatory ligand are selected from the group consisting of antibodies that bind to the co-stimulatory ligand, and fusion proteins that bind to the co-stimulatory ligand and regulate or modulate the expression and/or activity of the co-stimulatory ligand.

132. The method of any one of claims 129-131, wherein the regulator is an anti-CD80 antibody and the fusion polypeptide comprises an extracellular domain comprising a CD80 polypeptide and a transmembrane domain comprising a CD80 polypeptide.

133. The method of any one of claims 129-132, wherein the regulator is a fusion protein that binds to CD80 and modulates the activity of CD80 and the fusion polypeptide comprises an extracellular domain comprising a CD80 polypeptide and a transmembrane domain comprising a CD80 polypeptide.

134. The method of claim 133, wherein the fusion protein is a CTLA-4 fragment that binds to CD80.

135. The method of claim 134, wherein the CTLA-4 fragment that binds to CD80 is abatacept or belatacept.

136. The method of claim 129, wherein the molecules that are capable of regulating or modulating expression, activity of the co-stimulatory molecule are selected from the group consisting of antibodies that bind to the co-stimulatory molecule, fusion proteins that bind to the co-stimulatory molecule and regulate or modulate the expression, activity of the co-stimulatory molecule.

137. The method of any one of claims 129-136, wherein the regulator is capable of depleting the cell.

138. A method for producing a cell, the method comprising introducing into a cell the nucleic acid of any one of claims 62-69, the vector of claim 70, or the lipid nanoparticle of claim 71.

139. A kit comprising a cell of any one of claims 72-103, a composition of any one of claims 104-117, a nucleic acid of any one of claims 62-69, a vector of claim 70, or a lipid nanoparticle of claim 71.

140. The kit of claim 139, wherein the kit further comprises written instructions for treating and/or preventing a neoplasm, a pathogen infection, and/or an infectious disease.
